SEQUENCE LISTING (136 sequences)

SEQ ID NO: 1 - DNA, 20 bp, Unknown
  27f,16S, 16S rRNA gene
agagtttgatymtggctcag

SEQ ID NO: 2 - DNA, 19 bp, Unknown
  1492r,16S, 16S rRNA gene
ggttaccttgttacgactt

SEQ ID NO: 3 - DNA, 19 bp, Unknown
  515f,16S, 16S rRNA gene
gtgycagcmgccgcggtaa

SEQ ID NO: 4 - DNA, 20 bp, Unknown
  806r,16S, 16S rRNA gene
  misc_feature (8)..(8): n is a, c, g, or t
ggactacnvgggtwtctaat

SEQ ID NO: 5 - DNA, 22 bp, Unknown
  ITS1F,ITS, Internal transcribed spacer
cttggtcatttagaggaagtaa

SEQ ID NO: 6 - DNA, 17 bp, Unknown
  LR5,ITS, Internal transcribed spacer
tcctgagggaaacttcg

SEQ ID NO: 7 - DNA, 20 bp, Unknown
  ITS2,ITS, Internal transcribed spacer
gctgcgttcttcatcgatgc

SEQ ID NO: 8 - DNA, 20 bp, Unknown
  ITS3,ITS, Internal transcribed spacer
gcatcgatgaagaacgcagc

SEQ ID NO: 9 - DNA, 19 bp, Unknown
  ACT512f,ACT, Actin
atgtgcaaggccggtttcg

SEQ ID NO: 10 - DNA, 20 bp, Unknown
  ACT783r,ACT, Actin
tacgagtccttctggcccat

SEQ ID NO: 11 - DNA, 28 bp, Unknown
  CA14,ACT, Actin
aactgggatgacatggagaagatctggc

SEQ ID NO: 12 - DNA, 28 bp, Unknown
  CA5R,ACT, Actin
gtgaacaatggatggaccagattcgtcg

SEQ ID NO: 13 - DNA, 20 bp, Unknown
  Cmd5,CMD, Calmodulin
ccgagtacaaggargccttc

SEQ ID NO: 14 - DNA, 21 bp, Unknown
  Cmd6,CMD, Calmodulin
ccgatrgaggtcatracgtgg

SEQ ID NO: 15 - DNA, 17 bp, Unknown
  5.8S,ITS, Internal transcribed spacer
cgctgcgttcttcatcg

SEQ ID NO: 16 - DNA, 18 bp, Unknown
  5.8SR,ITS, Internal transcribed spacer
tcgatgaagaacgcagcg

SEQ ID NO: 17 - DNA, 19 bp, Unknown
  ITS1,ITS, Internal transcribed spacer
tccgtaggtgaacctgcgg

SEQ ID NO: 18 - DNA, 20 bp, Unknown
  ITS4,ITS, Internal transcribed spacer
tcctccgcttattgatatgc

SEQ ID NO: 19 - DNA, 22 bp, Unknown
  ITS5,ITS, Internal transcribed spacer
ggaagtaaaagtcgtaacaagg

SEQ ID NO: 20 - DNA, 22 bp, Unknown
  LS266,ITS, Internal transcribed spacer
gcattcccaaacaactcgactc

SEQ ID NO: 21 - DNA, 20 bp, Unknown
  V9G,ITS, Internal transcribed spacer
ttacgtccctgccctttgta

SEQ ID NO: 22 - DNA, 20 bp, Unknown
  5F_Eur,RPB2, Second largest subunit of RNA polymerase II
gaygaycgkgaycayttcgg

SEQ ID NO: 23 - DNA, 20 bp, Unknown
  7CR_Eur,RPB2, Second largest subunit of RNA polymerase II
cccatrgcytgyttrcccat

SEQ ID NO: 24 - DNA, 23 bp, Unknown
  bRPB2-7.1R,RPB2, Second largest subunit of RNA polymerase II
cccatrgcytgyttmcccatdgc

SEQ ID NO: 25 - DNA, 20 bp, Unknown
  fRPB2-5F,RPB2, Second largest subunit of RNA polymerase II
gaygaymgwgatcayttygg

SEQ ID NO: 26 - DNA, 20 bp, Unknown
  fRPB2-7R,RPB2, Second largest subunit of RNA polymerase II
cccatwgcytgcttmcccat

SEQ ID NO: 27 - DNA, 1417 bp, Pseudomonas glycinis
ctcagattgaacgctggcggcaggcctaacacatgcaagtcgagcggatgaaaggagcttgctcctggattcagcggcggacgggtgagtaatgcctaggaatctgcctggtagtgggggacaacgtttcgaaaggaacgctaataccgcatacgtcctacgggagaaagcaggggaccttcgggccttgcgctatcagatgagcctaggtcggattagctagttggtgaggtaatggctcaccaaggcgacgatccgtaactggtctgagaggatgatcagtcacactggaactgagacacggtccagactcctacgggaggcagcagtggggaatattggacaatgggcgaaagcctgatccagccatgccgcgtgtgtgaagaaggtcttcggattgtaaagcactttaagttgggaggaagggttgtagattaatactctgcaattttgacgttaccgacagaataagcaccggctaactctgtgccagcagccgcggtaatacagagggtgcaagcgttaatcggaattactgggcgtaaagcgcgcgtaggtggtttgttaagttggatgtgaaatccccgggctcaacctgggaactgcatccaaaactggcaagctagagtatggtagagggtggtggaatttcctgtgtagcggtgaaatgcgtagatataggaaggaacaccagtggcgaaggcgaccacctggactgatactgacactgaggtgcgaaagcgtggggagcaaacaggattagataccctggtagtccacgccgtaaacgatgtcaactagccgttgggagccttgagctcttagtggcgcagctaacgcattaagttgaccgcctggggagtacggccgcaaggttaaaactcaaatgaattgacgggggcccgcacaagcggtggagcatgtggtttaattcgaagcaacgcgaagaaccttaccaggccttgacatccaatgaactttccagagatggattggtgccttcgggagcattgagacaggtgctgcatggctgtcgtcagctcgtgtcgtgagatgttgggttaagtcccgtaacgagcgcaacccttgtccttagttaccagcacgtcatggtgggcactctaaggagactgccggtgacaaaccggaggaaggtggggatgacgtcaagtcatcatggcccttacggcctgggctacacacgtgctacaatggtcggtacagagggttgccaagccgcgaggtggagctaatcccacaaaaccgatcgtagtccggatcgcagtctgcaactcgactgcgtgaagtcggaatcgctagtaatcgcgaatcagaatgtcgcggtgaatacgttcccgggccttgtacacaccgcccgtcacaccatgggagtgggttgcaccagaagtagctagtc

SEQ ID NO: 28 - protein, 284 aa, Pseudomonas glycinis
MRLIIVSGRSGSGKSTALNVLEDNGYYCIDNLPAGLLPELAERALIHTELAQPLVAVSIDARNLPSHLSRFPELLEEVRARHIQCDVLYLDADEETLLKRFSETRRRHPLSTANRSLAEAIEDETALLGPIADLADLKVNTTNLNLYQLRDTIKLRLLNQPEPGTAFLVESFGFKRGMPVDADLVFDVRCLPNPYWKPELRAQSGLDAPVAEYLAAQPEVEEMFQDIYGYLYKWLPRFAASNRAYVTIAIGCTGGHHRSVYLTERLGQALQKTLKNVQVRHRDL

SEQ ID NO: 29 - protein, 215 aa, Pseudomonas glycinis
MRVILLGAPGAGKGTQAKFITEKFGIPQISTGDMLRAAVKAGTPLGVQAKSIMDAGGLVSDDLIIALVQDRIAQPDCANGFLFDGFPRTIPQAEALVTAGVELDAVVEIAVEDEEIVQRIAGRRVHEASGRVYHIVYNPPKIAGKDDITGEELVQRKDDTEETVRHRLSVYHSQTKPLVEFYQSLSAKAGKPKYSHIPGVGSVEAITAKVLEALS

SEQ ID NO: 30 - protein, 362 aa, Pseudomonas glycinis
MSGNTYGKLFTVTTAGESHGPALVAIVDGCPPGLEISLEDLQRDLDRRKPGTSRHTTQRQEADEVEILSGVFEGRTTGCSIGLLIRNTDQKSKDYSAIKDLFRPAHADYTYHHKYGERDYRGGGRSSARETAMRVAAGAIAKKYLASQGIVIRGYMSQLGPIEIPFKTWDSVEQNAFFSPDPDKVPELEAYMDQLRRDQDSVGAKITVVAEGVMPGLGEPIFDRLDAELAHALMSINAVKGVEIGAGFACVAQRGTEHRDELTPQGFLSNNAGGILGGISSGQPIVAHLALKPTSSITTPGRSIDIHGNPVDVITKGRHDPCVGIRATPIAEAMMAIVLMDHLLRHRGQNADVRVSTPVLGL

SEQ ID NO: 31 - protein, 201 aa, Pseudomonas glycinis
MTEPALLPRPQLRRLLRKARRSLTPGEQRQAAKGLFRQLAQDPHFRRAKHISLYLPTDGEIDPRLLLREAQRRGKATYLPVLSAWPRTKMVFQRIRPGEKLKPNRFRILEPRANLARQRKIWTLDLVLLPLVGFDDVGGRLGMGGGFYDRSLAYLARRKNWRKPTLLGLAHECQKVERLAQASWDVPLQGTVTDKAWYFAG

SEQ ID NO: 32 - DNA, 570 bp, Trichoderma hamatum
cggagggatcattaccgagtttacaactcccaaacccaatgtgaacgttaccaaactgttgcctcggcggggtcacgccccgggtgcgtaaaagccccggaaccaggcgcccgccggaggaaccaaccaaactctttctgtagtcccctcgcggacgtatttcttacagctctgagcaaaaattcaaaatgaatcaaaactttcaacaacggatctcttggttctggcatcgatgaagaacgcagcgaaatgcgataagtaatgtgaattgcagaattcagtgaatcatcgaatctttgaacgcacattgcgcccgccagtattctggcgggcatgcctgtccgagcgtcatttcaaccctcgaacccctccgggggatcggcgttggggatcgggacccctcaccgggtgccggccctgaaatacagtggcggtctcgccgcagcctctcctgcgcagtagtttgcacaactcgcaccgggagcgcggcgcgtccacgtccgtaaaacacccaacttctgaaatgttgacctcggatcaggtaggaatacccgctgaacttaagcatat

SEQ ID NO: 33 - DNA, 270 bp, Trichoderma hamatum
atgaaaatgcttattactgccgctcttttcaccctggccctcgccgcccctgtcgccgagactaagcctcatagcattgctgctcgcgatccctttacttgccccggcggcctgaccaacagcacccccatgtgctgcagcgttaatgtccttggcctgctagcccttgattgccagcaacctggcgcggacggctgtagtggcagctccaagcccaattgttgcacccttggagcggccggacagggtgttatttgcaacgcactgtaa

SEQ ID NO: 34 - DNA, 1917 bp, Trichoderma hamatum
atgaagtcggccattcttcttgggctcactggcctcgctgccaatgtgaatgcccatcctgctaagcagccagagaccggcaacggcctgactaagcgtggcgttgatattagcaagtatagcttgcctaatctttcaaagtacacgtcttcgccaaacatcgaacaagaggcctctctgcagtctttggctttcaagcgcaactatgtggatacagccacacgagctgtcaagaaagctgcccccaaggccgaattccgcgtcgtggatgaccactatatcgatgttgacggcattggtcacgtccactttaagcagacagtgcacggaattgacattgacaacggtgacttcaaggttaacattggcaaagacggtagagtcttctctcatggaaacagcttcttttctggcaagctgccccagcagaaccccttgaggaagcgtgatttctctgaccccaccactgcgctcaagggtgcaattgatatcctgggcctcccggttcaggctgatggcgccaccgccgaagctcaggaaggcactgagaagtacacgttgaagggtacatccggagctgtcagcgatcctaaggctcacctcgtttacctcgtcaagggagacggaactctggctctgacttggcgggtcgagaccgatatcatggacaactggcttctcacctacgttgacgcaacaaccaaccaagagattcatggtgttgtagactacgtctccgaccttgccaccttccaggtttacccctggggcctcaatgaccccaccgagggagatcgtaaagtcctgaccgacccgtggagaaccgatgcctccccattcacttggctcagcgacggaaccaccaactatactgtaactcgcggaaacaatgctattgcgcaggataatccttctgggggggacagttatctcaataatcaccgtccatccagctcgactcgggactttcaatatccctttacgctgactcagaccaaccctacagattatagggatgctgccatcacacagctcttttacacagtcaacaagtaccacgatttgctatatgtccttggcttcaacgaggtagcaggaaacttccaagcaaataacaacggaaagggcggcaaggccaatgatttcgttattgttaacgctcaagacggcagtggaacgaacaatgccaactttgctaccccggccgatggaagcaatggccgtatgagaatgtacatctggactactgctaaccccaagcgagatggcgatctcgaggcgggcattgttatccacgaatacactcacggattgtctacccgtcttaccggcggccctgccaactctggatgccttactggcactgaagccggcggcatgggtgaaggctggggtgatttcttcgccaccgctatccgcttaaaggctggtgacacccgcagcaaggactatcctatgggagtctgggccgataacaacgtcaagggtatccgccagtatccttattcaacaagccttactaccaatcctcttacttataaaaccgtcaatacgcaaaatgaagtccactcagccggtactacatgggccaccattttgtacgaagtcctgtggaacctcattgacaagtacggcaagaatgacgacgatttccctacctttgatagccagggcgtccccaccgacggcaagtatcttaccttgaaactcgtcctcaatggattggctctgcagccctgcacgccaacttttgtttctgcccgtgatgccatcttggatgctgatcgtgctttgaccggaggggagaatctttgtgaattatggacaggattcgccaagagaggtcttggatccggcgccaaatattcttccaccgctcgcgttgaaagcttcactatcccatctggggtttgctaa

SEQ ID NO: 35 - DNA, 297 bp, Trichoderma hamatum
atgaagttcttcgccgccaccgctctccttgccgccactaccattgctggacctcttgaggttcgcactggagacggtaacatttgtcctagcggcggcctctacggcaaccctcagtgctgctcctctctcctccttggtctcattggtcttgactgcaacgtccccaaccaaacgccccgtgacggcgctgactttagaaacatctgcgctaagactggtgacgaggccctttgctgtgttgctcccgttgccggccaagctcttctctgccaggtcgccgttggtgccagctaa

SEQ ID NO: 36 - DNA, 294 bp, Trichoderma hamatum
atgaagttctttgctgttgccactctctttgtcactggcgtcatctccgccccgtcacccaatgccctaaattcccgcagtcttctttgcagccctggcctttactccaccgcgcagtgctgtggtgttgatgtattgggagttgctgatttggactgtgcggctcctactggcactattaccaacgcccaaggatttcaggctgcctgtgctaagaaaggccaagaggctcgctgttgtgttctacctgttgccggccaagatgtcctgtgccaggacccccctggtttgtaa

SEQ ID NO: 37 - DNA, 1230 bp, Trichoderma hamatum
atgaccagcattcgtcgtcttgctctctatctcggagctttgctcccggctgtcctcgccgctccagcagcccttcacaagaagcctgaggctgcacctaacaagtttattgtcactcttaaagagggcgcttcaattgataccgactctcatctcgcctgggtgaatgacatccaccgtcgttctttgaccaagcgtagcactgctggtgttgaaaagacttataacattcatacttggagtgcttatgcgggtgaatttgatgcagagacgattgagcagatcaagtctagccccgatgtcgcgtctgtggagccagactacatcatgtacctgtcggacattgttgaagacaagcgtgctctgaccacacaatctggagctccttggggccttggcactgtttcccaccgcacatctgggtctacgagctacatttacgatagctcagctggctctggaacctttgcctatgtggttgactccggcatcaacacctctcatcagcaattcggcgggcgcgccagtcttggctacaatgctgcaggaggacagcacgtcgacactcttggccatggtactcatgtttctggaacaattggtggatctacatacggcgttgctaaacaggccagcctaatctccgtcaaagtctttgctggagagagcgctaccacctctgttatccttgacggctataactgggctgtaaacgacattgtctcgaggaatcgtgctagcaaatctgccattaacatgtcgcttggaggacctgcctcatccacctggacgaccgccattaacgcagcctttaaccagggtgtgcttaccattgtcgccgctggtaatggcgacagtttcggaaacccccagccagtttccggcacttctcctgctaatgttcctaatgctatcactgttgcggcgctggacatcaactggcgcactgcttccttcaccaactacggtgctggcgttgatgtctttgcccctggtgttaacattctgtcgtcatggattggatccaacactgctaccaacacaatcagcggcacctctatggcgacacctcacgttgttggtctggctctctatcttcaatcccttgaaggcctcaccactcctaccgctgtcactaatcggatcaaggctctggctaccgctggccgtataactggcagccttaacggcagccccaacactctcatcttcaacggaaacagtgcttaa

SEQ ID NO: 38 - DNA, 1185 bp, Trichoderma hamatum
atgacttctctataccttacttctgcccttgggctgctatgcctcctccccgctgcacaggcaggctggaatccgaactcaaagaacaacattgtcgtatactggggacaggatgcaggcagcattggccagaatagactgtcttactactgcgaaaatgcccctgatgttgatgttatcaatatctctttcttagttggcataacagatcttaacctgaaccttgccaatgttggcaacaactgtacctcttttgctcaggatcccaacctactcaactgcccccaagttgcggcagatatcgtcgagtgccaacagacatatggaaaaacaatcatgatgagcttgtttggctcgacttatactgagagtggtttcagttcgtcgtcaactgcagtgtccgccgctcaagaaatttgggccatgttcggtcctgtccagagcggcaacagcaccccccgaccttttggtaacgctgtgattgatggatttgactttgatcttgaagaccctatcgaaaacaacatggagccttttgcggcagagctgcgatctctcacaagtgctgctacctccaagaagttttacctttcggctgctcctcagtgtgtgtaccccgacgcgtctgatcagacgttcctccagggagaggtggcttttgactggttgaatatccaattttacaacaatggctgtggtacctcttactacccctcgggctacaactacgcaacttgggacaactgggtcaagaccgtcagtgctaacccaaacactaagctgcttgtcggcactcctgccagtgtccatgctgtaaactttgccaactactttcccaccaatgatcaactcgccggagctatctcgtcttccaagtcgtacggtagctttgctggtgtgatgctatgggatatggctcagctctttggaaatcctggatacctggacttgatcgtagcggacctgggtggcgcttccacccctcctccgccagcttccactactctgtccactgtgactaggtcctctactgctagcagcggacctacttctcctcctccctccggcggtggcagtgtccctcaatggggccagtgcggtggacagggatacacaggaccaactcaatgccagtctccctacacttgcgttgttgagagccagtggtgggcatcttgccagtaa

SEQ ID NO: 39 - DNA, 297 bp, Trichoderma hamatum
atgagaacatctaccgctattatatctcttgcccttgtggtgatgggaattgccgcccctgctgccgaggccggctctgttgtcagcgaagccggctttgttgtcagcgaagcccaattcaacgccatatttcccggtcgaaaccctttttactcctatagggcattgaccggcgccctcggatcttatcccagcttcaccaacactgctgataacgccactaggctccgcgaggccgccgctttcttcgccaatgtggatcatgaaaccgatgggctcaagttcgtcgtggaataa

SEQ ID NO: 40 - DNA, 1074 bp, Trichoderma hamatum
atgagcgccgcatcccgcaagtcacgcatagctacgagtgtggcgagagttatgtacaccaatgcagtctatttccctagcagcagaatatatcaaggtgattcgccgggaatgctcaattacagctgcatcaaccacgtgtattatgcttatgccagcgtgacggcggacggcaatgtgtttctcggcgatgagtgggccgatgcaagagcgccagtggatggtgttcagggtggcttaggatccttgatgcatctcaagcagaggcaccctcacctgcaggttgtcttatctattggtggcagcactgcatcagaggtatttcccattgttgcatcaagcactctcctcagagacaactttgcccggtctgcccttggcctcgtggaagcatccgggcttgatggtattgacattgcctgggagttcccatcccaagctaaacatggccacgatttcctagccttgctggcagcagtgcggattcatatgcctgaagaccgcttcattctaacagctgtcctccctgcggcgaaggaggttctacaactcatcgacctcagcacagcggccgagtaccttgactacatcaaccttgtggcatatgatttcttcggcacgtggacgtccaaaactggtcatcacgcacagttatatactatgaacaaggacgaaccctcggcatcgacaggtgtggcgtatgtcatgtcccaaggattccctgcgaagagtatcctgctcgggatcccaacctacggacgaagcttcctcaaggccaatgggccgggccaggattttaacggcgttggcggccaagatggcacattcgaatacggcgaattaccacgaaagggatgcaaagagattgtggataggcgctatattgcggcccaatgtgtcggtggtgatggggggtttgtcacgtacgacaaccccgagacggtcaaggttaaggcggaattttgcaagcaaaagggattaggggggcttttttactggaacggaccggctgactctcgagatcaagcacgaagtctgattgcggctggattccgcgctctacacacctcgtga

SEQ ID NO: 41 - DNA, 306 bp, Trichoderma hamatum
atgcaattgaccacagtcgtcgcgctcttcgcctctctggccggcgccgcgcctgcccctgaacccgctgctgagcttgttgctcgtgatggtccttgctcctctggtgtcaccaataacgtccctcaatgctgcggtactggtatcctcagtgttgtctacgtggactgcaagactcccactcaagctccctctgccactaaccagttgagcgccatctgcgctcgagtaggtctccaggccaagtgttgcaccgtcggcattgctggcgttggcgttctttgccaggatgctattccccagtaa

SEQ ID NO: 42 - DNA, 417 bp, Trichoderma hamatum
atgcaattgtccaacctcttcaagcttgctctcttcaccgccgctgtctctgctgataccgtctcgtacgatactggctacgatgacgcatctcgctctctgaccgtcgtctcctgctccgacggccccaacggcctcatcaccagataccactggcagacccagggccagatccctcgcttcccatacattggtggtgtccaggccgtcgccggctggaactcccctagctgcggcacctgctggaagctcacttacagcggcaagaccatctacgtcttggctgttgaccacgccggtgctggcttcaacattggcctcgacgccatgaatgctctcaccaacggccaggctgttgccctcggacgtgtctctgccactgcctctcaggtggctgtgagcaactgcggtctctag

SEQ ID NO: 43 - DNA, 1263 bp, Trichoderma hamatum
atgcattcaacaactttctttgccagtctgggactcgcaggcctggttgccggcgccccttcggccccgcacaacgttaaagctcgccaagcttccggtgcccaaaacgtcgtctattggggaggcactaacaatgaaagcgacgacctttctacctattgtacgcccaccgcaggcattgacatccttgttctgtctttcttggacatttacggcacaactggcaacattccagcaggtaatattggcaattcttgctatatcggaacaaatggcacacctcaactatgtgataatcttgctgcttcaatagccagctgccaggccgctggtattaaaattatcttgtctctcggaggagcggccggctcctattctctacaatcgcagtcacaggctgtggctattggccagtatctctgggaggcttacggcaactctggcagcacttctgtccagcggccctttggcaatgtctttgttaatggttttgactttgaccttgagctgaacgccggcaaccaatactaccagtatctgatatcaaccctccgttccaactttgccagtgacccgaaacatacgtattatatcactggagcgccccagtgtcctctcccgttgagtattcctcaaatccgtaaaccaaatatgggagagattataagtgcctcacagttcgactatctctggattcaattctataacaacaacgcttatgcaccagacccctgttctcttggtctgcctggcgatgcacccttcaactacaacaactggacgtcatttattgctactaccccttcaaaaaacgcaaaactatttattggagtccctgcgaatactttagctgctaatggcaattcaggtggtgccgtttactatgcttcgccatcccagcttgcttccattgtcgcgaatacaaaatctagcccagattttggaggaattatgatgtgggatgctgggtactctgatgctaatgtcaacaacggatgcaactacgctcaggaggccaaaaacatcctccttaccggcgctccctgtggaggctcacccccaccggttagcagcagcaagcctacatccactgcaaccaaatccgctactagcacttcatcagcctcgggaacaggcccgacaggaggcggcacagtacctcagtggggccagtgcggcggagagggatacacgggcccgacgcagtgcgtttctccctacaaatgcgtcgaatcaagtcaatggtggtcgtcttgccaataa

SEQ ID NO: 44 - DNA, 999 bp, Trichoderma hamatum
atgccctctttgatcacggttgcgagcgtgctcgctcttgttccatccgccttcgcgggctggaatgtcaactcgaagcaaaacattgctgtgtactggggacaaaactccgccaaccaacagagcacacagcagcgtctttcagcctactgcaatgcgaagattgaatctgacagttgcgatgtagatgccaacatcaatgtcattgacattgctttcctgaatggaattactcctcctatgaccaactttgccaatgctggtgaccgatgcactcccttctccgacaacccttggctcttgagctgccctgaaattgaggcggatatcaagacttgccaggctaatggcaagaccattctcatttctcttggtggtgattcttacactcaaggtggctggagctctaccagtgctgctcaggccgcagccaaacaggtctgggccatgtttggtcctgtccaatccggcagctccaccgagcgtccctttggcagcgccgttgttgacggttttgacttcgactttgaagcgacgactaacaatctcgcggcctttggcgctcagctcaagagcctctcgagcgctgccggtggcaagaagtactacttctctgctgctccccagtgcttcttccccgacgctgctgtcggtgcactgatcaacgccgtccccatggactggatccagattcaattctataacaacccttgcggcgtcagcggctacacccccggcaccagcaaccagaacaactacaactaccagacctgggatacctgggccaagacaagccccaaccccaacgtcaagcttcttgtcggcattcccgctggcccaggtgctggtcgcggctacgtgtctggctctcagctcacttcagtcttccagtactcgaagggcttcagcagcacttttgccggtgctatgatgtgggatatgtcccagctcttccagaacactggcttcgaggcccaggttgtcaatgctttgaaataa

SEQ ID NO: 45 - DNA, 1623 bp, Trichoderma hamatum
atgccgcttaaggtcttcgagtcagcttcgcctgcgccgtcgcatccctccttatcgcacctctgcctgctcgtcttcgaagctgttctagaagtagtctgtgtgagcttgccgggatacatcgttgcgcgccttggccatttcgatgccgataagcaaaagttcctggctaatctcaatgtgatgctcttcacgccctgtcttatcttcaccaagctagcatctcagctcaatgccgaaaaactatccgatctcgccatcatccccgccatctttatcgtgcaaactcttgtatcatggatagtttcgatcctggtcgccaaggggttccgattcaataagcgagcttccaacttcgttactgctatgggcgtctttggcaattcaaattcactgcccatctctctcgttctttctctctcgcaaacaatcaagggcctccactgggacaggataccaggcgacaacgatgacgaagtcggcgctcgcggtatcttatatctactgattttccagcaacttggccagcttgtcagatggagctggggatatcacgttctgctagcccccaaggataaatacgccgagtatcaagacgaaattgccgaagaaggccagtacagatacagagacgaagagccgaacgaacaggagccggagattctcattactggtttggacggtgataccgaagatgatggcgagagcaatgcttctgaagattatatacctgctggacgaacacctcttgcgagcaattcccgggcttcactggctggctcttccgtcgacaatgacgacatgctgaatttcaaaaagggcaactacactcgtggaagctcgctcgccaacacagacttggaagatgatattctttcattccctcgtattcgtcttcgagatgaggctgaagtcgagcacggagttactgctcgtatcaagaaatcactatattctctgaaagacaaagcttctgcggccatgactcgccaatatcagcgacttcctcaacctgttcaaacctgcctttccttcattcataagtcaatcacaaagactctcggatttgtatgggatttcatgaatccacctttgtgggctatgctcatcgcggtcatcgtggcgtccatcccgagtctccaacagttgttcttcgaagatggttcattcgtgaaaaatagcgtcaccaacgctgtttcgtctagtggaggcgttgcagtgcctttgatcttggttgttctcggcgccaacttggcgcgcaacactgccgctcacgattctcccatagacccggaggaggaaaagattggcaccaagctcttgatcgcttcgttgctgagcaggatggttctgccaactttgattatggcacccattctggctattacggcaaaatacttgcccatcagcattctagatgatcccatctttattgtcgtatgctttcttcttactggcgcacccagtgcgctccagcttgcgcaaatttgccaaatcaacaatgtttatgagcagactatgggaagaatcctctttcagagctatgttatctggattcttccctctactcttttccttgtcatgatggcactcgaggtgattgagtgggcaacagtgaattaa

SEQ ID NO: 46 - DNA, 717 bp, Trichoderma hamatum
atgctaccgtcgagcctttgccggattgccgctgttatcagcgtggcttctgcggaaatggtttcggtaacatttgataccgcttacgacgatccttctcgttcactgagtgaagtcgcttgttggaggaagggcacgggattcatgcctaacttggattggaaacttcaaaaagatgcattggaatttattggaatcaaagcaattcgtggcttcagttctgcccaatgtttttcctgctggaaaattgagtatggagataagcagatatcactgttcgcaattgacagcgccgactcgggcattgttctctctctgagcaccatgcaatatcttacaggtggccaggctcgcgagctggcccgaattgatgcaaaggcaacccaagtcgacgcatcaaactgtgggatttctgcggctgaactgcacaaatacgatttctataccaatggtagtggaaatagcgatggctctaggagcaacactggagctattgttggaggagttctcggtggtgtggctgggcttgctttaactgggcttgctgtttttttcttcccccgctataagaatttagctgctggacataagccagtgagccagaatactcctcctgctgcttatcaatcctctctgatgcagcaggaccccaaggctcactatgatcctaaatatttcgcagagatggatacacagccatacacgccccctgattag

SEQ ID NO: 47 - DNA, 1209 bp, Trichoderma hamatum
atgctccgtactcctacaatcccctggcttctaaatatcatcctagaagaaaaccctactatgattccttcaatgaaaaagtgcggccgcaagttatgggctggcctggcattgctagccttgcctgtaattgcaagcgcatcatgcgcgcttcctagcacctacaaatggacttcgactggcccgctggcaaaccccaagtcgggctgggtttcgcttaaagatttcagccatgttccatacaatggccagcatctggtctattcttcgactgtgaactcggcaggttcctatggctcgatgaattttggtctcgtctcaaactggaccaacctcagcacagccagccaaaacacaatgaaccttggcacggtagcgccaactctcttctatttctctcccaagaagatctgggttcttgcatacgaatgggctgctactccatttgcctatgtcacgtcgaccgatcctaccaatgccaatggttggtcagcttcgcaaccgctcttcagcggaagcatatctccctctagccctatcgacccagcactcatcagcgatggcacgaatatgtaccttttctttgcaggagataatggcaaaatctaccggtccagtatgcccattggccagttcccatccagcttcggcacatcgtacacaacgatcatgagcgccgcaaccaacgatctgtttgaagcgatacaggtgtacactgtctcaggccagaatcaatatcttatgattgttgagtgtatcggatctgttggacggtatttccgctcatttacggccacatccctgagcggtacatggacgccgcaagctgcaaccgagagcaatccctttgcaggccatgctaacagcggagctacttggactaacgatatcagctctggagatcttattcgcagcacaaacgatgagacgatgaccattgatccctgcaacctccagctgctgtaccaaggaatggccgttggttccaccggagactataactccctgccatggcgacccgccgtcttgactctaaccaaccccggctcgagcacaggcaacggcaacggcactggaagcggcggctcaggcggcagcggatctggacaagcatcgcagtatgctcagtgcggcggtttgggctatactggacccacgagcccgtacaaatgcacgtttgtcaacgaatactactcccagtgtctatag

SEQ ID NO: 48 - DNA, 978 bp, Trichoderma hamatum
atgctctacactgctgctctggccggccttatggccactgctgcccttgctgccccctaccgccgacaggctactggccaaaacgttgtatactggggccaaaacggcggcggcgttactgaaagtaacaatttgtctgattattgcgatgctgaagcaggcatcgatctacttgttcttagtttcctctacgagtatggcaatggaaatactattccttcaggcactatcggccagagctgctccattgatacctccggaaacccttcaaactgtgatgcccttgcctcggccattgctacctgcaagtccaatggagtcaaggttgtcttgtctctcggcggcgccgttggagcctactctctctcctctcagcaggaggccgagacaattggccaaaacctctgggacgcatacggtgccggcaatggaaccgtccccagaccgttcagaagcaccgttgtcgatggctgggactttgatatcgaagccagtagtggcaaccagtattatcaatacttgattgccaagcttcgctcaaatttcaacggcggcaactacgtgattaccggtgctcctcagtgccctattccggagcctaacatgcagcaaatcattaccacttctcagtttgactatctttgggtccagttctacaacaatccctcgtgctcagttggaacgtcgacaccaaacttccaagattgggtttccaacattgccaacactccttctgccaatgccaagatcttccttggtgtgcctgcaagcccgctcggcgctactggaactgaatctggcgcccaatattacctcgagccttctgctctcaacactctcgtcggccagttctcatccaaccctgcgtttggcggtatcatgatgtgggctgccggtttctccgatgccaacgtgaacaatggatgcacttatgctcaagaggctaagaagatccttacttctggtcaaatctgctaa

SEQ ID NO: 49 - DNA, 1278 bp, Trichoderma hamatum
atgctttcccacgctatccttgctggcctcggccttgctggtctcgctgccgctgcgcctaccaggacgattgcgactcgtcaagcttccggttatcagaacgctgtctactggggtgccactggcaaacagaaccctaacctcgctgaatactgcacgtccacctcgggcattgatatcctgatcctgtccttcttggacgtctacggtgccactggcaacttcccctctggcaactttggcaacgactgcttcgtcggcaccacgggtgttccccagtcatgctctgacctggcctcccagatcaagacctgccagaacgctggtgtcaagattattgtttctctcggtggtgccgccggttcatactctgtcacgtcccagcagcaggcccagaccatcggtcaatacctctgggatgcctacggtaactctggcagcacctctgtccagcgtccctttggcgatgtctttgttaacggttgggactttgacatcgaggccaacggcggcttcagccagtactaccagtacatgattggcaccctgcgctccaacttcgccaaggactccgccaacacgtactacatcaccggtgctccccagtgccccctgcccgaggagaacatgggcgatatgatccagcactccgtcttcgactacctcttcatccagttctacaacaacaaccccacctgctctcttggtctgtctggccaggctcccctcaactacgacgactggaccaactttgtctccaccacccagtccaagaacgcgaagctcttcctcggtgcccctgccggacctctcgcctccaacggaaacccaaacggtgccgtctactacgctaccccctcggatctggcccccatcgtcaacaaggccaagacgaagtcaaactttggcggtgtcatgctctgggacgctggttactctgatgagaactccagcggcggttgcaactacgcccagcaggtcaagagcatcctcaccaccggcgccccttgcaacggcacccccgtcagcggtggtggctctcctcccgctacttcttccactgcttcttctcctcccgctacttcttccactgcttcttctcctcctgctacttcttcccttcctgccgatggaagcaccggaagcaccggtaacagcggcgttggcagcgttgcccagtggggacagtgcggcggtattggttacactggtcctacccagtgccagtccccattcaagtgcgttaacgagggcccgtattggtcttcttgccaataa

SEQ ID NO: 50 - DNA, 1149 bp, Trichoderma hamatum
atggccggttcacttacagcagacgctcacgaagctgttcggtgcatcatgtatctcacagggcaacatgtggtagtcccttcagaccatgatctcgtgggttccataacacatgtgatactggcattcatgcgctctgatgtcttcaatgtggataaaacgcctgccgagttcccgttttttacaaccgttgctgaaacacggcagaagttcaatgcgaatactaaaatcatggtcgcaattgggggttggggggattctgcaggatttgaagaagctgcgcgtgacgattcgtcgagaaagcggtgggctaaccaagtaaaggccatggttgacctgacaggagctgatggcattgacattgactgggaatatccggggggaaatcgtgacgactataaacttattccgaattctcagcgggaatgggagatagaggcattcgtgcttcttcttcgagaacttcgtttggttttgggagaagaaaaactactcacaattgcggtgccagcgctagaacgcgatttgatggctttcacaaactcaactattccgtccatcgtgaatcaggttgacttcattagtgtaatgacttacgatatgatgaatcgacgtgataccattgtcaagcaccatagtggtgtggccgactctcaggaagcaatggagcgatatatagatcgtggggcccctccgcacaaattgaacttcggacttggttactatgccaaatggttcatgaccgagcaatgcgatttacagcacccattggggtgccgcactcaactgctagaagaccccgccaatggagccgaccttggcaagactgcagcttttagctggcatgacgaggttcccgtggaattggctaattctttcgagaaagctcatactcatggccgctactatgaagatggaagctacgggtattgggatgatgaagagaagagatggtggtcctacgacacgcctctcgccatcaaaactaaagtccctcggtttctcggcgaactgcaattgggcggtgtgtttgcctgggggctgggggaggatgctccgcagtttattcacttgaaggccactactgatgggattcgggctttgcgcggaggccagagctcacgggattcggtgaaagatgaactgtaa

SEQ ID NO: 51 - DNA, 1332 bp, Trichoderma hamatum
atggcgcctttcaatactatgcttgggtatctcctgttagtggtagcccctttggcggctgccctgccgttccgtggccatggccccaacacccataaccacatcaacttgaacttcaacctcaccgaaaccatcaacgagatcaatgaaaacttggctggcctagttggatacatcaccaaccctcacgccaagcacatcgtcgccaaccgctacattgtcgtctacaacaacaccttcggctccgaggccatcgccgccaagcaggccgagttcgctgccaccatccagaagcgcaaccttggaaagcgaagcctcggcggcaacatgctgtccactgaaatccactccttccagatgcacacctggcgcgccatggctctcgacgctgacgatgagatgatcaagtccattttcgccgcgaaggaggtcgcctatatcgaggctgataccgtcgtccagaccaaggctctcgttgcccagaccaacgctacccccggcctcatccgtctctctaaccaaaacattggtggcaagaactacatctttgacaactctgccggcgctggtatcactgcctacgttgtcgacactggtattagaatcacccacactgagtttgagggccgtgcttctttcggtgccaactttgtcaactccaacaacactgatgagaacggccacggcagccacgtttctggtaccattggtggtgctaccttcggcgttgccaagaacatcaagctcgttgccgtcaaggtccttgatgcctctggtgccggtagcaactctggcgttctgaacggcatgcagttcgtcgtcaacgacgtccaggccaagggcctctccggcaaggctgtcatgaacatgtctctcggtggctccctgtccgctgctgtcaacaacgctattgctgccattgccaacgctggtgtcgtccctgttgttgctgctggtaacgagaaccaagatactgccaacacctctcccggctctgcccctcaggccatcaccgtcggtgctatcgatgccaccaccgatatccgtgctagcttctccaactttggcgctgatgtcgatatctacgcccccggtgtcaatgttctcagcgttggcatcaagtctgataccgatactgccgttctcagcggcaccagcatggccactcctcacgttgccggtctcactggctacctgatggctctcaagggtgttaccaacgtcaacgatgtcaccaacctcatcaagaacctcgccactgcttccggcgcctcggtccagcagaacgttgccggaaccaccaacctcatcgccaacaacggtgaactgtaa

SEQ ID NO: 52 - DNA, 510 bp, Trichoderma hamatum
atgggctatagccgcaaagataaatcgaggtacatcaacacaattcaagaagcacttgacatctctacagctttcgttgccagcagccgaaaaagaatagctttaacttggaagattatcattcaaattcaatccaatctatctagaatgcaattcttcgctctcctccctctgttatacaccaccgctgcagcccttggcatcaattgtcgtgggaacagcaactgcgtcggtactccagaatgcagactcgccgatctgatcttgcaagtcagccagcaagatcccagcacatcatacagccctggtcagcacattgcttgttgcggcatacctggcggcaatatctgtgcattcactcagggcattagcaattcgattacagctggagaagccctgggtatgctgcaggggctatccgcccacggttgtggccaatgcggcagtattcccttcaaggataacaatgtcgctgagggacaattgacagtcaactggaccgatcactaa

SEQ ID NO: 53 - DNA, 1170 bp, Trichoderma hamatum
atgggctcctttaaagccattatcgcagcatctctagctgccctaggacagttcgctgcagctgctccagcggaaggccaatccaagcgagctggcatctcttccatcgtcaagggaacaccggtaggctttgcgtcttcagtcacgggtggtgggaccgttgccccggtatatccaaccacgatcgcgcagctgaagagctacctcacctcaactagcccgcagaacattgtcatctctggtactttcaactttgctggtactgagggcacaacaactcttccagcttgtaacgcatacccatgtacaccctccaacggtggacaggcgttgctaaacacactgggtggatgcgggtcgctatctacgtacaacgtaaatctcgacaccgctgcatataacgctatcaatgtccagtcagacaagacacttgttgggattaacggtgcaactctcaatggcaaaggcttacgcctttctggcgtttctaatgtcattatccaaaacattgcaatcactaatctaaacccgcagtatgtctggggtggtgatgccattagtctcagcaatactaacaatgtctggattgaccatgtgaagacatccaacctcggccgtcagcactatagcttcggtactggctccaataacgcagtcaccatctctaacagcttcatcagtggtcagacctcttactcagctagctgtgatggccactcgtactggggtctcgaactggttggaagcggtgaccaaatcacattttacaagaactatgtctactacacatctggacgtacccctgctttgtccggcaataccctcttccatgcagttaacaacgtctggtcctcaaactccggacacgcaattgaaggtacttcgaacggcatgggcttgtatgagggtaactactttgtgaacgttccgaccattgtcgcttcaggcttcgtaggtcgtctcttcagctcacagtcatctgctgtgtcgcagtgcgcgcaatatttaggacgcaactgtgtctcgaattctttatcaaactcgggtactttcaccaatagcgacacgagcttcctttacctgtttcaaggcaaggccaatattgtttccgccgcttctgcctcgtctattcagtcgagtgtcgtttcgtcggcaggaaatacgctttaa

SEQ ID NO: 54 - DNA, 1185 bp, Trichoderma hamatum
atggttcaatctgcggcttatctaggagccttgctggccaccctgcccttggcacgtgccggtttcaacgctagctcgacgcaaaacattgccgtgtactggggtcaaaactcggcaaatcaagcaacctctcagcagcgattgtctacttactgtgccaatgctgatatagatatcatcccaattgggttcatgaacggcatcagcccggtcattaccaactttgccaatgcaggcgacaattgcacggcttttgcagataatcagaatgcacttaattgtccccaaatcgagcaagacatcattacatgccagcaaacatatggcaagacaatcctgatttcgctggggggcggatcttatacccagggcggcttctcgtccactggtgtcgccacatcagcggctcagacggtttggaacatgtttggccctgtcaatccaaacagcaacgtagaccgtccatttggttccgcagtggttgatggcgttgactttgactttgaatctggcgtcaataacttggccacttttgccactgagctgcggagcttgatggacgcttctgcgtcttctgcgaacaggaagttctacctgtctgctgcgccgcaatgcgtctatcccgatttcgctgacaacccggccctgaatggttcggttttcttcgatttcattatgattcaatattacaataatggatgcggagtcagcagctatgtccctggagcaactacccagtggaactacaactttgatgtctgggacaactgggcgcataccgtcagcaagaaccctaacgtgaggattctgcttggtatcgccgccaacactggtgctgccagtggatacgtgtccggaacacagctttctgcggtcatctcatttaccaagcaatactcaagctttgcgggtatcatgatgtgggatatgtcccaattgtatcagaattccggcttcttgggccaggttgtcagtgatctcgccgcctctggttcaacccccccggctactacttctagtggagcttccaagactaccacttctagtggcggttctacaagcccaactggcggcagtgtgccgcaatggggtcaatgtggaggcgaaggatacactggccctacgcaatgccagtcgccttacaagtgcgtttttagcagtcagtggtggtcatcttgccagtaa

SEQ ID NO: 55 - DNA, 1983 bp, Trichoderma hamatum
atgttactgaaacagattctcgttgccacgttgctttccttgagctctgtcaaggctttccctaacccgaatcacgcccaagatctcgaggccagggaagctgaagataatctcgttgctcgcggcggtcacgactgcggctcttacgctacctggaactacggaaagaactgctgtgtctgcaaagataacggcaagaactacgattctaagtccaagacatgcagttgtccacacgggcaagtctggaatggcaagcaatgcgttgtggactgtggcaaagatgcgacttatgattggaagcaaaagaaatgcgtctgcaagaaacaaggcgaggtctataactctaatacgaagacttgcagctgcccgcctggaactgtttggaacggctacggatgtgttgtcgactgtggcaaggaagcgcactttgataaatggcagaagaagtgcgtctgcaataacaacggcgaagtctatgattccaagagcaaaacctgcagttgccctggtggccaatactggaacggcaagaaatgcgtatgcccttatggcaaggtctggaacggcaagcagtgcatagaggactgtggtaaagacgcacacttcgactataaccaaaagaagtgcgtatgcaacaagaatggacaggtctacaactccaacagcaagacctgcaaatgtccaggtggccagtattgggacggcaagcattgtgtctgcccatatggtcaagtctttaatggcaagcagtgtgttcctgattgcggtaaagaagcccactttgattataagcaaaagaagtgcgtatgcaacaactacggcgaaatctacaattccaagagcaagacttgcgcctgtcctgacggccagtattggaatggaaagcaatgcgtttgcccttacggcaagatctggaatggcaaacagtgtgttcctgattgcggtaaagacgcccactttgactacaaccaaaagaagtgtgtttgcaacaagaatggagagatctatgactctaagagcaagacttgcgcctgtcctgacggccagtattggaatggaaaacagtgtgcctgcccgtacggcaagatttggaatggcaaacagtgtgttcctgattgcggcaaagaagcccacttcgattacaaccagaagcagtgcgtttgcaacaataagggagagatctatgattctaagagcaagacttgcgcctgtcctgacggccaatactggaatggaaagcagtgtgcctgcccgtacggcaagatctggaacggcaagcagtgtgtagaagattgcggcaaagaagcacacttcgattacaaccagaagcaatgtgtttgcaataacaagggagaaatctatgattctaagagcaagacttgcaagtgccctgatggccaatactgggacggaaagcagtgcgcttgtccatacggaaagatctggaatggtaagcagtgtgtagaggactgtggcaaggacgcacacttcgattataaccagaagcagtgtgtttgcaataacaagggagagatttacgattctaagagcaagacttgcaagtgccctgacggccaatactgggacggaaaacaatgtgcttgtccctacggccaaatttgggacggaaagcaatgcacaccaaactgcggcaaagacgctacctatgacagtaagcagaagcaatgcgtgtgcaacaagaagggccaagtctttgattccaagcacttgacatgcagctgcccggctggaacgagttggaacggctatgcttgtgtcccagattgtggcaaggatgcgcactacgataccaatcagaagtgctgcgtgtgcaataacaagggccagatatttaattctggaagcaagacatgcagctgcccaggcaaccaatactgggacggcaataaatgcacatgcccctatggatcgacatgggacagcagtaagaagacgtgcaagcagacacccatacattaa

SEQ ID NO: 56 - DNA, 1275 bp, Trichoderma hamatum
atgttgggcttcctcggaaaatccgtggccttgcttgctgcgctgcaggccaccctcacctctgcatctcctctgtctacaaacgacgtcacagttgagaagagagccagcggatatgcgaacgctgtctacttcactaactggggtatctacggccgcaactttcaaccccaggacctggttgcgtcggacatcactcatgtcatctactcgttcatgaacttccaagcagacggcactgtcgtctctggagatgcttacgccgattaccagaagcactattccgatgattcttggaatgatgtcggcaacaacgcgtacggttgtgtcaagcaactgttcaagttgaagaaggccaaccgcaacttgaaggttatgctttctataggtggctggacctggtccaccaacttcccttctgccgcaagcaccgatgccaaccgcaagaactttgccaagacagccattactttcatgaaggactggggttttgatggtattgacgtcgactgggagtatcctgccgataacacccaggccaccaacatggttcttctgcttaaggagatccgatctcagctagatgcttatgcggcccaatacgctccaggataccacttccttctctccatcgctgcccccgctggcccagagcactactctgccctgcacctggccgaccttggtcaagttctcgactatatcaaccttatggcttatgactatgctggttcttggagcagcttctccggacatgatgccaacttgtttgccaacccctccaaccccaactcttcaccatacaacaccgatcaggctatcaaggcttatatcaacggaggtgttcctgcacgcaagatcgttcttggcatgcccatctatggacgatctttcgagagcaccggtggaattggccagtcttacagcggaattggatctggaagctgggagaacggtatctgggactacaaggttcttcccaaggccggtgctacagtccagtacgactctgtcgcacaggcatactacagctatgaccctagctccaaggagctcatctccttcgatacccctagcatgatcaacaccaaggtctcttacctcaagaacctcggcctgggaggcagtatgttctgggaggcttctgctgacaagactggctccgactccttgatcggaacaagccacagagcgcttggaagcctggactcaactcagaacttgctgagctaccccaactcccagtacgataacatccgaagcggtcttaactaa

SEQ ID NO: 57 - DNA, 1239 bp, Trichoderma hamatum
atgtttttcagcaaagcactggctgctaccggccttttggccactgctgctctcgctgcacccaccgtggagaagcgtgccgctggaggcaagctcgtcatctactggggtgctgaagacgacagcactactctcgccaacgtctgtgccgattcctcttacgacattgtcaacttggctttccttgacaagttctctgctggtggtggctacccctcgctctctctgagcaccctgggcggcccatctgccgctcagaaggctgctggtgccaccaacctccaggatggttcttctttggttcccgccatcaaggcctgccaggccgccggcaagctggtcatcctctctatgggtggtgctaccgacttctctgcagtctccctttcgggcgatgctcagggacagtctgttgccgacatggtttggaacctgttcttgggtggtactgccaaccccactctccgtcctttcggatcagtcaagctcgacggtgttgatctcgacaacgagactggcaaccccactggttacctggccatgactcaacgcttccgatccaactttgccaaggacaccagcaagaggtactacctcactgctgctccccagtgccccttccccgatgcctctgagcctctcaacgtctgccagctccttgattacgtctgggtccagttctacaacaacggcaactgcaacgttggccagtccggcttcaacactgctgtcaagaactggagcaagaacattggtaacgctactctgttcattggtgcgctggccagcggtgccgatggtgaccagggctacatctctcccagcgctctgatctctgcttacaacggtgtctctgctcttaacctgcccaacgttggcggtatcatgctttgggaggctcagctcgctgtcaagaacagcaacttccagaagaccatcaaggctgccatcggctccggctccactcctcctcctcctcctcccgcctcttccaccactcctgccggaagcacccccacctgctcttgggccggccactgcgctggcgctacttgcagcactgacaatgactgctccgactccctcacctgcaacagcggcaagtgcggtaccgctggcagcactgctcctcctcctaccacttgctcttgggcgggccactgcctcggcgcttcttgcggtaacgacaacgactgctctgacccttacttctgctccaacggcgtctgctctcagtaa

SEQ ID NO: 58 - DNA, 930 bp, Trichoderma hamatum
atgtttttccgcaacgccgtcgccgtaaccagccttttggctgcactttctagtgcccaaccctccggccccgaattggccgtgtactggggtgctgaagatgatagcacgactctcagcgacgtgtgttcagacaactcttatggcattgtgaatcttgccttcctcgacacattttttgctgcaggaggctttcctcaactcagcgtcagcgggttagatggcccatcccaagctcagcaaagcgctggtgccactggcctcaaggatggtagcagtcttgtagacgctattaagcagtgccagtcggccgggaagctggtccttctcagccttggtggcgctggcgccgatgtcactcttcaatcagactctgatggagagaaaattgctgatacactctggaatctgttcggtggtggcactgataaccaagagctgcgtccctttggagatgtcaagctcgatggcttcgacttggacaatgaatctggcaaccccacaggttacctagctatggtaaagcgtttcaagtccaacttccagtcggacacgagcaaaacttatttccttactgcggctcctcaatgcccattccccgatgcttcgcagcctcaagatgtttgcagtgaattggactttgtctgggtccagttttacaacaacggcgattgcaacattgcgcaatctgatttcttaaactccgtccagacttggagcagcggcattggcaacgctaagctctacatcggtgctttggctagtggtgctgacggcgatcagggctttgctgatgctgatacgctattgggtgctattcaggatgtcaagaatatgaaccttcccaattatggaggtgccatgctttgggaagcccaactggctgttaaaaacggcaactttcagcagaagattgcccccggtctataa

SEQ ID NO: 59 - DNA, 396 bp, Trichoderma hamatum
atgaaatcaagtctcttgtctcttattgccattgcaattaatggtgccttggctggcattaactgtaatggcagcggagactgtgccgggactcctggtactctaggcgatcttattgctgatgcataccaaattgacccaaatcgttggtataacaatggagaacatatcgcctgctcggataatcgaggcggcggtggcctttgtgctttcttccagaatactctcggtggccctggaagctcagttctgactttgcttcaaaacctgcaagctcacggctgcaataagtgcggcagtattccggtaaatttccctcagggagataacagtgaaaatcatggggagcttactgtcaactttgttgtttcggctggctgcactgggctttgctag

SEQ ID NO: 60 - DNA, 408 bp, Trichoderma hamatum
atgaagttcagctttacgtccgctatcgccattgcaagcattggaatcactggcgccagagccggcattaactgcaatggcagtggcaactgccctggagtagctggcgacctcagcacccttatttcatttggatggtcaatcgaccccaaccgttggtataacaacggggaacacattgtttgcgtgcaaagtcaacttgggactggactttgtgccttcctccagaacaccggcggtgcccctggtagttcgattcagccgttgctccaggcattgcaaggccacggatgcaataaatgtggcagtgttccgctgaacttccttcagggagataacagcgaagatcacggggagcttactgttaatgctgttggctcaactgccggctgcagtgggatttgctag

SEQ ID NO: 61 - DNA, 1035 bp, Trichoderma hamatum
atgacacgcctcctcgacgccagctttctgctgctacctgccatcgcatcgacgctatttggcactgcctctgcacaaaatgcgacatgtgcactcaagggaaagccggcaggcaaagtcctaatgggatattgggaaaactgggatggatcagccaacggtgtgcaccctggatttggctggacaccgatcgagaaccccatcattaaacagaatggttacaatgtgatcaacgccgccttccccgttattctatcagatggcacagtgttatgggaaaacgacatggctcctggcactcaggtcgcaaccccagctgaaatgtgcgcggctaaagcagctggagccacaattcttttgtcaattggaggtgctactgctggcatagatctcagctcaagtacagtcgctgataagttcatcgcgaccattgtaccaatcttgaagcaatacaattttgacggtattgatattgacattgagaccggcttggtcaacagcggcaatatcaaaacactttccacatcacaggccaacttgattcgcatcattgatggtgttcttgctcagatgccttccaacttcggcttgaccatggcacctgagacagcatacgttacaggtggtagcatcacgtacggctctatttggggatcctacctacctatcattcagaaatatgttcagaacggccggctgtggtggctaaacatgcaatattacaacggcgacatgtacggctgctctggcgactcatatgccgctggcactgttcaaggattcatcgctcagactgattgcttaaatgcaggtcttaccgtccaaggcaccacaatcaaggttccatatagcatgcaagtacccggtctacctgcgcaatcaggagctggcggtggctacatgaacccgagcttggttggacaagcctgggatcactacaacggtgctctgaaaggcttgatgacgtggtcaatcaactgggatggagcaggcggctggacgtttggcgacaatttgcttactcgtattggttag

SEQ ID NO: 62 - DNA, 846 bp, Trichoderma hamatum
atgcacctccactctctctttacggcgcttgcgctggcgagccctgcgctcgctgcttcaatccctcgcagcagcagcagctcctcatcatcaactatcaccgtctgggcgactccccacgacagctactcatcctccgtcggcgtcttgggctgcaaggtcgacaccaatcgcatcgcctactggccagactccgtcgactgcaccaacatctgcgtgtctctttcgtaccaggaccgacaggtctacctccttcgcgtcgaccaatcacagggcgcgcacgacatcagctacgatgcctggaattacctcgttaccggctatccagccacggaaaagcccgttgcgggagggcccatggaaatgacgacggaaaacgtcgatgcgtccaaatgcgccgacctcatctacacagcgggcggcaagcttcccctcagtgcggcaaatagcatgaatttcttggccagctgcttggagcaagagaattcatgggttgcaagcaactacgtgttatacaacatcttggatgcaatttgcaccgttggccaaaaccaggtctgctctttgaactggcccactgccaaccagcccacctgtccgggcaccctaggattgcccgacgcactcaaaggcgaacctgtctacaatatcgagtatccttccggcgtcaaggtacttgcaggggcgccaccaactgtaccaactggagtacctgttccagtaccggcgccttctaccaacgacgatgagaagagcgctgcacggagtttaaggcattcagactctctcgtctggataccagtcctttcattaacttccattatatattcttggatgctgtggtaa

SEQ ID NO: 63 - DNA, 459 bp, Trichoderma hamatum
atgcgtttcaacagtttcatcgccgtcgcggctgcgaccgctcaaagcgccgccggcctgggaatcaactgcctgggcgacactctctgcggaatctcgtacatgagcggcggacgcctgacccagttccagaccatcttcgacaacatctttgagaagcggatatacgacaacggcgacgacatcggctgcatcgaggtccactcaatcaacttcaagggctccttcaaaggcacatactgcgcctacgtccaaaacctggatggcagcgtcaacggcgccaccctcaagaccctttacaccgagctggtcaactatggctgtggaatctgcggctccatccccatccactacagcaagggcgacaacgactccaaccacggagagctctctttcaacatggtggacagcctgccggacaactgcgagctcggcaagccttgcgcagcgacctcctaa

SEQ ID NO: 64 - DNA, 447 bp, Trichoderma hamatum
atgtacttcgccaacaccctactctccatcgccggtgctgcatcaatagccaatgccttgggcagcaattgtcaagacagcggtgtttgcgcggatatcaacgctaacctgagttttgccattgaacaactaaaaggaatggaccagctccagagattctcggacggacaacacatcacctgcgttgatacggacagtgagggcaactcttccctctgtctctcataccaggacactggccgatcatggactgtattccagactgcctggtttgcccagtccttggtagagcagggatgccaggcctgtggaagcctgtctatgggctctcatcatggcgagctggtcgccagcgtcatcacaaagagtaccagcggcctggatacctctggagctcgtcgtgggatggacatggttcaactggctgtaagagctggggaccgatga

SEQ ID NO: 65 - DNA, 417 bp, Trichoderma hamatum
atgttgtttaaaattgccatcgttgtgatcgcctccacagcacgtgtttccgcctttggtatcaactgcgaaggcagtggctactgctctccattgttcaatcctggagccaacaaccacccgctgctagagatggtggacgttatcgactttggaattgatgacaatcgatggtatgcagcaggagagcatattgcctgtgatcagagttctggtgtctgcgcttttgtacagaagattggcggtgcttcaggtggcgatattgctcgagctgtccgctatctcgcagaccatggctgcactacctgtggaagcgtacccttggacttccctaacaccaacgatgtcaacaacggagaggtgactttcaactttgttgggttggaagatatgggtagctgcagcgatctttgctaa

SEQ ID NO: 66 - DNA, 415 bp, Trichoderma hamatum
atgaaaatgcttattactgccgctcttttcaccctggccctcgccgcccctgtcgccgagactaagcctcatagcattgctgctcgcgatccctttacttgccccggcggcctgaccaacagcacccccatgtgctgcagcgttaatgtccttggcctgctagcccttgattgccagcaacgtatgggttactttgcttcatatttatcctttcaatatatggggaacttgaatagctgacattttcatcgctcattagctggcgcggacggctgtagtggcagctccaagcccaattgttgcacccttggagcggtatgttttttaatgattggtcacgccaacagttttgagcatttactgatagaatttctcaatacaggccggacagggtgttatttgcaacgcactgtaa

SEQ ID NO: 67 - DNA, 2033 bp, Trichoderma hamatum
atgaagtcggccattcttcttgggctcactggcctcgctgccaatgtgaatgcccatcctgctaagcagccagagaccggcaacggcctgactaagcgtggcgttgatattagcaagtatagcttgcctaatctttcaaagtacacgtcttcgccaaacatcgaacaagaggcctctctgcagtctttggctttcaagcgcaactatgtggatacagccacacgagctgtcaagaaagctgcccccaaggccgaattccgcgtcgtggatgaccactatatcgatgttgacggcattggtcacgtccactttaagcagacagtgcacggaattgacattgacaacggtgacttcaaggttaacgtgagtggaaggcaaaatcacctcacctagaaaaccagaaacttacaaataatatgaacttagattggcaaagacggtagagtcttctctcatggaaacagcttcttttctggcaagctgccccagcagaaccccttgaggaagcgtgatttctctgaccccaccactgcgctcaagggtgcaattgatatcctgggcctcccggttcaggctgatggcgccaccgccgaagctcaggaaggcactgagaagtacacgttgaagggtacatccggagctgtcagcgatcctaaggctcacctcgtttacctcgtcaagggagacggaactctggctctgacttggcgggtcgagaccgatatcatggacaactggcttctcacctacgttgacgcaacaaccaaccaagagattcatggtgttgtagactacgtctccgaccttgccaccttccaggtttacccctggggcctcaatgaccccaccgagggagatcgtaaagtcctgaccgacccgtggagaaccgatgcctccccattcacttggctcagcgacggaaccaccaactatactgtaactcgcggaaacaatgctattgcgcaggataatccttctgggggggacagttatctcaataatcaccgtccatccagctcgactcgggactttcaatatccctttacgctgactcagaccaaccctacagattatagggatgctgccatcacacagctcttttacacagtcaacaagtaccacgatttgctatatgtccttggcttcaacgaggtagcaggaaacttccaagcaaataacaacggaaagggcggcaaggccaatgatttcgttattgttaacgctcaagacggcagtggaacgaacaatgccaactttgctaccccggccgatggaagcaatggccgtatgagaatgtacatctggactactgctaaccccaagcgagatggcgatctcgaggcgggcattgttatccacgaatacactcacggatgtaagtctctctcatttcaacttcgtatctactctttctaacgcttgaaacagtgtctacccgtcttaccggcggccctgccaactctggatgccttactggcactgaagccggcggcatgggtgaaggctggggtgatttcttcgccaccgctatccgcttaaaggctggtgacacccgcagcaaggactatcctatgggagtctgggccgataacaacgtcaagggtatccgccagtatccttattcaacaagccttactaccaatcctcttacttataaaaccgtcaatacgcaaaatgaagtccactcagccggtactacatgggccaccattttgtacgaagtcctgtggaacctcattgacaagtacggcaagaatgacgacgatttccctacctttgatagccagggcgtccccaccgacggcaagtatcttaccttgaaactcgtcctcaatggattggctctgcagccctgcacgccaacttttgtttctgcccgtgatgccatcttggatgctgatcgtgctttgaccggaggggagaatctttgtgaattatggacaggattcgccaagagaggtcttggatccggcgccaaatattcttccaccgctcgcgttgaaagcttcactatcccatctggggtttgctaa

SEQ ID NO: 68 - DNA, 438 bp, Trichoderma hamatum
atgaagttcttcgccgccaccgctctccttgccgccactaccattgctggacctcttgaggttcgcactggagacggtaacatttgtcctagcggcggcctctacggcaaccctcagtgctgctcctctctcctccttggtctcattggtcttgactgcaacgtccgtaagttaaaaacaaagctcattcaaagcctattgatgcaatacattctcatactggtgtcctatagccaaccaaacgccccgtgacggcgctgactttagaaacatctgcgctaagactggtgacgaggccctttgctgtgttgctcccgttgtaagtaatcacccagcttcaagttgcccttgcccttgcccttgtgctgttgctaatttctacactatctttaggccggccaagctcttctctgccaggtcgccgttggtgccagctaa

SEQ ID NO: 69 - DNA, 432 bp, Trichoderma hamatum
atgaagttctttgctgttgccactctctttgtcactggcgtcatctccgccccgtcacccaatgccctaaattcccgcagtcttctttgcagccctggcctttactccaccgcgcagtgctgtggtgttgatgtattgggagttgctgatttggactgtgcggctcgtaagtagtctctcaaagcccaatcatatttattgatttgaagatgctgatactgggaatttagctactggcactattaccaacgcccaaggatttcaggctgcctgtgctaagaaaggccaagaggctcgctgttgtgttctacctgttgtaagttatcctaagatacgtgtaatgcggctgcttttatagactggcaatactaacaatttttgtccctttaggccggccaagatgtcctgtgccaggacccccctggtttgtaa

SEQ ID NO: 70 - DNA, 1351 bp, Trichoderma hamatum
atgaccagcattcgtcgtcttgctctctatctcggagctttgctcccggctgtcctcgccgctccagcagcccttcacaagaagcctgaggctgcacctaacaagtttattgtcactcttaaagagggcgcttcaattgataccgactctcatctcgcctgggtgaatgacatccaccgtcgttctttgaccaagcgtagcactgctggtgttgaaaagacttataacattcatacttggagtgcttatgcgggtgaatttgatgcagagacgattgagcagatcaagtctagccccgatgtacgtgctctccgtatcctaacgaaaagaagaaaagctaatcacgctgtcaggtcgcgtctgtggagccagactacatcatgtacctgtcggacattgttgaagacaagcgtgctctgaccacacaatctggagctccttggggccttggcactgtttcccaccgcacatctgggtctacgagctacatttacgatagctcagctggctctggaacctttgcctatgtggttgactccggcatcaacacctctcatcagcaattcggcgggcgcgccagtcttggctacaatgctgcaggaggacagcacgtcgacactcttggccatggtactcatgtttctggaacaattggtggatctacatacggcgttgctaaacaggtgagtgattccaatgttgttgttatcacatacaagtgcattcaagactgactcggtactattcataggccagcctaatctccgtcaaagtctttgctggagagagcgctaccacctctgttatccttgacggctataactgggctgtaaacgacattgtctcgaggaatcgtgctagcaaatctgccattaacatgtcgcttggaggacctgcctcatccacctggacgaccgccattaacgcagcctttaaccagggtgtgcttaccattgtcgccgctggtaatggcgacagtttcggaaacccccagccagtttccggcacttctcctgctaatgttcctaatgctatcactgttgcggcgctggacatcaactggcgcactgcttccttcaccaactacggtgctggcgttgatgtctttgcccctggtgttaacattctgtcgtcatggattggatccaacactgctaccaacacaatcagcggcacctctatggcgacacctcacgttgttggtctggctctctatcttcaatcccttgaaggcctcaccactcctaccgctgtcactaatcggatcaaggctctggctaccgctggccgtataactggcagccttaacggcagccccaacactctcatcttcaacggaaacagtgcttaa

SEQ ID NO: 71 - DNA, 355 bp, Trichoderma hamatum
atgagaacatctaccgctattatatctcttgcccttgtggtgatgggaattgccgcccctgctggtcatgtatcgagcaaccagccgcggagcgtggaaggttgagctcatcaaaggagcagccgaggccggctctgttgtcagcgaagccggctttgttgtcagcgaagcccaattcaacgccatatttcccggtcgaaaccctttttactcctatagggcattgaccggcgccctcggatcttatcccagcttcaccaacactgctgataacgccactaggctccgcgaggccgccgctttcttcgccaatgtggatcatgaaaccgatgggctcaagttcgtcgtggaataa

SEQ ID NO: 72 - DNA, 1422 bp, Trichoderma hamatum
atgagcgccgcatcccgcaagtcacgcatagctacgagtgtggcgagagttatgtacaccaatgcagtctatttccctagcagcagaatatatcaaggtgattcgccgggaatgctcaattacagctgcatcaaccacgtgtattatgcttatgccagcgtgacggcggacggcaatgtgtttgtgagttttgatcaagtcccggagaccacacatatggcaaagtggcttcttttcccgtgccatggttcggaaaagttgagaatatttcacctgcatgatgttgctcggctgctgacttgtgacgtgtggcccagctcggcgatgagtgggccgatgcaagagcgccagtggatggtgttcagggtggcttaggatccttgatgcatctcaagcagaggcaccctcacctgcaggttgtcttatctattggtggcagcactgcatcagaggtatttcccattgttgcatcaagcactctcctcagagacaactttgcccggtctgcccttggcctcgtggaagcatccgggcttgatggtattgacagtaagtacgaacgaacaagaggaagacgaacaaagaaggaggtagaggaagcatggatgtcaagttcacacacgcgcgctcgcacatacacccgccacaaacaagagtcttcagctaattactcgtctccctaaatagttgcctgggagttcccatcccaagctaaacatggccacgatttcctagccttgctggcagcagtgcggattcatatgcctgaagaccgcttcattctaacagctgtcctccctgcggcgaaggaggttctacaactcatcgacctcagcacagcggccgagtaccttgactacatcaaccttgtggcatatgatttcttcggcacgtggacgtccaaaactggtcatcacgcacagttatatactatgaacaaggacgaaccctcggcatcgacaggtgtggcgtatgtcatgtcccaaggattccctgcgaagagtatcctgctcgggatcccaacctacggacgaagcttcctcaaggccaatgggccgggccaggattttaacggcgttggcggccaagatggcacattcgaatacggcgaattaccacgaaagggatgcaaagagattgtggataggcgctatattgcggcccaatgtgtcggtggtgatggggggtttgtcacgtacgacaaccccgagacggtcaaggttaaggcggaattttgcaagcaaaagggattaggggtttgtggagctcaaatttatccaatctctgtgagacacaaattggttgctaactatcaaatttttaatattgcaggggcttttttactggaacggaccggctgactctcgagatcaagcacgaagtctgattgcggctggattccgcgctctacacacctcgtga

SEQ ID NO: 73 - DNA, 424 bp, Trichoderma hamatum
atgcaattgaccacagtcgtcgcgctcttcgcctctctggccggcgccgcgcctgcccctgaacccgctgctgagcttgttgctcgtgatggtccttgctcctctggtgtcaccaataacgtccctcaatgctgcggtactggtatcctcagtgttgtctacgtggactgcaagactcgtaagtctttcaacgctctcgcttgatgagaaatccccattgcatgctaagccttgacttagccactcaagctccctctgccactaaccagttgagcgccatctgcgctcgagtaggtctccaggccaagtgttgcaccgtcggcattgtaagttcttcgtcctagaatcggatttgcaaaagggattctaattattgtatcaggctggcgttggcgttctttgccaggatgctattccccagtaa

SEQ ID NO: 74 - DNA, 480 bp, Trichoderma hamatum
atgcaattgtccaacctcttcaagcttgctctcttcaccgccgctgtctctgctgataccggtatgatgaatcactacctgtattcgatgctctcgttttgctaacacagattcactcacacagtctcgtacgatactggctacgatgacgcatctcgctctctgaccgtcgtctcctgctccgacggccccaacggcctcatcaccagataccactggcagacccagggccagatccctcgcttcccatacattggtggtgtccaggccgtcgccggctggaactcccctagctgcggcacctgctggaagctcacttacagcggcaagaccatctacgtcttggctgttgaccacgccggtgctggcttcaacattggcctcgacgccatgaatgctctcaccaacggccaggctgttgccctcggacgtgtctctgccactgcctctcaggtggctgtgagcaactgcggtctctag

SEQ ID NO: 75 - DNA, 1445 bp, Trichoderma hamatum
atgcattcaacaactttctttgccagtctgggactcgcaggcctggttgccggcgccccttcggccccgcacaacgttaaagctcgccaagcttccggtgcccaaaacgtcgtctattggggaggcactaacaatgaaagcgacgacctttctacctattgtacgcccaccgcaggcattgacatccttgttctgtctttcttggacatttacggcacaactggcaacattccagcaggtaatattggcaattcttgctatatcggaacaaatggcacacctcaactatgtgataatcttgctgcttcaatagccagctgccaggccgctggtattaaaattatcttgtctctcggaggagcggccggctcctattctctacaatcgcagtcacaggctgtggctattggccagtatctctgggaggcttacggcaactctggcagcacttctgtccagcggccctttggcaatgtctttgttaatggttttgactttgaccttgagctgaacgccggcaaccaatactaccagtatctgatatcaaccctccgttccaactttgccagtgacccgaaacatacgtattatatcactggagcgccccagtgtcctctcccgttgagtattcctcaaatccgtagttagatgggtgactcatggcttgctaacaacactaatggaacaaatagagaaccaaatatgggagagattataagtgcctcacagttcgactatctctggattcaattctataacaacaacgcttatgcaccagacccctgttctcttggtctgcctggcgatgcacccttcaactacaacaactggacgtcatttattgctactaccccttcaaaaaacgcagtatgtgtatttcggcccctggtattatcttgcataccaaatttctaacaatttctactagaaactatttattggagtccctgcgaatactttagctgctaatggcaattcaggtggtgccgtttactatgcttcgccatcccagcttgcttccattgtcgcgaatacaaaatctagcccagattttggaggaattatgatgtgggatgctgggtactctgatgctaatgtcaacaacggatgcaactacgctcaggaggccaaaaacatcctccttaccggcgctccctgtggaggctcacccccaccggttagcagcagcaagcctacatccactgcaaccaaatccgctactagcacttcatcagcctcgggaacaggcccgacaggaggcggcacagtacctcagtggggccaggtgagtcactagaggccttggggcttacatatgacctggggaaaagagccctagctaattgatgttttagtgcggcggagagggatacacgggcccgacgcagtgcgtttctccctacaaatgcgtcgaatcaagtcaatggtggtcgtcttgccaataa

SEQ ID NO: 76 - DNA, 1185 bp, Trichoderma hamatum
atgccctctttgatcacggttgcgagcgtgctcgctcttgttccatccgccttcgcgggctggaatgtcaactcgaagcaaaacattgctgtgtactggggtaatgactcttgtaaatatttctaatttatagaaagaaagcatctaacatgctatctgtataggacaaaactccgccaaccaacagagcacacagcagcgtctttcagcctactgcaatggtacgtttgtgctttatttcctgaccgaaacctacgattgggttgagaaacagcgaagattgaatctgacagttgcgatgtagatgccaacatcaatgtcattgacattgctttcctgaatggaattactcctcctatgaccaactttgccaatgctggtgaccgatgcactcccttctccgacaacccttggctcttgagctgccctgaaattgagtgagtcttctgttgaagaaatgcttttctgttgtaaatagatatggactaaccatggttgaattaaagggcggatatcaagacttgccaggctaatggcaagaccattctcatttctcttggtggtgattcttacactcaaggtggctggagctctaccagtgctgctcaggccgcagccaaacaggtctgggccatgtttggtcctgtccaatccggcagctccaccgagcgtccctttggcagcgccgttgttgacggttttgacttcgactttgaagcgacgactaacaatctcgcggcctttggcgctcagctcaagagcctctcgagcgctgccggtggcaagaagtactacttctctgctgctccccagtgcttcttccccgacgctgctgtcggtgcactgatcaacgccgtccccatggactggatccagattcaattctataacaacccttgcggcgtcagcggctacacccccggcaccagcaaccagaacaactacaactaccagacctgggatacctgggccaagacaagccccaaccccaacgtcaagcttcttgtcggcattcccgctggcccaggtgctggtcgcggctacgtgtctggctctcagctcacttcagtcttccagtactcgaagggcttcagcagcacttttgccggtgctatgatgtgggatatgtcccagctcttccagaacactggcttcgaggcccaggttgtcaatgctttgaaataa

SEQ ID NO: 77 - DNA, 1753 bp, Trichoderma hamatum
atgccgcttaaggtcttcgagtcagcttcgcctgcgccgtcgcatccctccttatcgcacctctgcctgctcgtcttcgaagctgttctagaagtagtctgtgtgagcttgccgggatacatcgttgcgcgccttggccatttcgatgccgataagcaaaagttcctggctaatctcaatgtgatgctcttcacgccctgtcttagtacgttgctccctcttgccatgtctctgttattctcctttgaacgccgtctgacccatttctctcctcagtcttcaccaagctagcatctcagctcaatgccgaaaaactatccgatctcgccatcatccccgccatctttatcgtgcaaactcttgtatcatggatagtttcgatcctggtcgccaaggggttccgattcaataagcgagcttccaacttcgttactgctatgggcgtctttggcaattcaaattcactgcccatctctctcgttctttctctctcgcaaacaatcaagggcctccactgggacaggataccaggcgacaacgatgacgaagtcggcgctcgcggtatcttatatctactgattttccagcaacttggccagcttgtcagatggagctggggatatcacgttctgctagcccccaaggataaatacgccgagtatcaagacgaaattgccgaagaaggccagtacagatacagagacgaagagccgaacgaacaggagccggagattctcattactggtttggacggtgataccgaagatgatggcgagagcaatgcttctgaagattatatacctgctggacgaacacctcttgcgagcaattcccgggcttcactggctggctcttccgtcgacaatgacgacatgctgaatttcaaaaagggcaactacactcgtggaagctcgctcgccaacacagacttggaagatgatattctttcattccctcgtattcgtcttcgagatgaggctgaagtcgagcacggagttactgctcgtatcaagaaatcactatattctctgaaagacaaagcttctgcggccatgactcgccaatatcagcgacttcctcaacctgttcaaacctgcctttccttcattcataagtcaatcacaaagactctcggatttgtatgggatttcatgaatccacctttgtgggctatgctcatcgcggtcatcgtggcgtccatcccgagtctccaacagttgttcttcgaagatggttcattcgtgaaaaatagcgtcaccaacgctgtttcgtctagtggaggcgttgcagtgcctttgatcttggttgttctcggcgccaacttggcgcgcaacactgccgctcacgattctcccatagacccggaggaggaaaagattggcaccaagctcttgatcgcttcgttgctgagcaggatggttctgccaactttgattatggcacccattctggctattacggcaaaatacttgcccatcagcattctagatgatcccatctttattgtcgtatgctttcttcttactggcgcacccagtgcgctccagcttgcgcaaatttgccaaatcaacaatgtttatgagcagactatgggaagaatcctctttcagagctatgttatctggtaagcatttcaacacccttaattttgtctatcgtgcatttcactaatacacatattaggattcttccctctactcttttccttgtcatgatggcactcgaggtgattgagtgggcaacagtgaattaa

SEQ ID NO: 78 - DNA, 1443 bp, Trichoderma hamatum
atgctaccgtcgagcctttgccggattgccgctgttatcagcgtggcttctgcggaaatggtttcgggtgagtacaatataaaccctccaacatgtgatctgtctcacacttatacatcatagtaacatttgataccgcttacgacgatccttctcgttcactgagtgaagtcgcttgttggaggaagggcacgggattcatgcctaacttggattggaaacttcaaaaagatgcattggaatttattggaatcaaagcaattcgtggcttcagttctgcccaatgtttttcctgctggaaaattgagtatggagataagcagatatcactgttcgcaattgacagcgccgactcgggcattgttctctctctgagcaccatgcaatatcttacaggtggccaggctcgcgagctggcccgaattgatgcaaaggcaacccaagtcgacgcatcaaactgtgggatttctgcggctgaactgcacaaatacgatttctagttagtcgtatccacttttaggctaccaagcaagcgttcaagtagtttcaaagataagtcaagctttgtatcttcgcccctattacctggagttgagactatttgttacgttttttttggtgagactacggctacatatagccccaaacgttcacttctactgcggcattagtttttatacaatgcattatttacgccaatatcttgtatcaaggtctgagtagtgtcaacctccactcttgagagagcatctatagcttaagagtatactcattacgaccgaaatatattgatattttattcaaacacataactagcaatgataaaagtcgccttagaggacgtaagagtaataaaatgtaaaaatttcagatctggacacatatactgcacacacatacatttataactccgcaattaaccatttttggttaagtacagctgagataacttactttcatacatcgattttaccgtagttgagaatatcttggtatttaattatctttacattcggttcgtcattcaacaaaagatgcgcaattgaagttgtcacattatctgtttaattatgagtcttatggacagatagagatgcaattttgtggtgaagagaggtatttttatcatcaacagcactaacagatcactgccttactgaaattagcagtaccaatggtagtggaaatagcgatggctctaggagcaacactggagctattgttggaggagttctcggtggtgtggctgggcttgctttaactgggcttgctgtttttttcttcccccgctataagaatttagctgctggacataagccagtgagccagaatactcctcctgctgcttatcaatcctctctgatgcagcaggaccccaaggctcactatgatcctaaatatttcgcagagatggatacacagccatacacgccccctgattag

SEQ ID NO: 79 - DNA, 1272 bp, Trichoderma hamatum
atgctccgtactcctacaatcccctggcttctaaatatcatcctagaagaaaaccctactatgattccttcaatgaaaaagtgcggccgcaagttatgggctggcctggcattgctagccttgcctgtaattgcaagcgcatcatgcgcgcttcctagcacctacaaatggacttcgactggcccgctggcaaaccccaagtcgggctgggtttcgcttaaagatttcagccatgttccatacaatggccagcatctggtctattcttcgactgtgaactcggcaggttcctatggctcgatgaattttggtctcgtctcaaactggaccaacctcagcacagccagccaaaacacaatgaaccttggcacggtagcgccaactctcttctatttctctcccaagaagatctgggttcttgcatacgaatgggctgctactccatttgcctatgtcacgtcgaccgatcctaccaatgccaatggttggtcagcttcgcaaccgctcttcagcggaagcatatctccctctagccctatcgacccagcactcatcagcgatggcacgaatatgtaccttttctttgcaggagataatggcaaaatctaccggtccagtatgcccattggccagttcccatccagcttcggcacatcgtacacaacgatcatgagcgccgcaaccaacgatctgtttgaagcgatacaggtgtacactgtctcaggccagaatcaatatcttatgattgttgagtgtatcggatctgttggacggtatttccgctcatttacggccacatccctgagcggtacatggacgccgcaagctgcaaccgagagcaatccctttgcaggccatgctaacagcggagctacttggactaacgatatcagctctggagatcttattcgcagcacaaacgatgagacgatgaccattgatccctgcaacctccagctgctgtaccaaggaatggccgttggttccaccggagactataactccctgccatggcgacccgccgtcttgactctaaccaaccccggctcgagcacaggcaacggcaacggcactggaagcggcggctcaggcggcagcggatctggacaagcatcgcagtatgctcagtgcggcggtttgggctatactggacccacggtttgccaggcacgtaatgctgttccaagtttcttgataatgaaattgctaacacgagatcagagcccgtacaaatgcacgtttgtcaacgaatactactcccagtgtctatag

SEQ ID NO: 80 - DNA, 1047 bp, Trichoderma hamatum
atgctctacactgctgctctggccggccttatggccactgctgcccttgctgccccctaccgccgacaggctactggccaaaacgttgtatactggggccaaaacggcggcggcgttactgaaagtaacaatttgtctgattattgcgatgctgaagcaggcatcgatctacttgttcttagtttcctctacgagtatggttagtttcaccccgttgtgcctttgcaaattgcaatcttgtgtactactcttactaaccattttgtaggcaatggaaatactattccttcaggcactatcggccagagctgctccattgatacctccggaaacccttcaaactgtgatgcccttgcctcggccattgctacctgcaagtccaatggagtcaaggttgtcttgtctctcggcggcgccgttggagcctactctctctcctctcagcaggaggccgagacaattggccaaaacctctgggacgcatacggtgccggcaatggaaccgtccccagaccgttcagaagcaccgttgtcgatggctgggactttgatatcgaagccagtagtggcaaccagtattatcaatacttgattgccaagcttcgctcaaatttcaacggcggcaactacgtgattaccggtgctcctcagtgccctattccggagcctaacatgcagcaaatcattaccacttctcagtttgactatctttgggtccagttctacaacaatccctcgtgctcagttggaacgtcgacaccaaacttccaagattgggtttccaacattgccaacactccttctgccaatgccaagatcttccttggtgtgcctgcaagcccgctcggcgctactggaactgaatctggcgcccaatattacctcgagccttctgctctcaacactctcgtcggccagttctcatccaaccctgcgtttggcggtatcatgatgtgggctgccggtttctccgatgccaacgtgaacaatggatgcacttatgctcaagaggctaagaagatccttacttctggtcaaatctgctaa

SEQ ID NO: 81 - DNA, 1342 bp, Trichoderma hamatum
atgctttcccacgctatccttgctggcctcggccttgctggtctcgctgccgctgcgcctaccaggacgattgcgactcgtcaagcttccggttatcagaacgctgtctactggggtgccactggcaaacagaaccctaacctcgctgaatactgcacgtccacctcgggcattgatatcctgatcctgtccttcttggacgtctacggtgccactggcaacttcccctctggcaactttggcaacgactgcttcgtcggcaccacgggtgttccccagtcatgctctgacctggcctcccagatcaagacctgccagaacgctggtgtcaagattattgtttctctcggtggtgccgccggttcatactctgtcacgtcccagcagcaggcccagaccatcggtcaatacctctgggatgcctacggtaactctggcagcacctctgtccagcgtccctttggcgatgtctttgttaacggttgggactttgacatcgaggccaacggcggcttcagccagtactaccagtacatgattggcaccctgcgctccaacttcgccaaggactccgccaacacgtactacatcaccggtgctccccagtgccccctgcccgaggagaacatgggcgatatgatccagcactccgtcttcgactacctcttcatccagttctacaacaacaaccccacctgctctcttggtctgtctggccaggctcccctcaactacgacgactggaccaactttgtctccaccacccagtccaagaacgcgaagctcttcctcggtgcccctgccggacctctcgcctccaacggaaacccaaacggtgccgtctactacgctaccccctcggatctggcccccatcgtcaacaaggccaagacgaagtcaaactttggcggtgtcatgctctgggacgctggttactctgatgagaactccagcggcggttgcaactacgcccagcaggtcaagagcatcctcaccaccggcgccccttgcaacggcacccccgtcagcggtggtggctctcctcccgctacttcttccactgcttcttctcctcccgctacttcttccactgcttcttctcctcctgctacttcttcccttcctgccgatggaagcaccggaagcaccggtaacagcggcgttggcagcgttgcccagtggggacaggtaagataattttcttcctattagttatagaaagacatttttgctaacagttgtttgataatagtgcggcggtattggttacactggtcctacccagtgccagtccccattcaagtgcgttaacgagggcccgtattggtcttcttgccaataa

SEQ ID NO: 82 - DNA, 1270 bp, Trichoderma hamatum
atggccggttcacttacagcagacgctcacgaagctgttcggtgcatcatgtatctcacagggtgagcttctttttggcccggcttctacttggcttacagaatatctaacgctttcgtaggcaacatgtggtagtcccttcagaccatgatctcgtgggttccataacacatgtgatactggcattcatgcgctctgatgtcttcaatgtggataaaacgcctgccgagttcccgttttttacaaccgttgctgaaacacggcagaagttcaatgcgaatactaaaatcatggtcgcaattgggggttggggggattctgcaggatttgaagaagctgcgcgtgacgattcgtcgagaaagcggtgggctaaccaagtaaaggccatggttgacctgacaggagctgatggcattgacattgactgggaatatccggggtaagttgatggtgaatcgcagtacccaccgcagagcagttttcatctaacattatttccaggggaaatcgtgacgactataaacttattccgaattctcagcgggaatgggagatagaggcattcgtgcttcttcttcgagaacttcgtttggttttgggagaagaaaaactactcacaattgcggtgccagcgctagaacgcgatttgatggctttcacaaactcaactattccgtccatcgtgaatcaggttgacttcattagtgtaatgacttacgatatgatgaatcgacgtgataccattgtcaagcaccatagtggtgtggccgactctcaggaagcaatggagcgatatatagatcgtggggcccctccgcacaaattgaacttcggacttggttactatgccaaatggttcatgaccgagcaatgcgatttacagcacccattggggtgccgcactcaactgctagaagaccccgccaatggagccgaccttggcaagactgcagcttttagctggcatgacgaggttcccgtggaattggctaattctttcgagaaagctcatactcatggccgctactatgaagatggaagctacgggtattgggatgatgaagagaagagatggtggtcctacgacacgcctctcgccatcaaaactaaagtccctcggtttctcggcgaactgcaattgggcggtgtgtttgcctgggggctgggggaggatgctccgcagtttattcacttgaaggccactactgatgggattcgggctttgcgcggaggccagagctcacgggattcggtgaaagatgaactgtaa

SEQ ID NO: 83 - DNA, 1545 bp, Trichoderma hamatum
atggcgcctttcaatactatgcttgggtatctcctgttagtggtagcccctttggcggctgccctgccgttccgtggccatggccccaacacccataaccacatcaacttgaacttcaacctcaccgaaaccatcaacgagatcaatgaaaacttggctggcctagttggatacatcaccaaccctcacgccaagcacatcgtcgccaaccgctacattgtcgtctacaacaacaccttcggctccgaggccatcgccgccaagcaggccgagttcgctgccaccatccagaagcgcaaccttggaaagcgaagcctcggcggcaacatgctgtccactgaaatccactccttccagatgcacacctggcgcgccatggctctcgacgctgacgatgagatgatcaagtccattttcgccgcgaaggaggtcgcctatatcgaggctgataccgtcgtccagaccaaggctctcgttgcccagaccaacgctacccccggcctcatccgtctctctaaccaaaacattggtggcaagaactacatctttgacaactctgccggcgctggtatcactgcctacgttgtcgacactggtattagaatcacccacactgagtttgagggccgtgcttctttcggtgccaactttgtcaactccaacgtatgtaacatgccctcctcatacgcccaaactggtgaatgaagtttgcccatcatgaacaaaaagctaacaatagcgcttagaacactgatgagaacggccacggcagccacgtttctggtaccattggtggtgctaccttcggcgttgccaagaacatcaagctcgttgccgtcaaggtccttgatgcctctggtgccggtagcaactctggcgttctgaacggcatgcagttcgtcgtcaacgacgtccaggccaagggcctctccggcaaggctgtcatgaacatgtctctcggtggctccctgtccgctgctgtcaacaacgctattgctgccattgccaacgctggtgtcgtccctgttgttgctgctggtaacgagaacgtacgtttatatccttcccactcgtctcctctgaaaaaaacgttgtttttaactcgcattatagcaagatactgccaacacctctcccggctctgcccctcaggccatcaccgtcggtgctatcgatgccaccaccgatatccgtgctagcttctccaactttggcgctgatgtcgatatctacgcccccggtgtcaatgttctcagcgttggcatcaagtctgataccgatactgccgttctcagcggcaccagcatgggtaagaacccaaacccttacataaaaaaacctttgtatcctaaactaatccgcgttcttcatatagccactcctcacgttgccggtctcactggctacctgatggctctcaagggtgttaccaacgtcaacgatgtcaccaacctcatcaagaacctcgccactgcttccggcgcctcggtccagcagaacgttgccggaaccaccaacctcatcgccaacaacggtgaactgtaa

SEQ ID NO: 84 - DNA, 555 bp, Trichoderma hamatum
atgggctatagccgcaaagataaatcgaggtacatcaacacaattcaagaagcacttgacatctctacagctttcgttgccagcagccgaaaaagaatagctttaacttggaagattatcattcaaattcaatccaagtaagaattacaatctgaagtattactaggattacatttaaacagtctatctagaatgcaattcttcgctctcctccctctgttatacaccaccgctgcagcccttggcatcaattgtcgtgggaacagcaactgcgtcggtactccagaatgcagactcgccgatctgatcttgcaagtcagccagcaagatcccagcacatcatacagccctggtcagcacattgcttgttgcggcatacctggcggcaatatctgtgcattcactcagggcattagcaattcgattacagctggagaagccctgggtatgctgcaggggctatccgcccacggttgtggccaatgcggcagtattcccttcaaggataacaatgtcgctgagggacaattgacagtcaactggaccgatcactaa

SEQ ID NO: 85 - DNA, 1411 bp, Trichoderma hamatum
atggttcaatctgcggcttatctaggagccttgctggccaccctgcccttggcacgtgccggtttcaacgctagctcgacgcaaaacattgccgtgtactggggtgagctgaacatattcttgctgaatatgagcttccaattaatacattgcgttcaggtcaaaactcggcaaatcaagcaacctctcagcagcgattgtctacttactgtgccagtaagttcaagatcgtcctagaacaattcatgacagctaacgattatttctagatgctgatatagatgtgggcattaccaaaaaccttggatgtcactctgtagcctggctgacctctcacagatcatcccaattgggttcatgaacggcatcagcccggtcattaccaactttgccaatgcaggcgacaattgcacggcttttgcagataatcagaatgcacttaattgtccccaaatcgagtaagttgcccccagactctcagtgaatttcgccagtatacgatgcttagatatcctttaggcaagacatcattacatgccagcaaacatatggcaagacaatcctgatttcgctggggggcggatcttatacccagggcggcttctcgtccactggtgtcgccacatcagcggctcagacggtttggaacatgtttggccctgtcaatccaaacagcaacgtagaccgtccatttggttccgcagtggttgatggcgttgactttgactttgaatctggcgtcaataacttggccacttttgccactgagctgcggagcttgatggacgcttctgcgtcttctgcgaacaggaagttctacctgtctgctgcgccgcaatgcgtctatcccgatttcgctgacaacccggccctgaatggttcggttttcttcgatttcattatgattcaatattacaataatggatgcggagtcagcagctatgtccctggagcaactacccagtggaactacaactttgatgtctgggacaactgggcgcataccgtcagcaagaaccctaacgtgaggattctgcttggtatcgccgccaacactggtgctgccagtggatacgtgtccggaacacagctttctgcggtcatctcatttaccaagcaatactcaagctttgcgggtatcatgatgtgggatatgtcccaattgtatcagaattccggcttcttgggccaggttgtcagtgatctcgccgcctctggttcaacccccccggctactacttctagtggagcttccaagactaccacttctagtggcggttctacaagcccaactggcggcagtgtgccgcaatggggtcaatgtggaggcgaaggatacactggccctacgcaatgccagtcgccttacaagtgcgtttttagcagtcagtggtggtcatcttgccagtaa

SEQ ID NO: 86 - DNA, 1983 bp, Trichoderma hamatum; identical to SEQ ID NO: 55

SEQ ID NO: 87 - DNA, 1468 bp, Trichoderma hamatum
atgttgggcttcctcggaaaatccgtggccttgcttgctgcgctgcaggccaccctcacctctgcatctcctctgtctacaaacgacgtcacagttgagaagagagccagcggatatgcgaacgctgtctacttcactaactggtgagtgaagctaatttgtgattatgaattttagtgctaactattggtgattaaaggggtatctacggccgcaactttcaaccccaggacctggttgcgtcggacatcactcatgtcatctactcgttcatgaacttccaagcagacggcactgtgtaagttttgtaaccaagagatggtgtatcctaaatatctattttcagttgctgatcgtttcctctatagcgtctctggagatgcttacgccgattaccagaagcactattccgatgattgtacgataaccccccctttaagtgctcttaattctaagctttgcaaatatactaacatctatctcagcttggaatgatgtcggcaacaacgcgtacggttgtgtcaagcaactgttcaagttgaagaaggccaaccgcaacttgaaggttatgctttctataggtggctggacctggtccaccaacttcccttctgccgcaagcaccgatgccaaccgcaagaactttgccaagacagccattactttcatgaaggactggggttttgatggtattgacgtcgactgggagtatcctgccgataacacccaggccaccaacatggttcttctgcttaaggagatccgatctcagctagatgcttatgcggcccaatacgctccaggataccacttccttctctccatcgctgcccccgctggcccagagcactactctgccctgcacctggccgaccttggtcaagttctcgactatatcaaccttatggcttatgactatgctggttcttggagcagcttctccggacatgatgccaacttgtttgccaacccctccaaccccaactcttcaccatacaacaccgatcaggctatcaaggcttatatcaacggaggtgttcctgcacgcaagatcgttcttggcatgcccatctatggacgatctttcgagagcaccggtggaattggccagtcttacagcggaattggatctggaagctgggagaacggtatctgggactacaaggttcttcccaaggccggtgctacagtccagtacgactctgtcgcacaggcatactacagctatgaccctagctccaaggagctcatctccttcgatacccctagcatgatcaacaccaaggtctcttacctcaagaacctcggcctgggaggcagtatgttctgggaggcttctgctgacaagactggctccgactccttgatcggaacaagccacagagcgcttggaagcctggactcaactcagaacttgctgagctaccccaactcccagtacgataacatccgaagcggtcttaactaa

SEQ ID NO: 88 - DNA, 1308 bp, Trichoderma hamatum
atgtttttcagcaaagcactggctgctaccggccttttggccactgctgctctcgctgcacccaccgtggagaagcgtgccgctggaggcaagctcgtcatctactggggtgctgaagacgacagcactactctcgccaacgtctgtgccgattcctcttacgacattgtcaacttggctttccttgacaagttctctgctggtggtggctacccctcgctctctctgagcaccctgggcggcccatctgccgctcagaaggctgctggtgccaccaacctccaggatggttcttctttggttcccgccatcaaggcctgccaggccgccggcaagctggtcatcctctctatgggtggtgctaccgacttctctgcagtctccctttcgggcgatgctcagggacagtctgttgccgacatggtttggaacctgttcttgggtggtactgccaaccccactctccgtcctttcggatcagtcaagctcgacggtgttgatctcggtgagtaaaagtcattcctaacatgatctatgaattctacctttactgacatctcatcctgcaatatagacaacgagactggcaaccccactggttacctggccatgactcaacgcttccgatccaactttgccaaggacaccagcaagaggtactacctcactgctgctccccagtgccccttccccgatgcctctgagcctctcaacgtctgccagctccttgattacgtctgggtccagttctacaacaacggcaactgcaacgttggccagtccggcttcaacactgctgtcaagaactggagcaagaacattggtaacgctactctgttcattggtgcgctggccagcggtgccgatggtgaccagggctacatctctcccagcgctctgatctctgcttacaacggtgtctctgctcttaacctgcccaacgttggcggtatcatgctttgggaggctcagctcgctgtcaagaacagcaacttccagaagaccatcaaggctgccatcggctccggctccactcctcctcctcctcctcccgcctcttccaccactcctgccggaagcacccccacctgctcttgggccggccactgcgctggcgctacttgcagcactgacaatgactgctccgactccctcacctgcaacagcggcaagtgcggtaccgctggcagcactgctcctcctcctaccacttgctcttgggcgggccactgcctcggcgcttcttgcggtaacgacaacgactgctctgacccttacttctgctccaacggcgtctgctctcagtaa

SEQ ID NO: 89 - DNA, 980 bp, Trichoderma hamatum
atgtttttccgcaacgccgtcgccgtaaccagccttttggctgcactttctagtgcccaaccctccggccccgaattggccgtgtactggggtgctgaagatgatagcacgactctcagcgacgtgtgttcagacaactcttatggcattgtgaatcttgccttcctcgacacattttttgctgcaggaggctttcctcaactcagcgtcagcgggttagatggcccatcccaagctcagcaaagcgctggtgccactggcctcaaggatggtagcagtcttgtagacgctattaagcagtgccagtcggccgggaagctggtccttctcagccttggtggcgctggcgccgatgtcactcttcaatcagactctgatggagagaaaattgctgatacactctggaatctgttcggtggtggcactgataaccaagagctgcgtccctttggagatgtcaagctcgatggcttcgacttgggtgagtaacattaatgaattaacacgatcaatgtggctaactctatgcagacaatgaatctggcaaccccacaggttacctagctatggtaaagcgtttcaagtccaacttccagtcggacacgagcaaaacttatttccttactgcggctcctcaatgcccattccccgatgcttcgcagcctcaagatgtttgcagtgaattggactttgtctgggtccagttttacaacaacggcgattgcaacattgcgcaatctgatttcttaaactccgtccagacttggagcagcggcattggcaacgctaagctctacatcggtgctttggctagtggtgctgacggcgatcagggctttgctgatgctgatacgctattgggtgctattcaggatgtcaagaatatgaaccttcccaattatggaggtgccatgctttgggaagcccaactggctgttaaaaacggcaactttcagcagaagattgcccccggtctataa

SEQ ID NO: 90 - DNA, 1309 bp, Trichoderma hamatum
catcgagaagttcgagaaggtaagctcatttcgctgcttttttcattccttttgggcacaattgtgccagacaattctgttctcagtcttgtcaccatttttgcccaccaagcatcgcaccccgctttgtctacctacccctcctttggcacagcaaaatttttctggctgcctgggttggtttttagtggggtgccaaatttttggcagtgaccccgccatcgccactgttcctcatctcatgcattacccaacataatcttcagtcaattgcttggttcattgtgctaatcatactttaatcaataggaagccgccgaactcggcaagggttccttcaagtatgcgtgggttcttgacaagctcaaggccgagcgtgagcgtggtatcaccatcgacattgccctgtggaagttcgagactcccaagtactatgtcaccgtcattggtatgttttcagtccgactggtcactatcccatcatcatcatgctaacgtgcgactctacagacgctcccggtcaccgtgatttcatcaagaacatgatcactggtacctcccaggccgattgcgctatcctcattatcgctgccggtactggtgagttcgaggctggtatctccaaggatggccagactcgtgagcacgctctgctcgcctacaccctgggtgtcaagcagctcattgttgccatcaacaagatggacactgccaactgggccgaggctcgttaccttgagatcatcaaggagacctccaacttcatcaagaaggtcggcttcaaccccaagaccgttgcctttgtccccatctctggcttcaacggtgacaacatgctccaggcctccaccaactgcccctggtacaagggttgggagaaggagaccaaggctggcaagtccaccggtaagaccctcctcgaggccattgacgccatcgagccccccaagcgtcccacagacaagcccctccgtctgccccttcaggatgtctacaagatcggtggtatcggaacagtccctgtcggccgtatcgagactggtgtcctcaagcccggtatggtcgttaccttcgctccttccaacgtcaccactgaagtcaagtccgtcgagatgcaccacgagcagctcgttgagggtgtccccggtgacaacgttggattcaacgtcaagaacgtctccgtcaaggatatccgccgtggtaacgttgccggtgactccaagaacgacccccccatgggtgccgcttctttcaacgcccaggtcatcgtcatgaaccaccctggccaggtcggtgccggatacgctcc

SEQ ID NO: 91 - DNA, 707 bp, Trichoderma hamatum
ccgaggagcaccccgtcctgctcaccgaggcccccatcaaccccaagtccaaccgtgagaagatgacccagatcgtcttcgagaccttcaacgctcccgctttctacgtctctatccaggccgttctgtccctgtacgcctctggtcgtaccaccggtatcgttctcgactccggtgatggtgttacccacgttgtccccatctacgagggtttcgctcttcctcacgccattgctcgtgttgacatggctggtcgtgatcttaccgactacctgatgaagatcctggctgagcgtggttacactttctccaccaccgccgagcgagaaattgttcgtgacatcaaggagaagctctgctacgtcgctctcgacttcgagcaggagatccagaccgctgctcagagctccagcctggagaagtcatacgagcttcccgacggtcaggtcatcaccatcggcaacgagcgattccgtgctcctgaggctctgttccagccttctgtccttggtcttgagagcggtggtatccacgtcaccactttcaactccatcatgaagtgcgatgttgacgtccgaaaggacctgtacggcaacattgtcatggtaagtgaatttccgcattcaacaattgatttttatagcggcgctaatgtatttctcaatctagtctggtggtaccaccatgtaccccggtctctccgaccg

SEQ ID NO: 92 - DNA, 469 bp, Trichoderma hamatum
tctctctttgtgagtctctagacagtcttttgcgacaaatctgctggcgatttgggtactaatctgctggctacaggacaaggacggcgatggtacgtagtacctagtgacgcgatactcctcttttcccctcctcctcaccgtccccctattttgtgcgagctatcactagagcgcctgaacctgcgataccgatcgaaatatccccggctggcaatttttatgacgaataaacggacaagaaactaacagacttgacttggcaggccagatcaccaccaaggagctgggcactgtcatgcgctctttgggccagaacccctccgagtcagagctgcaggacatgatcaacgaggttgatgccgacaacaacggatccatcgatttccctggtatgtcaatagcagaaacacatagacagctgccggatacaggctaatctagagcggtgaagagttccttaccatga

SEQ ID NO: 93 - DNA, 1069 bp, Trichoderma hamatum
tggccaagctgttccgtggtatcatgcgcaggatgaataccgagttggccaactacctgagacgatgtgttgagggcaaccgccacttcaaccttgctgttggcatcaagcccggcacactctccaacggattgaagtattcacttgctaccggaaactggggtgaccagaagaaggcaatgagctcgactgcaggtgtatcacaggtgcttaaccgttacacttttgcctcgacactttctcatttgcgtcgtaccaacacacccattggaagagatggtaagctggcgaagcctcgacagcttcacaacacacattggggtttggtgtgcccagccgagacccctgaaggacaagcttgtggtctggtcaaaaacttgtctctgatgtgctacgtcagtgtcggatctccttctgagcctttgatcgagtttatgatcaataggggtatggaggttgttgaggagtacgaaccactgaggtatccccatgctacaaagatctttgtgaatggtgtctgggttggaatccaccaagaccccaagcatctggtaaaccaagttttggacactcgtcgtaaatcctatctgcagtacgaagtctctctgatcagagatattcgtgaccaagaattcaaaatcttctctgacgccggtcgtgttatgcgtcctgtctttactgtacagcaagaagatgacccggaaacgggtatcaacaagggccacttggtattgaccaaggacctcgtcaacagacttgccaaagagcaggctgagcctccagaagacccaagcatgaagcttggatgggaggggttaattagggctggtgcggtggaatatctcgacgccgaggaagaagaaacggctatgatttgcatgacaccggaggaccttgaactttatcgtcttcagaaagctggtatttccacggatgaagacatgggagacgatccaaacaagcgtctcaagaccaagacaaatccgacaactcacatgtacacgcattgcgagattcacccaagtatgatcttaggtatctgtgctagtatcattcctttccccgatca

SEQ ID NO: 94 - protein, 382 aa, Trichoderma hamatum
MAGSLTADAHEAVRCIMYLTGQHVVVPSDHDLVGSITHVILAFMRSDVFNVDKTPAEFPFFTTVAETRQKFNANTKIMVAIGGWGDSAGFEEAARDDSSRKRWANQVKAMVDLTGADGIDIDWEYPGGNRDDYKLIPNSQREWEIEAFVLLLRELRLVLGEEKLLTIAVPALERDLMAFTNSTIPSIVNQVDFISVMTYDMMNRRDTIVKHHSGVADSQEAMERYIDRGAPPHKLNFGLGYYAKWFMTEQCDLQHPLGCRTQLLEDPANGADLGKTAAFSWHDEVPVELANSFEKAHTHGRYYEDGSYGYWDDEEKRWWSYDTPLAIKTKVPRFLGELQLGGVFAWGLGEDAPQFIHLKATTDGIRALRGGQSSRDSVKDEL

SEQ ID NO: 95 - protein, 443 aa, Trichoderma hamatum
MAPFNTMLGYLLLVVAPLAAALPFRGHGPNTHNHINLNFNLTETINEINENLAGLVGYITNPHAKHIVANRYIVVYNNTFGSEAIAAKQAEFAATIQKRNLGKRSLGGNMLSTEIHSFQMHTWRAMALDADDEMIKSIFAAKEVAYIEADTVVQTKALVAQTNATPGLIRLSNQNIGGKNYIFDNSAGAGITAYVVDTGIRITHTEFEGRASFGANFVNSNNTDENGHGSHVSGTIGGATFGVAKNIKLVAVKVLDASGAGSNSGVLNGMQFVVNDVQAKGLSGKAVMNMSLGGSLSAAVNNAIAAIANAGVVPVVAAGNENQDTANTSPGSAPQAITVGAIDATTDIRASFSNFGADVDIYAPGVNVLSVGIKSDTDTAVLSGTSMATPHVAGLTGYLMALKGVTNVNDVTNLIKNLATASGASVQQNVAGTTNLIANNGEL

SEQ ID NO: 96 - protein, 309 aa, Trichoderma hamatum
MFFRNAVAVTSLLAALSSAQPSGPELAVYWGAEDDSTTLSDVCSDNSYGIVNLAFLDTFFAAGGFPQLSVSGLDGPSQAQQSAGATGLKDGSSLVDAIKQCQSAGKLVLLSLGGAGADVTLQSDSDGEKIADTLWNLFGGGTDNQELRPFGDVKLDGFDLDNESGNPTGYLAMVKRFKSNFQSDTSKTYFLTAAPQCPFPDASQPQDVCSELDFVWVQFYNNGDCNIAQSDFLNSVQTWSSGIGNAKLYIGALASGADGDQGFADADTLLGAIQDVKNMNLPNYGGAMLWEAQLAVKNGNFQQKIAPGL

SEQ ID NO: 97 - protein, 412 aa, Trichoderma hamatum
MFFSKALAATGLLATAALAAPTVEKRAAGGKLVIYWGAEDDSTTLANVCADSSYDIVNLAFLDKFSAGGGYPSLSLSTLGGPSAAQKAAGATNLQDGSSLVPAIKACQAAGKLVILSMGGATDFSAVSLSGDAQGQSVADMVWNLFLGGTANPTLRPFGSVKLDGVDLDNETGNPTGYLAMTQRFRSNFAKDTSKRYYLTAAPQCPFPDASEPLNVCQLLDYVWVQFYNNGNCNVGQSGFNTAVKNWSKNIGNATLFIGALASGADGDQGYISPSALISAYNGVSALNLPNVGGIMLWEAQLAVKNSNFQKTIKAAIGSGSTPPPPPPASSTTPAGSTPTCSWAGHCAGATCSTDNDCSDSLTCNSGKCGTAGSTAPPPTTCSWAGHCLGASCGNDNDCSDPYFCSNGVCSQ

SEQ ID NO: 98 - protein, 389 aa, Trichoderma hamatum
MGSFKAIIAASLAALGQFAAAAPAEGQSKRAGISSIVKGTPVGFASSVTGGGTVAPVYPTTIAQLKSYLTSTSPQNIVISGTFNFAGTEGTTTLPACNAYPCTPSNGGQALLNTLGGCGSLSTYNVNLDTAAYNAINVQSDKTLVGINGATLNGKGLRLSGVSNVIIQNIAITNLNPQYVWGGDAISLSNTNNVWIDHVKTSNLGRQHYSFGTGSNNAVTISNSFISGQTSYSASCDGHSYWGLELVGSGDQITFYKNYVYYTSGRTPALSGNTLFHAVNNVWSSNSGHAIEGTSNGMGLYEGNYFVNVPTIVASGFVGRLFSSQSSAVSQCAQYLGRNCVSNSLSNSGTFTNSDTSFLYLFQGKANIVSAASASSIQSSVVSSAGNTL

SEQ ID NO: 99 - protein, 169 aa, Trichoderma hamatum
MGYSRKDKSRYINTIQEALDISTAFVASSRKRIALTWKIIIQIQSNLSRMQFFALLPLLYTTAAALGINCRGNSNCVGTPECRLADLILQVSQQDPSTSYSPGQHIACCGIPGGNICAFTQGISNSITAGEALGMLQGLSAHGCGQCGSIPFKDNNVAEGQLTVNWTDH

SEQ ID NO: 100 - protein, 281 aa, Trichoderma hamatum
MHLHSLFTALALASPALAASIPRSSSSSSSSTITVWATPHDSYSSSVGVLGCKVDTNRIAYWPDSVDCTNICVSLSYQDRQVYLLRVDQSQGAHDISYDAWNYLVTGYPATEKPVAGGPMEMTTENVDASKCADLIYTAGGKLPLSAANSMNFLASCLEQENSWVASNYVLYNILDAICTVGQNQVCSLNWPTANQPTCPGTLGLPDALKGEPVYNIEYPSGVKVLAGAPPTVPTGVPVPVPAPSTNDDEKSAARSLRHSDSLVWIPVLSLTSIIYSWMLW

SEQ ID NO: 101 - protein, 420 aa, Trichoderma hamatum
MHSTTFFASLGLAGLVAGAPSAPHNVKARQASGAQNVVYWGGTNNESDDLSTYCTPTAGIDILVLSFLDIYGTTGNIPAGNIGNSCYIGTNGTPQLCDNLAASIASCQAAGIKIILSLGGAAGSYSLQSQSQAVAIGQYLWEAYGNSGSTSVQRPFGNVFVNGFDFDLELNAGNQYYQYLISTLRSNFASDPKHTYYITGAPQCPLPLSIPQIRKPNMGEIISASQFDYLWIQFYNNNAYAPDPCSLGLPGDAPFNYNNWTSFIATTPSKNAKLFIGVPANTLAANGNSGGAVYYASPSQLASIVANTKSSPDFGGIMMWDAGYSDANVNNGCNYAQEAKNILLTGAPCGGSPPPVSSSKPTSTATKSATSTSSASGTGPTGGGTVPQWGQCGGEGYTGPTQCVSPYKCVESSQWWSSCQ

SEQ ID NO: 102 - protein, 98 aa, Trichoderma hamatum
MKFFAATALLAATTIAGPLEVRTGDGNICPSGGLYGNPQCCSSLLLGLIGLDCNVPNQTPRDGADFRNICAKTGDEALCCVAPVAGQALLCQVAVGAS

SEQ ID NO: 103 - protein, 97 aa, Trichoderma hamatum
MKFFAVATLFVTGVISAPSPNALNSRSLLCSPGLYSTAQCCGVDVLGVADLDCAAPTGTITNAQGFQAACAKKGQEARCCVLPVAGQDVLCQDPPGL

SEQ ID NO: 104 - protein, 135 aa, Trichoderma hamatum
MKFSFTSAIAIASIGITGARAGINCNGSGNCPGVAGDLSTLISFGWSIDPNRWYNNGEHIVCVQSQLGTGLCAFLQNTGGAPGSSIQPLLQALQGHGCNKCGSVPLNFLQGDNSEDHGELTVNAVGSTAGCSGIC

SEQ ID NO: 105 - protein, 89 aa, Trichoderma hamatum
MKMLITAALFTLALAAPVAETKPHSIAARDPFTCPGGLTNSTPMCCSVNVLGLLALDCQQPGADGCSGSSKPNCCTLGAAGQGVICNAL

SEQ ID NO: 106 - protein, 638 aa, Trichoderma hamatum
MKSAILLGLTGLAANVNAHPAKQPETGNGLTKRGVDISKYSLPNLSKYTSSPNIEQEASLQSLAFKRNYVDTATRAVKKAAPKAEFRVVDDHYIDVDGIGHVHFKQTVHGIDIDNGDFKVNIGKDGRVFSHGNSFFSGKLPQQNPLRKRDFSDPTTALKGAIDILGLPVQADGATAEAQEGTEKYTLKGTSGAVSDPKAHLVYLVKGDGTLALTWRVETDIMDNWLLTYVDATTNQEIHGVVDYVSDLATFQVYPWGLNDPTEGDRKVLTDPWRTDASPFTWLSDGTTNYTVTRGNNAIAQDNPSGGDSYLNNHRPSSSTRDFQYPFTLTQTNPTDYRDAAITQLFYTVNKYHDLLYVLGFNEVAGNFQANNNGKGGKANDFVIVNAQDGSGTNNANFATPADGSNGRMRMYIWTTANPKRDGDLEAGIVIHEYTHGLSTRLTGGPANSGCLTGTEAGGMGEGWGDFFATAIRLKAGDTRSKDYPMGVWADNNVKGIRQYPYSTSLTTNPLTYKTVNTQNEVHSAGTTWATILYEVLWNLIDKYGKNDDDFPTFDSQGVPTDGKYLTLKLVLNGLALQPCTPTFVSARDAILDADRALTGGENLCELWTGFAKRGLGSGAKYSSTARVESFTIPSGVC

SEQ ID NO: 107 - protein, 131 aa, Trichoderma hamatum
MKSSLLSLIAIAINGALAGINCNGSGDCAGTPGTLGDLIADAYQIDPNRWYNNGEHIACSDNRGGGGLCAFFQNTLGGPGSSVLTLLQNLQAHGCNKCGSIPVNFPQGDNSENHGELTVNFVVSAGCTGLC

SEQ ID NO: 108 - protein, 138 aa, Trichoderma hamatum
MLFKIAIVVIASTARVSAFGINCEGSGYCSPLFNPGANNHPLLEMVDVIDFGIDDNRWYAAGEHIACDQSSGVCAFVQKIGGASGGDIARAVRYLADHGCTTCGSVPLDFPNTNDVNNGEVTFNFVGLEDMGSCSDLC

SEQ ID NO: 109 - protein, 424 aa, Trichoderma hamatum
MLGFLGKSVALLAALQATLTSASPLSTNDVTVEKRASGYANAVYFTNWGIYGRNFQPQDLVASDITHVIYSFMNFQADGTVVSGDAYADYQKHYSDDSWNDVGNNAYGCVKQLFKLKKANRNLKVMLSIGGWTWSTNFPSAASTDANRKNFAKTAITFMKDWGFDGIDVDWEYPADNTQATNMVLLLKEIRSQLDAYAAQYAPGYHFLLSIAAPAGPEHYSALHLADLGQVLDYINLMAYDYAGSWSSFSGHDANLFANPSNPNSSPYNTDQAIKAYINGGVPARKIVLGMPIYGRSFESTGGIGQSYSGIGSGSWENGIWDYKVLPKAGATVQYDSVAQAYYSYDPSSKELISFDTPSMINTKVSYLKNLGLGGSMFWEASADKTGSDSLIGTSHRALGSLDSTQNLLSYPNSQYDNIRSGLN

SEQ ID NO: 110 - protein, 660 aa, Trichoderma hamatum
MLLKQILVATLLSLSSVKAFPNPNHAQDLEAREAEDNLVARGGHDCGSYATWNYGKNCCVCKDNGKNYDSKSKTCSCPHGQVWNGKQCVVDCGKDATYDWKQKKCVCKKQGEVYNSNTKTCSCPPGTVWNGYGCVVDCGKEAHFDKWQKKCVCNNNGEVYDSKSKTCSCPGGQYWNGKKCVCPYGKVWNGKQCIEDCGKDAHFDYNQKKCVCNKNGQVYNSNSKTCKCPGGQYWDGKHCVCPYGQVFNGKQCVPDCGKEAHFDYKQKKCVCNNYGEIYNSKSKTCACPDGQYWNGKQCVCPYGKIWNGKQCVPDCGKDAHFDYNQKKCVCNKNGEIYDSKSKTCACPDGQYWNGKQCACPYGKIWNGKQCVPDCGKEAHFDYNQKQCVCNNKGEIYDSKSKTCACPDGQYWNGKQCACPYGKIWNGKQCVEDCGKEAHFDYNQKQCVCNNKGEIYDSKSKTCKCPDGQYWDGKQCACPYGKIWNGKQCVEDCGKDAHFDYNQKQCVCNNKGEIYDSKSKTCKCPDGQYWDGKQCACPYGQIWDGKQCTPNCGKDATYDSKQKQCVCNKKGQVFDSKHLTCSCPAGTSWNGYACVPDCGKDAHYDTNQKCCVCNNKGQIFNSGSKTCSCPGNQYWDGNKCTCPYGSTWDSSKKTCKQTPIH

SEQ ID NO: 111 - protein, 238 aa, Trichoderma hamatum
MLPSSLCRIAAVISVASAEMVSVTFDTAYDDPSRSLSEVACWRKGTGFMPNLDWKLQKDALEFIGIKAIRGFSSAQCFSCWKIEYGDKQISLFAIDSADSGIVLSLSTMQYLTGGQARELARIDAKATQVDASNCGISAAELHKYDFYTNGSGNSDGSRSNTGAIVGGVLGGVAGLALTGLAVFFFPRYKNLAAGHKPVSQNTPPAAYQSSLMQQDPKAHYDPKYFAEMDTQPYTPPD

SEQ ID NO: 112 - protein, 402 aa, Trichoderma hamatum
MLRTPTIPWLLNIILEENPTMIPSMKKCGRKLWAGLALLALPVIASASCALPSTYKWTSTGPLANPKSGWVSLKDFSHVPYNGQHLVYSSTVNSAGSYGSMNFGLVSNWTNLSTASQNTMNLGTVAPTLFYFSPKKIWVLAYEWAATPFAYVTSTDPTNANGWSASQPLFSGSISPSSPIDPALISDGTNMYLFFAGDNGKIYRSSMPIGQFPSSFGTSYTTIMSAATNDLFEAIQVYTVSGQNQYLMIVECIGSVGRYFRSFTATSLSGTWTPQAATESNPFAGHANSGATWTNDISSGDLIRSTNDETMTIDPCNLQLLYQGMAVGSTGDYNSLPWRPAVLTLTNPGSSTGNGNGTGSGGSGGSGSGQASQYAQCGGLGYTGPTSPYKCTFVNEYYSQCL

SEQ ID NO: 113 - protein, 425 aa, Trichoderma hamatum
MLSHAILAGLGLAGLAAAAPTRTIATRQASGYQNAVYWGATGKQNPNLAEYCTSTSGIDILILSFLDVYGATGNFPSGNFGNDCFVGTTGVPQSCSDLASQIKTCQNAGVKIIVSLGGAAGSYSVTSQQQAQTIGQYLWDAYGNSGSTSVQRPFGDVFVNGWDFDIEANGGFSQYYQYMIGTLRSNFAKDSANTYYITGAPQCPLPEENMGDMIQHSVFDYLFIQFYNNNPTCSLGLSGQAPLNYDDWTNFVSTTQSKNAKLFLGAPAGPLASNGNPNGAVYYATPSDLAPIVNKAKTKSNFGGVMLWDAGYSDENSSGGCNYAQQVKSILTTGAPCNGTPVSGGGSPPATSSTASSPPATSSTASSPPATSSLPADGSTGSTGNSGVGSVAQWGQCGGIGYTGPTQCQSPFKCVNEGPYWSSCQ

SEQ ID NO: 114 - protein, 325 aa, Trichoderma hamatum
MLYTAALAGLMATAALAAPYRRQATGQNVVYWGQNGGGVTESNNLSDYCDAEAGIDLLVLSFLYEYGNGNTIPSGTIGQSCSIDTSGNPSNCDALASAIATCKSNGVKVVLSLGGAVGAYSLSSQQEAETIGQNLWDAYGAGNGTVPRPFRSTVVDGWDFDIEASSGNQYYQYLIAKLRSNFNGGNYVITGAPQCPIPEPNMQQIITTSQFDYLWVQFYNNPSCSVGTSTPNFQDWVSNIANTPSANAKIFLGVPASPLGATGTESGAQYYLEPSALNTLVGQFSSNPAFGGIMMWAAGFSDANVNNGCTYAQEAKKILTSGQIC

SEQ ID NO: 115 - protein, 540 aa, Trichoderma hamatum
MPLKVFESASPAPSHPSLSHLCLLVFEAVLEVVCVSLPGYIVARLGHFDADKQKFLANLNVMLFTPCLIFTKLASQLNAEKLSDLAIIPAIFIVQTLVSWIVSILVAKGFRFNKRASNFVTAMGVFGNSNSLPISLVLSLSQTIKGLHWDRIPGDNDDEVGARGILYLLIFQQLGQLVRWSWGYHVLLAPKDKYAEYQDEIAEEGQYRYRDEEPNEQEPEILITGLDGDTEDDGESNASEDYIPAGRTPLASNSRASLAGSSVDNDDMLNFKKGNYTRGSSLANTDLEDDILSFPRIRLRDEAEVEHGVTARIKKSLYSLKDKASAAMTRQYQRLPQPVQTCLSFIHKSITKTLGFVWDFMNPPLWAMLIAVIVASIPSLQQLFFEDGSFVKNSVTNAVSSSGGVAVPLILVVLGANLARNTAAHDSPIDPEEEKIGTKLLIASLLSRMVLPTLIMAPILAITAKYLPISILDDPIFIVVCFLLTGAPSALQLAQICQINNVYEQTMGRILFQSYVIWILPSTLFLVMMALEVIEWATVN

SEQ ID NO: 116 - protein, 331 aa, Trichoderma hamatum
MPSLITVASVLALVPSAFAGWNVNSKQNIAVYWGQNSANQQSTQQRLSAYCNAKIESDSCDVDANINVIDIAFLNGITPPMTNFANAGDRCTPFSDNPWLLSCPEIEADIKTCQANGKTILISLGGDSYTQGGWSSTSAAQAAAKQVWAMFGPVQSGSSTERPFGSAVVDGFDFDFEATTNNLAAFGAQLKSLSAAGGKKYYFSAAPQCFFPDAAVGALINAVPMDWIQIQFYNNPCGVSGYTPGTSNQNNYNYQTWDTWAKTSPNPNVKLLVGIPAGPGAGRGYVSGSQLTSVFQYSKGFSSTFAGAMMWDMSQLFQNTGFEAQVVNALK

SEQ ID NO: 117 - protein, 138 aa, Trichoderma hamatum
MQLSNLFKLALFTAAVSADTVSYDTGYDDASRSLTVVSCSDGPNGLITRYHWQTQGQIPRFPYIGGVQAVAGWNSPSCGTCWKLTYSGKTIYVLAVDHAGAGFNIGLDAMNALTNGQAVALGRVSATASQVAVSNCGL

SEQ ID NO: 118 - protein, 101 aa, Trichoderma hamatum
MQLTTVVALFASLAGAAPAPEPAAELVARDGPCSSGVTNNVPQCCGTGILSVVYVDCKTPTQAPSATNQLSAICARVGLQAKCCTVGIAGVGVLCQDAIPQ

SEQ ID NO: 119 - protein, 152 aa, Trichoderma hamatum
MRFNSFIAVAAATAQSAAGLGINCLGDTLCGISYMSGGRLTQFQTIFDNIFEKRIYDNGDDIGCIEVHSINFKGSFKGTYCAYVQNLDGSVNGATLKTLYTELVNYGCGICGSIPIHYSKGDNDSNHGELSFNMVDSLPDNCELGKPCAATS

SEQ ID NO: 120 - protein, 98 aa, Trichoderma hamatum
MRTSTAIISLALVVMGIAAPAAEAGSVVSEAGFVVSEAQFNAIFPGRNPFYSYRALTGALGSYPSFTNTADNATRLREAAAFFANVDHETDGLKFVVE

SEQ ID NO: 121 - protein, 357 aa, Trichoderma hamatum
MSAASRKSRIATSVARVMYTNAVYFPSSRIYQGDSPGMLNYSCINHVYYAYASVTADGNVFLGDEWADARAPVDGVQGGLGSLMHLKQRHPHLQVVLSIGGSTASEVFPIVASSTLLRDNFARSALGLVEASGLDGIDIAWEFPSQAKHGHDFLALLAAVRIHMPEDRFILTAVLPAAKEVLQLIDLSTAAEYLDYINLVAYDFFGTWTSKTGHHAQLYTMNKDEPSASTGVAYVMSQGFPAKSILLGIPTYGRSFLKANGPGQDFNGVGGQDGTFEYGELPRKGCKEIVDRRYIAAQCVGGDGGFVTYDNPETVKVKAEFCKQKGLGGLFYWNGPADSRDQARSLIAAGFRALHTS

SEQ ID NO: 122 - protein, 344 aa, Trichoderma hamatum
MTRLLDASFLLLPAIASTLFGTASAQNATCALKGKPAGKVLMGYWENWDGSANGVHPGFGWTPIENPIIKQNGYNVINAAFPVILSDGTVLWENDMAPGTQVATPAEMCAAKAAGATILLSIGGATAGIDLSSSTVADKFIATIVPILKQYNFDGIDIDIETGLVNSGNIKTLSTSQANLIRIIDGVLAQMPSNFGLTMAPETAYVTGGSITYGSIWGSYLPIIQKYVQNGRLWWLNMQYYNGDMYGCSGDSYAAGTVQGFIAQTDCLNAGLTVQGTTIKVPYSMQVPGLPAQSGAGGGYMNPSLVGQAWDHYNGALKGLMTWSINWDGAGGWTFGDNLLTRIG

SEQ ID NO: 123 - protein, 409 aa, Trichoderma hamatum
MTSIRRLALYLGALLPAVLAAPAALHKKPEAAPNKFIVTLKEGASIDTDSHLAWVNDIHRRSLTKRSTAGVEKTYNIHTWSAYAGEFDAETIEQIKSSPDVASVEPDYIMYLSDIVEDKRALTTQSGAPWGLGTVSHRTSGSTSYIYDSSAGSGTFAYVVDSGINTSHQQFGGRASLGYNAAGGQHVDTLGHGTHVSGTIGGSTYGVAKQASLISVKVFAGESATTSVILDGYNWAVNDIVSRNRASKSAINMSLGGPASSTWTTAINAAFNQGVLTIVAAGNGDSFGNPQPVSGTSPANVPNAITVAALDINWRTASFTNYGAGVDVFAPGVNILSSWIGSNTATNTISGTSMATPHVVGLALYLQSLEGLTTPTAVTNRIKALATAGRITGSLNGSPNTLIFNGNSA

SEQ ID NO: 124 - protein, 394 aa, Trichoderma hamatum
MTSLYLTSALGLLCLLPAAQAGWNPNSKNNIVVYWGQDAGSIGQNRLSYYCENAPDVDVINISFLVGITDLNLNLANVGNNCTSFAQDPNLLNCPQVAADIVECQQTYGKTIMMSLFGSTYTESGFSSSSTAVSAAQEIWAMFGPVQSGNSTPRPFGNAVIDGFDFDLEDPIENNMEPFAAELRSLTSAATSKKFYLSAAPQCVYPDASDQTFLQGEVAFDWLNIQFYNNGCGTSYYPSGYNYATWDNWVKTVSANPNTKLLVGTPASVHAVNFANYFPTNDQLAGAISSSKSYGSFAGVMLWDMAQLFGNPGYLDLIVADLGGASTPPPPASTTLSTVTRSSTASSGPTSPPPSGGGSVPQWGQCGGQGYTGPTQCQSPYTCVVESQWWASCQ

SEQ ID NO: 125 - protein, 394 aa, Trichoderma hamatum
MVQSAAYLGALLATLPLARAGFNASSTQNIAVYWGQNSANQATSQQRLSTYCANADIDIIPIGFMNGISPVITNFANAGDNCTAFADNQNALNCPQIEQDIITCQQTYGKTILISLGGGSYTQGGFSSTGVATSAAQTVWNMFGPVNPNSNVDRPFGSAVVDGVDFDFESGVNNLATFATELRSLMDASASSANRKFYLSAAPQCVYPDFADNPALNGSVFFDFIMIQYYNNGCGVSSYVPGATTQWNYNFDVWDNWAHTVSKNPNVRILLGIAANTGAASGYVSGTQLSAVISFTKQYSSFAGIMMWDMSQLYQNSGFLGQVVSDLAASGSTPPATTSSGASKTTTSSGGSTSPTGGSVPQWGQCGGEGYTGPTQCQSPYKCVFSSQWWSSCQ

SEQ ID NO: 126 - protein, 148 aa, Trichoderma hamatum
MYFANTLLSIAGAASIANALGSNCQDSGVCADINANLSFAIEQLKGMDQLQRFSDGQHITCVDTDSEGNSSLCLSYQDTGRSWTVFQTAWFAQSLVEQGCQACGSLSMGSHHGELVASVITKSTSGLDTSGARRGMDMVQLAVRAGDR

SEQ ID NO: 127 - DNA, 1380 bp, Chitinophaga oryzae
ttaccgacttcaggtccccccggctttcatggcttgacgggcggtgtgtacaaggtccgggaacgtattcaccgtatcattgctgatatacgattactagcgattccagcttcatgagggcgagttgcagccctcaattcgaactgagataggatttttgagattagcatcctgttaccaggtagcagccctttgttcctaccattgtagcacgtgtgtagccctgggcataaaggccatgatgacttgacatcatcccctccttcctcgcgtcttacgacggcagtttctttagagttcccagcttaacctgttggcaactaaagataggggttgcgctcgttgcgggacttaacccaacacctcacggcacgagctgacgacagccatgcagcaccttacaaaatgtgtattgctacaaaatctgctttcacagacgggcatcttgcattctagcccaggtaaggttcctcgcgtatcatcgaattaaaccacatgctccaccgcttgtgcggacccccgtcaattcctttgagtttcaaccttgcggtcgtacttcccaggtggattacttaatgctttcgctcagacacttactgtatatcgcaaatgtcgagtaatcatcgtttagggcgtggactaccagggtatctaatcctgtttgatccccacgctttcgtgcctcagcgtcaatatttgtgtagccagctgccttcgcaattggtgttctatgtcatatctatgcatttcaccgctacatgacatattccgctaacctccacaatattcaagacttatagtatccatggcagtttccaggttaagcctggagatttcaccacggacttacaaatccgcctacgcaccctttaaacccagtgaatccggataacgcttgcaccctccgtattaccgcggctgctggcacggagttagccggtgcttattcctctggtaccgtcaacactcttagaaaaaggtgatttcgtcccagataaaagaagtttacaatccagaggaccttcatccttcacgcggcatggctggttcagacttgcgtccattgaccaatattccttactgctgcctcccgtaggagtcgggcccgtgtctcagtgcccgtgtgactggtcgcgctctcacgccagttactgatcgtcggcttggtgagccgttacctcaccaactacctaatcaggcgcacgcccatcttcaagcgaaattctttaatcattaagtgatgccaccctgtgattttacgatgtattaatccgaatttcttcgggctatccatctcttgaaggaaggttgcgtacgtgttccgcacccgtttgccggtcgccacccagtattgctacctgtgctgcccctcgacttgca

SEQ ID NO: 128 - protein, 137 aa, Chitinophaga oryzae
MKIAIINGPNLNLLGKREPGIYGNEPFEDYFEKLKVLFADVELEYYQNNSEGNIIDILHEIGFTYDGILLNAGAYTHYSIAIRDAIAAIKTPVLEIHISNVHAREEFRHKSVIAPVCKGMIAGLGMKGYALGINYFL

SEQ ID NO: 129 - protein, 164 aa, Chitinophaga oryzae
MKQVEVGIIMGSSSDAPIMRQAIDVLKKFDIGYEFNVVSAHRSPQRMFDYAGTAEERGLKVIIAGAGGAAHLPGMVAAITTLPVVGVPIKSSNSLDGWDSLLSIVQMPGDIPVATVSVNGARNAGLLAVQILATCNPELRQKLADMKRENNEKVNQMNETLDRS

SEQ ID NO: 130 - protein, 331 aa, Chitinophaga oryzae
MNSFGRLFRVNVFGESHGASVGVNIDGVPAGIPLKQEDFLPDLDRRKAGAKGTTPRKEDDLPYIKSGVFNDHTTGAPITILFENNNTRSTDYEKLREFPRPGHADFVATHKYGGFEDYRGGGHFSGRLTLNLVAAGVIAKKILGESIKVTATLKEVAGLPDAEQGLEAAIAAKDSVGGIVECVVEGLPIGLGEPFFDSVESTIAHAVFAIPAIKGIEFGAGFAAAKMKGIEHNDAILDASGKTATNNAGGVVGGITNGNPLVFRVAVKPTSSTPKEQHTLNIKSGQVEAFSVKGRHDLCIALRVPVVLEAVAAMALADLMMVEQRSPRIWK

SEQ ID NO: 131 - protein, 298 aa, Chitinophaga oryzae
MSGQLKEVRNRIKSTQSNLQITKAMKMVSAAKLRRAQDAILLMRPYAVKLQEMLQNIVSNSEGSIDLALAAERPVEKVLLVVITSDRGLCGAYNSNLIKLTKQVIREKYQEQFEKGHVTILPIGKKGWEHFGKNGYKMNDTYWHLFAHLDFDHVKEAAAVATDGFTSGQYDAVEIIYSQFKNAATQFYISEQFLPIAKPQEVETGKGKKAAKADFIFEPEKQTLIAELMPKILNTQLYKAMLDANASEHGARMTAMDKATENANELLRNYKITYNRARQAAITTELTEIVSGAAALEG

SEQ ID NO: 132 - DNA, 1378 bp, Bacillus velenzensis
gggagcttgctccctgatgttagcggcggacgggtgagtaacacgtgggtaacctgcctgtaagactgggataactccgggaaaccggggctaataccggatggttgtttgaaccgcatggttcagacataaaaggtggcttcggctaccacttacagatggacccgcggcgcattagctagttggtgaggtaacggctcaccaaggcaacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaagctctgttgttagggaagaacaagtgccgttcaaatagggcggcaccttgacggtacctaaccagaaagccacggctaactacgtgccagcagccgcggtaatacgtaggtggcaagcgttgtccggaattattgggcgtaaagggctcgcaggcggtttcttaagtctgatgtgaaagcccccggctcaaccggggagggtcattggaaactggggaacttgagtgcagaagaggagagtggaattccacgtgtagcggtgaaatgcgtagagatgtggaggaacaccagtggcgaaggcgactctctggtctgtaactgacgctgaggagcgaaagcgtggggagcgaacaggattagataccctggtagtccacgccgtaaacgatgagtgctaagtgttagggggtttccgccccttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaatcctagagataggacgtccccttcgggggcagagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatgggcagaacaaagggcagcgaaaccgcgaggttaagccaatcccacaaatctgttctcagttcggatcgcagtctgcaactcgactgcgtgaagctggaatcgctagtaatcgcggatcagcatgccgcggtgaatacgttcccgggccttgtacacaccgcccgtcacaccacgagagtttgtaacacccgaagtcggtgaggta

SEQ ID NO: 133 - protein, 292 aa, Bacillus velenzensis
MAITAQQVKELRQKTGAGMMDCKKALTETDGDMDKAIDLLREKGIAKAAKKADRIAAEGSTLIKTDGNKGVILEVNSETDFVAKNEGFKELLNTLADHLLANAPADLEEAMGQKMENGSTVEEYITSNVAKIGEKITLRRFAVITKEDSEAFGAYLHMGGRIGVLSVLSGTDEDLAKDIAMHVAAVNPRYISRDQVSEEEANHERQILTQQALQEGKPENIVAKMVEGRLNKFFEEICLLDQAFVKNPDEKVKQVVAAKNASVKTYVRYEVGEGIEKRQENFAEEVMNQVKK

SEQ ID NO: 134 - protein, 132 aa, Bacillus velenzensis
MKTVKVNIVTPDGPVYDADIEMVSVRAESGDLGILPGHIPTVAPLKIGAVRLKKDGQTELAAVSGGFLEVRPDQVTILAQAAETAESIDKERALAAKKRAEDRLNKRSDDTDIRRAELALQRAVNRLDVAGN

SEQ ID NO: 135 - protein, 475 aa, Bacillus velenzensis
MNFETVIGLEVHVELKTKSKIFSSSPTPFGAEANTQTSVIDLGYPGVLPVLNKEAVEFAMKAAMALNCEIATDTKFDRKNYFYPDNPKAYQISQFDKPIGENGWIEIEVGGKTKKIGITRLHLEEDAGKLTHTGDGSLVDFNRQGTPLVEIVSEPDIRTPEEAYAYLEKLKSIIQYTGVSDCKMEEGSLRCDANISLRPIGQEKFGTKTELKNLNSFAFVQKGLEYEEKRQEQVLLSGGVIQQETRRYDEATKKTILMRVKEGSDDYRYFPEPDLVELYIDDEWKERVRATIPELPDERRKRYIEELGLPAYDAMVLTLTKEMADFFEETVNKGAEAKQASNWLMGEVSAYLNAEQKELEDVALTPEGLAGMIKLIEKGTISSKIAKKVFKELIEKGGDAEKIVKEKGLVQISDESVLLKLVTDALDSNPQSIEDFKNGKDRAIGFLVGQIMKASKGQANPPMVNKILLEEIKKR

SEQ ID NO: 136 - protein, 427 aa, Bacillus velenzensis
MSYLIKNGWMLNEQGEKVAQDIRVTGEFITETGLLTAKDGETVIDAEGLFVSPGLVDLHVHFREPGGEKKETIETGSKAAARGGFTTVAAMPNTRPVPDTKEQMEWLQNRIKETSSVRVLPYASITIRQIGEEMTDFEALHEAGAFAFTDDGVGVQTAGMMYEAMKRAASMNKAIVAHCEDNSLIYGGSVHEGSFSKANGLNGIPSICEAVHIARDVLLAEAADCHYHVCHISTKESVRAVRDAKKAGIRVTAEVSPHHLLLCDEDIPGLDTNFKMNPPLRSKEDREALIEGLLDGTIDFIATDHAPHTEEEKNTDMKLAPFGIVGLETAFPLLYTHFVKNGTWTLKQLVDFMTVKPCEAFGLPYGTLAPGAPADITLIDLEKEAAIDKDTFLSKGKNTPFNKMKCFGWPVATMAAGKLAYEEGRLV

(19) United States
(12) Patent Application Publication
(10) Pub. No.: US 2024/0180164 A1
(43) Pub. Date: Jun. 6, 2024

(54) ENDOPHYTE COMPOSITIONS AND METHODS FOR IMPROVED PLANT HEALTH

(71) Applicant: INDIGO AG, INC., Boston, MA (US)

(72) Inventors: David A. HUBERT, Durham, NC (US); Brandon M. SATINSKY, Brookline, MA (US); Shib BASU, Apex, NC (US)

(21) Appl. No.: 18/556,542

(22) PCT Filed: Apr. 22, 2022
(86) PCT No.: PCT/US2022/026051

§ 371 Date: Oct. 20, 2023

Related U.S. Application Data
(60) Provisional application No. 63/201,305, filed on Apr. 22, 2021.

(51) Int. Cl. A01N 63/22; A01N 25/08; A01N 25/24; A01N 63/27; A01N 63/38; A01P 3/00; C12R 1/07; C12R 1/38; C12R 1/885

(52) U.S. Cl. CPC A01N 63/22; A01N 25/08; A01N 25/24; A01N 63/27; A01N 63/38; A01P 3/00; C12R 2001/07; C12R 2001/38; C12R 2001/885

(57) ABSTRACT

This invention relates to compositions and methods for improving plant health, wherein a plant is heterologously disposed to one or more endophytes, or derived from a plant element heterologously disposed to one or more endophytes.

### CROSS REFERENCE TO RELATED APPLICATIONS

This application claims the benefit of and priority to U.S. Provisional Patent Application No. 63/201,305 filed Apr. 22, 2021, the entire disclosure of which is hereby incorporated by reference in its entirety for all purposes.

### SEQUENCE LISTING

The instant application contains a Sequence Listing with 136 sequences, which has been filed electronically in ASCII format is hereby incorporated by reference in its entirety. Said ASCII copy, created on Apr. 22, 2022, is named 10236PCTWO1_final_ST25.txt and is 215,474 bytes in size.

### BACKGROUND

According to the United Nations Food and Agriculture Organization, the world's population will exceed 9.6 billion people by the year 2050, which will require significant improvements in agriculture to meet growing food demands. There is a need for improved methods and agricultural plants that will enable a near doubling of food production with fewer resources and more environmentally sustainable inputs, and for plants with improved responses to various stresses.

### SUMMARY OF INVENTION

Disclosed herein are methods of improving plant health, comprising heterologously disposing one or more endophytes to a plant element in an effective amount to improve a trait of agronomic importance in a plant derived from the treated plant element relative to a reference plant derived from a reference plant element, wherein the one or more endophytes comprise at least one polynucleotide sequence that is at least 97% identical to one or more polynucleotide sequences selected from the group consisting of SEQ ID NOs. 27, 32-93, 127, 132, and combinations thereof. Optionally, the one or more endophytes are capable of producing a protein whose amino acid sequence is selected from the group consisting of SEQ ID NOs. 28-31, 94-126, 128-131, 133-136, and combinations thereof.

In some embodiments, the method additionally comprises the step of placing the plant element in or on a growth medium. In some embodiments, the one or more endophytes are heterologously disposed to a plant element prior to placing the treated plant element in or on a growth medium. In some embodiments, the one or more endophytes are heterologously disposed to a plant element after placing the plant elements in or on a growth medium. In some embodiments, the one or more endophytes are heterologously disposed to a plant element concurrently with placing the plant elements in or on a growth medium.

In some embodiments, the one or more endophytes are heterologously disposed to a plant element at least two times. In some embodiments, the one or more endophytes are heterologously disposed to a plant element via a seed treatment or soil pre-treatment and one or more foliar applications. In some embodiments, the one or more endophytes are heterologously disposed to a plant element via a seed treatment or soil pre-treatment and one or more floral applications. In some embodiments, the one or more endophytes are heterologously disposed to a plant element via one or more seed treatments or soil pre-treatments, one or more foliar applications, and one or more floral applications. In some embodiments, the one or more endophytes are heterologously disposed to a plant element via seed treatment, root wash, seedling soak, foliar application, floral application, soil inoculum, in-furrow application, sidedress application, soil pre-treatment, wound inoculation, drip tape irrigation, vector-mediation inoculation, injection, osmopriming, hydroponics, aquaponics, aeroponics, or combinations thereof.

In some embodiments, the one or more endophytes are heterologously disposed to a plant element of a different plant variety from the variety of the plant element from which the one or more endophytes were obtained. In some embodiments, the one or more endophytes are heterologously disposed to a plant element of the same plant variety as the variety of the plant element from which the one or more endophytes were obtained. In some embodiments, the one or more endophytes are heterologously disposed to a plant element of a different plant species from the species of the plant element from which the one or more endophytes were obtained. In some embodiments, the one or more endophytes are heterologously disposed to a plant element of the same plant species as the species of the plant element from which the one or more endophytes were obtained.

In some embodiments, the plant elements are allowed to germinate. In some embodiments, the plant elements are grown to yield.

In another aspect, disclosed herein are synthetic compositions, comprising one or more endophytes heterologously disposed to a treatment formulation, wherein the one or more endophytes comprise at least one polynucleotide sequence that is at least 97% identical to one or more polynucleotide sequences selected from the group consisting of SEQ ID NOs. 27, 32-93, 127, 132, and combinations thereof. Optionally, the one or more endophytes are capable of producing a protein whose amino acid sequence is selected from the group consisting of SEQ ID NOs. 28-31, 94-126, 128-131, 133-136, and combinations thereof. In some embodiments, the composition additionally comprises a plant element. In some embodiments, the one or more endophytes are capable of improving a trait of agronomic importance in a plant derived from the plant element (for example, when grown from a treated seed) relative to a plant derived from a reference plant element.

In some embodiments, the synthetic composition additionally comprises one or more of a surfactant, a buffer, a tackifier, a microbial stabilizer, a fungicide, an anticomplex agent, an herbicide, a nematicide, an insecticide, a plant growth regulator, a rodenticide, a desiccant, a nutrient, an excipient, a wetting agent, a salt, and a polymer. In some embodiments, the polymer is a biodegradable polymer selected from the group consisting of alginate, agarose, agar, gelatin, polyacrylamide, chitosan, polyvinyl alcohol, and combinations thereof. In some embodiments, the biodegradable polymer is alginate and the alginate is sodium alginate or calcium alginate.

In some embodiments, the synthetic composition comprises one or more endophytes of the present invention and one or more chemical or biological agents capable of killing a pest of a plant, impeding the feeding and or growth and or reproduction of a pest of a plant, repelling a pest of a plant, and or reducing the severity or extent of infection of a plant host by a pest of a plant, including without limitation chemical or biological agents that are acetylcholinesterase (AChE) inhibitors, GABA-gated chloride channel blockers, sodium channel modulators, nicotinic acetylcholine receptor (nAChR) competitive modulators, nicotinic acetylcholine receptor (nAChR) allosteric modulators—Site I, Glutamate-gated chloride channel (GluCl) allosteric modulators, Chordotonal organ TRPV channel modulators, Nicotinic acetylcholine receptor (nAChR) channel blockers, Octopamine receptor agonists, Voltage-dependent sodium channel blockers, multi-site inhibitors, Ryanodine receptor modulators, chordotonal organ modulators (wherein the chordotonal organ modulator does not bind to the Nan-lav TRPV channel complex), GABA-gated chloride channel allosteric modulators, GABA-gated chloride channel allosteric modulators—Site II, nicotinic acetylcholine receptor (nAChR) Allosteric Modulators—Site II, Juvenile hormone mimics, Mite growth inhibitors affecting CHS1, Inhibitors of chitin biosynthesis affecting CHS1, Inhibitors of chitin biosynthesis—type 1, Moulting disruptors—Dipteran, Ecdysone receptor agonists, Inhibitors of acetyl COA carboxylase, Inhibitors of mitochondrial ATP synthase, Uncouplers of oxidative phosphorylation via disruption of the proton gradient, Mitochondrial complex III electron transport inhibitors, Mitochondrial complex I electron transport inhibitors, Mitochondrial complex IV electron transport inhibitors, Mitochondrial complex II electron transport inhibitors, Microbial disruptors of insect midgut membranes, Host-specific occluded pathogenic viruses, other active compounds (such as Azadirachtin, Benzoximate, Bromopropylate, Chinomethionat, Dicofol, Lime sulfur, Mancozeb, Pyridalyl, Sulfur, Chlorantraniliprole, Clothianidin, Tioxazafen, Fluopyram, Triticonazole), other active bacterial agents (such as certain Burkholderia strains including without limitation Burkholderia rinojenses, Wolbachia pipientis), other active fungal agents (such as Beauveria bassiana strains, Metarhizium anisopliae strain F52, Paecilomyces fumosoroseus Apopka strain 97), biological essence including synthetics or extracts or refined or unrefined oils (such as Dysphania ambrosioides near ambrosioides extract, fatty acid monoesters with glycerol or propanediol, neem oil), non-specific mechanical disruptors (such as Diatomaceous earth), or combinations thereof. Examples of AChE inhibitors include without limitation Carbamates (such as Alanycarb, Aldicarb, Bendiocarb, Benfuracarb, Butocarboxim, Butoxycarboxim, Carbaryl, Carbofuran, Carbosulfan, Ethiofencarb, Fenobucarb, Formetanate, Furathiocarb, Isoprocarb, Methiocarb, Methomyl, Metolcarb, Oxamyl, Pirimicarb, Propoxur, Thiodicarb, Thiofanox, Triazamate, Trimethacarb, XMC, Xylylcarb) and Organophosphates (such as Acephate, Azamethiphos, Azinphos-ethyl, Azinphosmethyl, Cadusafos, Chlorethoxyfos, Chlorfenvinphos, Chlormephos, Chlorpyrifos, Chlorpyrifos-methyl, Coumaphos, Cyanophos, Demeton-S-methyl, Diazinon, Dichlorvos/DDVP, Dicrotophos, Dimethoate, Dimethylvinphos, Disulfoton, EPN, Ethion, Ethoprophos, Famphur, Fenamiphos, Fenitrothion, Fenthion, Fosthiazate, Heptenophos, Imicyafos, Isofenphos, Isopropyl O-(methoxyaminothio-phosphoryl) salicylate, Isoxathion, Malathion, Mecarbam, Methamidophos, Methidathion, Mevinphos, Monocrotophos, Naled, Omethoate, Oxydemeton-methyl, Parathion, Parathion-methyl, Phenthoate, Phorate, Phosalone, Phosmet, Phosphamidon, Phoxim, Pirimiphos-methyl, Profenofos, Propetamphos, Prothiofos, Pyraclofos, Pyridaphenthion, Quinalphos, Sulfotep, Tebupirimfos, Temephos, Terbufos, Tetrachlorvinphos, Thiometon, Triazophos, Trichlorfon, Vamidothion). Examples of GABA-gated chloride channel blockers include without limitation Cyclodiene Organochlorines (such as Chlordane, Endosulfan) and Phenylpyrazoles (Fiproles) (such as Ethiprole, Fipronil). Examples of sodium channel modulators include without limitation pyrethroids and pyrethrins (such as Acrinathrin, Allethrin, d-cis-trans Allethrin, d-trans Allethrin, Bifenthrin, Bioallethrin, Bioallethrin Scyclopentenyl isomer, Bioresmethrin, Cycloprothrin, Cyfluthrin, beta-Cyfluthrin, Cyhalothrin, lambda-Cyhalothrin, gamma-Cyhalothrin, Cypermethrin, alpha-Cypermethrin, beta-Cypermethrin, thetacypermethrin, zeta-Cypermethrin, Cyphenothrin, (1R)-trans-isomers], Deltamethrin, Empenthrin (EZ)-(1R)-isomers], Esfenvalerate, Etofenprox, Fenpropathrin, Fenvalerate, Flucythrinate, Flumethrin, tau-Fluvalinate, Halfenprox, Imiprothrin, Kadethrin, Permethrin, Phenothrin [(1R)-trans-isomer], Prallethrin, Pyrethrins (pyrethrum), Resmethrin, Silafluofen, Tefluthrin, Tetramethrin, Tetramethrin [(1R)-isomers], Tralomethrin, Transfluthrin) and Methoxychlor. Examples of nAChR competitive modulators include without limitation Neonicotinoids (such as Acetamiprid, Clothianidin, Dinotefuran, Imidacloprid, Nitenpyram, Thiacloprid, Thiamethoxam), nicotine, sulfoximines (such as Sulfoxaflor), Butenolides (such as Flupyradifurone), and Mesoionics (such as Triflumezopyrim). Examples of nAChR allosteric modulators—Site I include without limitation Spinosyns (such as Spinetoram, Spinosad). Examples of GluCl allosteric modulators include without limitation Avermectins and Milbemycins (such as Abamectin, Emamectin benzoate, Lepimectin, Milbemectin). Examples of multi-site inhibitors include without limitation Alkyl halides (such as Methyl bromide and other alkyl halides), Chloropicrin, Fluorides (such as Cryolite (Sodium aluminum fluoride), Sulfuryl fluoride), Borates (such as Borax, Boric acid, Disodium octaborate, Sodium borate, Sodium metaborate), Tartar emetic, Methyl isothiocyanate generators (such as Dazomet, Metam). Examples of chordotonal organ TRPV channel modulators include without limitation Pyridine azomethine derivatives (such as Pymetrozine, Pyrifluquinazon), and Pyropenes (such as Afidopyropen). Examples of juvenile hormone mimics include without limitation juvenile hormone analogues (such as Hydroprene, Kinoprene, Methoprene), fenoxycarb, and pyriproxyfen. Examples of mite growth inhibitors affecting CHS1 include without limitation Clofentezine, Diflovidazin, Hexythiazox, and Etoxazole. Examples of microbial disruptors of insect midgut membranes include without limitation Bacillus thuringiensis (such as Bacillus thuringiensis subsp. israelensis, Bacillus thuringiensis subsp. aizawai, Bacillus thuringiensis subsp. kurstaki, Bacillus thuringiensis subsp. tenebrionis, Bacillus thuringiensis strain EX297512) and the insecticidal proteins they produce (such as Cry1Ab, Cry1Ac, Cry1Fa, Cry1A.105, Cry2Ab, Vip3A, mCry3A, Cry3Ab, Cry3Bb, Cry34Ab1/Cry35Ab1) and Bacillus sphaericus. Examples of inhibitors of mitochondrial ATP synthase include without limitation Diafenthiuron, Organotin miticides (such as Azocyclotin, Cyhexatin, Fenbutatin oxide), Propargite, and Tetradifon. Examples of uncouplers of oxidative phosphorylation via disruption of the proton gradient include without limitation Pyrroles (such as Chlorfenapyr), Dinitrophenols, and Sulfluramid. Examples of nAChR channel blockers include without limitation Nereistoxin analogues (such as Bensultap, Cartap hydrochloride, Thiocyclam, Thiosultap-sodium). Examples of inhibitors of chitin biosynthesis affecting CHS1 include without limitation Benzoylureas (such as Bistrifluron, Chlorfluazuron, Diflubenzuron, Flucycloxuron, Flufenoxuron, Hexaflumuron, Lufenuron, Novaluron, Noviflumuron, Teflubenzuron, Triflumuron). Examples of inhibitors of chitin biosynthesis—type 1 include without limitation Buprofezin. Examples of moulting disruptors (Dipteran) include without limitation Cyromazine. Examples of ecdysone receptor agonists include without limitation Diacylhydrazines (such as Chromafenozide, Halofenozide, Methoxyfenozide, Tebufenozide). Examples of octopamine receptor agonists include without limitation Amitraz. Examples of mitochondrial complex III electron transport inhibitors include without limitation Hydramethylnon, Acequinocyl, Fluacrypyrim, and Bifenazate. Examples of mitochondrial complex I electron transport inhibitors include without limitation METI acaricides and insecticides such as Fenazaquin, Fenpyroximate, Pyridaben, Pyrimidifen, Tebufenpyrad, Tolfenpyrad) and Rotenone. Examples of voltage-dependent sodium channel blockers include without limitation Oxadiazines (such as Indoxacarb) and Semicarbazones (such as Metaflumizone). Examples of inhibitors of acetyl COA carboxylase include without limitation Tetronic and Tetramic acid derivatives (such as Spirodiclofen, Spiromesifen, Spiropidion, Spirotetramat). Examples of mitochondrial complex IV electron transport inhibitors include without limitation Phosphides (Aluminium phosphide, Calcium phosphide, Phosphine, Zinc phosphide), Cyanides (such as Calcium cyanide, Potassium cyanide, Sodium cyanide). Examples of mitochondrial complex II electron transport inhibitors include without limitation Beta-ketonitrile derivatives (such as Cyenopyrafen, Cyflumetofen) and Carboxanilides (such as Pyflubumide). Examples of ryanodine receptor modulators include without limitation such as Diamides (such as Chlorantraniliprole, Cyantraniliprole, Cyclaniliprole Flubendiamide, Tetraniliprole). Examples of chordotonal organ modulators include without limitation Flonicamid. Examples of GABA-gated chloride channel allosteric modulators include without limitation Meta-diamides (Broflanilide) and Isoxazolines (such as Fluxametamide). Examples of nicotinic acetylcholine receptor (nAChR) Allosteric Modulators—Site II include without limitation GS-omega/kappa HXTX-Hv1a peptide.

In some embodiments, the synthetic composition comprises one or more endophytes of the present invention and one or chemical or biological agent capable of killing a pathogen of a plant, impeding the feeding and or growth and or reproduction of a pathogen of a plant, repelling a pathogen of a plant, and or reducing the severity or extent of infection of a plant host by a pathogen of a plant, including without limitation chemical or biological agents that are PhenylAmides fungicides (acylalanines, oxazolidinones, butyrolactones), hydroxy-(2-amino-) pyrimidines, heteroaromatics (such as isoxazoles, isothiazolones), carboxylic acids, Methyl-Benzimidazole-Carbamates (MBC) fungicides (such as thiophanates, benzimidazoles), N-phenyl carbamates, benzamides (such as toluamides, pyridinylmethyl-benzamides), thiazole carboxamide (such as ethylamino-thiazole-carboxamide), phenylureas, cyanoacrylates (such as aminocyanoacrylates), aryl-phenyl-ketones (such as benzophenone, benzoylpyridine), pyrimidinamines, pyrazole-METI (such as pyrazole-5-carboxamides), quinazoline, succinate-dehydrogenase inhibitors (SDHI) (such as phenyl-benzamides, phenyl-oxo-ethyl thiophene amide, pyridinyl-ethyl-benzamides, phenyl-cyclobutyl-pyridineamide, furan-carboxamides, oxathiin-carboxamides, thiazole-carboxamides, pyrazole-4-carboxamides, N-cyclopropyl-N-benzyl-pyrazole-carboxamides, N-methoxy-(phenyl-ethyl)-pyrazole-carboxamides, pyridine-carboxamides, pyrazine-carboxamides, pydiflumetofen, fluxapyroxad), quinone outside inhibitors (such as methoxy-acrylates, methoxy-acetamide, methoxy-carbamates, oximino-acetates, oximino-acetamides, oxazolidine-diones, dihydro-dioxazines, imidazolinones, benzyl-carbamates, tetrazolinones), quinone inside inhibitors (such as cyano-imidazole, sulfamoyl-triazole, picolinamides), uncouplers of oxidative phosphorylation (such as dinitrophenyl-crotonates, 2,6-dinitro-anilines), organo tin compounds (tri-phenyl tin compounds), thiophene-carboxamides, Quinone outside Inhibitor—stigmatellin binding type (such as triazolo-pyrimidylamine), anilino-pyrimidines, enopyranuronic acid antibiotic, hexopyranosyl antibiotic, glucopyranosyl antibiotic, tetracycline antibiotic, aza-naphthalenes (such as aryloxyquinoline, quinazolinone), phenylpyrroles, dicarboximides, phosphoro-thiolates, dithiolanes, aromatic hydrocarbons, chlorophenyls, nitroanilines, heteroaromatics (such as 1,2,4-thiadiazoles), carbamates, demethylation inhibitors (such as piperazines, pyridines, pyrimidines, imidazoles, triazoles, triazolinthiones), amines (such as morpholines, piperidines, spiroketal-amines), ketoreductase inhibitors (such as hydroxyanilides, amino-pyrazolinone), thiocarbamates, allylamines, polyoxins (such as peptidyl pyrimidine nucleoside), Carboxylic Acid Amides (such as cinnamic acid amides, valinamide carbamates, mandelic acid amides), melanin biosynthesis inhibitors—reductase (such as isobenzo-furanone, pyrrolo-quinolinone, triazolobenzo-thiazole), melanin biosynthesis inhibitors—dehydratase (such as cyclopropane-carboxamide, carboxamide, propionamide), melanin biosynthesis inhibitors—polyketide synthase (such as trifluoroethyl-carbamate), benzo-thiadiazole, benzisothiazole, thiadiazole-carboxamide, polysaccharides (such as laminarin), plant ethanol extracts (such as anthraquinones, resveratrol, extract from Reynoutria sachalinensis), phosphonates (such as ethyl phosphonates, fosetyl-Al, phosphorous acid and salts), isothiazole (such as isothiazolylmethyl ether), cyanoacetamide-oxime, phthalamic acids, benzotriazines, benzene-sulphonamides, pyridazinones, phenyl-acetamide, guanidines, thiazolidine (such as cyano-methylene-thiazolidines), pyrimidinone-hydrazones, 4-quinolyl-acetates, tetrazolyloximes, glucopyranosyl antibiotics, copper salts, sulphur, dithio-carbamates and relatives (such as amobam, ferbam, mancozeb, maneb, metiram, propineb, thiram, zinc thiazole, zineb, ziram), phthalimides, chloronitriles (phthalonitriles), sulfamides (such as dichlofluanid, tolylfluanid), bis-guanidines (such as guazatine, iminoctadine), triazines (such as anilazine), quinones (anthraquinones) (such as dithianon), quinoxalines (such as chinomethionat, quinomethionate), maleimide (such as fluoroimide), thiocarbamate (such as methasulfocarb), polypeptide (lectin) plant extracts (such as extract from the cotyledons of lupine plantlets), phenol and sesquiterpene and triterpenoid and coumarin plant extracts (such as extract from Swinglea glutinosa), terpene hydrocarbon and terpene alcohol and terpene phenol extracts plant extracts (such as extract from Melaleuca alternifolia, plant oils such as eugenol, geraniol, thymol mixtures thereof), Polyene (such as amphoteric macrolide antifungal antibiotic from Streptomyces natalensis or Streptomyces chattanoogensis), oxysterol binding protein homologue inhibition (piperidinyl-thiazole-isoxazolines), other active compounds (such as Fludioxonil, Mefenoxam, Sedaxane, Azoxystrobin, Thiabendazole, Ethaboxam, metalaxyl (such as without limitation metalaxyl-M), Trifloxystrobin, Myclobutanil, Acibenzolar-S-methyl, Metconazole, tolclofos-methyl, Fluopyram, Ipconazole, Oxathiapiprolin, Difenoconazole, Prothyoconazol, Tebuconazole, Pyraclostrobin, Fluxapyroxad, Triticonazole, Fluaxapyroxad), and combinations thereof.

In some embodiments, the synthetic composition comprises one or more endophytes of the present invention and one or more biological agents (for example bacterial or fungal agents) including, but not limited to, those agents capable of killing a pest or pathogen of a plant, impeding the feeding and or growth and or reproduction of a pest or pathogen of a plant, repelling a pest or pathogen of a plant, and or reducing the severity or extent of infection of a plant host by a pathogen or pest of a plant. The one or more bacterial or fungal agents may be living or dead (including without limitation by heat inactivation) bacteria or fungi, extracts and or metabolites of bacteria or fungi (including without limitation extracts and or metabolites in spent growth media), or combinations thereof. Non-limiting examples of biological agents include Trichoderma species including without limitation Trichoderma atroviride strain I-1237, Trichoderma atroviride strain LU132, Trichoderma atroviride strain SC1, Trichoderma atroviride strain SKT-1, Trichoderma atroviride strain 77B, Trichoderma asperellum strain T34, Trichoderma asperellum strain kd, Trichoderma harzianum strain T-22, Trichoderma virens strain G-41; Clonostachys species including without limitation Gliocladium catenulatum strain J1446, Clonostachys rosea strain CR-7; Coniothyrium species including without limitation Coniothyrium minitans strain CON M 91-08; Talaromyces species including without limitation Talaromyces flavus strain SAY-Y-94-01; Saccharomyces species including without limitation Saccharomyces cerevisae strain LAS02; Bacillus species including without limitation Bacillus amyloliquefaciens strain QST713, Bacillus amyloliquefaciens strain FZB24, Bacillus amyloliquefaciens strain MBI600, Bacillus amyloliquefaciens strain D747, Bacillus amyloliquefaciens strain F727, Bacillus amyloliquefaciens strain AT-332, Bacillus amyloliquefaciens strain MBI 600 Bacillus mycoides isolate J, Bacillus subtilis strain AFS032321, Bacillus subtilis strain Y1336, Bacillus subtilis strain MBI 600, Bacillus subtilis strain HAI-0404, Bacillus firmus I-1582); Pseudomonas species including without limitation Pseudomonas chlororaphis strain AFS009; Streptomyces species including without limitation Streptomyces griseovirides strain K61, Streptomyces lydicus strain WYEC108; Penicillium species such as Penicillium bilaiae, Penicillium bilaiae; and Pasteuria species including without limitation Pasteuria nishizawae Pn1).

In some embodiments, one or more endophytes of the present invention and one or chemical or biological agents described herein are present in a synthetic composition at a weight ratio of between 1000:1 and 1:1000, 100:1 and 1:100, or 10:1 and 1:10.

In some embodiments, the synthetic composition may be stored at between 0 degrees Celsius and 4 degrees Celsius for 1 week with less than 1 log loss of CFU of the one or more endophytes. In some embodiments, the synthetic composition may be stored at between 4.1 degrees Celsius and 20 degrees Celsius for 1 week with less than 1 log loss of CFU of the one or more endophytes. In some embodiments, the synthetic composition may be stored at between 20.1 degrees Celsius and 35 degrees Celsius for 1 week with less than 1 log loss of CFU of the one or more endophytes.

In yet another aspect, described herein are methods of measuring plant health comprising determining the presence or abundance of one or more endophytes in a plant element, growth medium or growth environment, wherein the one or more endophytes comprise at least one polynucleotide sequence that is at least 97% identical to one or more polynucleotide sequences selected from the group consisting of SEQ ID NOs. 27, 32-93, 127, 132, and combinations thereof. In some embodiments, the presence or abundance of one or more endophytes is determined relative to a reference plant element, growth medium or growth environment. In some embodiments, the one or more endophytes are not present in the reference plant element, growth medium or growth environment. In some embodiments, the one or more endophytes are less abundant in the reference plant element, growth medium or growth environment. In some embodiments, the presence or abundance of one or more endophytes is determined in a plant element and modulation of one or more traits of agronomic importance is inferred from the presence or amount of the one or more endophytes in the plant element. In some embodiments, the presence or abundance of one or more endophytes is determined in a growth medium and the capacity of the growth medium to modulate one or more trait of agronomic importance in a plant element planted therein is inferred from the presence or amount of the one or more endophytes in the growth medium. In some embodiments, the presence or abundance of one or more endophytes is determined in a growth environment and the capacity of the growth environment to modulate one or more trait of agronomic importance in a plant element grown therein is inferred from the presence or amount of the one or more endophytes in the growth environment. In some embodiments, the presence or abundance of one or more endophytes is determined by polymerase chain reaction, fluorescence in situ hybridization, or isothermal amplification.

In some embodiments, a plurality of nucleic acid probes are used to determine the presence or abundance of one or more endophytes in a plant element, growth medium or growth environment, wherein the plurality comprises complementary or reverse complementary sequences to a region of at least 10 contiguous nucleotides within one or more polynucleotide sequences selected from the group consisting of SEQ ID NOs. 27, 32-93, 127, 132, and combinations thereof. In some embodiments, the complementary or reverse complementary region comprises at least 20 contiguous nucleotides. In some embodiments, the complementary or reverse complementary region comprises at least 30 contiguous nucleotides. In some embodiments, the complementary or reverse complementary region comprises at least 40 contiguous nucleotides. In some embodiments, the plurality of nucleic acid probes are single-stranded DNA. In some embodiments, the plurality of nucleic acid probes are attached to one or more solid supports. In some embodiments, the plurality of nucleic acid probes are attached to a plurality of beads. In some embodiments, the plurality of nucleic acid probes are attached to a contiguous solid support.

In some embodiments, the plant element is a monocot. In some embodiments, the monocot is a cereal. In some embodiments, the cereal is selected from the group consisting of wheat, rice, barley, buckwheat, rye, millet, oats, corn, sorghum, triticale and spelt. In some embodiments, the cereal is wheat.

In some embodiments, the plant element is a dicot. In some embodiments, the dicot is selected from the group consisting of cotton, tomato, lettuce, peppers, cucumber, endive, melon, potato, cannabis, and squash. In some embodiments, the dicot is a legume. In some embodiments, the legume is soy, peas or beans.

In some embodiments, the plant element is a whole plant, seedling, meristematic tissue, ground tissue, vascular tissue, dermal tissue, seed, leaf, root, shoot, stem, flower, fruit, stolon, bulb, tuber, corm, keikis, shoot, or bud. In some embodiments, the plant element is a seed.

In some embodiments, the trait of agronomic importance is selected from the group consisting of drought tolerance, heat tolerance, cold tolerance, salinity tolerance, metal tolerance, herbicide tolerance, improved water use efficiency, improved nitrogen utilization, improved nitrogen fixation, improved nutrient use efficiency, improved nutrient utilization, biotic stress tolerance, improved disease resistance, yield improvement, health enhancement, vigor improvement, decreased necrosis, decreased chlorosis, decreased area of necrotic tissue, decreased area of chlorotic tissue, growth improvement, photosynthetic capability improvement, nutrition enhancement, altered protein content, altered oil content, increased biomass, increased shoot height, increased root length, increased shoot biomass, increased root biomass, increased leaf area, increased shoot area, increased root area, improved root architecture, increased seed germination percentage, increased seed germination rate, increased seedling survival, increased survival, photosynthetic efficiency, transpiration rate, seed/fruit number or mass, plant grain or fruit yield, leaf chlorophyll content, photosynthetic rate, wilt recovery, turgor pressure, modulation of a metabolite, production of a volatile organic compound (VOC), modulation of the proteome, increased seed weight, altered seed carbohydrate composition, altered seed oil composition, altered seed protein composition, altered seed nutrient composition, and combinations thereof. In some embodiments, the trait of agronomic importance is biotic stress tolerance. In some embodiments, the trait of agronomic importance is improved nutrient use efficiency. In some embodiments, the trait of agronomic importance is drought tolerance.

In some embodiments, the one or more endophytes is a member of the Class Chitinophagia, Bacilli, Gammaproteobacteria, or Sordariomycetes. In some embodiments, the one or more endophytes is a member of the Order Chitinophagales, Bacillales, Pseudomonadales, or Hypocreales. In some embodiments, the one or more endophytes is a member of the Family Chitinophagaceae, Bacillaceae, Pseudomonadaceae, or Hypocreaceae. In some embodiments, the one or more endophytes is a member of the Genus Chitinophaga, Bacillus, Pseudomonas, or Trichoderma.

In some embodiments, the one or more endophytes is a member of the Genus Chitinophaga, wherein the one or more endophytes comprise at least one polynucleotide sequence that is at least 97%, at least 98%, at least 99%, or 100% identical to 16S ribosomal RNA gene having SEQ ID NO. 127. In some embodiments, the genomes of the one or more endophytes comprise one or more open reading frames encoding proteins whose amino acid sequences are at least 97%, at least 97%, at least 98%, at least 99%, or 100% identical to one or more of SEQ ID NOs. 128-131. In some embodiments, the one or more endophytes is a member of the Genus Chitinophaga, wherein the one or more endophytes are capable of producing a protein whose amino acid sequence is at least 95%, at least 96%, at least 97%, at least 97%, at least 98%, at least 99%, or 100% identical to one or more of SEQ ID NOs. 128-131.

In some embodiments, the one or more endophytes is a member of the Genus Bacillus, wherein the one or more endophytes comprise at least one polynucleotide sequence that is at least 97%, at least 98%, at least 99%, or 100% identical to 16S ribosomal RNA gene having SEQ ID 132. In some embodiments, the genomes of the one or more endophytes comprise one or more open reading frames encoding proteins whose amino acid sequences are at least 97%, at least 97%, at least 98%, at least 99%, or 100% identical to one or more of SEQ ID NOs. 133-136. In some embodiments, the one or more endophytes is a member of the Genus Bacillus, wherein the one or more endophytes are capable of producing a protein whose amino acid sequence is at least 95%, at least 96%, at least 97%, at least 97%, at least 98%, at least 99%, or 100% identical to one or more of SEQ ID NOs. 133-136.

In some embodiments, the one or more endophytes is a member of the Genus Pseudomonas, wherein the one or more endophytes comprise at least one polynucleotide sequence that is at least 97%, at least 98%, at least 99%, or 100% identical to 16S ribosomal RNA gene having SEQ ID NO. 27. In some embodiments, the genomes of the one or more endophytes comprise one or more open reading frames encoding proteins whose amino acid sequences are at least 97%, at least 97%, at least 98%, at least 99%, or 100% identical to one or more of SEQ ID NOs. 28-31. In some embodiments, the one or more endophytes is a member of the Genus Pseudomonas, wherein the one or more endophytes are capable of producing a protein whose amino acid sequence is at least 95%, at least 96%, at least 97%, at least 97%, at least 98%, at least 99%, or 100% identical to one or more of SEQ ID NOs. 28-31.

In some embodiments, the one or more endophytes is a member of the Genus Trichoderma, wherein the one or more endophytes comprise at least one polynucleotide sequence that is at least 97%, at least 98%, at least 99%, or 100% identical to internal transcribed spacer sequence having SEQ ID NO. 32. In some embodiments, the genomes of the one or more endophytes comprise one or more open reading frames encoding proteins whose amino acid sequences are at least 97%, at least 97%, at least 98%, at least 99%, or 100% identical to one or more of SEQ ID NOs. 95-127. In some embodiments, the one or more endophytes is a member of the Genus Trichoderma, wherein the one or more endophytes genomes comprise at least one polynucleotide sequence that is at least 97%, at least 98%, at least 99%, or 100% identical to one or more polynucleotide sequences selected from the group consisting of SEQ ID NOs. 59-94. In some embodiments, the genome of the one or more endophytes of the Genus Trichoderma comprises polynucleotide regions having 100% sequence identity to SEQ ID NOs. 32-93.

In some embodiments, the genome of the one or more endophytes of the Genus Trichoderma comprises at least 1, 2, 3, 4, 5, 6, 7, 8, 9, 10, 11, 12 polynucleotide regions having at least 97%, at least 98%, at least 99%, or 100% sequence identity to one or more polynucleotide sequences selected from the group consisting of SEQ ID NOs. 61, 71, 72, 75, 76, 80, 81, 82, 85, 87, 88, and 89. In some embodiments, the genome of the one or more endophytes of the Genus Trichoderma comprises at least one polynucleotide regions having at least 97%, at least 98%, at least 99%, or 100% sequence identity to one or more polynucleotide sequences selected from the group consisting of SEQ ID NOs. 61, 80, 87, 88, 89 and at least one polynucleotide regions having at least 97%, at least 98%, at least 99%, or 100% sequence identity to one or more polynucleotide sequences selected from the group consisting of SEQ ID NOs. 71, 72, 75, 76, 81, 82, and 85. In some embodiments, the genome of the one or more endophytes of the Genus Trichoderma comprises at least 1 polynucleotide regions having at least 97%, at least 98%, at least 99%, or 100% sequence identity to SEQ ID NOs. 87 and at least 1 polynucleotide regions having at least 97%, at least 98%, at least 99%, or 100% sequence identity to SEQ ID NOs. 71. In some embodiments, the genome of the one or more endophytes of the Genus Trichoderma comprises polynucleotide regions having at least 97%, at least 98%, at least 99%, or 100% sequence identity to one or more polynucleotide sequences selected from the group consisting of SEQ ID NOs. 61, 80, 87, 88, 89 and at least 1 polynucleotide regions having at least 97%, at least 98%, at least 99%, or 100% sequence identity to one or more polynucleotide sequences selected from the group consisting of SEQ ID NOs. 71, 72, 75, 76, 81, 82, and 85.

In some embodiments, the genome of the one or more endophytes of the Genus Trichoderma comprises at least 1, 2, 3, 4, 5, 6 polynucleotide regions having at least 97%, at least 98%, at least 99%, or 100% sequence identity to one or more polynucleotide sequences selected from the group consisting of SEQ ID NOs. 59, 60, 63, 64, 65, and 84. In some embodiments, the genome of the one or more endophytes of the Genus Trichoderma comprises polynucleotide regions having 100% sequence identity to SEQ ID NOs. 59, 60, 63, 64, 65, and 84.

In some embodiments, the genome of the one or more endophytes of the Genus Trichoderma comprises at least 1, 2, 3, 4, 5, 6 polynucleotide regions having at least 97%, at least 98%, at least 99%, or 100% sequence identity to one or more polynucleotide sequences selected from the group consisting of SEQ ID NOs. 67, 70, 79, and 83. In some embodiments, the genome of the one or more endophytes of the Genus Trichoderma comprises polynucleotide regions having 100% sequence identity to SEQ ID NOs. 67, 70, 79, and 83.

In some embodiments, the genome of the one or more endophytes of the Genus Trichoderma comprises a polynucleotide regions having at least 97%, at least 98%, at least 99%, or 100% sequence identity to SEQ ID NO. 77.

In some embodiments, the genome of the one or more endophytes of the Genus Trichoderma comprises at least 1, 2, 3, 4, 5, 6, 7, 8 polynucleotide regions having at least 97%, at least 98%, at least 99%, or 100% sequence identity to one or more polynucleotide sequences selected from the group consisting of SEQ ID NOs. 62, 66, 68, 69, 73, 74, 78, and 86. In some embodiments, the genome of the one or more endophytes of the Genus Trichoderma comprises polynucleotide regions having 100% sequence identity to SEQ ID NOs. 62, 66, 68, 69, 73, 74, 78, and 86.

In some embodiments, the genome of the one or more endophytes of the Genus Trichoderma comprises at least one polynucleotide region having at least 97%, at least 98%, at least 99%, or 100% sequence identity to one or more polynucleotide sequences selected from the group consisting of SEQ ID NOs. 61, 71, 72, 75, 76, 80, 81, 82, 85, 87, 88, and 89, and at least one polynucleotide regions having at least 97%, at least 98%, at least 99%, or 100% sequence identity to one or more polynucleotide sequences selected from the group consisting of SEQ ID NOs. 59, 60, 63, 64, 65, and 84, and at least one polynucleotide regions having at least 97%, at least 98%, at least 99%, or 100% sequence identity to one or more polynucleotide sequences selected from the group consisting of SEQ ID NOs. 67, 70, 79, and 83, and at least a polynucleotide region having at least 97%, at least 98%, at least 99%, or 100% sequence identity to SEQ ID NO. 77, and at least one polynucleotide regions having at least 97%, at least 98%, at least 99%, or 100% sequence identity to one or more polynucleotide sequences selected from the group consisting of SEQ ID NOs. 62, 66, 68, 69, 73, 74, 78, and 86.

In some embodiments, the one or more endophytes is a member of the Genus Trichoderma, wherein the one or more endophytes are capable of producing a protein whose amino acid sequence is at least 95%, at least 96%, at least 97%, at least 97%, at least 98%, at least 99%, or 100% identical to one or more of SEQ ID NOs. 94-126.

In some embodiments, the one or more endophytes is a member of the Genus Trichoderma, wherein the one or more endophytes are capable of producing a protein whose amino acid sequence is at least 95%, at least 96%, at least 97%, at least 97%, at least 98%, at least 99%, or 100% identical to one or more of SEQ ID NOs. 99, 104, 107, 108, 119, and 126.

In some embodiments, the one or more endophytes is a member of the Genus Trichoderma, wherein the one or more endophytes are capable of producing a protein whose amino acid sequence is at least 95%, at least 96%, at least 97%, at least 97%, at least 98%, at least 99%, or 100% identical to one or more of SEQ ID NOs. 94, 95, 96, 97, 98, 101, 106, 109, 112, 113, 114, 116, 120, 121, 122, 123, 124, and 125.

In some embodiments, the one or more endophytes is a member of the Genus Trichoderma, wherein the one or more endophytes are capable of producing a protein whose amino acid sequence is at least 95%, at least 96%, at least 97%, at least 97%, at least 98%, at least 99%, or 100% identical to one or more of SEQ ID NOs. 100, 102, 103, 105, 110, 111, 117, and 118.

In some embodiments, the one or more endophytes is a member of the Genus Trichoderma, wherein the one or more endophytes are capable of producing a protein whose amino acid sequence is at least 95%, at least 96%, at least 97%, at least 97%, at least 98%, at least 99%, or 100% identical to SEQ ID NO. 115.

In some embodiments, the one or more endophytes is a member of the Genus Trichoderma, wherein the one or more endophytes are capable of producing at least one protein whose amino acid sequence is at least 95%, at least 96%, at least 97%, at least 97%, at least 98%, at least 99%, or 100% identical to one or more of SEQ ID NOs. 94-126, and at least one protein whose amino acid sequence is at least 95%, at least 96%, at least 97%, at least 97%, at least 98%, at least 99%, or 100% identical to one or more of SEQ ID NOs. 99, 104, 107, 108, 119, and 126, and at least one protein whose amino acid sequence is at least 95%, at least 96%, at least 97%, at least 97%, at least 98%, at least 99%, or 100% identical to one or more of SEQ ID NOs. 94, 95, 96, 97, 98, 101, 106, 109, 112, 113, 114, 116, 120, 121, 122, 123, 124, and 125, and at least one protein whose amino acid sequence is at least 95%, at least 96%, at least 97%, at least 97%, at least 98%, at least 99%, or 100% identical to one or more of SEQ ID NOs. 100, 102, 103, 105, 110, 111, 117, and 118.

In some embodiments, the one or more endophytes comprises at least 2 endophytes. In some embodiments, the one or more endophytes comprises at least 3 endophytes. In some embodiments, the one or more endophytes comprises at least 4 endophytes. In some embodiments, the one or more endophytes comprises at least 5 endophytes. In some embodiments, the one or more endophytes comprises at least 10 endophytes.

In some embodiments, the one or more endophytes are encapsulated in polymeric beads. In some embodiments, the polymeric beads are less than 500 μm in diameter at their widest point. In some embodiments, the polymeric beads are less than 200 μm in diameter at their widest point. In some embodiments, the polymeric beads are less than 100 μm in diameter at their widest point. In some embodiments, the polymeric beads are less than 50 μm in diameter at their widest point. In some embodiments, the polymeric beads' average diameter at their widest point is between 500 μm and 250 μm. In some embodiments, the polymeric beads' average diameter at their widest point is between 249 μm and 100 μm. In some embodiments, the polymeric beads' average diameter at their widest point is between 100 μm and 50 μm.

In some embodiments, the one or more endophytes are encapsulated in inorganic or mineral particles. In some embodiments, the inorganic or mineral particles are silica, clay, talc, sand, silt, and magnetite. In some embodiments, the one or more endophytes are encapsulated in organic matter particles. In some embodiments, the organic matter particles are urea, humus, active carbons, proteins, biochar, carbohydrate, and lipids.

### DESCRIPTION OF THE DRAWINGS

FIG. 1A shows an exemplary photo a tryptic soy agar (TSA) plate inoculated with Fusarium graminearum (B), MIC-84302 (E), and metconazole (A) a chemical fungicide. The lack of growth of Fusarium graminearum around MIC-84302 is notable in comparison to the control plate shown in FIG. 1C.

FIG. 1B shows an exemplary photo a TSA plate inoculated with Fusarium graminearum (B), MIC-18905 (D), and metconazole (A) a chemical fungicide. The lack of growth of Fusarium graminearum around MIC-18905 is notable in comparison to the control plate shown in FIG. 1C.

FIG. 1C shows an exemplary photo a TSA plate inoculated with Fusarium graminearum (B) and metconazole (A) a chemical fungicide; of note is the uninterrupted edge of pathogen growth on the left side of the plate.

FIG. 2A shows an exemplary photo a Reasoner's 2A Agar (R2A) plate inoculated with Fusarium graminearum (B), MIC-67967 (C), and metconazole (A) a chemical fungicide. The lack of growth of Fusarium graminearum around MIC-67967 is notable in comparison to the control plate shown in FIG. 2B.

FIG. 2B shows an exemplary photo a R2A plate inoculated with Fusarium graminearum (B) and metconazole (A) a chemical fungicide; of note is the uninterrupted edge of pathogen growth on the left side of the plate.

FIG. 3A shows an exemplary photo a half strength potato dextrose agar (0.5×PDA) plate inoculated with Rhizoctonia solani (B), MIC-18905 (E), and metconazole (A) a chemical fungicide. The lack of growth of Rhizoctonia solani around MIC-18905 is notable in comparison to the control plate shown in FIG. 3C.

FIG. 3B shows an exemplary photo a 0.5×PDA plate inoculated with Rhizoctonia solani (B), MIC-84302 (D), and metconazole (A) a chemical fungicide. The lack of growth of Rhizoctonia solani around MIC-84302 is notable in comparison to the control plate shown in FIG. 3C.

FIG. 3C shows an exemplary photo a 0.5×PDA plate inoculated with Rhizoctonia solani (B) and metconazole (A) a chemical fungicide; of note is the uninterrupted edge of pathogen growth on the left side of the plate.

FIG. 4A shows an exemplary photo a half strength potato dextrose agar (0.5×PDA) plate inoculated with Rhizoctonia solani (B), MIC-67967 (C), and metconazole (A) a chemical fungicide. The lack of growth of Rhizoctonia solani around MIC-67967 is notable in comparison to the control plate shown in FIG. 4B.

FIG. 4B shows an exemplary photo a 0.5×PDA plate inoculated with Rhizoctonia solani (B) and metconazole (A) a chemical fungicide; of note is the uninterrupted edge of pathogen growth on the left side of the plate.

FIG. 5A shows an exemplary photo a half strength potato dextrose agar (0.5×PDA) plate inoculated with Pythium sp. (B), MIC-18905 (E), and mefenoxam (A) a chemical fungicide. The lack of growth of Pythium sp. around MIC-18905 is notable in comparison to the control plate shown in FIG. 5C.

FIG. 5B shows an exemplary photo a 0.5×PDA plate inoculated with Pythium sp. (B), MIC-84302 (D), and mefenoxam (A) a chemical fungicide. The lack of growth of Pythium sp. around MIC-84302 is notable in comparison to the control plate shown in FIG. 3C.

FIG. 5C shows an exemplary photo a 0.5×PDA plate inoculated with Pythium sp. (B) and mefenoxam (A) a chemical fungicide; of note is the uninterrupted edge of pathogen growth on the left side of the plate.

FIG. 6A shows an exemplary photo a yeast extract peptone dextrose agar (YEPD) plate inoculated with Pythium sp. (B), MIC-67967 (C), and mefenoxam (A) a chemical fungicide. The lack of growth of Pythium sp. around MIC-67967 is notable in comparison to the control plate shown in FIG. 4B.

FIG. 6B shows an exemplary photo a YEPD plate inoculated with Pythium sp. (B) and mefenoxam (A) a chemical fungicide; of note is the uninterrupted edge of pathogen growth on the left side of the plate.

### DETAILED DESCRIPTION

Terms used in the claims and specification are defined as set forth below unless otherwise specified.

It must be noted that, as used in the specification and the appended claims, the singular forms “a,” “an” and “the” include plural referents unless the context clearly dictates otherwise.

This invention relates to methods and compositions for improving plant health. The present invention includes methods for improving plant health, as well as synthetic compositions comprising endophytes capable of improving plant health, and nucleic acid probes and nucleic acid detection kits that may be used to identify endophytes of the present invention.

“Plant health” is demonstrated by the improvement of a trait of agronomic importance in a plant or plant element as compared to a reference plant or plant element. A trait of agronomic importance includes, but is not limited to, drought tolerance, heat tolerance, cold tolerance, salinity tolerance, metal tolerance, herbicide tolerance, improved water use efficiency, improved nitrogen utilization, improved nitrogen fixation, improved nutrient use efficiency, improved nutrient utilization, biotic stress tolerance, increased disease resistance, yield improvement, health enhancement, vigor improvement, decreased necrosis, decreased chlorosis, decreased area of necrotic tissue, increased stand count, decreased area of chlorotic tissue, decreased pathogen load of tissues, growth improvement, photosynthetic capability improvement, nutrition enhancement, altered protein content, altered oil content, increased biomass, increased shoot height, increased root length, increased shoot biomass, increased root biomass, increased leaf area, increased shoot area, increased root area, improved root architecture, increased seed germination percentage, increased seed germination rate, increased seedling survival, increased survival, photosynthetic efficiency, transpiration rate, seed/fruit number or mass, plant grain or fruit yield, leaf chlorophyll content, photosynthetic rate, wilt recovery, turgor pressure, modulation of a metabolite, production of a volatile organic compound (VOC), modulation of the proteome, increased seed weight, altered seed carbohydrate composition, altered seed oil composition, altered seed protein composition, altered seed nutrient composition, and combinations thereof. The phrase “biotic stress” refers to a growth environment comprising one or more pests or pathogens. Pests can be nematodes and/or insects. In some embodiments, a pest is of an order Lepidoptera, Hemiptera, Tylenchida/Rhabditida, Dorylaimida, Trichinellida, or Triplonchida. In some embodiments, a pest is of a genera Chrysodeixis, Trichoplusia, Nezara, Lygus, Aphis, Belonolaimus, Xiphenema, Trichodorus, Pratylenchus, Aphelenchoides, Meloidogyne, or Rotylenchulus. Pathogens can be fungal, viral, protist, or bacterial pathogens, for example of vertebrates or plants. In some embodiments, a pathogen is of a genera Pythium, Rhizoctonia, Phytophthora, Fusarium, Alternaria, Stagonospora, Aspergillus, Magnaporthe, Botrytis, Puccinia, Blumeria, Erysiphe, Leveillula, Mycosphaerella, or Colletotrichum.

“Biomass” means the total mass or weight (fresh or dry), at a given time (for example, age or stage of development), of a plant tissue, plant tissues, an entire plant, or population of plants. The term may also refer to all the plants or species in the community (“community biomass”).

An “increased yield” can refer to any increase in seed or fruit biomass; or seed, seed pod or ear, or fruit number per plant; or seed or fruit weight; or seed or fruit size per plant or unit of production area, e.g. acre or hectare. For example, increased yield of seed or fruit biomass may be measured in units of bushels per acre, pounds per acre, tons per acre, or kilos per hectare. An increased yield can also refer to an increase production of a component of, or product derived from, a plant or plant element or of a unit of measure thereof. For example, increased carbohydrate yield of a grain or increased oil yield of a seed. Typically, where yield indicates an increase in a particular component or product derived from a plant, the particular characteristic is designated when referring to increased yield, e.g., increased oil or grain yield or increased protein yield or seed size.

“Nutrition enhancement” refers to modulation of the presence, abundance or form of one or more substances in a plant element, wherein the modulation of the one or more substances provides a benefit to other organisms that consume or utilize said plant element.

Synthetic compositions and methods of use described herein may improve plant health by providing an improved benefit or tolerance to a plant that is of at least 0.1%, at least 0.5%, at least 1%, at least 2%, at least 3%, between 3% and 5%, at least 5%, between 5% and 10%, at least 10%, between 10% and 15%, for example at least 15%, between 15% and 20%, at least 20%, between 20% and 30%, at least 30%, between 30% and 40%, at least 40%, between 40% and 50%, at least 50%, between 50% and 60%, at least 60%, between 60% and 75%, at least 75%, between 75% and 100%, at least 100%, between 100% and 150%, at least 150%, between 150% and 200%, at least 200%, between 200% and 300%, at least 300% or more, when compared with a reference plant. A “reference plant”, “reference plant element”, “reference agricultural plant” or “reference seed” means a similarly situated plant or seed of the same species, strain, or cultivar to which a treatment, formulation, composition or endophyte preparation as described herein is not administered/contacted. A reference plant, therefore, is identical to the treated plant except for the presence of the active ingredient to be tested and can serve as a control for detecting the effects of the treatment conferred to the plant. A plurality of reference plants may be referred to as a “reference population”.

In some embodiments, one or more endophytes and or one or more compounds produced by one or more endophytes are heterologously disposed on a plant element in an effective amount to improve plant health. In some embodiments, an improvement of plant health is measured by an increase in a trait of agronomic importance, for example root length or yield. In some embodiments, an improvement of subject health is measured by a decrease in a trait of importance, for example necrosis or chlorosis. In some embodiments, improved plant health is demonstrated by an improvement of a trait of agronomic importance or tolerance in a treated plant by at least 0.1%, at least 0.5%, at least 1%, at least 2%, at least 3%, between 3% and 5%, at least 5%, between 5% and 10%, at least 10%, between 10% and 15%, for example at least 15%, between 15% and 20%, at least 20%, between 20% and 30%, at least 30%, between 30% and 40%, at least 40%, between 40% and 50%, at least 50%, between 50% and 60%, at least 60%, between 60% and 75%, at least 75%, between 75% and 100%, at least 100%, between 100% and 150%, at least 150%, between 150% and 200%, at least 200%, between 200% and 300%, at least 300% or more, as compared to a reference plant element not further comprising said endophyte. An “effective amount” of one or more endophytes is the amount capable of improving trait of agronomic importance or tolerance by at least 0.1%, at least 0.5%, at least 1%, at least 2%, at least 3%, between 3% and 5%, at least 5%, between 5% and 10%, at least 10%, between 10% and 15%, for example at least 15%, between 15% and 20%, at least 20%, between 20% and 30%, at least 30%, between 30% and 40%, at least 40%, between 40% and 50%, at least 50%, between 50% and 60%, at least 60%, between 60% and 75%, at least 75%, between 75% and 100%, at least 100%, between 100% and 150%, at least 150%, between 150% and 200%, at least 200%, between 200% and 300%, at least 300% or more, as compared to a reference plant element not further comprising said endophyte. In some embodiments, an effective amount of treatment comprising an endophyte is at least 10 CFU per unit of plant element, at least 10\hat{ }2 CFU per unit of plant element, between 10\hat{ }2 and 10\hat{ }3 CFU per unit of plant element, at least about 10\hat{ }3 CFU per unit of plant element, between 10\hat{ }3 and 10\hat{ }4 CFU per unit of plant element, at least about 10\hat{ }4 CFU per unit of plant element, between 10\hat{ }4 and 10\hat{ }5 CFU per unit of plant element, at least about 10\hat{ }5 CFU, between 10\hat{ }5 and 10\hat{ }6 CFU per unit of plant element, at least about 10\hat{ }6 CFU per unit of plant element, between 10\hat{ }6 and 10\hat{ }7 CFU per unit of plant element, at least about 10\hat{ }7 CFU per unit of plant element, between 10\hat{ }7 and 10\hat{ }8 CFU per unit of plant element, or even greater than 10\hat{ }8 CFU per unit of plant element. A unit of a plant element may be an individual plant element, e.g. an individual seed, or a unit of area surface area of a plant element, e.g. a square inch of leaf tissue, or unit of surface area of a plant element, e.g. a cubic centimeter of root.

The methods and compositions of the present invention are broadly applicable to cultivated plants, particularly plants that are cultivated by humans for food, feed, fiber, fuel, and/or industrial purposes. In some embodiments, plants (including seeds and other plant elements) are monocots or dicots. In some embodiments, plants used in the methods and compositions of the present invention include, but are not limited to: agricultural row, agricultural grass plants or other field crops: wheat, rice, barley, buckwheat, beans (for example: soybean, snap, dry), corn (for example: grain, seed, sweet corn, silage, popcorn, high oil), canola, peas (for example: dry, succulent), peanuts, safflower, sunflower, alfalfa hay, forage and cover crops (for example: alfalfa, clover, vetch, and trefoil), berries and small fruits (for example: blackberries, blueberries, currants, elderberries, gooseberries, huckleberries, loganberries, raspberries, strawberries, bananas and grapes), bulb crops (for example: garlic, leeks, onions, shallots, and ornamental bulbs), citrus fruits (for example: citrus hybrids, grapefruit, kumquat, lines, oranges, and pummelos), cucurbit vegetables (for example: cucumbers, melons, gourds, pumpkins, and squash), flowers (for example: ornamental, horticultural flowers including roses, daisies, tulips, freesias, carnations, heather, lilies, irises, orchids, snapdragons, and ornamental sunflowers), bedding plants, ornamentals, fruiting vegetables (for example: eggplant, sweet and hot peppers, tomatillos, and tomatoes), herbs, spices, mints, hydroponic crops (for example: cucumbers, tomatoes, lettuce, herbs, and spices), leafy vegetables and cole crops (for example: arugula, celery, chervil, endive, fennel, lettuce including head and leaf, parsley, radicchio, rhubarb, spinach, Swiss chard, broccoli, Brussels sprouts, cabbage, cauliflower, collards, kale, kohlrabi, and mustard greens), asparagus, legume vegetable and field crops (for example: snap and dry beans, lentils, succulent and dry peas, and peanuts), pome fruit (for example: pears and quince), root crops (for example: beets, sugarbeets, red beets, carrots, celeriac, chicory, horseradish, parsnip, radish, rutabaga, salsify, and turnips), deciduous trees (for example: maple and oak), evergreen trees (for example: pine, cedar, hemlock and spruce), small grains (for example: rye, wheat including spring and winter wheat, millet, oats, barley including spring and winter barley, and spelt), stone fruits (for example: apricots, cherries, nectarines, peaches, plums, and prunes), tree nuts (for example: almonds, beech nuts, Brazil nuts, butternuts, cashews, chestnuts, filberts, hickory nuts, macadamia nuts, pecans, pistachios, and walnuts), and tuber crops (for example: potatoes, sweet potatoes, yams, artichoke, cassava, and ginger). In a particular embodiment, the agricultural plant is selected from the group consisting of rice (Oryza sativa and related varieties), soy (Glycine max and related varieties), wheat (Triticum aestivum and related varieties), oats (Avena sativa and related varieties), barley (Hordeum vulgare and related varieties), corn (Zea mays and related varieties), peanuts (Arachis hypogaea and related varieties), canola (Brassica napus, Brassica rapa and related varieties), coffee (Coffea spp.), cocoa (Theobroma cacao), melons, and tomatoes (Solanum lycopsersicum and related varieties).

Plant health may be improved by treatment of a plant or plant element. A “plant element” is intended to generically reference either a whole plant or a plant component, including but not limited to plant tissues, parts, and cell types. A plant element is preferably one of the following: whole plant, seedling, meristematic tissue, ground tissue, vascular tissue, dermal tissue, seed, leaf, root, shoot, stem, flower, fruit, stolon, bulb, tuber, corm, keikis, shoot, or bud.

Plant health may be improved by treatment with a composition of the present invention, in particular compositions of the present invention comprising one or more endophytes. An “endophyte” is an organism capable of living on a plant element (e.g., rhizoplane or phyllosphere) or within a plant element, or on a surface in close physical proximity with a plant element, e.g., the phyllosphere and rhizosphere including soil surrounding roots. A “beneficial” endophytes does not cause disease or harm the host plant otherwise. Endophytes can occupy the intracellular or extracellular spaces of plant tissue, including the leaves, stems, flowers, fruits, seeds, or roots. An endophyte can be, for example, a bacterial or fungal organism, and can confer a beneficial property to the host plant such as an increase in yield, biomass, resistance, or fitness. An endophyte can be a fungus or a bacterium. As used herein, the term “microbe” is sometimes used to describe an endophyte. As used herein, the term “microbe” or “microorganism” refers to any species or taxon of microorganism, including, but not limited to, archaea, bacteria, microalgae, fungi (including mold and yeast species), mycoplasmas, microspores, nanobacteria, oomycetes, and protozoa. In some embodiments, a microbe or microorganism is an endophyte, for example a bacterial or fungal endophyte, which is capable of living within a plant.

The term “isolated” is intended to specifically reference an organism, cell, tissue, polynucleotide, or polypeptide that is removed from its original source and purified from additional components with which it was originally associated. For example, an endophyte may be considered isolated from a seed if it is removed from that seed source and purified so that it is isolated from one or more additional components with which it was originally associated. Similarly, an endophyte may be removed and purified from a plant or plant element so that it is isolated and no longer associated with its source plant or plant element.

As used herein, an isolated strain of a microbe is a strain that has been removed from its natural milieu. “Pure cultures” or “isolated cultures” are cultures in which the organisms present are only of one strain of a particular genus and species. This is in contrast to “mixed cultures,” which are cultures in which more than one genus and/or species of microorganism are present. As such, the term “isolated” does not necessarily reflect the extent to which the microbe has been purified. A “substantially pure culture” of the strain of microbe refers to a culture which contains substantially no other microbes than the desired strain or strains of microbe. In other words, a substantially pure culture of a strain of microbe is substantially free of other contaminants, which can include microbial contaminants. Further, as used herein, a “biologically pure” strain is intended to mean the strain was separated from materials with which it is normally associated in nature. A strain associated with other strains, or with compounds or materials that it is not normally found with in nature, is still defined as “biologically pure.” A monoculture of a particular strain is, of course, “biologically pure.” As used herein, the term “enriched culture” of an isolated microbial strain refers to a microbial culture that contains more that 50%, 60%, 70%, 80%, 90%, or 95% of the isolated strain.

A “population” of endophytes, or an “endophyte population”, refers to one or more endophytes that share a common genetic derivation, e.g., one or more propagules of a single endophyte, i.e., endophytes grown from a single picked colony. In some embodiments, a population refers to endophytes of identical taxonomy. In some cases, a population of endophytes refers to one or more endophytes of the same genus. In some cases, a population of endophytes refers to one or more endophytes of the same species or strain.

A “plurality of endophytes” means two or more types of endophyte entities, e.g., of bacteria or fungi, or combinations thereof. In some embodiments, the two or more types of endophyte entities are two or more individual endophytic organisms, regardless of genetic derivation or taxonomic relationship. In some embodiments, the two or more types of endophyte entities are two or more populations of endophytes. In other embodiments, the two or more types of endophyte entities are two or more species of endophytes. In yet other embodiments, the two or more types of endophyte entities are two or more genera of endophytes. In yet other embodiments, the two or more types of endophyte entities are two or more families of endophytes. In yet other embodiments, the two or more types of endophyte entities are two or more orders of endophytes. In yet other embodiments, the two or more types of endophyte entities are two or more classes of endophytes. In yet other embodiments, the two or more types of endophyte entities are two or more phyla of endophytes. In some embodiments, a plurality refers to three or more endophytes, either distinct individual organisms or distinct members of different genetic derivation or taxa. In some embodiments, a plurality refers to four or more either distinct individual endophytic organisms or distinct members of different genetic derivation or taxa. In some embodiments, a plurality refers to five or more, ten or more, or an even greater number of either distinct individual endophytic organisms or distinct members of different genetic derivation or taxa. In some embodiments, the term “consortium” or “consortia” may be used as a collective noun synonymous with “plurality”, when describing more than one population, species, genus, family, order, class, or phylum of endophytes.

In some embodiments, a treatment may comprise a modified microbe or plant or plant element. A microbe or plant or plant element is “modified” when it comprises an artificially introduced genetic or epigenetic modification. In some embodiments, the modification is introduced by a genome engineering or genome editing technology. In some embodiments, genome engineering or editing utilizes non-homologous end joining (NHEJ), homology directed repair (HDR), or combinations thereof. In some embodiments, genome engineering or genome editing is carried out with a Class I or Class II clustered regulatory interspaced short palindromic repeats (CRISPR) system. In some embodiments, the CRISPR system is CRISPR/Cas9. In some embodiments, the CRISPR system is CRISPR/Cpf1. In some embodiments, the modification is introduced by a targeted nuclease. In some embodiments, targeted nucleases include, but are not limited to, transcription activator-like effector nuclease (TALEN), zinc finger nuclease (ZNF), Cas9, Cas9 variants, Cas9 homologs, Cpf1, Cpf1 variants, Cpf1 homologs, and combinations thereof. In some embodiments, the modification is an epigenetic modification. In some embodiments, the modification is introduced by treatment with a DNA methyltransferase inhibitor such as 5-azacytidine, or a histone deacetylase inhibitor such as 2-amino-7-methoxy-3H-phenoxazin-3-one. In some embodiments, the modification is introduced via tissue culture. In some embodiments, a modified microbe or plant or plant element comprises a transgene.

As used herein, the term “bacterium” or “bacteria” refers in general to any prokaryotic organism and may reference an organism from either Kingdom Eubacteria (Bacteria), Kingdom Archaebacteria (Archaea), or both. In some cases, bacterial genera have been reassigned due to various reasons (such as, but not limited to, the evolving field of whole genome sequencing), and it is understood that such nomenclature reassignments are within the scope of any claimed genus.

As used herein, the term “fungus” or “fungi” refers in general to any organism from Kingdom Fungi. Historical taxonomic classification of fungi has been according to morphological presentation. Beginning in the mid-1800's, it was recognized that some fungi have a pleomorphic life cycle, and that different nomenclature designations were being used for different forms of the same fungus. With the development of genomic sequencing, it became evident that taxonomic classification based on molecular phylogenetics did not align with morphological-based nomenclature (Shenoy B D, Jeewon R, Hyde K D. Impact of DNA sequence-data on the taxonomy of anamorphic fungi. Fungal Diversity 26(10) 1-54. 2007). Systematics experts have not aligned on common nomenclature for all fungi, nor are all existing databases and information resources inclusive of updated taxonomies. As such, many fungi provided herein may be described by their anamorph form, but it is understood that based on identical genomic sequencing, any pleomorphic state of that fungus may be considered to be the same organism. In some cases, fungal genera have been reassigned due to various reasons, and it is understood that such nomenclature reassignments are within the scope of any claimed genus.

The degree of relatedness between microbes may be inferred from the sequence similarity of one or more homologous polynucleotide sequences of the microbes. In some embodiments, the one or more homologous polynucleotide sequences are marker genes. As used herein, the term “marker gene” refers to a conserved genomic region comprising sequence variation among related organisms. Examples of marker genes that may be used for the present invention, include but are not limited to: 16S ribosomal RNA gene (“16S”), internal transcribed spacer (“ITS”); fusA gene; largest subunit of RNA polymerase II (“RPB1”); second largest subunit of RNA polymerase II (“RPB2”); beta-tubulin or tubulin (“BTUB2” or “TUB2”); phosphoglycerate kinase (“PGK”); actin (“ACT”); long subunit rRNA gene (“LSU”); small subunit rRNA gene (“SSU”), 60S ribosomal protein L 10 (“60S_L10_L1”), atpD, Calmodulin (“CMD”), GDP gene (“GPD1_2”), etc.

The terms “sequence similarity”, “identity”, “percent identity”, “percent sequence identity” or “identical” in the context of polynucleotide sequences refer to the nucleotides in the two sequences that are the same when aligned for maximum correspondence. There are different algorithms known in the art that can be used to measure nucleotide sequence identity. Nucleotide sequence identity can be measured by a local or global alignment, preferably implementing an optimal local or optimal global alignment algorithm. For example, a global alignment may be generated using an implementation of the Needleman-Wunsch algorithm (Needleman, S. B. & Wunsch, C. D. (1970) Journal of Molecular Biology. 48(3):443-53). For example, a local alignment may be generated using an implementation of the Smith-Waterman algorithm (Smith T. F & Waterman, M. S. (1981) Journal of Molecular Biology. 147(1): 195-197). Optimal global alignments using the Needleman-Wunsch algorithm and optimal local alignments using the Smith-Waterman algorithm are implemented in USEARCH, for example USEARCH version v8.1.1756_i86osx32.

A gap is a region of an alignment wherein a sequence does not align to a position in the other sequence of the alignment. A terminal gap is a region beginning at the end of a sequence in an alignment wherein the nucleotide in the terminal position of that sequence does not correspond to a nucleotide position in the other sequence of the alignment and extending for all contiguous positions in that sequence wherein the nucleotides of that sequence do not correspond to a nucleotide position in the other sequence of the alignment. An internal gap is a gap in an alignment which is flanked on the 3′ and 5′ end by positions wherein the aligned sequences are identical. In global alignments, terminal gaps are discarded before identity is calculated. For both local and global alignments, internal gaps are counted as differences.

In some embodiments, the nucleic acid sequence to be aligned is a complete gene. In some embodiments, the nucleic acid sequence to be aligned is a gene fragment. In some embodiments, the nucleic acid sequence to be aligned is an intergenic sequence. In a preferred embodiment, inference of homology from a sequence alignment is made where the region of alignment is at least 85% of the length of the query sequence.

The term “substantial homology” or “substantial similarity,” when referring to a polynucleotide sequence or fragment thereof, indicates that, when optimally aligned with appropriate nucleotide insertions or deletions with another polynucleotide sequence (or its complementary strand), there is nucleotide sequence identity in at least about 76%, 80%, 85%, or at least about 90%, or at least about 95%, 96%, at least 97%, 98%, 99% or 100% of the positions of the alignment, wherein the region of alignment is at least about 50%, 60%, 70%, 75%, 85%, or at least about 90%, or at least about 95%, 96%, 97%, 98%, 99% or 100% of the length of the query sequence. In a preferred embodiment, the region of alignment contains at least 100 positions inclusive of any internal gaps. In some embodiments, the region of alignment comprises at least 100 nucleotides of the query sequence. In some embodiments, the region of alignment comprises at least 200 nucleotides of the query sequence. In some embodiments, the region of alignment comprises at least 300 nucleotides of the query sequence. In some embodiments, the region of alignment comprises at least 400 nucleotides of the query sequence. In some embodiments, the region of alignment comprises at least 500 nucleotides of the query sequence. In some embodiments, the terminal nucleotides are trimmed from one or both ends of the sequence prior to alignment. In some embodiments, at least the terminal 10, 15, 20, 25, 30, between 20-30, 35, 40, 45, 50, between 25-50 nucleotides are trimmed from the sequence prior to alignment.

### Synthetic Compositions for Improving Plant Health

In some embodiments, a synthetic composition comprises one or more endophytes capable of improving plant health. A “synthetic composition” comprises one or more endophytes combined by human endeavor with a heterologously disposed plant element or a treatment formulation, said combination which is not found in nature. In some embodiments, a synthetic composition comprises one or more plant elements or formulation components combined by human endeavor with an isolated, purified endophyte composition. In some embodiments, synthetic composition refers to a plurality of endophytes in a treatment formulation comprising additional components with which said endophytes are not found in nature. An endophyte is “heterologously disposed” when mechanically or manually applied, artificially inoculated or disposed onto or into a plant element, seedling, plant or onto or into a plant growth medium or onto or into a treatment formulation so that the endophyte exists on or in the plant element, seedling, plant, plant growth medium, or formulation in a manner not found in nature prior to the application of the treatment, e.g., said combination which is not found in nature in that plant variety, at that time in development, in that tissue, in that abundance, or in that growth condition (for example, drought, flood, cold, nutrient deficiency, etc.).

A “treatment formulation” refers to one or more compositions that facilitate the stability, storage, and/or application of one or more endophytes. Treatment formulations may comprise any one or more agents such as: a surfactant, a buffer, a tackifier, a microbial stabilizer, an antimicrobial, a fungicide, an anticomplex agent, an herbicide, a nematicide, an insecticide, a plant growth regulator, a rodenticide, a desiccant, a nutrient, an excipient, a wetting agent, a salt, a polymer. As used herein as a noun, a “treatment” may comprise one or more endophytes.

In some embodiments, a treatment formulation may comprise one or more polymeric beads comprising one or more endophytes. In some embodiments, a treatment formulation may consist of one or more polymeric beads comprising one or more endophytes. A polymeric bead may contain a biodegradable polymer such as alginate, agarose, agar, gelatin, polyacrylamide, chitosan, and polyvinyl alcohol. In some embodiments, the polymeric beads are less than 500 μm in diameter at their widest point. In some embodiments, the polymeric beads' average diameter at their widest point is between 500 μm and 250 μm, between 249 μm and 100 μm, 100 μm or less, between 100 μm and 50 μm, or 50 μm or less.

In some embodiments, an “agriculturally compatible carrier” can be used to formulate an agricultural formulation or other composition that includes a purified endophyte preparation. As used herein an “agriculturally compatible carrier” refers to any material, other than water, that can be added to a plant element without causing or having an adverse effect on the plant element (e.g., reducing seed germination) or the plant that grows from the plant element, or the like.

In some embodiments, the formulation can include a tackifier or adherent. Such agents are useful for combining the bacterial population of the invention with carriers that can contain other compounds (e.g., control agents that are not biologic), to yield a coating composition. Such compositions help create coatings around the plant or seed to maintain contact between the microbe and other agents with the plant or plant part. In some embodiments, adherents are selected from the group consisting of: alginate, gums, starches, lecithins, formononetin, polyvinyl alcohol, alkali formononetinate, hesperetin, polyvinyl acetate, cephalins, Gum Arabic, Xanthan Gum, Mineral Oil, Polyethylene Glycol (PEG), Polyvinyl pyrrolidone (PVP), Arabino-galactan, Methyl Cellulose, PEG 400, Chitosan, Polyacrylamide, Polyacrylate, Polyacrylonitrile, Glycerol, Triethylene glycol, Vinyl Acetate, Gellan Gum, Polystyrene, Polyvinyl, Carboxymethyl cellulose, Gum Ghatti, and polyoxyethylene-polyoxybutylene block copolymers.

The formulation can also contain a surfactant. Non-limiting examples of surfactants include nitrogen-surfactant blends such as Prefer 28 (Cenex), Surf-N(US), Inhance (Brandt), P-28 (Wilfarm) and Patrol (Helena); esterified seed oils include Sun-It II (AmCy), MSO (UAP), Scoil (Agsco), Hasten (Wilfarm) and Mes-100 (Drexel); and organo-silicone surfactants include Silwet L77 (UAP), Silikin (Terra), Dyne-Amie (Helena), Kinetic (Helena), Sylgard 309 (Wilbur-Ellis) and Century (Precision). In one embodiment, the surfactant is present at a concentration of between 0.01% v/v to 10% v/v. In another embodiment, the surfactant is present at a concentration of between 0.1% v/v to 1% v/v.

In certain cases, the formulation includes a microbial stabilizer. Such an agent can include a desiccant. As used herein, a “desiccant” can include any compound or mixture of compounds that can be classified as a desiccant regardless of whether the compound or compounds are used in such concentrations that they in fact have a desiccating effect on the liquid inoculant. Such desiccants are ideally compatible with the bacterial population used, and should promote the ability of the microbial population to survive application on the seeds and to survive desiccation. Examples of suitable desiccants include one or more of trehalose, sucrose, glycerol, and Methylene glycol. Other suitable desiccants include, but are not limited to, non reducing sugars and sugar alcohols (e.g., mannitol or sorbitol). The amount of desiccant introduced into the formulation can range from about 5% to about 50% by weight/volume, for example, between about 10% to about 40%, between about 15% and about 35%, or between about 20% and about 30%.

In some embodiments the formulation includes, for example, solid carriers such as talc, fullers earth, bentonite, kaolin clay, pyrophyllite, bentonite, montmorillonite, diatomaceous earth, acid white soil, vermiculite, and pearlite, and inorganic salts such as ammonium sulfate, ammonium phosphate, ammonium nitrate, urea, ammonium chloride, and calcium carbonate. Also, organic fine powders such as wheat flour, wheat bran, and rice bran maybe used. The liquid carriers include vegetable oils such as soybean oil and cottonseed oil, glycerol, ethylene glycol, polyethylene glycol, propylene glycol, polypropylene glycol, etc.

In some embodiments, the abundance of an endophyte can be estimated by methods well known in the art including, but not limited to, qPCR, community sequencing, flow cytometry, and/or counting colony-forming units. As used herein, a “colony-forming unit” (“CFU”) is used as a measure of viable microorganisms in a sample. A CFU is an individual viable cell capable of forming on a solid medium a visible colony whose individual cells are derived by cell division from one parental cell.

In some embodiments, the synthetic composition of the present invention comprises one or more of the following: antimicrobial, fungicide, nematicide, bactericide, insecticide, or herbicide.

In some embodiments, the time to 1 log loss in CFU of an endophyte in formulation is at greater than or equal to 168 days, greater than or equal to 150 days, greater than or equal to 125 days, greater than or equal to 100 days, greater than or equal to 75 days, greater than or equal to 50 days, greater than or equal to 20 days at 4 degrees Celsius. In some embodiments, the time to 1 log loss in CFU of an endophyte in formulation is at least 140 days, at least 90 days, at least 60 days, at least 50 days, at least 30 days, at least 20 days, at 22 degrees Celsius. In some embodiments, the time to 2 log loss in CFU of an endophyte on a seed is at least 3 days, at least 5 days, at least 10 days, at least 20 days, at least 21 days, at least 22 days, at least 23 days, at least 24 days, at least 25 days, at 22 degrees Celsius.

In some embodiments, a treatment is applied mechanically or manually or artificially inoculated to a plant element in a seed treatment, root wash, seedling soak, foliar application, floral application, soil inoculum, in-furrow application, sidedress application, soil pre-treatment, wound inoculation, drip tape irrigation, vector-mediation via a pollinator, injection, osmopriming, hydroponics, aquaponics, aeroponics, and combinations thereof. Application to the plant may be achieved, for example, as a powder for surface deposition onto plant leaves, as a spray to the whole plant or selected plant element, as part of a drip to the soil or the roots, or as a coating onto the plant element prior to or after planting. Such examples are meant to be illustrative and not limiting to the scope of the invention.

In some embodiments, the invention described herein provides a synthetic composition comprising one or more endophytes capable of improving plant health, wherein the one or more endophytes is a member of the Class Chitinophagia, Bacilli, Gammaproteobacteria, or Sordariomycetes. In some embodiments, the one or more endophytes is a member of the Order Chitinophagales, Bacillales, Pseudomonadales, or Hypocreales. In some embodiments, the one or more endophytes is a member of the Family Chitinophagaceae, Bacillaceae, Pseudomonadaceae, or Hypocreaceae. In some embodiments, the one or more endophytes is a member of the Genus Chitinophaga, Bacillus, Pseudomonas, or Trichoderma. In some embodiments, the one or more endophytes are selected from Table 3. In some embodiments, the one or more endophytes comprise one or more polynucleotide sequences at least 95%, at least 96%, at least 97%, at least 97%, at least 98%, at least 99%, or 100% identical to one or more of SEQ ID NOs. 27, 32-93, 127, or 132. In some embodiments, the one or more endophytes are capable of producing a protein whose amino acid sequence is at least 95%, at least 96%, at least 97%, at least 97%, at least 98%, at least 99%, or 100% identical to one or more of SEQ ID NOs. 28-31, 94-126, 128-131, 133-135, or 136.

In some embodiments of any of the synthetic compositions described herein, the synthetic compositions comprise at least 2, 3, 4, 5, 6, 7, 8, 9, 10, 15 or 20 or more endophytes. In some embodiments, the one or more endophytes comprise at least 2, 3, 4, 5, 6, 7, 8, 9, 10, 15 or 20 or more endophytes. In some embodiments, the one or more endophytes are distinct individual organisms or distinct members of different genetic derivation or taxa.

### Methods for Improving Plant Health

In some embodiments, the invention provides methods of improving plant health comprising heterologously disposing one or more endophytes to a plant element in an effective amount to increase a trait of agronomic importance in the plant derived from the treated plant element relative to a plant derived from a reference plant element. In some embodiments, the one or more endophytes are a component of a treatment formulation. In some embodiments, the one or more endophytes are a component of a synthetic composition.

In some embodiments, the invention provides methods of improving plant health comprising creating any of the synthetic compositions described herein, wherein the synthetic composition comprises any of the plant elements of any of the plants described herein and any of the one or more endophytes described herein. In some embodiments, the synthetic composition comprises any of the treatment formulations described herein and any of the one or more endophytes described herein. In some embodiments, the synthetic composition additionally comprises a growth medium or growth environment. A growth environment is a natural or artificially constructed surrounding capable of supporting life of a plant. In some embodiments, the growth medium is soil. In some embodiments, the growth medium is a culture fluid suitable for propagation of an endophyte or plant tissue culture. In some embodiments, the method comprises a step of applying the synthetic composition to a growth medium. In some embodiments, the synthetic composition is applied before one or more plant elements are placed in or on the growth medium. In some embodiments, the synthetic composition is applied after one or more plant elements are placed in or on the growth medium. In some embodiments, the method comprises a step of germinating the plants. In some embodiments, the method comprises a step of growing the plants. For example, the plants may be grown in the plant vigor assays, greenhouse assessments, or field trials described herein. In some embodiments, the method comprises a step of growing the plants to maturity. In some embodiments, where the plants are commercially produced, maturity is the stage at which the plant is normally harvested.

In some embodiments of any of the methods described herein, plant health may be improved for plants in a stress condition. In some embodiments, the stress condition is a biotic or abiotic stress, or a combination of one or more biotic or abiotic stresses. In some embodiments of any of the methods described herein, the stress condition is an abiotic stress selected from the group consisting of: drought stress, salt stress, metal stress, heat stress, cold stress, low nutrient stress (alternately referred to herein as nutrient deficiency or growth in nutrient deficient conditions), and excess water stress, and combinations thereof. In some embodiments of any of the methods described herein, the stress condition is a biotic stress selected from the group consisting of: insect infestation, nematode infestation, complex infection, fungal infection, bacterial infection, oomycete infection, protozoal infection, viral infection, herbivore grazing, and combinations thereof. Stress tolerance is exemplified by improvement of one or more other traits of agronomic importance when compared with a reference plant, reference plant element, or reference population. For example, biotic stress tolerance may be shown by decreased pathogen load of tissues, decreased area of chlorotic tissue, decreased necrosis, improved growth, increased survival, increased biomass, increased shoot height, increased root length, etc. relative to a reference.

### EXAMPLES

### Example 1. Isolation and Identification of Endophytes

### DNA Extraction

Endophytes of the present invention were isolated from the sources listed in Table 1.

TABLE 1
Sources of microbes of the present invention

| | | Isolation |
|---|---|---|
| MIC ID | Isolated From | Condition |
| MIC-67967 | Oryza sativa (rice) | Low nutrient |
| MIC-84302 | Lactuca serriola (Prickly lettuce) | |
| MIC-18905 | Glycine max (soybean) | Cold stress |
| MIC-54347 | Triticum aestivum (wheat) | |

Each sample was processed independently. Each sample was washed in a dilute water and detergent solution; tissue was collected from plants. Samples were surface sterilized by successive rinses: 2 minutes in 10% bleach solution, 2 minutes in 70% ethanol solution, and a rinse with sterile water. The series of rinses was repeated 3 times. The plant tissue was cut into small pieces with sterile scissors and blended with 3, 7 mm steel beads in 5-7.5 ml phosphate buffered solution (PBS). DNA was extracted from the ground tissues using the Magbind Plant DNA kit (Omega, Norcross, Georgia, USA) according to the manufacturer's instructions.

### Identification of Endophytes by Sequencing of Marker Genes

The endophytes were characterized by the sequences of genomic regions. Primers that amplify genomic regions of the endophytes of the present invention are listed in Table 2. Sanger sequencing was performed at Genewiz (South Plainfield, NJ). Raw chromatograms were converted to sequences, and corresponding quality scores were assigned using TraceTuner v3.0.6beta (U.S. Pat. No. 6,681,186). These sequences were quality filtered, aligned and a consensus sequence generated using Geneious v 8.1.8 (Biomatters Limited, Auckland NZ). The consensus sequences identifying the endophytes are listed in Table 3.

TABLE 2
Primer sequences useful in identifying microbes of the present invention

| Seq ID | Genomic Locus | Sequence |
|---|---|---|
| 1 | 27f, 16S, 16S IRNA gene | AGAGTTTGATYMTGGCTCAG |
| 2 | 1492r, 16S, 16S rRNA gene | GGTTACCTTGTTACGACTT |
| 3 | 515f, 16S, 16S rRNA gene | GTGYCAGCMGCCGCGGTAA |
| 4 | 806r, 16S, 16S rRNA gene | GGACTACNVGGGTWTCTAAT |
| 5 | ITSIF, ITS, Internal transcribed spacer | CTTGGTCATTTAGAGGAAGTAA |
| 6 | LR5, ITS, Internal transcribed spacer | TCCTGAGGGAAACTTCG |
| 7 | ITS2, ITS, Internal transcribed spacer | GCTGCGTTCTTCATCGATGC |
| 8 | ITS3, ITS, Internal transcribed spacer | GCATCGATGAAGAACGCAGC |
| 9 | ACT512f, ACT, Actin | ATGTGCAAGGCCGGTTTCG |
| 10 | ACT783r, ACT, Actin | TACGAGTCCTTCTGGCCCAT |
| 11 | CA14, ACT, Actin | AACTGGGATGACATGGAGAAGATCTGGC |
| 12 | CA5R, ACT, Actin | GTGAACAATGGATGGACCAGATTCGTCG |
| 13 | Cmd5, CMD, Calmodulin | CCGAGTACAAGGARGCCTTC |
| 14 | Cmd6, CMD, Calmodulin | CCGATRGAGGTCATRACGTGG |
| 15 | 5.8S, ITS, Internal transcribed spacer | CGCTGCGTTCTTCATCG |
| 16 | 5.8SR, ITS, Internal transcribed spacer | TCGATGAAGAACGCAGCG |
| 17 | ITS1, ITS, Internal transcribed spacer | TCCGTAGGTGAACCTGCGG |
| 18 | ITS4, ITS, Internal transcribed spacer | TCCTCCGCTTATTGATATGC |
| 19 | ITS5, ITS, Internal transcribed spacer | GGAAGTAAAAGTCGTAACAAGG |
| 20 | LS266, ITS, Internal transcribed spacer | GCATTCCCAAACAACTCGACTC |
| 21 | V9G, ITS, Internal transcribed spacer | TTACGTCCCTGCCCTTTGTA |
| 22 | 5F_Eur, RPB2, Second largest subunit of RNA | GAYGAYCGKGAYCAYTTCGG |
| | polymerase II | |
| 23 | 7CR_Eur, RPB2, Second largest subunit of RNA | CCCATRGCYTGYTTRCCCAT |
| | polymerase II | |
| 24 | bRPB2-7.1R, RPB2, Second largest subunit of | CCCATRGCYTGYTTMCCCATDGC |
| | RNA polymerase II | |
| 25 | fRPB2-5F, RPB2, Second largest subunit of RNA | GAYGAYMGWGATCAYTTYGG |
| | polymerase II | |
| 26 | fRPB2-7R, RPB2, Second largest subunit of RNA | CCCATWGCYTGCTTMCCCAT |
| | polymerase II | |

TABLE 3
Exemplary sequences of endophytes of the present invention

| SEQ | MIC | |
|---|---|---|
| ID | ID | Sequence |
| 27 | MIC-18905 | CTCAGATTGAACGCTGGCGGCAGGCCTAACACATGCAAGTCGAGCGGATGAAAGGAGCTTGC |
| | | TCCTGGATTCAGCGGCGGACGGGTGAGTAATGCCTAGGAATCTGCCTGGTAGTGGGGGACAA |
| | | CGTTTCGAAAGGAACGCTAATACCGCATACGTCCTACGGGAGAAAGCAGGGGACCTTCGGGC |
| | | CTTGCGCTATCAGATGAGCCTAGGTCGGATTAGCTAGTTGGTGAGGTAATGGCTCACCAAGG |
| | | CGACGATCCGTAACTGGTCTGAGAGGATGATCAGTCACACTGGAACTGAGACACGGTCCAGA |
| | | CTCCTACGGGAGGCAGCAGTGGGGAATATTGGACAATGGGCGAAAGCCTGATCCAGCCATGC |
| | | CGCGTGTGTGAAGAAGGTCTTCGGATTGTAAAGCACTTTAAGTTGGGAGGAAGGGTTGTAGA |
| | | TTAATACTCTGCAATTTTGACGTTACCGACAGAATAAGCACCGGCTAACTCTGTGCCAGCAG |
| | | CCGCGGTAATACAGAGGGTGCAAGCGTTAATCGGAATTACTGGGCGTAAAGCGCGCGTAGGT |
| | | GGTTTGTTAAGTTGGATGTGAAATCCCCGGGCTCAACCTGGGAACTGCATCCAAAACTGGCA |
| | | AGCTAGAGTATGGTAGAGGGTGGTGGAATTTCCTGTGTAGCGGTGAAATGCGTAGATATAGG |
| | | AAGGAACACCAGTGGCGAAGGCGACCACCTGGACTGATACTGACACTGAGGTGCGAAAGCGT |
| | | GGGGAGCAAACAGGATTAGATACCCTGGTAGTCCACGCCGTAAACGATGTCAACTAGCCGTT |
| | | GGGAGCCTTGAGCTCTTAGTGGCGCAGCTAACGCATTAAGTTGACCGCCTGGGGAGTACGGC |
| | | CGCAAGGTTAAAACTCAAATGAATTGACGGGGGCCCGCACAAGCGGTGGAGCATGTGGTTTA |
| | | ATTCGAAGCAACGCGAAGAACCTTACCAGGCCTTGACATCCAATGAACTTTCCAGAGATGGA |
| | | TTGGTGCCTTCGGGAGCATTGAGACAGGTGCTGCATGGCTGTCGTCAGCTCGTGTCGTGAGA |
| | | TGTTGGGTTAAGTCCCGTAACGAGCGCAACCCTTGTCCTTAGTTACCAGCACGTCATGGTGG |
| | | GCACTCTAAGGAGACTGCCGGTGACAAACCGGAGGAAGGTGGGGATGACGTCAAGTCATCAT |
| | | GGCCCTTACGGCCTGGGCTACACACGTGCTACAATGGTCGGTACAGAGGGTTGCCAAGCCGC |
| | | GAGGTGGAGCTAATCCCACAAAACCGATCGTAGTCCGGATCGCAGTCTGCAACTCGACTGCG |
| | | TGAAGTCGGAATCGCTAGTAATCGCGAATCAGAATGTCGCGGTGAATACGTTCCCGGGCCTT |
| | | GTACACACCGCCCGTCACACCATGGGAGTGGGTTGCACCAGAAGTAGCTAGTC |
| 28 | MIC-18905 | MRLIIVSGRSGSGKSTALNVLEDNGYYCIDNLPAGLLPELAERALIHTELAQPLVAVSIDAR |
| | | NLPSHLSRFPELLEEVRARHIQCDVLYLDADEETLLKRFSETRRRHPLSTANRSLAEAIEDE |
| | | TALLGPIADLADLKVNTTNLNLYQLRDTIKLRLLNQPEPGTAFLVESFGFKRGMPVDADLVF |
| | | DVRCLPNPYWKPELRAQSGLDAPVAEYLAAQPEVEEMFQDIYGYLYKWLPRFAASNRAYVTI |
| | | AIGCTGGHHRSVYLTERLGQALQKTLKNVQVRHRDL |
| 29 | MIC-18905 | MRVILLGAPGAGKGTQAKFITEKFGIPQISTGDMLRAAVKAGTPLGVQAKSIMDAGGLVSDD |
| | | LIIALVQDRIAQPDCANGFLFDGFPRTIPQAEALVTAGVELDAVVEIAVEDEEIVQRIAGRR |
| | | VHEASGRVYHIVYNPPKIAGKDDITGEELVQRKDDTEETVRHRLSVYHSQTKPLVEFYQSLS |
| | | AKAGKPKYSHIPGVGSVEAITAKVLEALS |
| 30 | MIC-18905 | MSGNTYGKLFTVTTAGESHGPALVAIVDGCPPGLEISLEDLQRDLDRRKPGTSRHTTQRQEA |
| | | DEVEILSGVFEGRTTGCSIGLLIRNTDQKSKDYSAIKDLFRPAHADYTYHHKYGERDYRGGG |
| | | RSSARETAMRVAAGAIAKKYLASQGIVIRGYMSQLGPIEIPFKTWDSVEQNAFFSPDPDKVP |
| | | ELEAYMDQLRRDQDSVGAKITVVAEGVMPGLGEPIFDRLDAELAHALMSINAVKGVEIGAGF |
| | | ACVAQRGTEHRDELTPQGFLSNNAGGILGGISSGQPIVAHLALKPTSSITTPGRSIDIHGNP |
| | | VDVITKGRHDPCVGIRATPIAEAMMAIVLMDHLLRHRGQNADVRVSTPVLGL |
| 31 | MIC-18905 | MTEPALLPRPQLRRLLRKARRSLTPGEQRQAAKGLFRQLAQDPHFRRAKHISLYLPTDGEID |
| | | PRLLLREAQRRGKATYLPVLSAWPRTKMVFQRIRPGEKLKPNRFRILEPRANLARQRKIWTL |
| | | DLVLLPLVGFDDVGGRLGMGGGFYDRSLAYLARRKNWRKPTLLGLAHECQKVERLAQASWDV |
| | | PLQGTVTDKAWYFAG |
| 32 | MIC-54347 | CGGAGGGATCATTACCGAGTTTACAACTCCCAAACCCAATGTGAACGTTACCAAACTGTTGC |
| | | CTCGGCGGGGTCACGCCCCGGGTGCGTAAAAGCCCCGGAACCAGGCGCCCGCCGGAGGAACC |
| | | AACCAAACTCTTTCTGTAGTCCCCTCGCGGACGTATTTCTTACAGCTCTGAGCAAAAATTCA |
| | | AAATGAATCAAAACTTTCAACAACGGATCTCTTGGTTCTGGCATCGATGAAGAACGCAGCGA |
| | | AATGCGATAAGTAATGTGAATTGCAGAATTCAGTGAATCATCGAATCTTTGAACGCACATTG |
| | | CGCCCGCCAGTATTCTGGCGGGCATGCCTGTCCGAGCGTCATTTCAACCCTCGAACCCCTCC |
| | | GGGGGATCGGCGTTGGGGATCGGGACCCCTCACCGGGTGCCGGCCCTGAAATACAGTGGCGG |
| | | TCTCGCCGCAGCCTCTCCTGCGCAGTAGTTTGCACAACTCGCACCGGGAGCGCGGCGCGTCC |
| | | ACGTCCGTAAAACACCCAACTTCTGAAATGTTGACCTCGGATCAGGTAGGAATACCCGCTGA |
| | | ACTTAAGCATAT |
| 33 | MIC-54347 | ATGAAAATGCTTATTACTGCCGCTCTTTTCACCCTGGCCCTCGCCGCCCCTGTCGCCGAGAC |
| | | TAAGCCTCATAGCATTGCTGCTCGCGATCCCTTTACTTGCCCCGGCGGCCTGACCAACAGCA |
| | | CCCCCATGTGCTGCAGCGTTAATGTCCTTGGCCTGCTAGCCCTTGATTGCCAGCAACCTGGC |
| | | GCGGACGGCTGTAGTGGCAGCTCCAAGCCCAATTGTTGCACCCTTGGAGCGGCCGGACAGGG |
| | | TGTTATTTGCAACGCACTGTAA |
| 34 | MIC-54347 | ATGAAGTCGGCCATTCTTCTTGGGCTCACTGGCCTCGCTGCCAATGTGAATGCCCATCCTGC |
| | | TAAGCAGCCAGAGACCGGCAACGGCCTGACTAAGCGTGGCGTTGATATTAGCAAGTATAGCT |
| | | TGCCTAATCTTTCAAAGTACACGTCTTCGCCAAACATCGAACAAGAGGCCTCTCTGCAGTCT |
| | | TTGGCTTTCAAGCGCAACTATGTGGATACAGCCACACGAGCTGTCAAGAAAGCTGCCCCCAA |
| | | GGCCGAATTCCGCGTCGTGGATGACCACTATATCGATGTTGACGGCATTGGTCACGTCCACT |
| | | TTAAGCAGACAGTGCACGGAATTGACATTGACAACGGTGACTTCAAGGTTAACATTGGCAAA |
| | | GACGGTAGAGTCTTCTCTCATGGAAACAGCTTCTTTTCTGGCAAGCTGCCCCAGCAGAACCC |
| | | CTTGAGGAAGCGTGATTTCTCTGACCCCACCACTGCGCTCAAGGGTGCAATTGATATCCTGG |
| | | GCCTCCCGGTTCAGGCTGATGGCGCCACCGCCGAAGCTCAGGAAGGCACTGAGAAGTACACG |
| | | TTGAAGGGTACATCCGGAGCTGTCAGCGATCCTAAGGCTCACCTCGTTTACCTCGTCAAGGG |
| | | AGACGGAACTCTGGCTCTGACTTGGCGGGTCGAGACCGATATCATGGACAACTGGCTTCTCA |
| | | CCTACGTTGACGCAACAACCAACCAAGAGATTCATGGTGTTGTAGACTACGTCTCCGACCTT |
| | | GCCACCTTCCAGGTTTACCCCTGGGGCCTCAATGACCCCACCGAGGGAGATCGTAAAGTCCT |
| | | GACCGACCCGTGGAGAACCGATGCCTCCCCATTCACTTGGCTCAGCGACGGAACCACCAACT |
| | | ATACTGTAACTCGCGGAAACAATGCTATTGCGCAGGATAATCCTTCTGGGGGGGACAGTTAT |
| | | CTCAATAATCACCGTCCATCCAGCTCGACTCGGGACTTTCAATATCCCTTTACGCTGACTCA |
| | | GACCAACCCTACAGATTATAGGGATGCTGCCATCACACAGCTCTTTTACACAGTCAACAAGT |
| | | ACCACGATTTGCTATATGTCCTTGGCTTCAACGAGGTAGCAGGAAACTTCCAAGCAAATAAC |
| | | AACGGAAAGGGCGGCAAGGCCAATGATTTCGTTATTGTTAACGCTCAAGACGGCAGTGGAAC |
| | | GAACAATGCCAACTTTGCTACCCCGGCCGATGGAAGCAATGGCCGTATGAGAATGTACATCT |
| | | GGACTACTGCTAACCCCAAGCGAGATGGCGATCTCGAGGCGGGCATTGTTATCCACGAATAC |
| | | ACTCACGGATTGTCTACCCGTCTTACCGGCGGCCCTGCCAACTCTGGATGCCTTACTGGCAC |
| | | TGAAGCCGGCGGCATGGGTGAAGGCTGGGGTGATTTCTTCGCCACCGCTATCCGCTTAAAGG |
| | | CTGGTGACACCCGCAGCAAGGACTATCCTATGGGAGTCTGGGCCGATAACAACGTCAAGGGT |
| | | ATCCGCCAGTATCCTTATTCAACAAGCCTTACTACCAATCCTCTTACTTATAAAACCGTCAA |
| | | TACGCAAAATGAAGTCCACTCAGCCGGTACTACATGGGCCACCATTTTGTACGAAGTCCTGT |
| | | GGAACCTCATTGACAAGTACGGCAAGAATGACGACGATTTCCCTACCTTTGATAGCCAGGGC |
| | | GTCCCCACCGACGGCAAGTATCTTACCTTGAAACTCGTCCTCAATGGATTGGCTCTGCAGCC |
| | | CTGCACGCCAACTTTTGTTTCTGCCCGTGATGCCATCTTGGATGCTGATCGTGCTTTGACCG |
| | | GAGGGGAGAATCTTTGTGAATTATGGACAGGATTCGCCAAGAGAGGTCTTGGATCCGGCGCC |
| | | AAATATTCTTCCACCGCTCGCGTTGAAAGCTTCACTATCCCATCTGGGGTTTGCTAA |
| 35 | MIC-54347 | ATGAAGTTCTTCGCCGCCACCGCTCTCCTTGCCGCCACTACCATTGCTGGACCTCTTGAGGT |
| | | TCGCACTGGAGACGGTAACATTTGTCCTAGCGGCGGCCTCTACGGCAACCCTCAGTGCTGCT |
| | | CCTCTCTCCTCCTTGGTCTCATTGGTCTTGACTGCAACGTCCCCAACCAAACGCCCCGTGAC |
| | | GGCGCTGACTTTAGAAACATCTGCGCTAAGACTGGTGACGAGGCCCTTTGCTGTGTTGCTCC |
| | | CGTTGCCGGCCAAGCTCTTCTCTGCCAGGTCGCCGTTGGTGCCAGCTAA |
| 36 | MIC-54347 | ATGAAGTTCTTTGCTGTTGCCACTCTCTTTGTCACTGGCGTCATCTCCGCCCCGTCACCCAA |
| | | TGCCCTAAATTCCCGCAGTCTTCTTTGCAGCCCTGGCCTTTACTCCACCGCGCAGTGCTGTG |
| | | GTGTTGATGTATTGGGAGTTGCTGATTTGGACTGTGCGGCTCCTACTGGCACTATTACCAAC |
| | | GCCCAAGGATTTCAGGCTGCCTGTGCTAAGAAAGGCCAAGAGGCTCGCTGTTGTGTTCTACC |
| | | TGTTGCCGGCCAAGATGTCCTGTGCCAGGACCCCCCTGGTTTGTAA |
| 37 | MIC-54347 | ATGACCAGCATTCGTCGTCTTGCTCTCTATCTCGGAGCTTTGCTCCCGGCTGTCCTCGCCGC |
| | | TCCAGCAGCCCTTCACAAGAAGCCTGAGGCTGCACCTAACAAGTTTATTGTCACTCTTAAAG |
| | | AGGGCGCTTCAATTGATACCGACTCTCATCTCGCCTGGGTGAATGACATCCACCGTCGTTCT |
| | | TTGACCAAGCGTAGCACTGCTGGTGTTGAAAAGACTTATAACATTCATACTTGGAGTGCTTA |
| | | TGCGGGTGAATTTGATGCAGAGACGATTGAGCAGATCAAGTCTAGCCCCGATGTCGCGTCTG |
| | | TGGAGCCAGACTACATCATGTACCTGTCGGACATTGTTGAAGACAAGCGTGCTCTGACCACA |
| | | CAATCTGGAGCTCCTTGGGGCCTTGGCACTGTTTCCCACCGCACATCTGGGTCTACGAGCTA |
| | | CATTTACGATAGCTCAGCTGGCTCTGGAACCTTTGCCTATGTGGTTGACTCCGGCATCAACA |
| | | CCTCTCATCAGCAATTCGGCGGGCGCGCCAGTCTTGGCTACAATGCTGCAGGAGGACAGCAC |
| | | GTCGACACTCTTGGCCATGGTACTCATGTTTCTGGAACAATTGGTGGATCTACATACGGCGT |
| | | TGCTAAACAGGCCAGCCTAATCTCCGTCAAAGTCTTTGCTGGAGAGAGCGCTACCACCTCTG |
| | | TTATCCTTGACGGCTATAACTGGGCTGTAAACGACATTGTCTCGAGGAATCGTGCTAGCAAA |
| | | TCTGCCATTAACATGTCGCTTGGAGGACCTGCCTCATCCACCTGGACGACCGCCATTAACGC |
| | | AGCCTTTAACCAGGGTGTGCTTACCATTGTCGCCGCTGGTAATGGCGACAGTTTCGGAAACC |
| | | CCCAGCCAGTTTCCGGCACTTCTCCTGCTAATGTTCCTAATGCTATCACTGTTGCGGCGCTG |
| | | GACATCAACTGGCGCACTGCTTCCTTCACCAACTACGGTGCTGGCGTTGATGTCTTTGCCCC |
| | | TGGTGTTAACATTCTGTCGTCATGGATTGGATCCAACACTGCTACCAACACAATCAGCGGCA |
| | | CCTCTATGGCGACACCTCACGTTGTTGGTCTGGCTCTCTATCTTCAATCCCTTGAAGGCCTC |
| | | ACCACTCCTACCGCTGTCACTAATCGGATCAAGGCTCTGGCTACCGCTGGCCGTATAACTGG |
| | | CAGCCTTAACGGCAGCCCCAACACTCTCATCTTCAACGGAAACAGTGCTTAA |
| 38 | MIC-54347 | ATGACTTCTCTATACCTTACTTCTGCCCTTGGGCTGCTATGCCTCCTCCCCGCTGCACAGGC |
| | | AGGCTGGAATCCGAACTCAAAGAACAACATTGTCGTATACTGGGGACAGGATGCAGGCAGCA |
| | | TTGGCCAGAATAGACTGTCTTACTACTGCGAAAATGCCCCTGATGTTGATGTTATCAATATC |
| | | TCTTTCTTAGTTGGCATAACAGATCTTAACCTGAACCTTGCCAATGTTGGCAACAACTGTAC |
| | | CTCTTTTGCTCAGGATCCCAACCTACTCAACTGCCCCCAAGTTGCGGCAGATATCGTCGAGT |
| | | GCCAACAGACATATGGAAAAACAATCATGATGAGCTTGTTTGGCTCGACTTATACTGAGAGT |
| | | GGTTTCAGTTCGTCGTCAACTGCAGTGTCCGCCGCTCAAGAAATTTGGGCCATGTTCGGTCC |
| | | TGTCCAGAGCGGCAACAGCACCCCCCGACCTTTTGGTAACGCTGTGATTGATGGATTTGACT |
| | | TTGATCTTGAAGACCCTATCGAAAACAACATGGAGCCTTTTGCGGCAGAGCTGCGATCTCTC |
| | | ACAAGTGCTGCTACCTCCAAGAAGTTTTACCTTTCGGCTGCTCCTCAGTGTGTGTACCCCGA |
| | | CGCGTCTGATCAGACGTTCCTCCAGGGAGAGGTGGCTTTTGACTGGTTGAATATCCAATTTT |
| | | ACAACAATGGCTGTGGTACCTCTTACTACCCCTCGGGCTACAACTACGCAACTTGGGACAAC |
| | | TGGGTCAAGACCGTCAGTGCTAACCCAAACACTAAGCTGCTTGTCGGCACTCCTGCCAGTGT |
| | | CCATGCTGTAAACTTTGCCAACTACTTTCCCACCAATGATCAACTCGCCGGAGCTATCTCGT |
| | | CTTCCAAGTCGTACGGTAGCTTTGCTGGTGTGATGCTATGGGATATGGCTCAGCTCTTTGGA |
| | | AATCCTGGATACCTGGACTTGATCGTAGCGGACCTGGGTGGCGCTTCCACCCCTCCTCCGCC |
| | | AGCTTCCACTACTCTGTCCACTGTGACTAGGTCCTCTACTGCTAGCAGCGGACCTACTTCTC |
| | | CTCCTCCCTCCGGCGGTGGCAGTGTCCCTCAATGGGGCCAGTGCGGTGGACAGGGATACACA |
| | | GGACCAACTCAATGCCAGTCTCCCTACACTTGCGTTGTTGAGAGCCAGTGGTGGGCATCTTG |
| | | CCAGTAA |
| 39 | MIC-54347 | ATGAGAACATCTACCGCTATTATATCTCTTGCCCTTGTGGTGATGGGAATTGCCGCCCCTGC |
| | | TGCCGAGGCCGGCTCTGTTGTCAGCGAAGCCGGCTTTGTTGTCAGCGAAGCCCAATTCAACG |
| | | CCATATTTCCCGGTCGAAACCCTTTTTACTCCTATAGGGCATTGACCGGCGCCCTCGGATCT |
| | | TATCCCAGCTTCACCAACACTGCTGATAACGCCACTAGGCTCCGCGAGGCCGCCGCTTTCTT |
| | | CGCCAATGTGGATCATGAAACCGATGGGCTCAAGTTCGTCGTGGAATAA |
| 40 | MIC-54347 | ATGAGCGCCGCATCCCGCAAGTCACGCATAGCTACGAGTGTGGCGAGAGTTATGTACACCAA |
| | | TGCAGTCTATTTCCCTAGCAGCAGAATATATCAAGGTGATTCGCCGGGAATGCTCAATTACA |
| | | GCTGCATCAACCACGTGTATTATGCTTATGCCAGCGTGACGGCGGACGGCAATGTGTTTCTC |
| | | GGCGATGAGTGGGCCGATGCAAGAGCGCCAGTGGATGGTGTTCAGGGTGGCTTAGGATCCTT |
| | | GATGCATCTCAAGCAGAGGCACCCTCACCTGCAGGTTGTCTTATCTATTGGTGGCAGCACTG |
| | | CATCAGAGGTATTTCCCATTGTTGCATCAAGCACTCTCCTCAGAGACAACTTTGCCCGGTCT |
| | | GCCCTTGGCCTCGTGGAAGCATCCGGGCTTGATGGTATTGACATTGCCTGGGAGTTCCCATC |
| | | CCAAGCTAAACATGGCCACGATTTCCTAGCCTTGCTGGCAGCAGTGCGGATTCATATGCCTG |
| | | AAGACCGCTTCATTCTAACAGCTGTCCTCCCTGCGGCGAAGGAGGTTCTACAACTCATCGAC |
| | | CTCAGCACAGCGGCCGAGTACCTTGACTACATCAACCTTGTGGCATATGATTTCTTCGGCAC |
| | | GTGGACGTCCAAAACTGGTCATCACGCACAGTTATATACTATGAACAAGGACGAACCCTCGG |
| | | CATCGACAGGTGTGGCGTATGTCATGTCCCAAGGATTCCCTGCGAAGAGTATCCTGCTCGGG |
| | | ATCCCAACCTACGGACGAAGCTTCCTCAAGGCCAATGGGCCGGGCCAGGATTTTAACGGCGT |
| | | TGGCGGCCAAGATGGCACATTCGAATACGGCGAATTACCACGAAAGGGATGCAAAGAGATTG |
| | | TGGATAGGCGCTATATTGCGGCCCAATGTGTCGGTGGTGATGGGGGGTTTGTCACGTACGAC |
| | | AACCCCGAGACGGTCAAGGTTAAGGCGGAATTTTGCAAGCAAAAGGGATTAGGGGGGCTTTT |
| | | TTACTGGAACGGACCGGCTGACTCTCGAGATCAAGCACGAAGTCTGATTGCGGCTGGATTCC |
| | | GCGCTCTACACACCTCGTGA |
| 41 | MIC-54347 | ATGCAATTGACCACAGTCGTCGCGCTCTTCGCCTCTCTGGCCGGCGCCGCGCCTGCCCCTGA |
| | | ACCCGCTGCTGAGCTTGTTGCTCGTGATGGTCCTTGCTCCTCTGGTGTCACCAATAACGTCC |
| | | CTCAATGCTGCGGTACTGGTATCCTCAGTGTTGTCTACGTGGACTGCAAGACTCCCACTCAA |
| | | GCTCCCTCTGCCACTAACCAGTTGAGCGCCATCTGCGCTCGAGTAGGTCTCCAGGCCAAGTG |
| | | TTGCACCGTCGGCATTGCTGGCGTTGGCGTTCTTTGCCAGGATGCTATTCCCCAGTAA |
| 42 | MIC-54347 | ATGCAATTGTCCAACCTCTTCAAGCTTGCTCTCTTCACCGCCGCTGTCTCTGCTGATACCGT |
| | | CTCGTACGATACTGGCTACGATGACGCATCTCGCTCTCTGACCGTCGTCTCCTGCTCCGACG |
| | | GCCCCAACGGCCTCATCACCAGATACCACTGGCAGACCCAGGGCCAGATCCCTCGCTTCCCA |
| | | TACATTGGTGGTGTCCAGGCCGTCGCCGGCTGGAACTCCCCTAGCTGCGGCACCTGCTGGAA |
| | | GCTCACTTACAGCGGCAAGACCATCTACGTCTTGGCTGTTGACCACGCCGGTGCTGGCTTCA |
| | | ACATTGGCCTCGACGCCATGAATGCTCTCACCAACGGCCAGGCTGTTGCCCTCGGACGTGTC |
| | | TCTGCCACTGCCTCTCAGGTGGCTGTGAGCAACTGCGGTCTCTAG |
| 43 | MIC-54347 | ATGCATTCAACAACTTTCTTTGCCAGTCTGGGACTCGCAGGCCTGGTTGCCGGCGCCCCTTC |
| | | GGCCCCGCACAACGTTAAAGCTCGCCAAGCTTCCGGTGCCCAAAACGTCGTCTATTGGGGAG |
| | | GCACTAACAATGAAAGCGACGACCTTTCTACCTATTGTACGCCCACCGCAGGCATTGACATC |
| | | CTTGTTCTGTCTTTCTTGGACATTTACGGCACAACTGGCAACATTCCAGCAGGTAATATTGG |
| | | CAATTCTTGCTATATCGGAACAAATGGCACACCTCAACTATGTGATAATCTTGCTGCTTCAA |
| | | TAGCCAGCTGCCAGGCCGCTGGTATTAAAATTATCTTGTCTCTCGGAGGAGCGGCCGGCTCC |
| | | TATTCTCTACAATCGCAGTCACAGGCTGTGGCTATTGGCCAGTATCTCTGGGAGGCTTACGG |
| | | CAACTCTGGCAGCACTTCTGTCCAGCGGCCCTTTGGCAATGTCTTTGTTAATGGTTTTGACT |
| | | TTGACCTTGAGCTGAACGCCGGCAACCAATACTACCAGTATCTGATATCAACCCTCCGTTCC |
| | | AACTTTGCCAGTGACCCGAAACATACGTATTATATCACTGGAGCGCCCCAGTGTCCTCTCCC |
| | | GTTGAGTATTCCTCAAATCCGTAAACCAAATATGGGAGAGATTATAAGTGCCTCACAGTTCG |
| | | ACTATCTCTGGATTCAATTCTATAACAACAACGCTTATGCACCAGACCCCTGTTCTCTTGGT |
| | | CTGCCTGGCGATGCACCCTTCAACTACAACAACTGGACGTCATTTATTGCTACTACCCCTTC |
| | | AAAAAACGCAAAACTATTTATTGGAGTCCCTGCGAATACTTTAGCTGCTAATGGCAATTCAG |
| | | GTGGTGCCGTTTACTATGCTTCGCCATCCCAGCTTGCTTCCATTGTCGCGAATACAAAATCT |
| | | AGCCCAGATTTTGGAGGAATTATGATGTGGGATGCTGGGTACTCTGATGCTAATGTCAACAA |
| | | CGGATGCAACTACGCTCAGGAGGCCAAAAACATCCTCCTTACCGGCGCTCCCTGTGGAGGCT |
| | | CACCCCCACCGGTTAGCAGCAGCAAGCCTACATCCACTGCAACCAAATCCGCTACTAGCACT |
| | | TCATCAGCCTCGGGAACAGGCCCGACAGGAGGCGGCACAGTACCTCAGTGGGGCCAGTGCGG |
| | | CGGAGAGGGATACACGGGCCCGACGCAGTGCGTTTCTCCCTACAAATGCGTCGAATCAAGTC |
| | | AATGGTGGTCGTCTTGCCAATAA |
| 44 | MIC-54347 | ATGCCCTCTTTGATCACGGTTGCGAGCGTGCTCGCTCTTGTTCCATCCGCCTTCGCGGGCTG |
| | | GAATGTCAACTCGAAGCAAAACATTGCTGTGTACTGGGGACAAAACTCCGCCAACCAACAGA |
| | | GCACACAGCAGCGTCTTTCAGCCTACTGCAATGCGAAGATTGAATCTGACAGTTGCGATGTA |
| | | GATGCCAACATCAATGTCATTGACATTGCTTTCCTGAATGGAATTACTCCTCCTATGACCAA |
| | | CTTTGCCAATGCTGGTGACCGATGCACTCCCTTCTCCGACAACCCTTGGCTCTTGAGCTGCC |
| | | CTGAAATTGAGGCGGATATCAAGACTTGCCAGGCTAATGGCAAGACCATTCTCATTTCTCTT |
| | | GGTGGTGATTCTTACACTCAAGGTGGCTGGAGCTCTACCAGTGCTGCTCAGGCCGCAGCCAA |
| | | ACAGGTCTGGGCCATGTTTGGTCCTGTCCAATCCGGCAGCTCCACCGAGCGTCCCTTTGGCA |
| | | GCGCCGTTGTTGACGGTTTTGACTTCGACTTTGAAGCGACGACTAACAATCTCGCGGCCTTT |
| | | GGCGCTCAGCTCAAGAGCCTCTCGAGCGCTGCCGGTGGCAAGAAGTACTACTTCTCTGCTGC |
| | | TCCCCAGTGCTTCTTCCCCGACGCTGCTGTCGGTGCACTGATCAACGCCGTCCCCATGGACT |
| | | GGATCCAGATTCAATTCTATAACAACCCTTGCGGCGTCAGCGGCTACACCCCCGGCACCAGC |
| | | AACCAGAACAACTACAACTACCAGACCTGGGATACCTGGGCCAAGACAAGCCCCAACCCCAA |
| | | CGTCAAGCTTCTTGTCGGCATTCCCGCTGGCCCAGGTGCTGGTCGCGGCTACGTGTCTGGCT |
| | | CTCAGCTCACTTCAGTCTTCCAGTACTCGAAGGGCTTCAGCAGCACTTTTGCCGGTGCTATG |
| | | ATGTGGGATATGTCCCAGCTCTTCCAGAACACTGGCTTCGAGGCCCAGGTTGTCAATGCTTT |
| | | GAAATAA |
| 45 | MIC-54347 | ATGCCGCTTAAGGTCTTCGAGTCAGCTTCGCCTGCGCCGTCGCATCCCTCCTTATCGCACCT |
| | | CTGCCTGCTCGTCTTCGAAGCTGTTCTAGAAGTAGTCTGTGTGAGCTTGCCGGGATACATCG |
| | | TTGCGCGCCTTGGCCATTTCGATGCCGATAAGCAAAAGTTCCTGGCTAATCTCAATGTGATG |
| | | CTCTTCACGCCCTGTCTTATCTTCACCAAGCTAGCATCTCAGCTCAATGCCGAAAAACTATC |
| | | CGATCTCGCCATCATCCCCGCCATCTTTATCGTGCAAACTCTTGTATCATGGATAGTTTCGA |
| | | TCCTGGTCGCCAAGGGGTTCCGATTCAATAAGCGAGCTTCCAACTTCGTTACTGCTATGGGC |
| | | GTCTTTGGCAATTCAAATTCACTGCCCATCTCTCTCGTTCTTTCTCTCTCGCAAACAATCAA |
| | | GGGCCTCCACTGGGACAGGATACCAGGCGACAACGATGACGAAGTCGGCGCTCGCGGTATCT |
| | | TATATCTACTGATTTTCCAGCAACTTGGCCAGCTTGTCAGATGGAGCTGGGGATATCACGTT |
| | | CTGCTAGCCCCCAAGGATAAATACGCCGAGTATCAAGACGAAATTGCCGAAGAAGGCCAGTA |
| | | CAGATACAGAGACGAAGAGCCGAACGAACAGGAGCCGGAGATTCTCATTACTGGTTTGGACG |
| | | GTGATACCGAAGATGATGGCGAGAGCAATGCTTCTGAAGATTATATACCTGCTGGACGAACA |
| | | CCTCTTGCGAGCAATTCCCGGGCTTCACTGGCTGGCTCTTCCGTCGACAATGACGACATGCT |
| | | GAATTTCAAAAAGGGCAACTACACTCGTGGAAGCTCGCTCGCCAACACAGACTTGGAAGATG |
| | | ATATTCTTTCATTCCCTCGTATTCGTCTTCGAGATGAGGCTGAAGTCGAGCACGGAGTTACT |
| | | GCTCGTATCAAGAAATCACTATATTCTCTGAAAGACAAAGCTTCTGCGGCCATGACTCGCCA |
| | | ATATCAGCGACTTCCTCAACCTGTTCAAACCTGCCTTTCCTTCATTCATAAGTCAATCACAA |
| | | AGACTCTCGGATTTGTATGGGATTTCATGAATCCACCTTTGTGGGCTATGCTCATCGCGGTC |
| | | ATCGTGGCGTCCATCCCGAGTCTCCAACAGTTGTTCTTCGAAGATGGTTCATTCGTGAAAAA |
| | | TAGCGTCACCAACGCTGTTTCGTCTAGTGGAGGCGTTGCAGTGCCTTTGATCTTGGTTGTTC |
| | | TCGGCGCCAACTTGGCGCGCAACACTGCCGCTCACGATTCTCCCATAGACCCGGAGGAGGAA |
| | | AAGATTGGCACCAAGCTCTTGATCGCTTCGTTGCTGAGCAGGATGGTTCTGCCAACTTTGAT |
| | | TATGGCACCCATTCTGGCTATTACGGCAAAATACTTGCCCATCAGCATTCTAGATGATCCCA |
| | | TCTTTATTGTCGTATGCTTTCTTCTTACTGGCGCACCCAGTGCGCTCCAGCTTGCGCAAATT |
| | | TGCCAAATCAACAATGTTTATGAGCAGACTATGGGAAGAATCCTCTTTCAGAGCTATGTTAT |
| | | CTGGATTCTTCCCTCTACTCTTTTCCTTGTCATGATGGCACTCGAGGTGATTGAGTGGGCAA |
| | | CAGTGAATTAA |
| 46 | MIC-54347 | ATGCTACCGTCGAGCCTTTGCCGGATTGCCGCTGTTATCAGCGTGGCTTCTGCGGAAATGGT |
| | | TTCGGTAACATTTGATACCGCTTACGACGATCCTTCTCGTTCACTGAGTGAAGTCGCTTGTT |
| | | GGAGGAAGGGCACGGGATTCATGCCTAACTTGGATTGGAAACTTCAAAAAGATGCATTGGAA |
| | | TTTATTGGAATCAAAGCAATTCGTGGCTTCAGTTCTGCCCAATGTTTTTCCTGCTGGAAAAT |
| | | TGAGTATGGAGATAAGCAGATATCACTGTTCGCAATTGACAGCGCCGACTCGGGCATTGTTC |
| | | TCTCTCTGAGCACCATGCAATATCTTACAGGTGGCCAGGCTCGCGAGCTGGCCCGAATTGAT |
| | | GCAAAGGCAACCCAAGTCGACGCATCAAACTGTGGGATTTCTGCGGCTGAACTGCACAAATA |
| | | CGATTTCTATACCAATGGTAGTGGAAATAGCGATGGCTCTAGGAGCAACACTGGAGCTATTG |
| | | TTGGAGGAGTTCTCGGTGGTGTGGCTGGGCTTGCTTTAACTGGGCTTGCTGTTTTTTTCTTC |
| | | CCCCGCTATAAGAATTTAGCTGCTGGACATAAGCCAGTGAGCCAGAATACTCCTCCTGCTGC |
| | | TTATCAATCCTCTCTGATGCAGCAGGACCCCAAGGCTCACTATGATCCTAAATATTTCGCAG |
| | | AGATGGATACACAGCCATACACGCCCCCTGATTAG |
| 47 | MIC-54347 | ATGCTCCGTACTCCTACAATCCCCTGGCTTCTAAATATCATCCTAGAAGAAAACCCTACTAT |
| | | GATTCCTTCAATGAAAAAGTGCGGCCGCAAGTTATGGGCTGGCCTGGCATTGCTAGCCTTGC |
| | | CTGTAATTGCAAGCGCATCATGCGCGCTTCCTAGCACCTACAAATGGACTTCGACTGGCCCG |
| | | CTGGCAAACCCCAAGTCGGGCTGGGTTTCGCTTAAAGATTTCAGCCATGTTCCATACAATGG |
| | | CCAGCATCTGGTCTATTCTTCGACTGTGAACTCGGCAGGTTCCTATGGCTCGATGAATTTTG |
| | | GTCTCGTCTCAAACTGGACCAACCTCAGCACAGCCAGCCAAAACACAATGAACCTTGGCACG |
| | | GTAGCGCCAACTCTCTTCTATTTCTCTCCCAAGAAGATCTGGGTTCTTGCATACGAATGGGC |
| | | TGCTACTCCATTTGCCTATGTCACGTCGACCGATCCTACCAATGCCAATGGTTGGTCAGCTT |
| | | CGCAACCGCTCTTCAGCGGAAGCATATCTCCCTCTAGCCCTATCGACCCAGCACTCATCAGC |
| | | GATGGCACGAATATGTACCTTTTCTTTGCAGGAGATAATGGCAAAATCTACCGGTCCAGTAT |
| | | GCCCATTGGCCAGTTCCCATCCAGCTTCGGCACATCGTACACAACGATCATGAGCGCCGCAA |
| | | CCAACGATCTGTTTGAAGCGATACAGGTGTACACTGTCTCAGGCCAGAATCAATATCTTATG |
| | | ATTGTTGAGTGTATCGGATCTGTTGGACGGTATTTCCGCTCATTTACGGCCACATCCCTGAG |
| | | CGGTACATGGACGCCGCAAGCTGCAACCGAGAGCAATCCCTTTGCAGGCCATGCTAACAGCG |
| | | GAGCTACTTGGACTAACGATATCAGCTCTGGAGATCTTATTCGCAGCACAAACGATGAGACG |
| | | ATGACCATTGATCCCTGCAACCTCCAGCTGCTGTACCAAGGAATGGCCGTTGGTTCCACCGG |
| | | AGACTATAACTCCCTGCCATGGCGACCCGCCGTCTTGACTCTAACCAACCCCGGCTCGAGCA |
| | | CAGGCAACGGCAACGGCACTGGAAGCGGCGGCTCAGGCGGCAGCGGATCTGGACAAGCATCG |
| | | CAGTATGCTCAGTGCGGCGGTTTGGGCTATACTGGACCCACGAGCCCGTACAAATGCACGTT |
| | | TGTCAACGAATACTACTCCCAGTGTCTATAG |
| 48 | MIC-54347 | ATGCTCTACACTGCTGCTCTGGCCGGCCTTATGGCCACTGCTGCCCTTGCTGCCCCCTACCG |
| | | CCGACAGGCTACTGGCCAAAACGTTGTATACTGGGGCCAAAACGGCGGCGGCGTTACTGAAA |
| | | GTAACAATTTGTCTGATTATTGCGATGCTGAAGCAGGCATCGATCTACTTGTTCTTAGTTTC |
| | | CTCTACGAGTATGGCAATGGAAATACTATTCCTTCAGGCACTATCGGCCAGAGCTGCTCCAT |
| | | TGATACCTCCGGAAACCCTTCAAACTGTGATGCCCTTGCCTCGGCCATTGCTACCTGCAAGT |
| | | CCAATGGAGTCAAGGTTGTCTTGTCTCTCGGCGGCGCCGTTGGAGCCTACTCTCTCTCCTCT |
| | | CAGCAGGAGGCCGAGACAATTGGCCAAAACCTCTGGGACGCATACGGTGCCGGCAATGGAAC |
| | | CGTCCCCAGACCGTTCAGAAGCACCGTTGTCGATGGCTGGGACTTTGATATCGAAGCCAGTA |
| | | GTGGCAACCAGTATTATCAATACTTGATTGCCAAGCTTCGCTCAAATTTCAACGGCGGCAAC |
| | | TACGTGATTACCGGTGCTCCTCAGTGCCCTATTCCGGAGCCTAACATGCAGCAAATCATTAC |
| | | CACTTCTCAGTTTGACTATCTTTGGGTCCAGTTCTACAACAATCCCTCGTGCTCAGTTGGAA |
| | | CGTCGACACCAAACTTCCAAGATTGGGTTTCCAACATTGCCAACACTCCTTCTGCCAATGCC |
| | | AAGATCTTCCTTGGTGTGCCTGCAAGCCCGCTCGGCGCTACTGGAACTGAATCTGGCGCCCA |
| | | ATATTACCTCGAGCCTTCTGCTCTCAACACTCTCGTCGGCCAGTTCTCATCCAACCCTGCGT |
| | | TTGGCGGTATCATGATGTGGGCTGCCGGTTTCTCCGATGCCAACGTGAACAATGGATGCACT |
| | | TATGCTCAAGAGGCTAAGAAGATCCTTACTTCTGGTCAAATCTGCTAA |
| 49 | MIC-54347 | ATGCTTTCCCACGCTATCCTTGCTGGCCTCGGCCTTGCTGGTCTCGCTGCCGCTGCGCCTAC |
| | | CAGGACGATTGCGACTCGTCAAGCTTCCGGTTATCAGAACGCTGTCTACTGGGGTGCCACTG |
| | | GCAAACAGAACCCTAACCTCGCTGAATACTGCACGTCCACCTCGGGCATTGATATCCTGATC |
| | | CTGTCCTTCTTGGACGTCTACGGTGCCACTGGCAACTTCCCCTCTGGCAACTTTGGCAACGA |
| | | CTGCTTCGTCGGCACCACGGGTGTTCCCCAGTCATGCTCTGACCTGGCCTCCCAGATCAAGA |
| | | CCTGCCAGAACGCTGGTGTCAAGATTATTGTTTCTCTCGGTGGTGCCGCCGGTTCATACTCT |
| | | GTCACGTCCCAGCAGCAGGCCCAGACCATCGGTCAATACCTCTGGGATGCCTACGGTAACTC |
| | | TGGCAGCACCTCTGTCCAGCGTCCCTTTGGCGATGTCTTTGTTAACGGTTGGGACTTTGACA |
| | | TCGAGGCCAACGGCGGCTTCAGCCAGTACTACCAGTACATGATTGGCACCCTGCGCTCCAAC |
| | | TTCGCCAAGGACTCCGCCAACACGTACTACATCACCGGTGCTCCCCAGTGCCCCCTGCCCGA |
| | | GGAGAACATGGGCGATATGATCCAGCACTCCGTCTTCGACTACCTCTTCATCCAGTTCTACA |
| | | ACAACAACCCCACCTGCTCTCTTGGTCTGTCTGGCCAGGCTCCCCTCAACTACGACGACTGG |
| | | ACCAACTTTGTCTCCACCACCCAGTCCAAGAACGCGAAGCTCTTCCTCGGTGCCCCTGCCGG |
| | | ACCTCTCGCCTCCAACGGAAACCCAAACGGTGCCGTCTACTACGCTACCCCCTCGGATCTGG |
| | | CCCCCATCGTCAACAAGGCCAAGACGAAGTCAAACTTTGGCGGTGTCATGCTCTGGGACGCT |
| | | GGTTACTCTGATGAGAACTCCAGCGGCGGTTGCAACTACGCCCAGCAGGTCAAGAGCATCCT |
| | | CACCACCGGCGCCCCTTGCAACGGCACCCCCGTCAGCGGTGGTGGCTCTCCTCCCGCTACTT |
| | | CTTCCACTGCTTCTTCTCCTCCCGCTACTTCTTCCACTGCTTCTTCTCCTCCTGCTACTTCT |
| | | TCCCTTCCTGCCGATGGAAGCACCGGAAGCACCGGTAACAGCGGCGTTGGCAGCGTTGCCCA |
| | | GTGGGGACAGTGCGGCGGTATTGGTTACACTGGTCCTACCCAGTGCCAGTCCCCATTCAAGT |
| | | GCGTTAACGAGGGCCCGTATTGGTCTTCTTGCCAATAA |
| 50 | MIC-54347 | ATGGCCGGTTCACTTACAGCAGACGCTCACGAAGCTGTTCGGTGCATCATGTATCTCACAGG |
| | | GCAACATGTGGTAGTCCCTTCAGACCATGATCTCGTGGGTTCCATAACACATGTGATACTGG |
| | | CATTCATGCGCTCTGATGTCTTCAATGTGGATAAAACGCCTGCCGAGTTCCCGTTTTTTACA |
| | | ACCGTTGCTGAAACACGGCAGAAGTTCAATGCGAATACTAAAATCATGGTCGCAATTGGGGG |
| | | TTGGGGGGATTCTGCAGGATTTGAAGAAGCTGCGCGTGACGATTCGTCGAGAAAGCGGTGGG |
| | | CTAACCAAGTAAAGGCCATGGTTGACCTGACAGGAGCTGATGGCATTGACATTGACTGGGAA |
| | | TATCCGGGGGGAAATCGTGACGACTATAAACTTATTCCGAATTCTCAGCGGGAATGGGAGAT |
| | | AGAGGCATTCGTGCTTCTTCTTCGAGAACTTCGTTTGGTTTTGGGAGAAGAAAAACTACTCA |
| | | CAATTGCGGTGCCAGCGCTAGAACGCGATTTGATGGCTTTCACAAACTCAACTATTCCGTCC |
| | | ATCGTGAATCAGGTTGACTTCATTAGTGTAATGACTTACGATATGATGAATCGACGTGATAC |
| | | CATTGTCAAGCACCATAGTGGTGTGGCCGACTCTCAGGAAGCAATGGAGCGATATATAGATC |
| | | GTGGGGCCCCTCCGCACAAATTGAACTTCGGACTTGGTTACTATGCCAAATGGTTCATGACC |
| | | GAGCAATGCGATTTACAGCACCCATTGGGGTGCCGCACTCAACTGCTAGAAGACCCCGCCAA |
| | | TGGAGCCGACCTTGGCAAGACTGCAGCTTTTAGCTGGCATGACGAGGTTCCCGTGGAATTGG |
| | | CTAATTCTTTCGAGAAAGCTCATACTCATGGCCGCTACTATGAAGATGGAAGCTACGGGTAT |
| | | TGGGATGATGAAGAGAAGAGATGGTGGTCCTACGACACGCCTCTCGCCATCAAAACTAAAGT |
| | | CCCTCGGTTTCTCGGCGAACTGCAATTGGGCGGTGTGTTTGCCTGGGGGCTGGGGGAGGATG |
| | | CTCCGCAGTTTATTCACTTGAAGGCCACTACTGATGGGATTCGGGCTTTGCGCGGAGGCCAG |
| | | AGCTCACGGGATTCGGTGAAAGATGAACTGTAA |
| 51 | MIC-54347 | ATGGCGCCTTTCAATACTATGCTTGGGTATCTCCTGTTAGTGGTAGCCCCTTTGGCGGCTGC |
| | | CCTGCCGTTCCGTGGCCATGGCCCCAACACCCATAACCACATCAACTTGAACTTCAACCTCA |
| | | CCGAAACCATCAACGAGATCAATGAAAACTTGGCTGGCCTAGTTGGATACATCACCAACCCT |
| | | CACGCCAAGCACATCGTCGCCAACCGCTACATTGTCGTCTACAACAACACCTTCGGCTCCGA |
| | | GGCCATCGCCGCCAAGCAGGCCGAGTTCGCTGCCACCATCCAGAAGCGCAACCTTGGAAAGC |
| | | GAAGCCTCGGCGGCAACATGCTGTCCACTGAAATCCACTCCTTCCAGATGCACACCTGGCGC |
| | | GCCATGGCTCTCGACGCTGACGATGAGATGATCAAGTCCATTTTCGCCGCGAAGGAGGTCGC |
| | | CTATATCGAGGCTGATACCGTCGTCCAGACCAAGGCTCTCGTTGCCCAGACCAACGCTACCC |
| | | CCGGCCTCATCCGTCTCTCTAACCAAAACATTGGTGGCAAGAACTACATCTTTGACAACTCT |
| | | GCCGGCGCTGGTATCACTGCCTACGTTGTCGACACTGGTATTAGAATCACCCACACTGAGTT |
| | | TGAGGGCCGTGCTTCTTTCGGTGCCAACTTTGTCAACTCCAACAACACTGATGAGAACGGCC |
| | | ACGGCAGCCACGTTTCTGGTACCATTGGTGGTGCTACCTTCGGCGTTGCCAAGAACATCAAG |
| | | CTCGTTGCCGTCAAGGTCCTTGATGCCTCTGGTGCCGGTAGCAACTCTGGCGTTCTGAACGG |
| | | CATGCAGTTCGTCGTCAACGACGTCCAGGCCAAGGGCCTCTCCGGCAAGGCTGTCATGAACA |
| | | TGTCTCTCGGTGGCTCCCTGTCCGCTGCTGTCAACAACGCTATTGCTGCCATTGCCAACGCT |
| | | GGTGTCGTCCCTGTTGTTGCTGCTGGTAACGAGAACCAAGATACTGCCAACACCTCTCCCGG |
| | | CTCTGCCCCTCAGGCCATCACCGTCGGTGCTATCGATGCCACCACCGATATCCGTGCTAGCT |
| | | TCTCCAACTTTGGCGCTGATGTCGATATCTACGCCCCCGGTGTCAATGTTCTCAGCGTTGGC |
| | | ATCAAGTCTGATACCGATACTGCCGTTCTCAGCGGCACCAGCATGGCCACTCCTCACGTTGC |
| | | CGGTCTCACTGGCTACCTGATGGCTCTCAAGGGTGTTACCAACGTCAACGATGTCACCAACC |
| | | TCATCAAGAACCTCGCCACTGCTTCCGGCGCCTCGGTCCAGCAGAACGTTGCCGGAACCACC |
| | | AACCTCATCGCCAACAACGGTGAACTGTAA |
| 52 | MIC-54347 | ATGGGCTATAGCCGCAAAGATAAATCGAGGTACATCAACACAATTCAAGAAGCACTTGACAT |
| | | CTCTACAGCTTTCGTTGCCAGCAGCCGAAAAAGAATAGCTTTAACTTGGAAGATTATCATTC |
| | | AAATTCAATCCAATCTATCTAGAATGCAATTCTTCGCTCTCCTCCCTCTGTTATACACCACC |
| | | GCTGCAGCCCTTGGCATCAATTGTCGTGGGAACAGCAACTGCGTCGGTACTCCAGAATGCAG |
| | | ACTCGCCGATCTGATCTTGCAAGTCAGCCAGCAAGATCCCAGCACATCATACAGCCCTGGTC |
| | | AGCACATTGCTTGTTGCGGCATACCTGGCGGCAATATCTGTGCATTCACTCAGGGCATTAGC |
| | | AATTCGATTACAGCTGGAGAAGCCCTGGGTATGCTGCAGGGGCTATCCGCCCACGGTTGTGG |
| | | CCAATGCGGCAGTATTCCCTTCAAGGATAACAATGTCGCTGAGGGACAATTGACAGTCAACT |
| | | GGACCGATCACTAA |
| 53 | MIC-54347 | ATGGGCTCCTTTAAAGCCATTATCGCAGCATCTCTAGCTGCCCTAGGACAGTTCGCTGCAGC |
| | | TGCTCCAGCGGAAGGCCAATCCAAGCGAGCTGGCATCTCTTCCATCGTCAAGGGAACACCGG |
| | | TAGGCTTTGCGTCTTCAGTCACGGGTGGTGGGACCGTTGCCCCGGTATATCCAACCACGATC |
| | | GCGCAGCTGAAGAGCTACCTCACCTCAACTAGCCCGCAGAACATTGTCATCTCTGGTACTTT |
| | | CAACTTTGCTGGTACTGAGGGCACAACAACTCTTCCAGCTTGTAACGCATACCCATGTACAC |
| | | CCTCCAACGGTGGACAGGCGTTGCTAAACACACTGGGTGGATGCGGGTCGCTATCTACGTAC |
| | | AACGTAAATCTCGACACCGCTGCATATAACGCTATCAATGTCCAGTCAGACAAGACACTTGT |
| | | TGGGATTAACGGTGCAACTCTCAATGGCAAAGGCTTACGCCTTTCTGGCGTTTCTAATGTCA |
| | | TTATCCAAAACATTGCAATCACTAATCTAAACCCGCAGTATGTCTGGGGTGGTGATGCCATT |
| | | AGTCTCAGCAATACTAACAATGTCTGGATTGACCATGTGAAGACATCCAACCTCGGCCGTCA |
| | | GCACTATAGCTTCGGTACTGGCTCCAATAACGCAGTCACCATCTCTAACAGCTTCATCAGTG |
| | | GTCAGACCTCTTACTCAGCTAGCTGTGATGGCCACTCGTACTGGGGTCTCGAACTGGTTGGA |
| | | AGCGGTGACCAAATCACATTTTACAAGAACTATGTCTACTACACATCTGGACGTACCCCTGC |
| | | TTTGTCCGGCAATACCCTCTTCCATGCAGTTAACAACGTCTGGTCCTCAAACTCCGGACACG |
| | | CAATTGAAGGTACTTCGAACGGCATGGGCTTGTATGAGGGTAACTACTTTGTGAACGTTCCG |
| | | ACCATTGTCGCTTCAGGCTTCGTAGGTCGTCTCTTCAGCTCACAGTCATCTGCTGTGTCGCA |
| | | GTGCGCGCAATATTTAGGACGCAACTGTGTCTCGAATTCTTTATCAAACTCGGGTACTTTCA |
| | | CCAATAGCGACACGAGCTTCCTTTACCTGTTTCAAGGCAAGGCCAATATTGTTTCCGCCGCT |
| | | TCTGCCTCGTCTATTCAGTCGAGTGTCGTTTCGTCGGCAGGAAATACGCTTTAA |
| 54 | MIC-54347 | ATGGTTCAATCTGCGGCTTATCTAGGAGCCTTGCTGGCCACCCTGCCCTTGGCACGTGCCGG |
| | | TTTCAACGCTAGCTCGACGCAAAACATTGCCGTGTACTGGGGTCAAAACTCGGCAAATCAAG |
| | | CAACCTCTCAGCAGCGATTGTCTACTTACTGTGCCAATGCTGATATAGATATCATCCCAATT |
| | | GGGTTCATGAACGGCATCAGCCCGGTCATTACCAACTTTGCCAATGCAGGCGACAATTGCAC |
| | | GGCTTTTGCAGATAATCAGAATGCACTTAATTGTCCCCAAATCGAGCAAGACATCATTACAT |
| | | GCCAGCAAACATATGGCAAGACAATCCTGATTTCGCTGGGGGGCGGATCTTATACCCAGGGC |
| | | GGCTTCTCGTCCACTGGTGTCGCCACATCAGCGGCTCAGACGGTTTGGAACATGTTTGGCCC |
| | | TGTCAATCCAAACAGCAACGTAGACCGTCCATTTGGTTCCGCAGTGGTTGATGGCGTTGACT |
| | | TTGACTTTGAATCTGGCGTCAATAACTTGGCCACTTTTGCCACTGAGCTGCGGAGCTTGATG |
| | | GACGCTTCTGCGTCTTCTGCGAACAGGAAGTTCTACCTGTCTGCTGCGCCGCAATGCGTCTA |
| | | TCCCGATTTCGCTGACAACCCGGCCCTGAATGGTTCGGTTTTCTTCGATTTCATTATGATTC |
| | | AATATTACAATAATGGATGCGGAGTCAGCAGCTATGTCCCTGGAGCAACTACCCAGTGGAAC |
| | | TACAACTTTGATGTCTGGGACAACTGGGCGCATACCGTCAGCAAGAACCCTAACGTGAGGAT |
| | | TCTGCTTGGTATCGCCGCCAACACTGGTGCTGCCAGTGGATACGTGTCCGGAACACAGCTTT |
| | | CTGCGGTCATCTCATTTACCAAGCAATACTCAAGCTTTGCGGGTATCATGATGTGGGATATG |
| | | TCCCAATTGTATCAGAATTCCGGCTTCTTGGGCCAGGTTGTCAGTGATCTCGCCGCCTCTGG |
| | | TTCAACCCCCCCGGCTACTACTTCTAGTGGAGCTTCCAAGACTACCACTTCTAGTGGCGGTT |
| | | CTACAAGCCCAACTGGCGGCAGTGTGCCGCAATGGGGTCAATGTGGAGGCGAAGGATACACT |
| | | GGCCCTACGCAATGCCAGTCGCCTTACAAGTGCGTTTTTAGCAGTCAGTGGTGGTCATCTTG |
| | | CCAGTAA |
| 55 | MIC-54347 | ATGTTACTGAAACAGATTCTCGTTGCCACGTTGCTTTCCTTGAGCTCTGTCAAGGCTTTCCC |
| | | TAACCCGAATCACGCCCAAGATCTCGAGGCCAGGGAAGCTGAAGATAATCTCGTTGCTCGCG |
| | | GCGGTCACGACTGCGGCTCTTACGCTACCTGGAACTACGGAAAGAACTGCTGTGTCTGCAAA |
| | | GATAACGGCAAGAACTACGATTCTAAGTCCAAGACATGCAGTTGTCCACACGGGCAAGTCTG |
| | | GAATGGCAAGCAATGCGTTGTGGACTGTGGCAAAGATGCGACTTATGATTGGAAGCAAAAGA |
| | | AATGCGTCTGCAAGAAACAAGGCGAGGTCTATAACTCTAATACGAAGACTTGCAGCTGCCCG |
| | | CCTGGAACTGTTTGGAACGGCTACGGATGTGTTGTCGACTGTGGCAAGGAAGCGCACTTTGA |
| | | TAAATGGCAGAAGAAGTGCGTCTGCAATAACAACGGCGAAGTCTATGATTCCAAGAGCAAAA |
| | | CCTGCAGTTGCCCTGGTGGCCAATACTGGAACGGCAAGAAATGCGTATGCCCTTATGGCAAG |
| | | GTCTGGAACGGCAAGCAGTGCATAGAGGACTGTGGTAAAGACGCACACTTCGACTATAACCA |
| | | AAAGAAGTGCGTATGCAACAAGAATGGACAGGTCTACAACTCCAACAGCAAGACCTGCAAAT |
| | | GTCCAGGTGGCCAGTATTGGGACGGCAAGCATTGTGTCTGCCCATATGGTCAAGTCTTTAAT |
| | | GGCAAGCAGTGTGTTCCTGATTGCGGTAAAGAAGCCCACTTTGATTATAAGCAAAAGAAGTG |
| | | CGTATGCAACAACTACGGCGAAATCTACAATTCCAAGAGCAAGACTTGCGCCTGTCCTGACG |
| | | GCCAGTATTGGAATGGAAAGCAATGCGTTTGCCCTTACGGCAAGATCTGGAATGGCAAACAG |
| | | TGTGTTCCTGATTGCGGTAAAGACGCCCACTTTGACTACAACCAAAAGAAGTGTGTTTGCAA |
| | | CAAGAATGGAGAGATCTATGACTCTAAGAGCAAGACTTGCGCCTGTCCTGACGGCCAGTATT |
| | | GGAATGGAAAACAGTGTGCCTGCCCGTACGGCAAGATTTGGAATGGCAAACAGTGTGTTCCT |
| | | GATTGCGGCAAAGAAGCCCACTTCGATTACAACCAGAAGCAGTGCGTTTGCAACAATAAGGG |
| | | AGAGATCTATGATTCTAAGAGCAAGACTTGCGCCTGTCCTGACGGCCAATACTGGAATGGAA |
| | | AGCAGTGTGCCTGCCCGTACGGCAAGATCTGGAACGGCAAGCAGTGTGTAGAAGATTGCGGC |
| | | AAAGAAGCACACTTCGATTACAACCAGAAGCAATGTGTTTGCAATAACAAGGGAGAAATCTA |
| | | TGATTCTAAGAGCAAGACTTGCAAGTGCCCTGATGGCCAATACTGGGACGGAAAGCAGTGCG |
| | | CTTGTCCATACGGAAAGATCTGGAATGGTAAGCAGTGTGTAGAGGACTGTGGCAAGGACGCA |
| | | CACTTCGATTATAACCAGAAGCAGTGTGTTTGCAATAACAAGGGAGAGATTTACGATTCTAA |
| | | GAGCAAGACTTGCAAGTGCCCTGACGGCCAATACTGGGACGGAAAACAATGTGCTTGTCCCT |
| | | ACGGCCAAATTTGGGACGGAAAGCAATGCACACCAAACTGCGGCAAAGACGCTACCTATGAC |
| | | AGTAAGCAGAAGCAATGCGTGTGCAACAAGAAGGGCCAAGTCTTTGATTCCAAGCACTTGAC |
| | | ATGCAGCTGCCCGGCTGGAACGAGTTGGAACGGCTATGCTTGTGTCCCAGATTGTGGCAAGG |
| | | ATGCGCACTACGATACCAATCAGAAGTGCTGCGTGTGCAATAACAAGGGCCAGATATTTAAT |
| | | TCTGGAAGCAAGACATGCAGCTGCCCAGGCAACCAATACTGGGACGGCAATAAATGCACATG |
| | | CCCCTATGGATCGACATGGGACAGCAGTAAGAAGACGTGCAAGCAGACACCCATACATTAA |
| 56 | MIC-54347 | ATGTTGGGCTTCCTCGGAAAATCCGTGGCCTTGCTTGCTGCGCTGCAGGCCACCCTCACCTC |
| | | TGCATCTCCTCTGTCTACAAACGACGTCACAGTTGAGAAGAGAGCCAGCGGATATGCGAACG |
| | | CTGTCTACTTCACTAACTGGGGTATCTACGGCCGCAACTTTCAACCCCAGGACCTGGTTGCG |
| | | TCGGACATCACTCATGTCATCTACTCGTTCATGAACTTCCAAGCAGACGGCACTGTCGTCTC |
| | | TGGAGATGCTTACGCCGATTACCAGAAGCACTATTCCGATGATTCTTGGAATGATGTCGGCA |
| | | ACAACGCGTACGGTTGTGTCAAGCAACTGTTCAAGTTGAAGAAGGCCAACCGCAACTTGAAG |
| | | GTTATGCTTTCTATAGGTGGCTGGACCTGGTCCACCAACTTCCCTTCTGCCGCAAGCACCGA |
| | | TGCCAACCGCAAGAACTTTGCCAAGACAGCCATTACTTTCATGAAGGACTGGGGTTTTGATG |
| | | GTATTGACGTCGACTGGGAGTATCCTGCCGATAACACCCAGGCCACCAACATGGTTCTTCTG |
| | | CTTAAGGAGATCCGATCTCAGCTAGATGCTTATGCGGCCCAATACGCTCCAGGATACCACTT |
| | | CCTTCTCTCCATCGCTGCCCCCGCTGGCCCAGAGCACTACTCTGCCCTGCACCTGGCCGACC |
| | | TTGGTCAAGTTCTCGACTATATCAACCTTATGGCTTATGACTATGCTGGTTCTTGGAGCAGC |
| | | TTCTCCGGACATGATGCCAACTTGTTTGCCAACCCCTCCAACCCCAACTCTTCACCATACAA |
| | | CACCGATCAGGCTATCAAGGCTTATATCAACGGAGGTGTTCCTGCACGCAAGATCGTTCTTG |
| | | GCATGCCCATCTATGGACGATCTTTCGAGAGCACCGGTGGAATTGGCCAGTCTTACAGCGGA |
| | | ATTGGATCTGGAAGCTGGGAGAACGGTATCTGGGACTACAAGGTTCTTCCCAAGGCCGGTGC |
| | | TACAGTCCAGTACGACTCTGTCGCACAGGCATACTACAGCTATGACCCTAGCTCCAAGGAGC |
| | | TCATCTCCTTCGATACCCCTAGCATGATCAACACCAAGGTCTCTTACCTCAAGAACCTCGGC |
| | | CTGGGAGGCAGTATGTTCTGGGAGGCTTCTGCTGACAAGACTGGCTCCGACTCCTTGATCGG |
| | | AACAAGCCACAGAGCGCTTGGAAGCCTGGACTCAACTCAGAACTTGCTGAGCTACCCCAACT |
| | | CCCAGTACGATAACATCCGAAGCGGTCTTAACTAA |
| 57 | MIC-54347 | ATGTTTTTCAGCAAAGCACTGGCTGCTACCGGCCTTTTGGCCACTGCTGCTCTCGCTGCACC |
| | | CACCGTGGAGAAGCGTGCCGCTGGAGGCAAGCTCGTCATCTACTGGGGTGCTGAAGACGACA |
| | | GCACTACTCTCGCCAACGTCTGTGCCGATTCCTCTTACGACATTGTCAACTTGGCTTTCCTT |
| | | GACAAGTTCTCTGCTGGTGGTGGCTACCCCTCGCTCTCTCTGAGCACCCTGGGCGGCCCATC |
| | | TGCCGCTCAGAAGGCTGCTGGTGCCACCAACCTCCAGGATGGTTCTTCTTTGGTTCCCGCCA |
| | | TCAAGGCCTGCCAGGCCGCCGGCAAGCTGGTCATCCTCTCTATGGGTGGTGCTACCGACTTC |
| | | TCTGCAGTCTCCCTTTCGGGCGATGCTCAGGGACAGTCTGTTGCCGACATGGTTTGGAACCT |
| | | GTTCTTGGGTGGTACTGCCAACCCCACTCTCCGTCCTTTCGGATCAGTCAAGCTCGACGGTG |
| | | TTGATCTCGACAACGAGACTGGCAACCCCACTGGTTACCTGGCCATGACTCAACGCTTCCGA |
| | | TCCAACTTTGCCAAGGACACCAGCAAGAGGTACTACCTCACTGCTGCTCCCCAGTGCCCCTT |
| | | CCCCGATGCCTCTGAGCCTCTCAACGTCTGCCAGCTCCTTGATTACGTCTGGGTCCAGTTCT |
| | | ACAACAACGGCAACTGCAACGTTGGCCAGTCCGGCTTCAACACTGCTGTCAAGAACTGGAGC |
| | | AAGAACATTGGTAACGCTACTCTGTTCATTGGTGCGCTGGCCAGCGGTGCCGATGGTGACCA |
| | | GGGCTACATCTCTCCCAGCGCTCTGATCTCTGCTTACAACGGTGTCTCTGCTCTTAACCTGC |
| | | CCAACGTTGGCGGTATCATGCTTTGGGAGGCTCAGCTCGCTGTCAAGAACAGCAACTTCCAG |
| | | AAGACCATCAAGGCTGCCATCGGCTCCGGCTCCACTCCTCCTCCTCCTCCTCCCGCCTCTTC |
| | | CACCACTCCTGCCGGAAGCACCCCCACCTGCTCTTGGGCCGGCCACTGCGCTGGCGCTACTT |
| | | GCAGCACTGACAATGACTGCTCCGACTCCCTCACCTGCAACAGCGGCAAGTGCGGTACCGCT |
| | | GGCAGCACTGCTCCTCCTCCTACCACTTGCTCTTGGGCGGGCCACTGCCTCGGCGCTTCTTG |
| | | CGGTAACGACAACGACTGCTCTGACCCTTACTTCTGCTCCAACGGCGTCTGCTCTCAGTAA |
| 58 | MIC-54347 | ATGTTTTTCCGCAACGCCGTCGCCGTAACCAGCCTTTTGGCTGCACTTTCTAGTGCCCAACC |
| | | CTCCGGCCCCGAATTGGCCGTGTACTGGGGTGCTGAAGATGATAGCACGACTCTCAGCGACG |
| | | TGTGTTCAGACAACTCTTATGGCATTGTGAATCTTGCCTTCCTCGACACATTTTTTGCTGCA |
| | | GGAGGCTTTCCTCAACTCAGCGTCAGCGGGTTAGATGGCCCATCCCAAGCTCAGCAAAGCGC |
| | | TGGTGCCACTGGCCTCAAGGATGGTAGCAGTCTTGTAGACGCTATTAAGCAGTGCCAGTCGG |
| | | CCGGGAAGCTGGTCCTTCTCAGCCTTGGTGGCGCTGGCGCCGATGTCACTCTTCAATCAGAC |
| | | TCTGATGGAGAGAAAATTGCTGATACACTCTGGAATCTGTTCGGTGGTGGCACTGATAACCA |
| | | AGAGCTGCGTCCCTTTGGAGATGTCAAGCTCGATGGCTTCGACTTGGACAATGAATCTGGCA |
| | | ACCCCACAGGTTACCTAGCTATGGTAAAGCGTTTCAAGTCCAACTTCCAGTCGGACACGAGC |
| | | AAAACTTATTTCCTTACTGCGGCTCCTCAATGCCCATTCCCCGATGCTTCGCAGCCTCAAGA |
| | | TGTTTGCAGTGAATTGGACTTTGTCTGGGTCCAGTTTTACAACAACGGCGATTGCAACATTG |
| | | CGCAATCTGATTTCTTAAACTCCGTCCAGACTTGGAGCAGCGGCATTGGCAACGCTAAGCTC |
| | | TACATCGGTGCTTTGGCTAGTGGTGCTGACGGCGATCAGGGCTTTGCTGATGCTGATACGCT |
| | | ATTGGGTGCTATTCAGGATGTCAAGAATATGAACCTTCCCAATTATGGAGGTGCCATGCTTT |
| | | GGGAAGCCCAACTGGCTGTTAAAAACGGCAACTTTCAGCAGAAGATTGCCCCCGGTCTATAA |
| 59 | MIC-54347 | ATGAAATCAAGTCTCTTGTCTCTTATTGCCATTGCAATTAATGGTGCCTTGGCTGGCATTAA |
| | | CTGTAATGGCAGCGGAGACTGTGCCGGGACTCCTGGTACTCTAGGCGATCTTATTGCTGATG |
| | | CATACCAAATTGACCCAAATCGTTGGTATAACAATGGAGAACATATCGCCTGCTCGGATAAT |
| | | CGAGGCGGCGGTGGCCTTTGTGCTTTCTTCCAGAATACTCTCGGTGGCCCTGGAAGCTCAGT |
| | | TCTGACTTTGCTTCAAAACCTGCAAGCTCACGGCTGCAATAAGTGCGGCAGTATTCCGGTAA |
| | | ATTTCCCTCAGGGAGATAACAGTGAAAATCATGGGGAGCTTACTGTCAACTTTGTTGTTTCG |
| | | GCTGGCTGCACTGGGCTTTGCTAG |
| 60 | MIC-54347 | ATGAAGTTCAGCTTTACGTCCGCTATCGCCATTGCAAGCATTGGAATCACTGGCGCCAGAGC |
| | | CGGCATTAACTGCAATGGCAGTGGCAACTGCCCTGGAGTAGCTGGCGACCTCAGCACCCTTA |
| | | TTTCATTTGGATGGTCAATCGACCCCAACCGTTGGTATAACAACGGGGAACACATTGTTTGC |
| | | GTGCAAAGTCAACTTGGGACTGGACTTTGTGCCTTCCTCCAGAACACCGGCGGTGCCCCTGG |
| | | TAGTTCGATTCAGCCGTTGCTCCAGGCATTGCAAGGCCACGGATGCAATAAATGTGGCAGTG |
| | | TTCCGCTGAACTTCCTTCAGGGAGATAACAGCGAAGATCACGGGGAGCTTACTGTTAATGCT |
| | | GTTGGCTCAACTGCCGGCTGCAGTGGGATTTGCTAG |
| 61 | MIC-54347 | ATGACACGCCTCCTCGACGCCAGCTTTCTGCTGCTACCTGCCATCGCATCGACGCTATTTGG |
| | | CACTGCCTCTGCACAAAATGCGACATGTGCACTCAAGGGAAAGCCGGCAGGCAAAGTCCTAA |
| | | TGGGATATTGGGAAAACTGGGATGGATCAGCCAACGGTGTGCACCCTGGATTTGGCTGGACA |
| | | CCGATCGAGAACCCCATCATTAAACAGAATGGTTACAATGTGATCAACGCCGCCTTCCCCGT |
| | | TATTCTATCAGATGGCACAGTGTTATGGGAAAACGACATGGCTCCTGGCACTCAGGTCGCAA |
| | | CCCCAGCTGAAATGTGCGCGGCTAAAGCAGCTGGAGCCACAATTCTTTTGTCAATTGGAGGT |
| | | GCTACTGCTGGCATAGATCTCAGCTCAAGTACAGTCGCTGATAAGTTCATCGCGACCATTGT |
| | | ACCAATCTTGAAGCAATACAATTTTGACGGTATTGATATTGACATTGAGACCGGCTTGGTCA |
| | | ACAGCGGCAATATCAAAACACTTTCCACATCACAGGCCAACTTGATTCGCATCATTGATGGT |
| | | GTTCTTGCTCAGATGCCTTCCAACTTCGGCTTGACCATGGCACCTGAGACAGCATACGTTAC |
| | | AGGTGGTAGCATCACGTACGGCTCTATTTGGGGATCCTACCTACCTATCATTCAGAAATATG |
| | | TTCAGAACGGCCGGCTGTGGTGGCTAAACATGCAATATTACAACGGCGACATGTACGGCTGC |
| | | TCTGGCGACTCATATGCCGCTGGCACTGTTCAAGGATTCATCGCTCAGACTGATTGCTTAAA |
| | | TGCAGGTCTTACCGTCCAAGGCACCACAATCAAGGTTCCATATAGCATGCAAGTACCCGGTC |
| | | TACCTGCGCAATCAGGAGCTGGCGGTGGCTACATGAACCCGAGCTTGGTTGGACAAGCCTGG |
| | | GATCACTACAACGGTGCTCTGAAAGGCTTGATGACGTGGTCAATCAACTGGGATGGAGCAGG |
| | | CGGCTGGACGTTTGGCGACAATTTGCTTACTCGTATTGGTTAG |
| 62 | MIC-54347 | ATGCACCTCCACTCTCTCTTTACGGCGCTTGCGCTGGCGAGCCCTGCGCTCGCTGCTTCAAT |
| | | CCCTCGCAGCAGCAGCAGCTCCTCATCATCAACTATCACCGTCTGGGCGACTCCCCACGACA |
| | | GCTACTCATCCTCCGTCGGCGTCTTGGGCTGCAAGGTCGACACCAATCGCATCGCCTACTGG |
| | | CCAGACTCCGTCGACTGCACCAACATCTGCGTGTCTCTTTCGTACCAGGACCGACAGGTCTA |
| | | CCTCCTTCGCGTCGACCAATCACAGGGCGCGCACGACATCAGCTACGATGCCTGGAATTACC |
| | | TCGTTACCGGCTATCCAGCCACGGAAAAGCCCGTTGCGGGAGGGCCCATGGAAATGACGACG |
| | | GAAAACGTCGATGCGTCCAAATGCGCCGACCTCATCTACACAGCGGGCGGCAAGCTTCCCCT |
| | | CAGTGCGGCAAATAGCATGAATTTCTTGGCCAGCTGCTTGGAGCAAGAGAATTCATGGGTTG |
| | | CAAGCAACTACGTGTTATACAACATCTTGGATGCAATTTGCACCGTTGGCCAAAACCAGGTC |
| | | TGCTCTTTGAACTGGCCCACTGCCAACCAGCCCACCTGTCCGGGCACCCTAGGATTGCCCGA |
| | | CGCACTCAAAGGCGAACCTGTCTACAATATCGAGTATCCTTCCGGCGTCAAGGTACTTGCAG |
| | | GGGCGCCACCAACTGTACCAACTGGAGTACCTGTTCCAGTACCGGCGCCTTCTACCAACGAC |
| | | GATGAGAAGAGCGCTGCACGGAGTTTAAGGCATTCAGACTCTCTCGTCTGGATACCAGTCCT |
| | | TTCATTAACTTCCATTATATATTCTTGGATGCTGTGGTAA |
| 63 | MIC-54347 | ATGCGTTTCAACAGTTTCATCGCCGTCGCGGCTGCGACCGCTCAAAGCGCCGCCGGCCTGGG |
| | | AATCAACTGCCTGGGCGACACTCTCTGCGGAATCTCGTACATGAGCGGCGGACGCCTGACCC |
| | | AGTTCCAGACCATCTTCGACAACATCTTTGAGAAGCGGATATACGACAACGGCGACGACATC |
| | | GGCTGCATCGAGGTCCACTCAATCAACTTCAAGGGCTCCTTCAAAGGCACATACTGCGCCTA |
| | | CGTCCAAAACCTGGATGGCAGCGTCAACGGCGCCACCCTCAAGACCCTTTACACCGAGCTGG |
| | | TCAACTATGGCTGTGGAATCTGCGGCTCCATCCCCATCCACTACAGCAAGGGCGACAACGAC |
| | | TCCAACCACGGAGAGCTCTCTTTCAACATGGTGGACAGCCTGCCGGACAACTGCGAGCTCGG |
| | | CAAGCCTTGCGCAGCGACCTCCTAA |
| 64 | MIC-54347 | ATGTACTTCGCCAACACCCTACTCTCCATCGCCGGTGCTGCATCAATAGCCAATGCCTTGGG |
| | | CAGCAATTGTCAAGACAGCGGTGTTTGCGCGGATATCAACGCTAACCTGAGTTTTGCCATTG |
| | | AACAACTAAAAGGAATGGACCAGCTCCAGAGATTCTCGGACGGACAACACATCACCTGCGTT |
| | | GATACGGACAGTGAGGGCAACTCTTCCCTCTGTCTCTCATACCAGGACACTGGCCGATCATG |
| | | GACTGTATTCCAGACTGCCTGGTTTGCCCAGTCCTTGGTAGAGCAGGGATGCCAGGCCTGTG |
| | | GAAGCCTGTCTATGGGCTCTCATCATGGCGAGCTGGTCGCCAGCGTCATCACAAAGAGTACC |
| | | AGCGGCCTGGATACCTCTGGAGCTCGTCGTGGGATGGACATGGTTCAACTGGCTGTAAGAGC |
| | | TGGGGACCGATGA |
| 65 | MIC-54347 | ATGTTGTTTAAAATTGCCATCGTTGTGATCGCCTCCACAGCACGTGTTTCCGCCTTTGGTAT |
| | | CAACTGCGAAGGCAGTGGCTACTGCTCTCCATTGTTCAATCCTGGAGCCAACAACCACCCGC |
| | | TGCTAGAGATGGTGGACGTTATCGACTTTGGAATTGATGACAATCGATGGTATGCAGCAGGA |
| | | GAGCATATTGCCTGTGATCAGAGTTCTGGTGTCTGCGCTTTTGTACAGAAGATTGGCGGTGC |
| | | TTCAGGTGGCGATATTGCTCGAGCTGTCCGCTATCTCGCAGACCATGGCTGCACTACCTGTG |
| | | GAAGCGTACCCTTGGACTTCCCTAACACCAACGATGTCAACAACGGAGAGGTGACTTTCAAC |
| | | TTTGTTGGGTTGGAAGATATGGGTAGCTGCAGCGATCTTTGCTAA |
| 66 | MIC-54347 | ATGAAAATGCTTATTACTGCCGCTCTTTTCACCCTGGCCCTCGCCGCCCCTGTCGCCGAGAC |
| | | TAAGCCTCATAGCATTGCTGCTCGCGATCCCTTTACTTGCCCCGGCGGCCTGACCAACAGCA |
| | | CCCCCATGTGCTGCAGCGTTAATGTCCTTGGCCTGCTAGCCCTTGATTGCCAGCAACGTATG |
| | | GGTTACTTTGCTTCATATTTATCCTTTCAATATATGGGGAACTTGAATAGCTGACATTTTCA |
| | | TCGCTCATTAGCTGGCGCGGACGGCTGTAGTGGCAGCTCCAAGCCCAATTGTTGCACCCTTG |
| | | GAGCGGTATGTTTTTTAATGATTGGTCACGCCAACAGTTTTGAGCATTTACTGATAGAATTT |
| | | CTCAATACAGGCCGGACAGGGTGTTATTTGCAACGCACTGTAA |
| 67 | MIC-54347 | ATGAAGTCGGCCATTCTTCTTGGGCTCACTGGCCTCGCTGCCAATGTGAATGCCCATCCTGC |
| | | TAAGCAGCCAGAGACCGGCAACGGCCTGACTAAGCGTGGCGTTGATATTAGCAAGTATAGCT |
| | | TGCCTAATCTTTCAAAGTACACGTCTTCGCCAAACATCGAACAAGAGGCCTCTCTGCAGTCT |
| | | TTGGCTTTCAAGCGCAACTATGTGGATACAGCCACACGAGCTGTCAAGAAAGCTGCCCCCAA |
| | | GGCCGAATTCCGCGTCGTGGATGACCACTATATCGATGTTGACGGCATTGGTCACGTCCACT |
| | | TTAAGCAGACAGTGCACGGAATTGACATTGACAACGGTGACTTCAAGGTTAACGTGAGTGGA |
| | | AGGCAAAATCACCTCACCTAGAAAACCAGAAACTTACAAATAATATGAACTTAGATTGGCAA |
| | | AGACGGTAGAGTCTTCTCTCATGGAAACAGCTTCTTTTCTGGCAAGCTGCCCCAGCAGAACC |
| | | CCTTGAGGAAGCGTGATTTCTCTGACCCCACCACTGCGCTCAAGGGTGCAATTGATATCCTG |
| | | GGCCTCCCGGTTCAGGCTGATGGCGCCACCGCCGAAGCTCAGGAAGGCACTGAGAAGTACAC |
| | | GTTGAAGGGTACATCCGGAGCTGTCAGCGATCCTAAGGCTCACCTCGTTTACCTCGTCAAGG |
| | | GAGACGGAACTCTGGCTCTGACTTGGCGGGTCGAGACCGATATCATGGACAACTGGCTTCTC |
| | | ACCTACGTTGACGCAACAACCAACCAAGAGATTCATGGTGTTGTAGACTACGTCTCCGACCT |
| | | TGCCACCTTCCAGGTTTACCCCTGGGGCCTCAATGACCCCACCGAGGGAGATCGTAAAGTCC |
| | | TGACCGACCCGTGGAGAACCGATGCCTCCCCATTCACTTGGCTCAGCGACGGAACCACCAAC |
| | | TATACTGTAACTCGCGGAAACAATGCTATTGCGCAGGATAATCCTTCTGGGGGGGACAGTTA |
| | | TCTCAATAATCACCGTCCATCCAGCTCGACTCGGGACTTTCAATATCCCTTTACGCTGACTC |
| | | AGACCAACCCTACAGATTATAGGGATGCTGCCATCACACAGCTCTTTTACACAGTCAACAAG |
| | | TACCACGATTTGCTATATGTCCTTGGCTTCAACGAGGTAGCAGGAAACTTCCAAGCAAATAA |
| | | CAACGGAAAGGGCGGCAAGGCCAATGATTTCGTTATTGTTAACGCTCAAGACGGCAGTGGAA |
| | | CGAACAATGCCAACTTTGCTACCCCGGCCGATGGAAGCAATGGCCGTATGAGAATGTACATC |
| | | TGGACTACTGCTAACCCCAAGCGAGATGGCGATCTCGAGGCGGGCATTGTTATCCACGAATA |
| | | CACTCACGGATGTAAGTCTCTCTCATTTCAACTTCGTATCTACTCTTTCTAACGCTTGAAAC |
| | | AGTGTCTACCCGTCTTACCGGCGGCCCTGCCAACTCTGGATGCCTTACTGGCACTGAAGCCG |
| | | GCGGCATGGGTGAAGGCTGGGGTGATTTCTTCGCCACCGCTATCCGCTTAAAGGCTGGTGAC |
| | | ACCCGCAGCAAGGACTATCCTATGGGAGTCTGGGCCGATAACAACGTCAAGGGTATCCGCCA |
| | | GTATCCTTATTCAACAAGCCTTACTACCAATCCTCTTACTTATAAAACCGTCAATACGCAAA |
| | | ATGAAGTCCACTCAGCCGGTACTACATGGGCCACCATTTTGTACGAAGTCCTGTGGAACCTC |
| | | ATTGACAAGTACGGCAAGAATGACGACGATTTCCCTACCTTTGATAGCCAGGGCGTCCCCAC |
| | | CGACGGCAAGTATCTTACCTTGAAACTCGTCCTCAATGGATTGGCTCTGCAGCCCTGCACGC |
| | | CAACTTTTGTTTCTGCCCGTGATGCCATCTTGGATGCTGATCGTGCTTTGACCGGAGGGGAG |
| | | AATCTTTGTGAATTATGGACAGGATTCGCCAAGAGAGGTCTTGGATCCGGCGCCAAATATTC |
| | | TTCCACCGCTCGCGTTGAAAGCTTCACTATCCCATCTGGGGTTTGCTAA |
| 68 | MIC-54347 | ATGAAGTTCTTCGCCGCCACCGCTCTCCTTGCCGCCACTACCATTGCTGGACCTCTTGAGGT |
| | | TCGCACTGGAGACGGTAACATTTGTCCTAGCGGCGGCCTCTACGGCAACCCTCAGTGCTGCT |
| | | CCTCTCTCCTCCTTGGTCTCATTGGTCTTGACTGCAACGTCCGTAAGTTAAAAACAAAGCTC |
| | | ATTCAAAGCCTATTGATGCAATACATTCTCATACTGGTGTCCTATAGCCAACCAAACGCCCC |
| | | GTGACGGCGCTGACTTTAGAAACATCTGCGCTAAGACTGGTGACGAGGCCCTTTGCTGTGTT |
| | | GCTCCCGTTGTAAGTAATCACCCAGCTTCAAGTTGCCCTTGCCCTTGCCCTTGTGCTGTTGC |
| | | TAATTTCTACACTATCTTTAGGCCGGCCAAGCTCTTCTCTGCCAGGTCGCCGTTGGTGCCAG |
| | | CTAA |
| 69 | MIC-54347 | ATGAAGTTCTTTGCTGTTGCCACTCTCTTTGTCACTGGCGTCATCTCCGCCCCGTCACCCAA |
| | | TGCCCTAAATTCCCGCAGTCTTCTTTGCAGCCCTGGCCTTTACTCCACCGCGCAGTGCTGTG |
| | | GTGTTGATGTATTGGGAGTTGCTGATTTGGACTGTGCGGCTCGTAAGTAGTCTCTCAAAGCC |
| | | CAATCATATTTATTGATTTGAAGATGCTGATACTGGGAATTTAGCTACTGGCACTATTACCA |
| | | ACGCCCAAGGATTTCAGGCTGCCTGTGCTAAGAAAGGCCAAGAGGCTCGCTGTTGTGTTCTA |
| | | CCTGTTGTAAGTTATCCTAAGATACGTGTAATGCGGCTGCTTTTATAGACTGGCAATACTAA |
| | | CAATTTTTGTCCCTTTAGGCCGGCCAAGATGTCCTGTGCCAGGACCCCCCTGGTTTGTAA |
| 70 | MIC-54347 | ATGACCAGCATTCGTCGTCTTGCTCTCTATCTCGGAGCTTTGCTCCCGGCTGTCCTCGCCGC |
| | | TCCAGCAGCCCTTCACAAGAAGCCTGAGGCTGCACCTAACAAGTTTATTGTCACTCTTAAAG |
| | | AGGGCGCTTCAATTGATACCGACTCTCATCTCGCCTGGGTGAATGACATCCACCGTCGTTCT |
| | | TTGACCAAGCGTAGCACTGCTGGTGTTGAAAAGACTTATAACATTCATACTTGGAGTGCTTA |
| | | TGCGGGTGAATTTGATGCAGAGACGATTGAGCAGATCAAGTCTAGCCCCGATGTACGTGCTC |
| | | TCCGTATCCTAACGAAAAGAAGAAAAGCTAATCACGCTGTCAGGTCGCGTCTGTGGAGCCAG |
| | | ACTACATCATGTACCTGTCGGACATTGTTGAAGACAAGCGTGCTCTGACCACACAATCTGGA |
| | | GCTCCTTGGGGCCTTGGCACTGTTTCCCACCGCACATCTGGGTCTACGAGCTACATTTACGA |
| | | TAGCTCAGCTGGCTCTGGAACCTTTGCCTATGTGGTTGACTCCGGCATCAACACCTCTCATC |
| | | AGCAATTCGGCGGGCGCGCCAGTCTTGGCTACAATGCTGCAGGAGGACAGCACGTCGACACT |
| | | CTTGGCCATGGTACTCATGTTTCTGGAACAATTGGTGGATCTACATACGGCGTTGCTAAACA |
| | | GGTGAGTGATTCCAATGTTGTTGTTATCACATACAAGTGCATTCAAGACTGACTCGGTACTA |
| | | TTCATAGGCCAGCCTAATCTCCGTCAAAGTCTTTGCTGGAGAGAGCGCTACCACCTCTGTTA |
| | | TCCTTGACGGCTATAACTGGGCTGTAAACGACATTGTCTCGAGGAATCGTGCTAGCAAATCT |
| | | GCCATTAACATGTCGCTTGGAGGACCTGCCTCATCCACCTGGACGACCGCCATTAACGCAGC |
| | | CTTTAACCAGGGTGTGCTTACCATTGTCGCCGCTGGTAATGGCGACAGTTTCGGAAACCCCC |
| | | AGCCAGTTTCCGGCACTTCTCCTGCTAATGTTCCTAATGCTATCACTGTTGCGGCGCTGGAC |
| | | ATCAACTGGCGCACTGCTTCCTTCACCAACTACGGTGCTGGCGTTGATGTCTTTGCCCCTGG |
| | | TGTTAACATTCTGTCGTCATGGATTGGATCCAACACTGCTACCAACACAATCAGCGGCACCT |
| | | CTATGGCGACACCTCACGTTGTTGGTCTGGCTCTCTATCTTCAATCCCTTGAAGGCCTCACC |
| | | ACTCCTACCGCTGTCACTAATCGGATCAAGGCTCTGGCTACCGCTGGCCGTATAACTGGCAG |
| | | CCTTAACGGCAGCCCCAACACTCTCATCTTCAACGGAAACAGTGCTTAA |
| 71 | MIC-54347 | ATGAGAACATCTACCGCTATTATATCTCTTGCCCTTGTGGTGATGGGAATTGCCGCCCCTGC |
| | | TGGTCATGTATCGAGCAACCAGCCGCGGAGCGTGGAAGGTTGAGCTCATCAAAGGAGCAGCC |
| | | GAGGCCGGCTCTGTTGTCAGCGAAGCCGGCTTTGTTGTCAGCGAAGCCCAATTCAACGCCAT |
| | | ATTTCCCGGTCGAAACCCTTTTTACTCCTATAGGGCATTGACCGGCGCCCTCGGATCTTATC |
| | | CCAGCTTCACCAACACTGCTGATAACGCCACTAGGCTCCGCGAGGCCGCCGCTTTCTTCGCC |
| | | AATGTGGATCATGAAACCGATGGGCTCAAGTTCGTCGTGGAATAA |
| 72 | MIC-54347 | ATGAGCGCCGCATCCCGCAAGTCACGCATAGCTACGAGTGTGGCGAGAGTTATGTACACCAA |
| | | TGCAGTCTATTTCCCTAGCAGCAGAATATATCAAGGTGATTCGCCGGGAATGCTCAATTACA |
| | | GCTGCATCAACCACGTGTATTATGCTTATGCCAGCGTGACGGCGGACGGCAATGTGTTTGTG |
| | | AGTTTTGATCAAGTCCCGGAGACCACACATATGGCAAAGTGGCTTCTTTTCCCGTGCCATGG |
| | | TTCGGAAAAGTTGAGAATATTTCACCTGCATGATGTTGCTCGGCTGCTGACTTGTGACGTGT |
| | | GGCCCAGCTCGGCGATGAGTGGGCCGATGCAAGAGCGCCAGTGGATGGTGTTCAGGGTGGCT |
| | | TAGGATCCTTGATGCATCTCAAGCAGAGGCACCCTCACCTGCAGGTTGTCTTATCTATTGGT |
| | | GGCAGCACTGCATCAGAGGTATTTCCCATTGTTGCATCAAGCACTCTCCTCAGAGACAACTT |
| | | TGCCCGGTCTGCCCTTGGCCTCGTGGAAGCATCCGGGCTTGATGGTATTGACAGTAAGTACG |
| | | AACGAACAAGAGGAAGACGAACAAAGAAGGAGGTAGAGGAAGCATGGATGTCAAGTTCACAC |
| | | ACGCGCGCTCGCACATACACCCGCCACAAACAAGAGTCTTCAGCTAATTACTCGTCTCCCTA |
| | | AATAGTTGCCTGGGAGTTCCCATCCCAAGCTAAACATGGCCACGATTTCCTAGCCTTGCTGG |
| | | CAGCAGTGCGGATTCATATGCCTGAAGACCGCTTCATTCTAACAGCTGTCCTCCCTGCGGCG |
| | | AAGGAGGTTCTACAACTCATCGACCTCAGCACAGCGGCCGAGTACCTTGACTACATCAACCT |
| | | TGTGGCATATGATTTCTTCGGCACGTGGACGTCCAAAACTGGTCATCACGCACAGTTATATA |
| | | CTATGAACAAGGACGAACCCTCGGCATCGACAGGTGTGGCGTATGTCATGTCCCAAGGATTC |
| | | CCTGCGAAGAGTATCCTGCTCGGGATCCCAACCTACGGACGAAGCTTCCTCAAGGCCAATGG |
| | | GCCGGGCCAGGATTTTAACGGCGTTGGCGGCCAAGATGGCACATTCGAATACGGCGAATTAC |
| | | CACGAAAGGGATGCAAAGAGATTGTGGATAGGCGCTATATTGCGGCCCAATGTGTCGGTGGT |
| | | GATGGGGGGTTTGTCACGTACGACAACCCCGAGACGGTCAAGGTTAAGGCGGAATTTTGCAA |
| | | GCAAAAGGGATTAGGGGTTTGTGGAGCTCAAATTTATCCAATCTCTGTGAGACACAAATTGG |
| | | TTGCTAACTATCAAATTTTTAATATTGCAGGGGCTTTTTTACTGGAACGGACCGGCTGACTC |
| | | TCGAGATCAAGCACGAAGTCTGATTGCGGCTGGATTCCGCGCTCTACACACCTCGTGA |
| 73 | MIC-54347 | ATGCAATTGACCACAGTCGTCGCGCTCTTCGCCTCTCTGGCCGGCGCCGCGCCTGCCCCTGA |
| | | ACCCGCTGCTGAGCTTGTTGCTCGTGATGGTCCTTGCTCCTCTGGTGTCACCAATAACGTCC |
| | | CTCAATGCTGCGGTACTGGTATCCTCAGTGTTGTCTACGTGGACTGCAAGACTCGTAAGTCT |
| | | TTCAACGCTCTCGCTTGATGAGAAATCCCCATTGCATGCTAAGCCTTGACTTAGCCACTCAA |
| | | GCTCCCTCTGCCACTAACCAGTTGAGCGCCATCTGCGCTCGAGTAGGTCTCCAGGCCAAGTG |
| | | TTGCACCGTCGGCATTGTAAGTTCTTCGTCCTAGAATCGGATTTGCAAAAGGGATTCTAATT |
| | | ATTGTATCAGGCTGGCGTTGGCGTTCTTTGCCAGGATGCTATTCCCCAGTAA |
| 74 | MIC-54347 | ATGCAATTGTCCAACCTCTTCAAGCTTGCTCTCTTCACCGCCGCTGTCTCTGCTGATACCGG |
| | | TATGATGAATCACTACCTGTATTCGATGCTCTCGTTTTGCTAACACAGATTCACTCACACAG |
| | | TCTCGTACGATACTGGCTACGATGACGCATCTCGCTCTCTGACCGTCGTCTCCTGCTCCGAC |
| | | GGCCCCAACGGCCTCATCACCAGATACCACTGGCAGACCCAGGGCCAGATCCCTCGCTTCCC |
| | | ATACATTGGTGGTGTCCAGGCCGTCGCCGGCTGGAACTCCCCTAGCTGCGGCACCTGCTGGA |
| | | AGCTCACTTACAGCGGCAAGACCATCTACGTCTTGGCTGTTGACCACGCCGGTGCTGGCTTC |
| | | AACATTGGCCTCGACGCCATGAATGCTCTCACCAACGGCCAGGCTGTTGCCCTCGGACGTGT |
| | | CTCTGCCACTGCCTCTCAGGTGGCTGTGAGCAACTGCGGTCTCTAG |
| 75 | MIC-54347 | ATGCATTCAACAACTTTCTTTGCCAGTCTGGGACTCGCAGGCCTGGTTGCCGGCGCCCCTTC |
| | | GGCCCCGCACAACGTTAAAGCTCGCCAAGCTTCCGGTGCCCAAAACGTCGTCTATTGGGGAG |
| | | GCACTAACAATGAAAGCGACGACCTTTCTACCTATTGTACGCCCACCGCAGGCATTGACATC |
| | | CTTGTTCTGTCTTTCTTGGACATTTACGGCACAACTGGCAACATTCCAGCAGGTAATATTGG |
| | | CAATTCTTGCTATATCGGAACAAATGGCACACCTCAACTATGTGATAATCTTGCTGCTTCAA |
| | | TAGCCAGCTGCCAGGCCGCTGGTATTAAAATTATCTTGTCTCTCGGAGGAGCGGCCGGCTCC |
| | | TATTCTCTACAATCGCAGTCACAGGCTGTGGCTATTGGCCAGTATCTCTGGGAGGCTTACGG |
| | | CAACTCTGGCAGCACTTCTGTCCAGCGGCCCTTTGGCAATGTCTTTGTTAATGGTTTTGACT |
| | | TTGACCTTGAGCTGAACGCCGGCAACCAATACTACCAGTATCTGATATCAACCCTCCGTTCC |
| | | AACTTTGCCAGTGACCCGAAACATACGTATTATATCACTGGAGCGCCCCAGTGTCCTCTCCC |
| | | GTTGAGTATTCCTCAAATCCGTAGTTAGATGGGTGACTCATGGCTTGCTAACAACACTAATG |
| | | GAACAAATAGAGAACCAAATATGGGAGAGATTATAAGTGCCTCACAGTTCGACTATCTCTGG |
| | | ATTCAATTCTATAACAACAACGCTTATGCACCAGACCCCTGTTCTCTTGGTCTGCCTGGCGA |
| | | TGCACCCTTCAACTACAACAACTGGACGTCATTTATTGCTACTACCCCTTCAAAAAACGCAG |
| | | TATGTGTATTTCGGCCCCTGGTATTATCTTGCATACCAAATTTCTAACAATTTCTACTAGAA |
| | | ACTATTTATTGGAGTCCCTGCGAATACTTTAGCTGCTAATGGCAATTCAGGTGGTGCCGTTT |
| | | ACTATGCTTCGCCATCCCAGCTTGCTTCCATTGTCGCGAATACAAAATCTAGCCCAGATTTT |
| | | GGAGGAATTATGATGTGGGATGCTGGGTACTCTGATGCTAATGTCAACAACGGATGCAACTA |
| | | CGCTCAGGAGGCCAAAAACATCCTCCTTACCGGCGCTCCCTGTGGAGGCTCACCCCCACCGG |
| | | TTAGCAGCAGCAAGCCTACATCCACTGCAACCAAATCCGCTACTAGCACTTCATCAGCCTCG |
| | | GGAACAGGCCCGACAGGAGGCGGCACAGTACCTCAGTGGGGCCAGGTGAGTCACTAGAGGCC |
| | | TTGGGGCTTACATATGACCTGGGGAAAAGAGCCCTAGCTAATTGATGTTTTAGTGCGGCGGA |
| | | GAGGGATACACGGGCCCGACGCAGTGCGTTTCTCCCTACAAATGCGTCGAATCAAGTCAATG |
| | | GTGGTCGTCTTGCCAATAA |
| 76 | MIC-54347 | ATGCCCTCTTTGATCACGGTTGCGAGCGTGCTCGCTCTTGTTCCATCCGCCTTCGCGGGCTG |
| | | GAATGTCAACTCGAAGCAAAACATTGCTGTGTACTGGGGTAATGACTCTTGTAAATATTTCT |
| | | AATTTATAGAAAGAAAGCATCTAACATGCTATCTGTATAGGACAAAACTCCGCCAACCAACA |
| | | GAGCACACAGCAGCGTCTTTCAGCCTACTGCAATGGTACGTTTGTGCTTTATTTCCTGACCG |
| | | AAACCTACGATTGGGTTGAGAAACAGCGAAGATTGAATCTGACAGTTGCGATGTAGATGCCA |
| | | ACATCAATGTCATTGACATTGCTTTCCTGAATGGAATTACTCCTCCTATGACCAACTTTGCC |
| | | AATGCTGGTGACCGATGCACTCCCTTCTCCGACAACCCTTGGCTCTTGAGCTGCCCTGAAAT |
| | | TGAGTGAGTCTTCTGTTGAAGAAATGCTTTTCTGTTGTAAATAGATATGGACTAACCATGGT |
| | | TGAATTAAAGGGCGGATATCAAGACTTGCCAGGCTAATGGCAAGACCATTCTCATTTCTCTT |
| | | GGTGGTGATTCTTACACTCAAGGTGGCTGGAGCTCTACCAGTGCTGCTCAGGCCGCAGCCAA |
| | | ACAGGTCTGGGCCATGTTTGGTCCTGTCCAATCCGGCAGCTCCACCGAGCGTCCCTTTGGCA |
| | | GCGCCGTTGTTGACGGTTTTGACTTCGACTTTGAAGCGACGACTAACAATCTCGCGGCCTTT |
| | | GGCGCTCAGCTCAAGAGCCTCTCGAGCGCTGCCGGTGGCAAGAAGTACTACTTCTCTGCTGC |
| | | TCCCCAGTGCTTCTTCCCCGACGCTGCTGTCGGTGCACTGATCAACGCCGTCCCCATGGACT |
| | | GGATCCAGATTCAATTCTATAACAACCCTTGCGGCGTCAGCGGCTACACCCCCGGCACCAGC |
| | | AACCAGAACAACTACAACTACCAGACCTGGGATACCTGGGCCAAGACAAGCCCCAACCCCAA |
| | | CGTCAAGCTTCTTGTCGGCATTCCCGCTGGCCCAGGTGCTGGTCGCGGCTACGTGTCTGGCT |
| | | CTCAGCTCACTTCAGTCTTCCAGTACTCGAAGGGCTTCAGCAGCACTTTTGCCGGTGCTATG |
| | | ATGTGGGATATGTCCCAGCTCTTCCAGAACACTGGCTTCGAGGCCCAGGTTGTCAATGCTTT |
| | | GAAATAA |
| 77 | MIC-54347 | ATGCCGCTTAAGGTCTTCGAGTCAGCTTCGCCTGCGCCGTCGCATCCCTCCTTATCGCACCT |
| | | CTGCCTGCTCGTCTTCGAAGCTGTTCTAGAAGTAGTCTGTGTGAGCTTGCCGGGATACATCG |
| | | TTGCGCGCCTTGGCCATTTCGATGCCGATAAGCAAAAGTTCCTGGCTAATCTCAATGTGATG |
| | | CTCTTCACGCCCTGTCTTAGTACGTTGCTCCCTCTTGCCATGTCTCTGTTATTCTCCTTTGA |
| | | ACGCCGTCTGACCCATTTCTCTCCTCAGTCTTCACCAAGCTAGCATCTCAGCTCAATGCCGA |
| | | AAAACTATCCGATCTCGCCATCATCCCCGCCATCTTTATCGTGCAAACTCTTGTATCATGGA |
| | | TAGTTTCGATCCTGGTCGCCAAGGGGTTCCGATTCAATAAGCGAGCTTCCAACTTCGTTACT |
| | | GCTATGGGCGTCTTTGGCAATTCAAATTCACTGCCCATCTCTCTCGTTCTTTCTCTCTCGCA |
| | | AACAATCAAGGGCCTCCACTGGGACAGGATACCAGGCGACAACGATGACGAAGTCGGCGCTC |
| | | GCGGTATCTTATATCTACTGATTTTCCAGCAACTTGGCCAGCTTGTCAGATGGAGCTGGGGA |
| | | TATCACGTTCTGCTAGCCCCCAAGGATAAATACGCCGAGTATCAAGACGAAATTGCCGAAGA |
| | | AGGCCAGTACAGATACAGAGACGAAGAGCCGAACGAACAGGAGCCGGAGATTCTCATTACTG |
| | | GTTTGGACGGTGATACCGAAGATGATGGCGAGAGCAATGCTTCTGAAGATTATATACCTGCT |
| | | GGACGAACACCTCTTGCGAGCAATTCCCGGGCTTCACTGGCTGGCTCTTCCGTCGACAATGA |
| | | CGACATGCTGAATTTCAAAAAGGGCAACTACACTCGTGGAAGCTCGCTCGCCAACACAGACT |
| | | TGGAAGATGATATTCTTTCATTCCCTCGTATTCGTCTTCGAGATGAGGCTGAAGTCGAGCAC |
| | | GGAGTTACTGCTCGTATCAAGAAATCACTATATTCTCTGAAAGACAAAGCTTCTGCGGCCAT |
| | | GACTCGCCAATATCAGCGACTTCCTCAACCTGTTCAAACCTGCCTTTCCTTCATTCATAAGT |
| | | CAATCACAAAGACTCTCGGATTTGTATGGGATTTCATGAATCCACCTTTGTGGGCTATGCTC |
| | | ATCGCGGTCATCGTGGCGTCCATCCCGAGTCTCCAACAGTTGTTCTTCGAAGATGGTTCATT |
| | | CGTGAAAAATAGCGTCACCAACGCTGTTTCGTCTAGTGGAGGCGTTGCAGTGCCTTTGATCT |
| | | TGGTTGTTCTCGGCGCCAACTTGGCGCGCAACACTGCCGCTCACGATTCTCCCATAGACCCG |
| | | GAGGAGGAAAAGATTGGCACCAAGCTCTTGATCGCTTCGTTGCTGAGCAGGATGGTTCTGCC |
| | | AACTTTGATTATGGCACCCATTCTGGCTATTACGGCAAAATACTTGCCCATCAGCATTCTAG |
| | | ATGATCCCATCTTTATTGTCGTATGCTTTCTTCTTACTGGCGCACCCAGTGCGCTCCAGCTT |
| | | GCGCAAATTTGCCAAATCAACAATGTTTATGAGCAGACTATGGGAAGAATCCTCTTTCAGAG |
| | | CTATGTTATCTGGTAAGCATTTCAACACCCTTAATTTTGTCTATCGTGCATTTCACTAATAC |
| | | ACATATTAGGATTCTTCCCTCTACTCTTTTCCTTGTCATGATGGCACTCGAGGTGATTGAGT |
| | | GGGCAACAGTGAATTAA |
| 78 | MIC-54347 | ATGCTACCGTCGAGCCTTTGCCGGATTGCCGCTGTTATCAGCGTGGCTTCTGCGGAAATGGT |
| | | TTCGGGTGAGTACAATATAAACCCTCCAACATGTGATCTGTCTCACACTTATACATCATAGT |
| | | AACATTTGATACCGCTTACGACGATCCTTCTCGTTCACTGAGTGAAGTCGCTTGTTGGAGGA |
| | | AGGGCACGGGATTCATGCCTAACTTGGATTGGAAACTTCAAAAAGATGCATTGGAATTTATT |
| | | GGAATCAAAGCAATTCGTGGCTTCAGTTCTGCCCAATGTTTTTCCTGCTGGAAAATTGAGTA |
| | | TGGAGATAAGCAGATATCACTGTTCGCAATTGACAGCGCCGACTCGGGCATTGTTCTCTCTC |
| | | TGAGCACCATGCAATATCTTACAGGTGGCCAGGCTCGCGAGCTGGCCCGAATTGATGCAAAG |
| | | GCAACCCAAGTCGACGCATCAAACTGTGGGATTTCTGCGGCTGAACTGCACAAATACGATTT |
| | | CTAGTTAGTCGTATCCACTTTTAGGCTACCAAGCAAGCGTTCAAGTAGTTTCAAAGATAAGT |
| | | CAAGCTTTGTATCTTCGCCCCTATTACCTGGAGTTGAGACTATTTGTTACGTTTTTTTTGGT |
| | | GAGACTACGGCTACATATAGCCCCAAACGTTCACTTCTACTGCGGCATTAGTTTTTATACAA |
| | | TGCATTATTTACGCCAATATCTTGTATCAAGGTCTGAGTAGTGTCAACCTCCACTCTTGAGA |
| | | GAGCATCTATAGCTTAAGAGTATACTCATTACGACCGAAATATATTGATATTTTATTCAAAC |
| | | ACATAACTAGCAATGATAAAAGTCGCCTTAGAGGACGTAAGAGTAATAAAATGTAAAAATTT |
| | | CAGATCTGGACACATATACTGCACACACATACATTTATAACTCCGCAATTAACCATTTTTGG |
| | | TTAAGTACAGCTGAGATAACTTACTTTCATACATCGATTTTACCGTAGTTGAGAATATCTTG |
| | | GTATTTAATTATCTTTACATTCGGTTCGTCATTCAACAAAAGATGCGCAATTGAAGTTGTCA |
| | | CATTATCTGTTTAATTATGAGTCTTATGGACAGATAGAGATGCAATTTTGTGGTGAAGAGAG |
| | | GTATTTTTATCATCAACAGCACTAACAGATCACTGCCTTACTGAAATTAGCAGTACCAATGG |
| | | TAGTGGAAATAGCGATGGCTCTAGGAGCAACACTGGAGCTATTGTTGGAGGAGTTCTCGGTG |
| | | GTGTGGCTGGGCTTGCTTTAACTGGGCTTGCTGTTTTTTTCTTCCCCCGCTATAAGAATTTA |
| | | GCTGCTGGACATAAGCCAGTGAGCCAGAATACTCCTCCTGCTGCTTATCAATCCTCTCTGAT |
| | | GCAGCAGGACCCCAAGGCTCACTATGATCCTAAATATTTCGCAGAGATGGATACACAGCCAT |
| | | ACACGCCCCCTGATTAG |
| 79 | MIC-54347 | ATGCTCCGTACTCCTACAATCCCCTGGCTTCTAAATATCATCCTAGAAGAAAACCCTACTAT |
| | | GATTCCTTCAATGAAAAAGTGCGGCCGCAAGTTATGGGCTGGCCTGGCATTGCTAGCCTTGC |
| | | CTGTAATTGCAAGCGCATCATGCGCGCTTCCTAGCACCTACAAATGGACTTCGACTGGCCCG |
| | | CTGGCAAACCCCAAGTCGGGCTGGGTTTCGCTTAAAGATTTCAGCCATGTTCCATACAATGG |
| | | CCAGCATCTGGTCTATTCTTCGACTGTGAACTCGGCAGGTTCCTATGGCTCGATGAATTTTG |
| | | GTCTCGTCTCAAACTGGACCAACCTCAGCACAGCCAGCCAAAACACAATGAACCTTGGCACG |
| | | GTAGCGCCAACTCTCTTCTATTTCTCTCCCAAGAAGATCTGGGTTCTTGCATACGAATGGGC |
| | | TGCTACTCCATTTGCCTATGTCACGTCGACCGATCCTACCAATGCCAATGGTTGGTCAGCTT |
| | | CGCAACCGCTCTTCAGCGGAAGCATATCTCCCTCTAGCCCTATCGACCCAGCACTCATCAGC |
| | | GATGGCACGAATATGTACCTTTTCTTTGCAGGAGATAATGGCAAAATCTACCGGTCCAGTAT |
| | | GCCCATTGGCCAGTTCCCATCCAGCTTCGGCACATCGTACACAACGATCATGAGCGCCGCAA |
| | | CCAACGATCTGTTTGAAGCGATACAGGTGTACACTGTCTCAGGCCAGAATCAATATCTTATG |
| | | ATTGTTGAGTGTATCGGATCTGTTGGACGGTATTTCCGCTCATTTACGGCCACATCCCTGAG |
| | | CGGTACATGGACGCCGCAAGCTGCAACCGAGAGCAATCCCTTTGCAGGCCATGCTAACAGCG |
| | | GAGCTACTTGGACTAACGATATCAGCTCTGGAGATCTTATTCGCAGCACAAACGATGAGACG |
| | | ATGACCATTGATCCCTGCAACCTCCAGCTGCTGTACCAAGGAATGGCCGTTGGTTCCACCGG |
| | | AGACTATAACTCCCTGCCATGGCGACCCGCCGTCTTGACTCTAACCAACCCCGGCTCGAGCA |
| | | CAGGCAACGGCAACGGCACTGGAAGCGGCGGCTCAGGCGGCAGCGGATCTGGACAAGCATCG |
| | | CAGTATGCTCAGTGCGGCGGTTTGGGCTATACTGGACCCACGGTTTGCCAGGCACGTAATGC |
| | | TGTTCCAAGTTTCTTGATAATGAAATTGCTAACACGAGATCAGAGCCCGTACAAATGCACGT |
| | | TTGTCAACGAATACTACTCCCAGTGTCTATAG |
| 80 | MIC-54347 | ATGCTCTACACTGCTGCTCTGGCCGGCCTTATGGCCACTGCTGCCCTTGCTGCCCCCTACCG |
| | | CCGACAGGCTACTGGCCAAAACGTTGTATACTGGGGCCAAAACGGCGGCGGCGTTACTGAAA |
| | | GTAACAATTTGTCTGATTATTGCGATGCTGAAGCAGGCATCGATCTACTTGTTCTTAGTTTC |
| | | CTCTACGAGTATGGTTAGTTTCACCCCGTTGTGCCTTTGCAAATTGCAATCTTGTGTACTAC |
| | | TCTTACTAACCATTTTGTAGGCAATGGAAATACTATTCCTTCAGGCACTATCGGCCAGAGCT |
| | | GCTCCATTGATACCTCCGGAAACCCTTCAAACTGTGATGCCCTTGCCTCGGCCATTGCTACC |
| | | TGCAAGTCCAATGGAGTCAAGGTTGTCTTGTCTCTCGGCGGCGCCGTTGGAGCCTACTCTCT |
| | | CTCCTCTCAGCAGGAGGCCGAGACAATTGGCCAAAACCTCTGGGACGCATACGGTGCCGGCA |
| | | ATGGAACCGTCCCCAGACCGTTCAGAAGCACCGTTGTCGATGGCTGGGACTTTGATATCGAA |
| | | GCCAGTAGTGGCAACCAGTATTATCAATACTTGATTGCCAAGCTTCGCTCAAATTTCAACGG |
| | | CGGCAACTACGTGATTACCGGTGCTCCTCAGTGCCCTATTCCGGAGCCTAACATGCAGCAAA |
| | | TCATTACCACTTCTCAGTTTGACTATCTTTGGGTCCAGTTCTACAACAATCCCTCGTGCTCA |
| | | GTTGGAACGTCGACACCAAACTTCCAAGATTGGGTTTCCAACATTGCCAACACTCCTTCTGC |
| | | CAATGCCAAGATCTTCCTTGGTGTGCCTGCAAGCCCGCTCGGCGCTACTGGAACTGAATCTG |
| | | GCGCCCAATATTACCTCGAGCCTTCTGCTCTCAACACTCTCGTCGGCCAGTTCTCATCCAAC |
| | | CCTGCGTTTGGCGGTATCATGATGTGGGCTGCCGGTTTCTCCGATGCCAACGTGAACAATGG |
| | | ATGCACTTATGCTCAAGAGGCTAAGAAGATCCTTACTTCTGGTCAAATCTGCTAA |
| 81 | MIC-54347 | ATGCTTTCCCACGCTATCCTTGCTGGCCTCGGCCTTGCTGGTCTCGCTGCCGCTGCGCCTAC |
| | | CAGGACGATTGCGACTCGTCAAGCTTCCGGTTATCAGAACGCTGTCTACTGGGGTGCCACTG |
| | | GCAAACAGAACCCTAACCTCGCTGAATACTGCACGTCCACCTCGGGCATTGATATCCTGATC |
| | | CTGTCCTTCTTGGACGTCTACGGTGCCACTGGCAACTTCCCCTCTGGCAACTTTGGCAACGA |
| | | CTGCTTCGTCGGCACCACGGGTGTTCCCCAGTCATGCTCTGACCTGGCCTCCCAGATCAAGA |
| | | CCTGCCAGAACGCTGGTGTCAAGATTATTGTTTCTCTCGGTGGTGCCGCCGGTTCATACTCT |
| | | GTCACGTCCCAGCAGCAGGCCCAGACCATCGGTCAATACCTCTGGGATGCCTACGGTAACTC |
| | | TGGCAGCACCTCTGTCCAGCGTCCCTTTGGCGATGTCTTTGTTAACGGTTGGGACTTTGACA |
| | | TCGAGGCCAACGGCGGCTTCAGCCAGTACTACCAGTACATGATTGGCACCCTGCGCTCCAAC |
| | | TTCGCCAAGGACTCCGCCAACACGTACTACATCACCGGTGCTCCCCAGTGCCCCCTGCCCGA |
| | | GGAGAACATGGGCGATATGATCCAGCACTCCGTCTTCGACTACCTCTTCATCCAGTTCTACA |
| | | ACAACAACCCCACCTGCTCTCTTGGTCTGTCTGGCCAGGCTCCCCTCAACTACGACGACTGG |
| | | ACCAACTTTGTCTCCACCACCCAGTCCAAGAACGCGAAGCTCTTCCTCGGTGCCCCTGCCGG |
| | | ACCTCTCGCCTCCAACGGAAACCCAAACGGTGCCGTCTACTACGCTACCCCCTCGGATCTGG |
| | | CCCCCATCGTCAACAAGGCCAAGACGAAGTCAAACTTTGGCGGTGTCATGCTCTGGGACGCT |
| | | GGTTACTCTGATGAGAACTCCAGCGGCGGTTGCAACTACGCCCAGCAGGTCAAGAGCATCCT |
| | | CACCACCGGCGCCCCTTGCAACGGCACCCCCGTCAGCGGTGGTGGCTCTCCTCCCGCTACTT |
| | | CTTCCACTGCTTCTTCTCCTCCCGCTACTTCTTCCACTGCTTCTTCTCCTCCTGCTACTTCT |
| | | TCCCTTCCTGCCGATGGAAGCACCGGAAGCACCGGTAACAGCGGCGTTGGCAGCGTTGCCCA |
| | | GTGGGGACAGGTAAGATAATTTTCTTCCTATTAGTTATAGAAAGACATTTTTGCTAACAGTT |
| | | GTTTGATAATAGTGCGGCGGTATTGGTTACACTGGTCCTACCCAGTGCCAGTCCCCATTCAA |
| | | GTGCGTTAACGAGGGCCCGTATTGGTCTTCTTGCCAATAA |
| 82 | MIC-54347 | ATGGCCGGTTCACTTACAGCAGACGCTCACGAAGCTGTTCGGTGCATCATGTATCTCACAGG |
| | | GTGAGCTTCTTTTTGGCCCGGCTTCTACTTGGCTTACAGAATATCTAACGCTTTCGTAGGCA |
| | | ACATGTGGTAGTCCCTTCAGACCATGATCTCGTGGGTTCCATAACACATGTGATACTGGCAT |
| | | TCATGCGCTCTGATGTCTTCAATGTGGATAAAACGCCTGCCGAGTTCCCGTTTTTTACAACC |
| | | GTTGCTGAAACACGGCAGAAGTTCAATGCGAATACTAAAATCATGGTCGCAATTGGGGGTTG |
| | | GGGGGATTCTGCAGGATTTGAAGAAGCTGCGCGTGACGATTCGTCGAGAAAGCGGTGGGCTA |
| | | ACCAAGTAAAGGCCATGGTTGACCTGACAGGAGCTGATGGCATTGACATTGACTGGGAATAT |
| | | CCGGGGTAAGTTGATGGTGAATCGCAGTACCCACCGCAGAGCAGTTTTCATCTAACATTATT |
| | | TCCAGGGGAAATCGTGACGACTATAAACTTATTCCGAATTCTCAGCGGGAATGGGAGATAGA |
| | | GGCATTCGTGCTTCTTCTTCGAGAACTTCGTTTGGTTTTGGGAGAAGAAAAACTACTCACAA |
| | | TTGCGGTGCCAGCGCTAGAACGCGATTTGATGGCTTTCACAAACTCAACTATTCCGTCCATC |
| | | GTGAATCAGGTTGACTTCATTAGTGTAATGACTTACGATATGATGAATCGACGTGATACCAT |
| | | TGTCAAGCACCATAGTGGTGTGGCCGACTCTCAGGAAGCAATGGAGCGATATATAGATCGTG |
| | | GGGCCCCTCCGCACAAATTGAACTTCGGACTTGGTTACTATGCCAAATGGTTCATGACCGAG |
| | | CAATGCGATTTACAGCACCCATTGGGGTGCCGCACTCAACTGCTAGAAGACCCCGCCAATGG |
| | | AGCCGACCTTGGCAAGACTGCAGCTTTTAGCTGGCATGACGAGGTTCCCGTGGAATTGGCTA |
| | | ATTCTTTCGAGAAAGCTCATACTCATGGCCGCTACTATGAAGATGGAAGCTACGGGTATTGG |
| | | GATGATGAAGAGAAGAGATGGTGGTCCTACGACACGCCTCTCGCCATCAAAACTAAAGTCCC |
| | | TCGGTTTCTCGGCGAACTGCAATTGGGCGGTGTGTTTGCCTGGGGGCTGGGGGAGGATGCTC |
| | | CGCAGTTTATTCACTTGAAGGCCACTACTGATGGGATTCGGGCTTTGCGCGGAGGCCAGAGC |
| | | TCACGGGATTCGGTGAAAGATGAACTGTAA |
| 83 | MIC-54347 | ATGGCGCCTTTCAATACTATGCTTGGGTATCTCCTGTTAGTGGTAGCCCCTTTGGCGGCTGC |
| | | CCTGCCGTTCCGTGGCCATGGCCCCAACACCCATAACCACATCAACTTGAACTTCAACCTCA |
| | | CCGAAACCATCAACGAGATCAATGAAAACTTGGCTGGCCTAGTTGGATACATCACCAACCCT |
| | | CACGCCAAGCACATCGTCGCCAACCGCTACATTGTCGTCTACAACAACACCTTCGGCTCCGA |
| | | GGCCATCGCCGCCAAGCAGGCCGAGTTCGCTGCCACCATCCAGAAGCGCAACCTTGGAAAGC |
| | | GAAGCCTCGGCGGCAACATGCTGTCCACTGAAATCCACTCCTTCCAGATGCACACCTGGCGC |
| | | GCCATGGCTCTCGACGCTGACGATGAGATGATCAAGTCCATTTTCGCCGCGAAGGAGGTCGC |
| | | CTATATCGAGGCTGATACCGTCGTCCAGACCAAGGCTCTCGTTGCCCAGACCAACGCTACCC |
| | | CCGGCCTCATCCGTCTCTCTAACCAAAACATTGGTGGCAAGAACTACATCTTTGACAACTCT |
| | | GCCGGCGCTGGTATCACTGCCTACGTTGTCGACACTGGTATTAGAATCACCCACACTGAGTT |
| | | TGAGGGCCGTGCTTCTTTCGGTGCCAACTTTGTCAACTCCAACGTATGTAACATGCCCTCCT |
| | | CATACGCCCAAACTGGTGAATGAAGTTTGCCCATCATGAACAAAAAGCTAACAATAGCGCTT |
| | | AGAACACTGATGAGAACGGCCACGGCAGCCACGTTTCTGGTACCATTGGTGGTGCTACCTTC |
| | | GGCGTTGCCAAGAACATCAAGCTCGTTGCCGTCAAGGTCCTTGATGCCTCTGGTGCCGGTAG |
| | | CAACTCTGGCGTTCTGAACGGCATGCAGTTCGTCGTCAACGACGTCCAGGCCAAGGGCCTCT |
| | | CCGGCAAGGCTGTCATGAACATGTCTCTCGGTGGCTCCCTGTCCGCTGCTGTCAACAACGCT |
| | | ATTGCTGCCATTGCCAACGCTGGTGTCGTCCCTGTTGTTGCTGCTGGTAACGAGAACGTACG |
| | | TTTATATCCTTCCCACTCGTCTCCTCTGAAAAAAACGTTGTTTTTAACTCGCATTATAGCAA |
| | | GATACTGCCAACACCTCTCCCGGCTCTGCCCCTCAGGCCATCACCGTCGGTGCTATCGATGC |
| | | CACCACCGATATCCGTGCTAGCTTCTCCAACTTTGGCGCTGATGTCGATATCTACGCCCCCG |
| | | GTGTCAATGTTCTCAGCGTTGGCATCAAGTCTGATACCGATACTGCCGTTCTCAGCGGCACC |
| | | AGCATGGGTAAGAACCCAAACCCTTACATAAAAAAACCTTTGTATCCTAAACTAATCCGCGT |
| | | TCTTCATATAGCCACTCCTCACGTTGCCGGTCTCACTGGCTACCTGATGGCTCTCAAGGGTG |
| | | TTACCAACGTCAACGATGTCACCAACCTCATCAAGAACCTCGCCACTGCTTCCGGCGCCTCG |
| | | GTCCAGCAGAACGTTGCCGGAACCACCAACCTCATCGCCAACAACGGTGAACTGTAA |
| 84 | MIC-54347 | ATGGGCTATAGCCGCAAAGATAAATCGAGGTACATCAACACAATTCAAGAAGCACTTGACAT |
| | | CTCTACAGCTTTCGTTGCCAGCAGCCGAAAAAGAATAGCTTTAACTTGGAAGATTATCATTC |
| | | AAATTCAATCCAAGTAAGAATTACAATCTGAAGTATTACTAGGATTACATTTAAACAGTCTA |
| | | TCTAGAATGCAATTCTTCGCTCTCCTCCCTCTGTTATACACCACCGCTGCAGCCCTTGGCAT |
| | | CAATTGTCGTGGGAACAGCAACTGCGTCGGTACTCCAGAATGCAGACTCGCCGATCTGATCT |
| | | TGCAAGTCAGCCAGCAAGATCCCAGCACATCATACAGCCCTGGTCAGCACATTGCTTGTTGC |
| | | GGCATACCTGGCGGCAATATCTGTGCATTCACTCAGGGCATTAGCAATTCGATTACAGCTGG |
| | | AGAAGCCCTGGGTATGCTGCAGGGGCTATCCGCCCACGGTTGTGGCCAATGCGGCAGTATTC |
| | | CCTTCAAGGATAACAATGTCGCTGAGGGACAATTGACAGTCAACTGGACCGATCACTAA |
| 85 | MIC-54347 | ATGGTTCAATCTGCGGCTTATCTAGGAGCCTTGCTGGCCACCCTGCCCTTGGCACGTGCCGG |
| | | TTTCAACGCTAGCTCGACGCAAAACATTGCCGTGTACTGGGGTGAGCTGAACATATTCTTGC |
| | | TGAATATGAGCTTCCAATTAATACATTGCGTTCAGGTCAAAACTCGGCAAATCAAGCAACCT |
| | | CTCAGCAGCGATTGTCTACTTACTGTGCCAGTAAGTTCAAGATCGTCCTAGAACAATTCATG |
| | | ACAGCTAACGATTATTTCTAGATGCTGATATAGATGTGGGCATTACCAAAAACCTTGGATGT |
| | | CACTCTGTAGCCTGGCTGACCTCTCACAGATCATCCCAATTGGGTTCATGAACGGCATCAGC |
| | | CCGGTCATTACCAACTTTGCCAATGCAGGCGACAATTGCACGGCTTTTGCAGATAATCAGAA |
| | | TGCACTTAATTGTCCCCAAATCGAGTAAGTTGCCCCCAGACTCTCAGTGAATTTCGCCAGTA |
| | | TACGATGCTTAGATATCCTTTAGGCAAGACATCATTACATGCCAGCAAACATATGGCAAGAC |
| | | AATCCTGATTTCGCTGGGGGGCGGATCTTATACCCAGGGCGGCTTCTCGTCCACTGGTGTCG |
| | | CCACATCAGCGGCTCAGACGGTTTGGAACATGTTTGGCCCTGTCAATCCAAACAGCAACGTA |
| | | GACCGTCCATTTGGTTCCGCAGTGGTTGATGGCGTTGACTTTGACTTTGAATCTGGCGTCAA |
| | | TAACTTGGCCACTTTTGCCACTGAGCTGCGGAGCTTGATGGACGCTTCTGCGTCTTCTGCGA |
| | | ACAGGAAGTTCTACCTGTCTGCTGCGCCGCAATGCGTCTATCCCGATTTCGCTGACAACCCG |
| | | GCCCTGAATGGTTCGGTTTTCTTCGATTTCATTATGATTCAATATTACAATAATGGATGCGG |
| | | AGTCAGCAGCTATGTCCCTGGAGCAACTACCCAGTGGAACTACAACTTTGATGTCTGGGACA |
| | | ACTGGGCGCATACCGTCAGCAAGAACCCTAACGTGAGGATTCTGCTTGGTATCGCCGCCAAC |
| | | ACTGGTGCTGCCAGTGGATACGTGTCCGGAACACAGCTTTCTGCGGTCATCTCATTTACCAA |
| | | GCAATACTCAAGCTTTGCGGGTATCATGATGTGGGATATGTCCCAATTGTATCAGAATTCCG |
| | | GCTTCTTGGGCCAGGTTGTCAGTGATCTCGCCGCCTCTGGTTCAACCCCCCCGGCTACTACT |
| | | TCTAGTGGAGCTTCCAAGACTACCACTTCTAGTGGCGGTTCTACAAGCCCAACTGGCGGCAG |
| | | TGTGCCGCAATGGGGTCAATGTGGAGGCGAAGGATACACTGGCCCTACGCAATGCCAGTCGC |
| | | CTTACAAGTGCGTTTTTAGCAGTCAGTGGTGGTCATCTTGCCAGTAA |
| 86 | MIC-54347 | ATGTTACTGAAACAGATTCTCGTTGCCACGTTGCTTTCCTTGAGCTCTGTCAAGGCTTTCCC |
| | | TAACCCGAATCACGCCCAAGATCTCGAGGCCAGGGAAGCTGAAGATAATCTCGTTGCTCGCG |
| | | GCGGTCACGACTGCGGCTCTTACGCTACCTGGAACTACGGAAAGAACTGCTGTGTCTGCAAA |
| | | GATAACGGCAAGAACTACGATTCTAAGTCCAAGACATGCAGTTGTCCACACGGGCAAGTCTG |
| | | GAATGGCAAGCAATGCGTTGTGGACTGTGGCAAAGATGCGACTTATGATTGGAAGCAAAAGA |
| | | AATGCGTCTGCAAGAAACAAGGCGAGGTCTATAACTCTAATACGAAGACTTGCAGCTGCCCG |
| | | CCTGGAACTGTTTGGAACGGCTACGGATGTGTTGTCGACTGTGGCAAGGAAGCGCACTTTGA |
| | | TAAATGGCAGAAGAAGTGCGTCTGCAATAACAACGGCGAAGTCTATGATTCCAAGAGCAAAA |
| | | CCTGCAGTTGCCCTGGTGGCCAATACTGGAACGGCAAGAAATGCGTATGCCCTTATGGCAAG |
| | | GTCTGGAACGGCAAGCAGTGCATAGAGGACTGTGGTAAAGACGCACACTTCGACTATAACCA |
| | | AAAGAAGTGCGTATGCAACAAGAATGGACAGGTCTACAACTCCAACAGCAAGACCTGCAAAT |
| | | GTCCAGGTGGCCAGTATTGGGACGGCAAGCATTGTGTCTGCCCATATGGTCAAGTCTTTAAT |
| | | GGCAAGCAGTGTGTTCCTGATTGCGGTAAAGAAGCCCACTTTGATTATAAGCAAAAGAAGTG |
| | | CGTATGCAACAACTACGGCGAAATCTACAATTCCAAGAGCAAGACTTGCGCCTGTCCTGACG |
| | | GCCAGTATTGGAATGGAAAGCAATGCGTTTGCCCTTACGGCAAGATCTGGAATGGCAAACAG |
| | | TGTGTTCCTGATTGCGGTAAAGACGCCCACTTTGACTACAACCAAAAGAAGTGTGTTTGCAA |
| | | CAAGAATGGAGAGATCTATGACTCTAAGAGCAAGACTTGCGCCTGTCCTGACGGCCAGTATT |
| | | GGAATGGAAAACAGTGTGCCTGCCCGTACGGCAAGATTTGGAATGGCAAACAGTGTGTTCCT |
| | | GATTGCGGCAAAGAAGCCCACTTCGATTACAACCAGAAGCAGTGCGTTTGCAACAATAAGGG |
| | | AGAGATCTATGATTCTAAGAGCAAGACTTGCGCCTGTCCTGACGGCCAATACTGGAATGGAA |
| | | AGCAGTGTGCCTGCCCGTACGGCAAGATCTGGAACGGCAAGCAGTGTGTAGAAGATTGCGGC |
| | | AAAGAAGCACACTTCGATTACAACCAGAAGCAATGTGTTTGCAATAACAAGGGAGAAATCTA |
| | | TGATTCTAAGAGCAAGACTTGCAAGTGCCCTGATGGCCAATACTGGGACGGAAAGCAGTGCG |
| | | CTTGTCCATACGGAAAGATCTGGAATGGTAAGCAGTGTGTAGAGGACTGTGGCAAGGACGCA |
| | | CACTTCGATTATAACCAGAAGCAGTGTGTTTGCAATAACAAGGGAGAGATTTACGATTCTAA |
| | | GAGCAAGACTTGCAAGTGCCCTGACGGCCAATACTGGGACGGAAAACAATGTGCTTGTCCCT |
| | | ACGGCCAAATTTGGGACGGAAAGCAATGCACACCAAACTGCGGCAAAGACGCTACCTATGAC |
| | | AGTAAGCAGAAGCAATGCGTGTGCAACAAGAAGGGCCAAGTCTTTGATTCCAAGCACTTGAC |
| | | ATGCAGCTGCCCGGCTGGAACGAGTTGGAACGGCTATGCTTGTGTCCCAGATTGTGGCAAGG |
| | | ATGCGCACTACGATACCAATCAGAAGTGCTGCGTGTGCAATAACAAGGGCCAGATATTTAAT |
| | | TCTGGAAGCAAGACATGCAGCTGCCCAGGCAACCAATACTGGGACGGCAATAAATGCACATG |
| | | CCCCTATGGATCGACATGGGACAGCAGTAAGAAGACGTGCAAGCAGACACCCATACATTAA |
| 87 | MIC-54347 | ATGTTGGGCTTCCTCGGAAAATCCGTGGCCTTGCTTGCTGCGCTGCAGGCCACCCTCACCTC |
| | | TGCATCTCCTCTGTCTACAAACGACGTCACAGTTGAGAAGAGAGCCAGCGGATATGCGAACG |
| | | CTGTCTACTTCACTAACTGGTGAGTGAAGCTAATTTGTGATTATGAATTTTAGTGCTAACTA |
| | | TTGGTGATTAAAGGGGTATCTACGGCCGCAACTTTCAACCCCAGGACCTGGTTGCGTCGGAC |
| | | ATCACTCATGTCATCTACTCGTTCATGAACTTCCAAGCAGACGGCACTGTGTAAGTTTTGTA |
| | | ACCAAGAGATGGTGTATCCTAAATATCTATTTTCAGTTGCTGATCGTTTCCTCTATAGCGTC |
| | | TCTGGAGATGCTTACGCCGATTACCAGAAGCACTATTCCGATGATTGTACGATAACCCCCCC |
| | | TTTAAGTGCTCTTAATTCTAAGCTTTGCAAATATACTAACATCTATCTCAGCTTGGAATGAT |
| | | GTCGGCAACAACGCGTACGGTTGTGTCAAGCAACTGTTCAAGTTGAAGAAGGCCAACCGCAA |
| | | CTTGAAGGTTATGCTTTCTATAGGTGGCTGGACCTGGTCCACCAACTTCCCTTCTGCCGCAA |
| | | GCACCGATGCCAACCGCAAGAACTTTGCCAAGACAGCCATTACTTTCATGAAGGACTGGGGT |
| | | TTTGATGGTATTGACGTCGACTGGGAGTATCCTGCCGATAACACCCAGGCCACCAACATGGT |
| | | TCTTCTGCTTAAGGAGATCCGATCTCAGCTAGATGCTTATGCGGCCCAATACGCTCCAGGAT |
| | | ACCACTTCCTTCTCTCCATCGCTGCCCCCGCTGGCCCAGAGCACTACTCTGCCCTGCACCTG |
| | | GCCGACCTTGGTCAAGTTCTCGACTATATCAACCTTATGGCTTATGACTATGCTGGTTCTTG |
| | | GAGCAGCTTCTCCGGACATGATGCCAACTTGTTTGCCAACCCCTCCAACCCCAACTCTTCAC |
| | | CATACAACACCGATCAGGCTATCAAGGCTTATATCAACGGAGGTGTTCCTGCACGCAAGATC |
| | | GTTCTTGGCATGCCCATCTATGGACGATCTTTCGAGAGCACCGGTGGAATTGGCCAGTCTTA |
| | | CAGCGGAATTGGATCTGGAAGCTGGGAGAACGGTATCTGGGACTACAAGGTTCTTCCCAAGG |
| | | CCGGTGCTACAGTCCAGTACGACTCTGTCGCACAGGCATACTACAGCTATGACCCTAGCTCC |
| | | AAGGAGCTCATCTCCTTCGATACCCCTAGCATGATCAACACCAAGGTCTCTTACCTCAAGAA |
| | | CCTCGGCCTGGGAGGCAGTATGTTCTGGGAGGCTTCTGCTGACAAGACTGGCTCCGACTCCT |
| | | TGATCGGAACAAGCCACAGAGCGCTTGGAAGCCTGGACTCAACTCAGAACTTGCTGAGCTAC |
| | | CCCAACTCCCAGTACGATAACATCCGAAGCGGTCTTAACTAA |
| 88 | MIC-54347 | ATGTTTTTCAGCAAAGCACTGGCTGCTACCGGCCTTTTGGCCACTGCTGCTCTCGCTGCACC |
| | | CACCGTGGAGAAGCGTGCCGCTGGAGGCAAGCTCGTCATCTACTGGGGTGCTGAAGACGACA |
| | | GCACTACTCTCGCCAACGTCTGTGCCGATTCCTCTTACGACATTGTCAACTTGGCTTTCCTT |
| | | GACAAGTTCTCTGCTGGTGGTGGCTACCCCTCGCTCTCTCTGAGCACCCTGGGCGGCCCATC |
| | | TGCCGCTCAGAAGGCTGCTGGTGCCACCAACCTCCAGGATGGTTCTTCTTTGGTTCCCGCCA |
| | | TCAAGGCCTGCCAGGCCGCCGGCAAGCTGGTCATCCTCTCTATGGGTGGTGCTACCGACTTC |
| | | TCTGCAGTCTCCCTTTCGGGCGATGCTCAGGGACAGTCTGTTGCCGACATGGTTTGGAACCT |
| | | GTTCTTGGGTGGTACTGCCAACCCCACTCTCCGTCCTTTCGGATCAGTCAAGCTCGACGGTG |
| | | TTGATCTCGGTGAGTAAAAGTCATTCCTAACATGATCTATGAATTCTACCTTTACTGACATC |
| | | TCATCCTGCAATATAGACAACGAGACTGGCAACCCCACTGGTTACCTGGCCATGACTCAACG |
| | | CTTCCGATCCAACTTTGCCAAGGACACCAGCAAGAGGTACTACCTCACTGCTGCTCCCCAGT |
| | | GCCCCTTCCCCGATGCCTCTGAGCCTCTCAACGTCTGCCAGCTCCTTGATTACGTCTGGGTC |
| | | CAGTTCTACAACAACGGCAACTGCAACGTTGGCCAGTCCGGCTTCAACACTGCTGTCAAGAA |
| | | CTGGAGCAAGAACATTGGTAACGCTACTCTGTTCATTGGTGCGCTGGCCAGCGGTGCCGATG |
| | | GTGACCAGGGCTACATCTCTCCCAGCGCTCTGATCTCTGCTTACAACGGTGTCTCTGCTCTT |
| | | AACCTGCCCAACGTTGGCGGTATCATGCTTTGGGAGGCTCAGCTCGCTGTCAAGAACAGCAA |
| | | CTTCCAGAAGACCATCAAGGCTGCCATCGGCTCCGGCTCCACTCCTCCTCCTCCTCCTCCCG |
| | | CCTCTTCCACCACTCCTGCCGGAAGCACCCCCACCTGCTCTTGGGCCGGCCACTGCGCTGGC |
| | | GCTACTTGCAGCACTGACAATGACTGCTCCGACTCCCTCACCTGCAACAGCGGCAAGTGCGG |
| | | TACCGCTGGCAGCACTGCTCCTCCTCCTACCACTTGCTCTTGGGCGGGCCACTGCCTCGGCG |
| | | CTTCTTGCGGTAACGACAACGACTGCTCTGACCCTTACTTCTGCTCCAACGGCGTCTGCTCT |
| | | CAGTAA |
| 89 | MIC-54347 | ATGTTTTTCCGCAACGCCGTCGCCGTAACCAGCCTTTTGGCTGCACTTTCTAGTGCCCAACC |
| | | CTCCGGCCCCGAATTGGCCGTGTACTGGGGTGCTGAAGATGATAGCACGACTCTCAGCGACG |
| | | TGTGTTCAGACAACTCTTATGGCATTGTGAATCTTGCCTTCCTCGACACATTTTTTGCTGCA |
| | | GGAGGCTTTCCTCAACTCAGCGTCAGCGGGTTAGATGGCCCATCCCAAGCTCAGCAAAGCGC |
| | | TGGTGCCACTGGCCTCAAGGATGGTAGCAGTCTTGTAGACGCTATTAAGCAGTGCCAGTCGG |
| | | CCGGGAAGCTGGTCCTTCTCAGCCTTGGTGGCGCTGGCGCCGATGTCACTCTTCAATCAGAC |
| | | TCTGATGGAGAGAAAATTGCTGATACACTCTGGAATCTGTTCGGTGGTGGCACTGATAACCA |
| | | AGAGCTGCGTCCCTTTGGAGATGTCAAGCTCGATGGCTTCGACTTGGGTGAGTAACATTAAT |
| | | GAATTAACACGATCAATGTGGCTAACTCTATGCAGACAATGAATCTGGCAACCCCACAGGTT |
| | | ACCTAGCTATGGTAAAGCGTTTCAAGTCCAACTTCCAGTCGGACACGAGCAAAACTTATTTC |
| | | CTTACTGCGGCTCCTCAATGCCCATTCCCCGATGCTTCGCAGCCTCAAGATGTTTGCAGTGA |
| | | ATTGGACTTTGTCTGGGTCCAGTTTTACAACAACGGCGATTGCAACATTGCGCAATCTGATT |
| | | TCTTAAACTCCGTCCAGACTTGGAGCAGCGGCATTGGCAACGCTAAGCTCTACATCGGTGCT |
| | | TTGGCTAGTGGTGCTGACGGCGATCAGGGCTTTGCTGATGCTGATACGCTATTGGGTGCTAT |
| | | TCAGGATGTCAAGAATATGAACCTTCCCAATTATGGAGGTGCCATGCTTTGGGAAGCCCAAC |
| | | TGGCTGTTAAAAACGGCAACTTTCAGCAGAAGATTGCCCCCGGTCTATAA |
| 90 | MIC-54347 | CATCGAGAAGTTCGAGAAGGTAAGCTCATTTCGCTGCTTTTTTCATTCCTTTTGGGCACAAT |
| | | TGTGCCAGACAATTCTGTTCTCAGTCTTGTCACCATTTTTGCCCACCAAGCATCGCACCCCG |
| | | CTTTGTCTACCTACCCCTCCTTTGGCACAGCAAAATTTTTCTGGCTGCCTGGGTTGGTTTTT |
| | | AGTGGGGTGCCAAATTTTTGGCAGTGACCCCGCCATCGCCACTGTTCCTCATCTCATGCATT |
| | | ACCCAACATAATCTTCAGTCAATTGCTTGGTTCATTGTGCTAATCATACTTTAATCAATAGG |
| | | AAGCCGCCGAACTCGGCAAGGGTTCCTTCAAGTATGCGTGGGTTCTTGACAAGCTCAAGGCC |
| | | GAGCGTGAGCGTGGTATCACCATCGACATTGCCCTGTGGAAGTTCGAGACTCCCAAGTACTA |
| | | TGTCACCGTCATTGGTATGTTTTCAGTCCGACTGGTCACTATCCCATCATCATCATGCTAAC |
| | | GTGCGACTCTACAGACGCTCCCGGTCACCGTGATTTCATCAAGAACATGATCACTGGTACCT |
| | | CCCAGGCCGATTGCGCTATCCTCATTATCGCTGCCGGTACTGGTGAGTTCGAGGCTGGTATC |
| | | TCCAAGGATGGCCAGACTCGTGAGCACGCTCTGCTCGCCTACACCCTGGGTGTCAAGCAGCT |
| | | CATTGTTGCCATCAACAAGATGGACACTGCCAACTGGGCCGAGGCTCGTTACCTTGAGATCA |
| | | TCAAGGAGACCTCCAACTTCATCAAGAAGGTCGGCTTCAACCCCAAGACCGTTGCCTTTGTC |
| | | CCCATCTCTGGCTTCAACGGTGACAACATGCTCCAGGCCTCCACCAACTGCCCCTGGTACAA |
| | | GGGTTGGGAGAAGGAGACCAAGGCTGGCAAGTCCACCGGTAAGACCCTCCTCGAGGCCATTG |
| | | ACGCCATCGAGCCCCCCAAGCGTCCCACAGACAAGCCCCTCCGTCTGCCCCTTCAGGATGTC |
| | | TACAAGATCGGTGGTATCGGAACAGTCCCTGTCGGCCGTATCGAGACTGGTGTCCTCAAGCC |
| | | CGGTATGGTCGTTACCTTCGCTCCTTCCAACGTCACCACTGAAGTCAAGTCCGTCGAGATGC |
| | | ACCACGAGCAGCTCGTTGAGGGTGTCCCCGGTGACAACGTTGGATTCAACGTCAAGAACGTC |
| | | TCCGTCAAGGATATCCGCCGTGGTAACGTTGCCGGTGACTCCAAGAACGACCCCCCCATGGG |
| | | TGCCGCTTCTTTCAACGCCCAGGTCATCGTCATGAACCACCCTGGCCAGGTCGGTGCCGGAT |
| | | ACGCTCC |
| 91 | MIC-54347 | CCGAGGAGCACCCCGTCCTGCTCACCGAGGCCCCCATCAACCCCAAGTCCAACCGTGAGAAG |
| | | ATGACCCAGATCGTCTTCGAGACCTTCAACGCTCCCGCTTTCTACGTCTCTATCCAGGCCGT |
| | | TCTGTCCCTGTACGCCTCTGGTCGTACCACCGGTATCGTTCTCGACTCCGGTGATGGTGTTA |
| | | CCCACGTTGTCCCCATCTACGAGGGTTTCGCTCTTCCTCACGCCATTGCTCGTGTTGACATG |
| | | GCTGGTCGTGATCTTACCGACTACCTGATGAAGATCCTGGCTGAGCGTGGTTACACTTTCTC |
| | | CACCACCGCCGAGCGAGAAATTGTTCGTGACATCAAGGAGAAGCTCTGCTACGTCGCTCTCG |
| | | ACTTCGAGCAGGAGATCCAGACCGCTGCTCAGAGCTCCAGCCTGGAGAAGTCATACGAGCTT |
| | | CCCGACGGTCAGGTCATCACCATCGGCAACGAGCGATTCCGTGCTCCTGAGGCTCTGTTCCA |
| | | GCCTTCTGTCCTTGGTCTTGAGAGCGGTGGTATCCACGTCACCACTTTCAACTCCATCATGA |
| | | AGTGCGATGTTGACGTCCGAAAGGACCTGTACGGCAACATTGTCATGGTAAGTGAATTTCCG |
| | | CATTCAACAATTGATTTTTATAGCGGCGCTAATGTATTTCTCAATCTAGTCTGGTGGTACCA |
| | | CCATGTACCCCGGTCTCTCCGACCG |
| 92 | MIC-54347 | TCTCTCTTTGTGAGTCTCTAGACAGTCTTTTGCGACAAATCTGCTGGCGATTTGGGTACTAA |
| | | TCTGCTGGCTACAGGACAAGGACGGCGATGGTACGTAGTACCTAGTGACGCGATACTCCTCT |
| | | TTTCCCCTCCTCCTCACCGTCCCCCTATTTTGTGCGAGCTATCACTAGAGCGCCTGAACCTG |
| | | CGATACCGATCGAAATATCCCCGGCTGGCAATTTTTATGACGAATAAACGGACAAGAAACTA |
| | | ACAGACTTGACTTGGCAGGCCAGATCACCACCAAGGAGCTGGGCACTGTCATGCGCTCTTTG |
| | | GGCCAGAACCCCTCCGAGTCAGAGCTGCAGGACATGATCAACGAGGTTGATGCCGACAACAA |
| | | CGGATCCATCGATTTCCCTGGTATGTCAATAGCAGAAACACATAGACAGCTGCCGGATACAG |
| | | GCTAATCTAGAGCGGTGAAGAGTTCCTTACCATGA |
| 93 | MIC-54347 | TGGCCAAGCTGTTCCGTGGTATCATGCGCAGGATGAATACCGAGTTGGCCAACTACCTGAGA |
| | | CGATGTGTTGAGGGCAACCGCCACTTCAACCTTGCTGTTGGCATCAAGCCCGGCACACTCTC |
| | | CAACGGATTGAAGTATTCACTTGCTACCGGAAACTGGGGTGACCAGAAGAAGGCAATGAGCT |
| | | CGACTGCAGGTGTATCACAGGTGCTTAACCGTTACACTTTTGCCTCGACACTTTCTCATTTG |
| | | CGTCGTACCAACACACCCATTGGAAGAGATGGTAAGCTGGCGAAGCCTCGACAGCTTCACAA |
| | | CACACATTGGGGTTTGGTGTGCCCAGCCGAGACCCCTGAAGGACAAGCTTGTGGTCTGGTCA |
| | | AAAACTTGTCTCTGATGTGCTACGTCAGTGTCGGATCTCCTTCTGAGCCTTTGATCGAGTTT |
| | | ATGATCAATAGGGGTATGGAGGTTGTTGAGGAGTACGAACCACTGAGGTATCCCCATGCTAC |
| | | AAAGATCTTTGTGAATGGTGTCTGGGTTGGAATCCACCAAGACCCCAAGCATCTGGTAAACC |
| | | AAGTTTTGGACACTCGTCGTAAATCCTATCTGCAGTACGAAGTCTCTCTGATCAGAGATATT |
| | | CGTGACCAAGAATTCAAAATCTTCTCTGACGCCGGTCGTGTTATGCGTCCTGTCTTTACTGT |
| | | ACAGCAAGAAGATGACCCGGAAACGGGTATCAACAAGGGCCACTTGGTATTGACCAAGGACC |
| | | TCGTCAACAGACTTGCCAAAGAGCAGGCTGAGCCTCCAGAAGACCCAAGCATGAAGCTTGGA |
| | | TGGGAGGGGTTAATTAGGGCTGGTGCGGTGGAATATCTCGACGCCGAGGAAGAAGAAACGGC |
| | | TATGATTTGCATGACACCGGAGGACCTTGAACTTTATCGTCTTCAGAAAGCTGGTATTTCCA |
| | | CGGATGAAGACATGGGAGACGATCCAAACAAGCGTCTCAAGACCAAGACAAATCCGACAACT |
| | | CACATGTACACGCATTGCGAGATTCACCCAAGTATGATCTTAGGTATCTGTGCTAGTATCAT |
| | | TCCTTTCCCCGATCA |
| 94 | MIC-54347 | MAGSLTADAHEAVRCIMYLTGQHVVVPSDHDLVGSITHVILAFMRSDVENVDKTPAEFPFFT |
| | | TVAETRQKFNANTKIMVAIGGWGDSAGFEEAARDDSSRKRWANQVKAMVDLTGADGIDIDWE |
| | | YPGGNRDDYKLIPNSQREWEIEAFVLLLRELRLVLGEEKLLTIAVPALERDLMAFTNSTIPS |
| | | IVNQVDFISVMTYDMMNRRDTIVKHHSGVADSQEAMERYIDRGAPPHKLNFGLGYYAKWEMT |
| | | EQCDLQHPLGCRTQLLEDPANGADLGKTAAFSWHDEVPVELANSFEKAHTHGRYYEDGSYGY |
| | | WDDEEKRWWSYDTPLAIKTKVPRFLGELQLGGVFAWGLGEDAPQFIHLKATTDGIRALRGGQ |
| | | SSRDSVKDEL |
| 95 | MIC-54347 | MAPFNTMLGYLLLVVAPLAAALPERGHGPNTHNHINLNENLTETINEINENLAGLVGYITNP |
| | | HAKHIVANRYIVVYNNTFGSEAIAAKQAEFAATIQKRNLGKRSLGGNMLSTEIHSFQMHTWR |
| | | AMALDADDEMIKSIFAAKEVAYIEADTVVQTKALVAQTNATPGLIRLSNQNIGGKNYIFDNS |
| | | AGAGITAYVVDTGIRITHTEFEGRASFGANFVNSNNTDENGHGSHVSGTIGGATFGVAKNIK |
| | | LVAVKVLDASGAGSNSGVLNGMQFVVNDVQAKGLSGKAVMNMSLGGSLSAAVNNAIAAIANA |
| | | GVVPVVAAGNENQDTANTSPGSAPQAITVGAIDATTDIRASFSNFGADVDIYAPGVNVLSVG |
| | | IKSDTDTAVLSGTSMATPHVAGLTGYLMALKGVTNVNDVTNLIKNLATASGASVQQNVAGTT |
| | | NLIANNGEL |
| 96 | MIC-54347 | MFFRNAVAVTSLLAALSSAQPSGPELAVYWGAEDDSTTLSDVCSDNSYGIVNLAFLDTFFAA |
| | | GGFPQLSVSGLDGPSQAQQSAGATGLKDGSSLVDAIKQCQSAGKLVLLSLGGAGADVTLQSD |
| | | SDGEKIADTLWNLFGGGTDNQELRPFGDVKLDGFDLDNESGNPTGYLAMVKRFKSNFQSDTS |
| | | KTYFLTAAPQCPFPDASQPQDVCSELDFVWVQFYNNGDCNIAQSDELNSVQTWSSGIGNAKL |
| | | YIGALASGADGDQGFADADTLLGAIQDVKNMNLPNYGGAMLWEAQLAVKNGNFQQKIAPGL |
| 97 | MIC-54347 | MFFSKALAATGLLATAALAAPTVEKRAAGGKLVIYWGAEDDSTTLANVCADSSYDIVNLAFL |
| | | DKFSAGGGYPSLSLSTLGGPSAAQKAAGATNLQDGSSLVPAIKACQAAGKLVILSMGGATDF |
| | | SAVSLSGDAQGQSVADMVWNLFLGGTANPTLRPFGSVKLDGVDLDNETGNPTGYLAMTQRER |
| | | SNFAKDTSKRYYLTAAPQCPFPDASEPLNVCQLLDYVWVQFYNNGNCNVGQSGENTAVKNWS |
| | | KNIGNATLFIGALASGADGDQGYISPSALISAYNGVSALNLPNVGGIMLWEAQLAVKNSNFQ |
| | | KTIKAAIGSGSTPPPPPPASSTTPAGSTPTCSWAGHCAGATCSTDNDCSDSLTCNSGKCGTA |
| | | GSTAPPPTTCSWAGHCLGASCGNDNDCSDPYFCSNGVCSQ |
| 98 | MIC-54347 | MGSFKAIIAASLAALGQFAAAAPAEGQSKRAGISSIVKGTPVGFASSVTGGGTVAPVYPTTI |
| | | AQLKSYLTSTSPQNIVISGTFNFAGTEGTTTLPACNAYPCTPSNGGQALLNTLGGCGSLSTY |
| | | NVNLDTAAYNAINVQSDKTLVGINGATLNGKGLRLSGVSNVIIQNIAITNLNPQYVWGGDAI |
| | | SLSNTNNVWIDHVKTSNLGRQHYSFGTGSNNAVTISNSFISGQTSYSASCDGHSYWGLELVG |
| | | SGDQITFYKNYVYYTSGRTPALSGNTLFHAVNNVWSSNSGHAIEGTSNGMGLYEGNYFVNVP |
| | | TIVASGFVGRLFSSQSSAVSQCAQYLGRNCVSNSLSNSGTFTNSDTSFLYLFQGKANIVSAA |
| | | SASSIQSSVVSSAGNTL |
| 99 | MIC-54347 | MGYSRKDKSRYINTIQEALDISTAFVASSRKRIALTWKIIIQIQSNLSRMQFFALLPLLYTT |
| | | AAALGINCRGNSNCVGTPECRLADLILQVSQQDPSTSYSPGQHIACCGIPGGNICAFTQGIS |
| | | NSITAGEALGMLQGLSAHGCGQCGSIPFKDNNVAEGQLTVNWTDH |
| 100 | MIC-54347 | MHLHSLFTALALASPALAASIPRSSSSSSSSTITVWATPHDSYSSSVGVLGCKVDTNRIAYW |
| | | PDSVDCTNICVSLSYQDRQVYLLRVDQSQGAHDISYDAWNYLVTGYPATEKPVAGGPMEMTT |
| | | ENVDASKCADLIYTAGGKLPLSAANSMNFLASCLEQENSWVASNYVLYNILDAICTVGQNQV |
| | | CSLNWPTANQPTCPGTLGLPDALKGEPVYNIEYPSGVKVLAGAPPTVPTGVPVPVPAPSTND |
| | | DEKSAARSLRHSDSLVWIPVLSLTSIIYSWMLW |
| 101 | MIC-54347 | MHSTTFFASLGLAGLVAGAPSAPHNVKARQASGAQNVVYWGGTNNESDDLSTYCTPTAGIDI |
| | | LVLSFLDIYGTTGNIPAGNIGNSCYIGTNGTPQLCDNLAASIASCQAAGIKIILSLGGAAGS |
| | | YSLQSQSQAVAIGQYLWEAYGNSGSTSVQRPFGNVFVNGFDFDLELNAGNQYYQYLISTLRS |
| | | NFASDPKHTYYITGAPQCPLPLSIPQIRKPNMGEIISASQFDYLWIQFYNNNAYAPDPCSLG |
| | | LPGDAPFNYNNWTSFIATTPSKNAKLFIGVPANTLAANGNSGGAVYYASPSQLASIVANTKS |
| | | SPDFGGIMMWDAGYSDANVNNGCNYAQEAKNILLTGAPCGGSPPPVSSSKPTSTATKSATST |
| | | SSASGTGPTGGGTVPQWGQCGGEGYTGPTQCVSPYKCVESSQWWSSCQ |
| 102 | MIC-54347 | MKFFAATALLAATTIAGPLEVRTGDGNICPSGGLYGNPQCCSSLLLGLIGLDCNVPNQTP |
| | | RDGADERNICAKTGDEALCCVAPVAGQALLCQVAVGAS |
| 103 | MIC-54347 | MKFFAVATLFVTGVISAPSPNALNSRSLLCSPGLYSTAQCCGVDVLGVADLDCAAPTGTITN |
| | | AQGFQAACAKKGQEARCCVLPVAGQDVLCQDPPGL |
| 104 | MIC-54347 | MKFSFTSAIAIASIGITGARAGINCNGSGNCPGVAGDLSTLISFGWSIDPNRWYNNGEHIVC |
| | | VQSQLGTGLCAFLQNTGGAPGSSIQPLLQALQGHGCNKCGSVPLNFLQGDNSEDHGELTVNA |
| | | VGSTAGCSGIC |
| 105 | MIC-54347 | MKMLITAALFTLALAAPVAETKPHSIAARDPFTCPGGLTNSTPMCCSVNVLGLLALDCQQPG |
| | | ADGCSGSSKPNCCTLGAAGQGVICNAL |
| 106 | MIC-54347 | MKSAILLGLTGLAANVNAHPAKQPETGNGLTKRGVDISKYSLPNLSKYTSSPNIEQEASLQS |
| | | LAFKRNYVDTATRAVKKAAPKAEFRVVDDHYIDVDGIGHVHFKQTVHGIDIDNGDFKVNIGK |
| | | DGRVFSHGNSFFSGKLPQQNPLRKRDFSDPTTALKGAIDILGLPVQADGATAEAQEGTEKYT |
| | | LKGTSGAVSDPKAHLVYLVKGDGTLALTWRVETDIMDNWLLTYVDATTNQEIHGVVDYVSDL |
| | | ATFQVYPWGLNDPTEGDRKVLTDPWRTDASPFTWLSDGTTNYTVTRGNNAIAQDNPSGGDSY |
| | | LNNHRPSSSTRDFQYPFTLTQTNPTDYRDAAITQLFYTVNKYHDLLYVLGENEVAGNFQANN |
| | | NGKGGKANDFVIVNAQDGSGTNNANFATPADGSNGRMRMYIWTTANPKRDGDLEAGIVIHEY |
| | | THGLSTRLTGGPANSGCLTGTEAGGMGEGWGDFFATAIRLKAGDTRSKDYPMGVWADNNVKG |
| | | IRQYPYSTSLTTNPLTYKTVNTQNEVHSAGTTWATILYEVLWNLIDKYGKNDDDFPTFDSQG |
| | | VPTDGKYLTLKLVLNGLALQPCTPTFVSARDAILDADRALTGGENLCELWTGFAKRGLGSGA |
| | | KYSSTARVESFTIPSGVC |
| 107 | MIC-54347 | MKSSLLSLIAIAINGALAGINCNGSGDCAGTPGTLGDLIADAYQIDPNRWYNNGEHIACSDN |
| | | RGGGGLCAFFQNTLGGPGSSVLTLLQNLQAHGCNKCGSIPVNFPQGDNSENHGELTVNFVVS |
| | | AGCTGLC |
| 108 | MIC-54347 | MLFKIAIVVIASTARVSAFGINCEGSGYCSPLFNPGANNHPLLEMVDVIDFGIDDNRWYAAG |
| | | EHIACDQSSGVCAFVQKIGGASGGDIARAVRYLADHGCTTCGSVPLDFPNTNDVNNGEVTEN |
| | | FVGLEDMGSCSDLC |
| 109 | MIC-54347 | MLGFLGKSVALLAALQATLTSASPLSTNDVTVEKRASGYANAVYFTNWGIYGRNFQPQDLVA |
| | | SDITHVIYSFMNFQADGTVVSGDAYADYQKHYSDDSWNDVGNNAYGCVKQLFKLKKANRNLK |
| | | VMLSIGGWTWSTNFPSAASTDANRKNFAKTAITFMKDWGFDGIDVDWEYPADNTQATNMVLL |
| | | LKEIRSQLDAYAAQYAPGYHFLLSIAAPAGPEHYSALHLADLGQVLDYINLMAYDYAGSWSS |
| | | FSGHDANLFANPSNPNSSPYNTDQAIKAYINGGVPARKIVLGMPIYGRSFESTGGIGQSYSG |
| | | IGSGSWENGIWDYKVLPKAGATVQYDSVAQAYYSYDPSSKELISFDTPSMINTKVSYLKNLG |
| | | LGGSMFWEASADKTGSDSLIGTSHRALGSLDSTQNLLSYPNSQYDNIRSGLN |
| 110 | MIC-54347 | MLLKQILVATLLSLSSVKAFPNPNHAQDLEAREAEDNLVARGGHDCGSYATWNYGKNCCVCK |
| | | DNGKNYDSKSKTCSCPHGQVWNGKQCVVDCGKDATYDWKQKKCVCKKQGEVYNSNTKTCSCP |
| | | PGTVWNGYGCVVDCGKEAHFDKWQKKCVCNNNGEVYDSKSKTCSCPGGQYWNGKKCVCPYGK |
| | | VWNGKQCIEDCGKDAHFDYNQKKCVCNKNGQVYNSNSKTCKCPGGQYWDGKHCVCPYGQVEN |
| | | GKQCVPDCGKEAHFDYKQKKCVCNNYGEIYNSKSKTCACPDGQYWNGKQCVCPYGKIWNGKQ |
| | | CVPDCGKDAHFDYNQKKCVCNKNGEIYDSKSKTCACPDGQYWNGKQCACPYGKIWNGKQCVP |
| | | DCGKEAHFDYNQKQCVCNNKGEIYDSKSKTCACPDGQYWNGKQCACPYGKIWNGKQCVEDCG |
| | | KEAHFDYNQKQCVCNNKGEIYDSKSKTCKCPDGQYWDGKQCACPYGKIWNGKQCVEDCGKDA |
| | | HFDYNQKQCVCNNKGEIYDSKSKTCKCPDGQYWDGKQCACPYGQIWDGKQCTPNCGKDATYD |
| | | SKQKQCVCNKKGQVFDSKHLTCSCPAGTSWNGYACVPDCGKDAHYDTNQKCCVCNNKGQIEN |
| | | SGSKTCSCPGNQYWDGNKCTCPYGSTWDSSKKTCKQTPIH |
| 111 | MIC-54347 | MLPSSLCRIAAVISVASAEMVSVTFDTAYDDPSRSLSEVACWRKGTGFMPNLDWKLQKDALE |
| | | FIGIKAIRGFSSAQCFSCWKIEYGDKQISLFAIDSADSGIVLSLSTMQYLTGGQARELARID |
| | | AKATQVDASNCGISAAELHKYDFYTNGSGNSDGSRSNTGAIVGGVLGGVAGLALTGLAVFFF |
| | | PRYKNLAAGHKPVSQNTPPAAYQSSLMQQDPKAHYDPKYFAEMDTQPYTPPD |
| 112 | MIC-54347 | MLRTPTIPWLLNIILEENPTMIPSMKKCGRKLWAGLALLALPVIASASCALPSTYKWTSTGP |
| | | LANPKSGWVSLKDFSHVPYNGQHLVYSSTVNSAGSYGSMNFGLVSNWTNLSTASQNTMNLGT |
| | | VAPTLFYFSPKKIWVLAYEWAATPFAYVTSTDPTNANGWSASQPLFSGSISPSSPIDPALIS |
| | | DGTNMYLFFAGDNGKIYRSSMPIGQFPSSFGTSYTTIMSAATNDLFEAIQVYTVSGQNQYLM |
| | | IVECIGSVGRYFRSFTATSLSGTWTPQAATESNPFAGHANSGATWTNDISSGDLIRSTNDET |
| | | MTIDPCNLQLLYQGMAVGSTGDYNSLPWRPAVLTLTNPGSSTGNGNGTGSGGSGGSGSGQAS |
| | | QYAQCGGLGYTGPTSPYKCTFVNEYYSQCL |
| 113 | MIC-54347 | MLSHAILAGLGLAGLAAAAPTRTIATRQASGYQNAVYWGATGKQNPNLAEYCTSTSGIDILI |
| | | LSFLDVYGATGNFPSGNFGNDCFVGTTGVPQSCSDLASQIKTCQNAGVKIIVSLGGAAGSYS |
| | | VTSQQQAQTIGQYLWDAYGNSGSTSVQRPFGDVFVNGWDFDIEANGGFSQYYQYMIGTLRSN |
| | | FAKDSANTYYITGAPQCPLPEENMGDMIQHSVFDYLFIQFYNNNPTCSLGLSGQAPLNYDDW |
| | | TNFVSTTQSKNAKLFLGAPAGPLASNGNPNGAVYYATPSDLAPIVNKAKTKSNFGGVMLWDA |
| | | GYSDENSSGGCNYAQQVKSILTTGAPCNGTPVSGGGSPPATSSTASSPPATSSTASSPPATS |
| | | SLPADGSTGSTGNSGVGSVAQWGQCGGIGYTGPTQCQSPFKCVNEGPYWSSCQ |
| 114 | MIC-54347 | MLYTAALAGLMATAALAAPYRRQATGQNVVYWGQNGGGVTESNNLSDYCDAEAGIDLLVLSF |
| | | LYEYGNGNTIPSGTIGQSCSIDTSGNPSNCDALASAIATCKSNGVKVVLSLGGAVGAYSLSS |
| | | QQEAETIGQNLWDAYGAGNGTVPRPFRSTVVDGWDFDIEASSGNQYYQYLIAKLRSNENGGN |
| | | YVITGAPQCPIPEPNMQQIITTSQFDYLWVQFYNNPSCSVGTSTPNFQDWVSNIANTPSANA |
| | | KIFLGVPASPLGATGTESGAQYYLEPSALNTLVGQFSSNPAFGGIMMWAAGESDANVNNGCT |
| | | YAQEAKKILTSGQIC |
| 115 | MIC-54347 | MPLKVFESASPAPSHPSLSHLCLLVFEAVLEVVCVSLPGYIVARLGHFDADKQKFLANLNVM |
| | | LFTPCLIFTKLASQLNAEKLSDLAIIPAIFIVQTLVSWIVSILVAKGFRENKRASNFVTAMG |
| | | VFGNSNSLPISLVLSLSQTIKGLHWDRIPGDNDDEVGARGILYLLIFQQLGQLVRWSWGYHV |
| | | LLAPKDKYAEYQDEIAEEGQYRYRDEEPNEQEPEILITGLDGDTEDDGESNASEDYIPAGRT |
| | | PLASNSRASLAGSSVDNDDMLNFKKGNYTRGSSLANTDLEDDILSFPRIRLRDEAEVEHGVT |
| | | ARIKKSLYSLKDKASAAMTRQYQRLPQPVQTCLSFIHKSITKTLGFVWDFMNPPLWAMLIAV |
| | | IVASIPSLQQLFFEDGSFVKNSVTNAVSSSGGVAVPLILVVLGANLARNTAAHDSPIDPEEE |
| | | KIGTKLLIASLLSRMVLPTLIMAPILAITAKYLPISILDDPIFIVVCELLTGAPSALQLAQI |
| | | CQINNVYEQTMGRILFQSYVIWILPSTLFLVMMALEVIEWATVN |
| 116 | MIC-54347 | MPSLITVASVLALVPSAFAGWNVNSKQNIAVYWGQNSANQQSTQQRLSAYCNAKIESDSCDV |
| | | DANINVIDIAFLNGITPPMTNFANAGDRCTPFSDNPWLLSCPEIEADIKTCQANGKTILISL |
| | | GGDSYTQGGWSSTSAAQAAAKQVWAMFGPVQSGSSTERPFGSAVVDGFDFDFEATTNNLAAF |
| | | GAQLKSLSAAGGKKYYFSAAPQCFFPDAAVGALINAVPMDWIQIQFYNNPCGVSGYTPGTSN |
| | | QNNYNYQTWDTWAKTSPNPNVKLLVGIPAGPGAGRGYVSGSQLTSVFQYSKGFSSTFAGAMM |
| | | WDMSQLFQNTGFEAQVVNALK |
| 117 | MIC-54347 | MQLSNLFKLALFTAAVSADTVSYDTGYDDASRSLTVVSCSDGPNGLITRYHWQTQGQIPRFP |
| | | YIGGVQAVAGWNSPSCGTCWKLTYSGKTIYVLAVDHAGAGENIGLDAMNALTNGQAVALGRV |
| | | SATASQVAVSNCGL |
| 118 | MIC-54347 | MQLTTVVALFASLAGAAPAPEPAAELVARDGPCSSGVTNNVPQCCGTGILSVVYVDCKTPTQ |
| | | APSATNQLSAICARVGLQAKCCTVGIAGVGVLCQDAIPQ |
| 119 | MIC-54347 | MRFNSFIAVAAATAQSAAGLGINCLGDTLCGISYMSGGRLTQFQTIFDNIFEKRIYDNGDDI |
| | | GCIEVHSINFKGSFKGTYCAYVQNLDGSVNGATLKTLYTELVNYGCGICGSIPIHYSKGDND |
| | | SNHGELSFNMVDSLPDNCELGKPCAATS |
| 120 | MIC-54347 | MRTSTAIISLALVVMGIAAPAAEAGSVVSEAGFVVSEAQFNAIFPGRNPFYSYRALTGALGS |
| | | YPSFTNTADNATRLREAAAFFANVDHETDGLKFVVE |
| 121 | MIC-54347 | MSAASRKSRIATSVARVMYTNAVYFPSSRIYQGDSPGMLNYSCINHVYYAYASVTADGNVEL |
| | | GDEWADARAPVDGVQGGLGSLMHLKQRHPHLQVVLSIGGSTASEVFPIVASSTLLRDNFARS |
| | | ALGLVEASGLDGIDIAWEFPSQAKHGHDFLALLAAVRIHMPEDRFILTAVLPAAKEVLQLID |
| | | LSTAAEYLDYINLVAYDFFGTWTSKTGHHAQLYTMNKDEPSASTGVAYVMSQGFPAKSILLG |
| | | IPTYGRSFLKANGPGQDFNGVGGQDGTFEYGELPRKGCKEIVDRRYIAAQCVGGDGGFVTYD |
| | | NPETVKVKAEFCKQKGLGGLFYWNGPADSRDQARSLIAAGFRALHTS |
| 122 | MIC-54347 | MTRLLDASFLLLPAIASTLFGTASAQNATCALKGKPAGKVLMGYWENWDGSANGVHPGFGWT |
| | | PIENPIIKQNGYNVINAAFPVILSDGTVLWENDMAPGTQVATPAEMCAAKAAGATILLSIGG |
| | | ATAGIDLSSSTVADKFIATIVPILKQYNEDGIDIDIETGLVNSGNIKTLSTSQANLIRIIDG |
| | | VLAQMPSNFGLTMAPETAYVTGGSITYGSIWGSYLPIIQKYVQNGRLWWLNMQYYNGDMYGC |
| | | SGDSYAAGTVQGFIAQTDCLNAGLTVQGTTIKVPYSMQVPGLPAQSGAGGGYMNPSLVGQAW |
| | | DHYNGALKGLMTWSINWDGAGGWTFGDNLLTRIG |
| 123 | MIC-54347 | MTSIRRLALYLGALLPAVLAAPAALHKKPEAAPNKFIVTLKEGASIDTDSHLAWVNDIHRRS |
| | | LTKRSTAGVEKTYNIHTWSAYAGEFDAETIEQIKSSPDVASVEPDYIMYLSDIVEDKRALTT |
| | | QSGAPWGLGTVSHRTSGSTSYIYDSSAGSGTFAYVVDSGINTSHQQFGGRASLGYNAAGGQH |
| | | VDTLGHGTHVSGTIGGSTYGVAKQASLISVKVFAGESATTSVILDGYNWAVNDIVSRNRASK |
| | | SAINMSLGGPASSTWTTAINAAFNQGVLTIVAAGNGDSFGNPQPVSGTSPANVPNAITVAAL |
| | | DINWRTASFTNYGAGVDVFAPGVNILSSWIGSNTATNTISGTSMATPHVVGLALYLQSLEGL |
| | | TTPTAVTNRIKALATAGRITGSLNGSPNTLIFNGNSA |
| 124 | MIC-54347 | MTSLYLTSALGLLCLLPAAQAGWNPNSKNNIVVYWGQDAGSIGQNRLSYYCENAPDVDVINI |
| | | SFLVGITDLNLNLANVGNNCTSFAQDPNLLNCPQVAADIVECQQTYGKTIMMSLFGSTYTES |
| | | GFSSSSTAVSAAQEIWAMFGPVQSGNSTPRPFGNAVIDGFDFDLEDPIENNMEPFAAELRSL |
| | | TSAATSKKFYLSAAPQCVYPDASDQTFLQGEVAFDWLNIQFYNNGCGTSYYPSGYNYATWDN |
| | | WVKTVSANPNTKLLVGTPASVHAVNFANYFPTNDQLAGAISSSKSYGSFAGVMLWDMAQLFG |
| | | NPGYLDLIVADLGGASTPPPPASTTLSTVTRSSTASSGPTSPPPSGGGSVPQWGQCGGQGYT |
| | | GPTQCQSPYTCVVESQWWASCQ |
| 125 | MIC-54347 | MVQSAAYLGALLATLPLARAGFNASSTQNIAVYWGQNSANQATSQQRLSTYCANADIDIIPI |
| | | GFMNGISPVITNFANAGDNCTAFADNQNALNCPQIEQDIITCQQTYGKTILISLGGGSYTQG |
| | | GFSSTGVATSAAQTVWNMFGPVNPNSNVDRPFGSAVVDGVDFDFESGVNNLATFATELRSLM |
| | | DASASSANRKFYLSAAPQCVYPDFADNPALNGSVFFDFIMIQYYNNGCGVSSYVPGATTQWN |
| | | YNFDVWDNWAHTVSKNPNVRILLGIAANTGAASGYVSGTQLSAVISFTKQYSSFAGIMMWDM |
| | | SQLYQNSGFLGQVVSDLAASGSTPPATTSSGASKTTTSSGGSTSPTGGSVPQWGQCGGEGYT |
| | | GPTQCQSPYKCVESSQWWSSCQ |
| 126 | MIC-54347 | MYFANTLLSIAGAASIANALGSNCQDSGVCADINANLSFAIEQLKGMDQLQRFSDGQHITCV |
| | | DTDSEGNSSLCLSYQDTGRSWTVFQTAWFAQSLVEQGCQACGSLSMGSHHGELVASVITKST |
| | | SGLDTSGARRGMDMVQLAVRAGDR |
| 127 | MIC-67967 | TTACCGACTTCAGGTCCCCCCGGCTTTCATGGCTTGACGGGCGGTGTGTACAAGGTCCGGGA |
| | | ACGTATTCACCGTATCATTGCTGATATACGATTACTAGCGATTCCAGCTTCATGAGGGCGAG |
| | | TTGCAGCCCTCAATTCGAACTGAGATAGGATTTTTGAGATTAGCATCCTGTTACCAGGTAGC |
| | | AGCCCTTTGTTCCTACCATTGTAGCACGTGTGTAGCCCTGGGCATAAAGGCCATGATGACTT |
| | | GACATCATCCCCTCCTTCCTCGCGTCTTACGACGGCAGTTTCTTTAGAGTTCCCAGCTTAAC |
| | | CTGTTGGCAACTAAAGATAGGGGTTGCGCTCGTTGCGGGACTTAACCCAACACCTCACGGCA |
| | | CGAGCTGACGACAGCCATGCAGCACCTTACAAAATGTGTATTGCTACAAAATCTGCTTTCAC |
| | | AGACGGGCATCTTGCATTCTAGCCCAGGTAAGGTTCCTCGCGTATCATCGAATTAAACCACA |
| | | TGCTCCACCGCTTGTGCGGACCCCCGTCAATTCCTTTGAGTTTCAACCTTGCGGTCGTACTT |
| | | CCCAGGTGGATTACTTAATGCTTTCGCTCAGACACTTACTGTATATCGCAAATGTCGAGTAA |
| | | TCATCGTTTAGGGCGTGGACTACCAGGGTATCTAATCCTGTTTGATCCCCACGCTTTCGTGC |
| | | CTCAGCGTCAATATTTGTGTAGCCAGCTGCCTTCGCAATTGGTGTTCTATGTCATATCTATG |
| | | CATTTCACCGCTACATGACATATTCCGCTAACCTCCACAATATTCAAGACTTATAGTATCCA |
| | | TGGCAGTTTCCAGGTTAAGCCTGGAGATTTCACCACGGACTTACAAATCCGCCTACGCACCC |
| | | TTTAAACCCAGTGAATCCGGATAACGCTTGCACCCTCCGTATTACCGCGGCTGCTGGCACGG |
| | | AGTTAGCCGGTGCTTATTCCTCTGGTACCGTCAACACTCTTAGAAAAAGGTGATTTCGTCCC |
| | | AGATAAAAGAAGTTTACAATCCAGAGGACCTTCATCCTTCACGCGGCATGGCTGGTTCAGAC |
| | | TTGCGTCCATTGACCAATATTCCTTACTGCTGCCTCCCGTAGGAGTCGGGCCCGTGTCTCAG |
| | | TGCCCGTGTGACTGGTCGCGCTCTCACGCCAGTTACTGATCGTCGGCTTGGTGAGCCGTTAC |
| | | CTCACCAACTACCTAATCAGGCGCACGCCCATCTTCAAGCGAAATTCTTTAATCATTAAGTG |
| | | ATGCCACCCTGTGATTTTACGATGTATTAATCCGAATTTCTTCGGGCTATCCATCTCTTGAA |
| | | GGAAGGTTGCGTACGTGTTCCGCACCCGTTTGCCGGTCGCCACCCAGTATTGCTACCTGTGC |
| | | TGCCCCTCGACTTGCA |
| 128 | MIC-67967 | MKIAIINGPNLNLLGKREPGIYGNEPFEDYFEKLKVLFADVELEYYQNNSEGNIIDILHEIG |
| | | FTYDGILLNAGAYTHYSIAIRDAIAAIKTPVLEIHISNVHAREEFRHKSVIAPVCKGMIAGL |
| | | GMKGYALGINYFL |
| 129 | MIC-67967 | MKQVEVGIIMGSSSDAPIMRQAIDVLKKFDIGYEFNVVSAHRSPQRMEDYAGTAEERGLKVI |
| | | IAGAGGAAHLPGMVAAITTLPVVGVPIKSSNSLDGWDSLLSIVQMPGDIPVATVSVNGARNA |
| | | GLLAVQILATCNPELRQKLADMKRENNEKVNQMNETLDRS |
| 130 | MIC-67967 | MNSFGRLFRVNVFGESHGASVGVNIDGVPAGIPLKQEDFLPDLDRRKAGAKGTTPRKEDDLP |
| | | YIKSGVFNDHTTGAPITILFENNNTRSTDYEKLREFPRPGHADFVATHKYGGFEDYRGGGHF |
| | | SGRLTLNLVAAGVIAKKILGESIKVTATLKEVAGLPDAEQGLEAAIAAKDSVGGIVECVVEG |
| | | LPIGLGEPFFDSVESTIAHAVFAIPAIKGIEFGAGFAAAKMKGIEHNDAILDASGKTATNNA |
| | | GGVVGGITNGNPLVFRVAVKPTSSTPKEQHTLNIKSGQVEAFSVKGRHDLCIALRVPVVLEA |
| | | VAAMALADLMMVEQRSPRIWK |
| 131 | MIC-67967 | MSGQLKEVRNRIKSTQSNLQITKAMKMVSAAKLRRAQDAILLMRPYAVKLQEMLQNIVSNSE |
| | | GSIDLALAAERPVEKVLLVVITSDRGLCGAYNSNLIKLTKQVIREKYQEQFEKGHVTILPIG |
| | | KKGWEHFGKNGYKMNDTYWHLFAHLDEDHVKEAAAVATDGETSGQYDAVEIIYSQFKNAATQ |
| | | FYISEQFLPIAKPQEVETGKGKKAAKADFIFEPEKQTLIAELMPKILNTQLYKAMLDANASE |
| | | HGARMTAMDKATENANELLRNYKITYNRARQAAITTELTEIVSGAAALEG |
| 132 | MIC-84302 | GGGAGCTTGCTCCCTGATGTTAGCGGCGGACGGGTGAGTAACACGTGGGTAACCTGCCTGTA |
| | | AGACTGGGATAACTCCGGGAAACCGGGGCTAATACCGGATGGTTGTTTGAACCGCATGGTTC |
| | | AGACATAAAAGGTGGCTTCGGCTACCACTTACAGATGGACCCGCGGCGCATTAGCTAGTTGG |
| | | TGAGGTAACGGCTCACCAAGGCAACGATGCGTAGCCGACCTGAGAGGGTGATCGGCCACACT |
| | | GGGACTGAGACACGGCCCAGACTCCTACGGGAGGCAGCAGTAGGGAATCTTCCGCAATGGAC |
| | | GAAAGTCTGACGGAGCAACGCCGCGTGAGTGATGAAGGTTTTCGGATCGTAAAGCTCTGTTG |
| | | TTAGGGAAGAACAAGTGCCGTTCAAATAGGGCGGCACCTTGACGGTACCTAACCAGAAAGCC |
| | | ACGGCTAACTACGTGCCAGCAGCCGCGGTAATACGTAGGTGGCAAGCGTTGTCCGGAATTAT |
| | | TGGGCGTAAAGGGCTCGCAGGCGGTTTCTTAAGTCTGATGTGAAAGCCCCCGGCTCAACCGG |
| | | GGAGGGTCATTGGAAACTGGGGAACTTGAGTGCAGAAGAGGAGAGTGGAATTCCACGTGTAG |
| | | CGGTGAAATGCGTAGAGATGTGGAGGAACACCAGTGGCGAAGGCGACTCTCTGGTCTGTAAC |
| | | TGACGCTGAGGAGCGAAAGCGTGGGGAGCGAACAGGATTAGATACCCTGGTAGTCCACGCCG |
| | | TAAACGATGAGTGCTAAGTGTTAGGGGGTTTCCGCCCCTTAGTGCTGCAGCTAACGCATTAA |
| | | GCACTCCGCCTGGGGAGTACGGTCGCAAGACTGAAACTCAAAGGAATTGACGGGGGCCCGCA |
| | | CAAGCGGTGGAGCATGTGGTTTAATTCGAAGCAACGCGAAGAACCTTACCAGGTCTTGACAT |
| | | CCTCTGACAATCCTAGAGATAGGACGTCCCCTTCGGGGGCAGAGTGACAGGTGGTGCATGGT |
| | | TGTCGTCAGCTCGTGTCGTGAGATGTTGGGTTAAGTCCCGCAACGAGCGCAACCCTTGATCT |
| | | TAGTTGCCAGCATTCAGTTGGGCACTCTAAGGTGACTGCCGGTGACAAACCGGAGGAAGGTG |
| | | GGGATGACGTCAAATCATCATGCCCCTTATGACCTGGGCTACACACGTGCTACAATGGGCAG |
| | | AACAAAGGGCAGCGAAACCGCGAGGTTAAGCCAATCCCACAAATCTGTTCTCAGTTCGGATC |
| | | GCAGTCTGCAACTCGACTGCGTGAAGCTGGAATCGCTAGTAATCGCGGATCAGCATGCCGCG |
| | | GTGAATACGTTCCCGGGCCTTGTACACACCGCCCGTCACACCACGAGAGTTTGTAACACCCG |
| | | AAGTCGGTGAGGTA |
| 133 | MIC-84302 | MAITAQQVKELRQKTGAGMMDCKKALTETDGDMDKAIDLLREKGIAKAAKKADRIAAEGSTL |
| | | IKTDGNKGVILEVNSETDFVAKNEGFKELLNTLADHLLANAPADLEEAMGQKMENGSTVEEY |
| | | ITSNVAKIGEKITLRRFAVITKEDSEAFGAYLHMGGRIGVLSVLSGTDEDLAKDIAMHVAAV |
| | | NPRYISRDQVSEEEANHERQILTQQALQEGKPENIVAKMVEGRLNKFFEEICLLDQAFVKNP |
| | | DEKVKQVVAAKNASVKTYVRYEVGEGIEKRQENFAEEVMNQVKK |
| 134 | MIC-84302 | MKTVKVNIVTPDGPVYDADIEMVSVRAESGDLGILPGHIPTVAPLKIGAVRLKKDGQTELAA |
| | | VSGGFLEVRPDQVTILAQAAETAESIDKERALAAKKRAEDRINKRSDDTDIRRAELALQRAV |
| | | NRLDVAGN |
| 135 | MIC-84302 | MNFETVIGLEVHVELKTKSKIFSSSPTPFGAEANTQTSVIDLGYPGVLPVLNKEAVEFAMKA |
| | | AMALNCEIATDTKFDRKNYFYPDNPKAYQISQFDKPIGENGWIEIEVGGKTKKIGITRLHLE |
| | | EDAGKLTHTGDGSLVDENRQGTPLVEIVSEPDIRTPEEAYAYLEKLKSIIQYTGVSDCKMEE |
| | | GSLRCDANISLRPIGQEKFGTKTELKNLNSFAFVQKGLEYEEKRQEQVLLSGGVIQQETRRY |
| | | DEATKKTILMRVKEGSDDYRYFPEPDLVELYIDDEWKERVRATIPELPDERRKRYIEELGLP |
| | | AYDAMVLTLTKEMADFFEETVNKGAEAKQASNWLMGEVSAYLNAEQKELEDVALTPEGLAGM |
| | | IKLIEKGTISSKIAKKVFKELIEKGGDAEKIVKEKGLVQISDESVLLKLVTDALDSNPQSIE |
| | | DFKNGKDRAIGFLVGQIMKASKGQANPPMVNKILLEEIKKR |
| 136 | MIC-84302 | MSYLIKNGWMLNEQGEKVAQDIRVTGEFITETGLLTAKDGETVIDAEGLFVSPGLVDLHVHF |
| | | REPGGEKKETIETGSKAAARGGFTTVAAMPNTRPVPDTKEQMEWLQNRIKETSSVRVLPYAS |
| | | ITIRQIGEEMTDFEALHEAGAFAFTDDGVGVQTAGMMYEAMKRAASMNKAIVAHCEDNSLIY |
| | | GGSVHEGSFSKANGLNGIPSICEAVHIARDVLLAEAADCHYHVCHISTKESVRAVRDAKKAG |
| | | IRVTAEVSPHHLLLCDEDIPGLDTNFKMNPPLRSKEDREALIEGLLDGTIDFIATDHAPHTE |
| | | EEKNTDMKLAPFGIVGLETAFPLLYTHFVKNGTWTLKQLVDFMTVKPCEAFGLPYGTLAPGA |
| | | PADITLIDLEKEAAIDKDTFLSKGKNTPFNKMKCFGWPVATMAAGKLAYEEGRLV |

### Example 2. Taxonomic Classification of Endophytes

Classification of strains was done by the following methodology.

To accurately characterize isolated bacterial endophytes, colonies were submitted for marker gene sequencing, and the sequences were analyzed to provide taxonomic classifications. Colonies were subjected to 16S rRNA gene PCR amplification using a primer pair 27f (5′-AGAGTTTGATYMTGGCTCAG-3′) (SEQ ID NO: 1) and 1492r (5′-GGTTACCTTGTTACGACTT-3′) (SEQ ID NO: 2). Sequencing reactions were performed using primers: 27f (5′-AGAGTTTGATYMTGGCTCAG-3′) (SEQ ID NO: 1), 515f (5′-GTGYCAGCMGCCGCGGTAA-3′) (SEQ ID NO: 3), 806r (5′-GGACTACNVGGGTWTCTAAT-3′) (SEQ ID NO: 4), and 1492r (5′-GGTTACCTTGTTACGACTT-3′) (SEQ ID NO: 2). To accurately characterize isolated fungal endophytes, genomic DNA isolated as above was submitted for marker gene sequencing, and the sequences were analyzed to provide taxonomic classifications. PCR was used to amplify the nuclear ribosomal internal transcribed spacers (ITS) region using the primer pair ITS_1 (5′-CTTGGTCATTTAGAGGAAGTAA-3′) (SEQ ID NO: 5) and LR5 (5′-TCCTGAGGGAAACTTCG-3′) (SEQ ID NO: 6). Each 25 microliter-reaction mixture included 22.5 microliters of Invitrogen Platinum Taq supermix, 0.5 microliter of each primer (10 micromolar), and 1.5 microliters of DNA template (˜2-4 ng). Cycling reactions were run with MJ Research PTC thermocyclers and consisted of 94° C. for 5 min, 35 cycles of 94° C. for 30 s, 54° C. for 30 s, and 72° C. for 1 min, and 72° C. for 10 min. Sanger sequencing of was performed at Genewiz (South Plainfield, NJ) using primers: ITS_1 (5′-CTTGGTCATTTAGAGGAAGTAA-3′) (SEQ ID NO: 5), ITS_2 (5′-GCTGCGTTCTTCATCGATGC-3′) (SEQ ID NO: 7), ITS_3 (5′-GCATCGATGAAGAACGCAGC-3′) (SEQ ID NO: 8), and LR5 (5′-TCCTGAGGGAAACTTCG-3′) (SEQ ID NO: 6). Preferably sequencing primers were chosen so that overlapping regions are sequenced. Sanger sequencing of marker genes was performed at Genewiz (South Plainfield, NJ). Raw chromatograms were converted to sequences, and corresponding quality scores were assigned using TraceTuner v3.0.6beta (U.S. Pat. No. 6,681,186). These sequences were quality filtered, aligned and a consensus sequence generated using Geneious v 8.1.8 (Biomatters Limited, Auckland NZ).

Taxonomic classifications were assigned to the sequences using the highest probability of assignment based on the results of industry standard taxonomic classification tools: LCA (runs USEARCH (Edgar, R. C., 2010) with option search_global, then for all best match hits, returns lowest taxonomic rank shared by all best hits for a query), RDP Naive Bayesian rRNA Classifier version 2.11, September 2015 (Wang et al., 2007), SPINGO version 1.3 (32 bit) (Allard et al. (2015) BMC Bioinformatics 16:324 DOI: 10.1186/s12859-015-0747-1), and UTAX version v8.1.1861_i86linux64 (Edgar, R. C. (2016) available online at drive5.com/usearch/manual/utax_algo.html), using reference databases: RDP 16S rRNA training set 15 (Cole et al. (2014) Nucleic Acid Research, 42 (Database issue): D633-D642), and SILVA version 119 (Quast et al. (2013) Nucleic Acid Research, 41 (Database issue): D590-D596). The classifier and database combinations listed in Table 4 were used to assign taxonomy to bacterial sequences.

TABLE 4
The classifier and database combinations used
to classify 16S rRNA gene or ITS sequences

| Classifier | Database |
|---|---|
| LCA (Bacteria) | SILVA, version 119 |
| RDP (Bacteria) | RDP, 16S rRNA training set 15 |
| SPINGO (Bacteria) | RDP, 16S rRNA training set 15 |
| UTAX (Bacteria) | RDP, 16S rRNA training set 15; SILVA, |
| | version 119 |
| LCA (Fungi) | UNITE, Fungal ITS trainset Jul. 4. 2014 |
| RDP (Fungi) | UNITE, Fungal ITS trainset Jul. 4. 2014; |
| | WARCUP, Fungal ITS trainset 1 |
| SPINGO (Fungi) | UNITE, Fungal ITS trainset Jul. 4. 2014 |
| UTAX (Fungi) | UNITE, Fungal ITS trainset Jul. 4. 2014; |
| | WARCUP, Fungal ITS trainset 1 |

TABLE 5
Taxonomic classification of endophytes of the present invention

| MIC ID | MIC-67967 | MIC-84302 | MIC-18905 | MIC-54347 |
|---|---|---|---|---|
| Kingdom | Bacteria | Bacteria | Bacteria | Fungi |
| Phylum | Bacteroidetes | Firmicutes | Proteobacteria | Ascomycota |
| Class | Chitinophagia | Bacilli | Gammaproteobacteria | Sordariomycetes |
| Order | Chitinophagales | Bacillales | Pseudomonadales | Hypocreales |
| Family | Chitinophagaceae | Bacillaceae | Pseudomonadaceae | Hypocreaceae |
| Genus | Chitinophaga | Bacillus | Pseudomonas | Trichoderma |
| Species | oryzae | velenzensis | glycinis | hamatum |

### Example 3. Assessment of Improved Plant Characteristics: Vigor Assay

### Assay of Soy Seedling Vigor

Seed preparation: The lot quality of soybean seeds is first assessed by testing germination of 100 seeds. Seeds are placed, 8 seeds per petri dish, on filter paper in petri dishes, 12 ml of water is added to each plate and plates are incubated for 3 days at 24° C. The process should be repeated with a fresh seed lot if fewer than 95% of the seeds have germinated. One thousand soybean seeds are then surface sterilized by co-incubation with chlorine gas in a 20×30 cm container placed in a chemical fume hood for 16 hours. Percent germination of 50 seeds, per sterilization batch, is tested as above and confirmed to be greater than 95%.

Preparation of endophyte treatments: Spore solutions are made by rinsing and scraping spores from agar slants which have been growing for about 1 month. Rinsing is done with 0.05% Silwet. Solutions are passed through Miracloth to filter out mycelia. Spores per ml are counted under a microscope using a hemocytometer. The stock suspension is then diluted into 10\hat{ }6 spores/ml utilizing water. 3 μl of spore suspension is used per soy seed (˜10\hat{ }3 CFUs/seed is obtained). Control treatments are prepared by adding equivalent volumes of sterile water to seeds.

Assay of seedling vigor: Two rolled pieces of germination paper are placed in a sterile glass gar with 50 ml sterile water, then removed when completely saturated. Then the papers are separated, and inoculated seeds are placed at approximately 1 cm intervals along the length of one sheet of moistened germination paper, at least 2.5 cm from the top of the paper and 3.8 cm from the edge of the paper. The second sheet of is placed on top of the soy seeds and the layered papers and seeds are loosely rolled into a tube. Each tube is secured with a rubber band around the middle and placed in a single sterile glass jar and covered loosely with a lid. For each treatment, three jars with 15 seeds per jar are prepared. The position of jars within the growth chamber is randomized. Jars are incubated at 60% relative humidity, and 22° C. day, 18° ° C. night with 12 hours light and 12 hours dark for 4 days and then the lids are removed, and the jars incubated for an additional 7 days. Then the germinated soy seedlings are weighed and photographed, and root length and root surface area are measured.

Dirt, excess water, seed coats and other debris is removed from seedlings to allow accurate scanning of the roots. Individual seedlings are laid out on clear plastic trays and trays are arranged on an Epson Expression 11000XL scanner (Epson America, Inc., Long Beach CA). Roots are manually arranged to reduce the amount of overlap. For root measurements, shoots are removed if the shape of the shoot causes it to overlap the roots.

The WinRHIZO software version Arabidopsis Pro2016a (Regents Instruments, Quebec Canada) is used with the following acquisition settings: greyscale 4000 dpi image, speed priority, overlapping (1 object), Root Morphology: Precision (standard), Crossing Detection (normal). The scanning area is set to the maximum scanner area. When the scan is completed, the root area is selected, and root length and root surface area are measured.

Statistical analysis is performed using R (R Core Team, 2016. R: A language and environment for statistical computing. R Foundation for Statistical Computing, Vienna, Austria. R-project.org/) or a similar statistical software program.

### Assay of Rice Seedling Vigor

Seed preparation: The lot of rice seeds is first evaluated for germination by transfer of 100 seeds and with 8 ml of water to a filter paper lined petri dish. Seeds are incubated for 3 days at 24° C. The process should be repeated with a fresh seed lot if fewer than 95% of the seeds have germinated. Rice seeds are then surface sterilized by co-incubation with chlorine gas in a 20×30 cm container in a chemical fume hood for 12 hours. Percent germination of 50 seeds, per sterilization batch, is tested as above and confirmed to be greater than 95%.

Optional reagent preparation: 7.5% polyethylene glycol (PEG) is prepared by adding 75 g of PEG to 1000 ml of water, then stirring on a warm hot plate until the PEG is fully dissolved. The solution is then autoclaved.

Preparation of endophyte treatments: Spore solutions are made by rinsing and scraping spores from agar slants which have been growing for about 1 month. Rinsing was done with 0.05% Silwet. Solutions are passed through Miracloth to filter out mycelia. Spores per ml are counted under a microscope using a hemocytometer. The stock suspension is then diluted into 10\hat{ }6 spores/ml utilizing water. 3 μl of spore suspension is used per rice seed (˜10\hat{ }3 CFUs/seed was obtained). Seeds and spores are combined in a 50 ml falcon tube and gently shaken for 5-10 seconds until thoroughly coated. Control treatments are prepared by adding equivalent volumes of sterile water to seeds.

Assay of seedling vigor: Petri dishes are prepared by adding four sheets of sterile heavy weight seed germination paper, then adding either 50 ml of sterile water or, optionally, 50 ml of PEG solution as prepared above, to each plate then allowing the liquid to thoroughly soak into all sheets. The sheets are positioned and then creased so that the back of the plate and one side wall are covered, two sheets are then removed and placed on a sterile surface. Along the edge of the plate across from the covered side wall 15 inoculated rice seeds are placed evenly at least one inch from the top of the plate and half an inch from the sides. Seeds are placed smooth side up and with the pointed end of the seed pointing toward the side wall of the plate covered by germination paper. The seeds are then covered by the two reserved sheets, and the moist paper layers smoothed together to remove air bubbles and secure the seeds, and then the lid is replaced. For each treatment, at least three plates with 15 seeds per plate are prepared. The plates are then randomly distributed into stacks of 8-12 plates and a plate without seeds is placed on the top. The stacks are incubated at 60% relative humidity, and 22° C. day, 18° C. night with 12 hours light and 12 hours dark for 24 hours, then each plate is turned to a semi-vertical position with the side wall covered by paper at the bottom. The plates are incubated for an additional 5 days, then rice seeds are scored manually for germination, root and shoot length.

Statistical analysis is performed using R or a similar statistical software program.

### Assay of Corn Seedling Vigor

Seed preparation: The lot quality of corn seeds is first evaluated for germination by transfer of 100 seeds with 3.5 ml of water to a filter paper lined petri dish. Seeds are incubated for 3 days at 24° C. The process should be repeated with a fresh seed lot if fewer than 95% of the seeds have germinated. One thousand corn seeds are then surface sterilized by co-incubation with chlorine gas in a 20×30 cm container in a chemical fume hood for 12 hours. Percent germination of 50 seeds, per sterilization batch, is tested as above and confirmed to be greater than 95%.

Optional reagent preparation: 7.5% PEG 6000 (Calbiochem, San Diego, CA) is prepared by adding 75 g of PEG to 1000 ml of water, then stirred on a warm hot plate until the PEG is fully dissolved. The solution is then autoclaved.

Preparation of endophyte treatments: Spore solutions are made by rinsing and scraping spores from agar slants which have been growing for about 1 month. Rinsing is done with 0.05% Silwet. Solutions are passed through Miracloth to filter out mycelia. Spores per ml are counted under a microscope using a hemocytometer. The stock suspension is then diluted into 10\hat{ }6 spores/ml utilizing water. 3 μl of spore suspension is used per corn seed (˜10\hat{ }3 CFUs/seed is obtained). Control treatments are prepared by adding equivalent volumes of sterile water to seeds.

Assay of seedling vigor: Either 25 ml of sterile water or, optionally, 25 ml of PEG solution as prepared above, is added to each Cyg™ germination pouch (Mega International, Newport, MN) and place into pouch rack (Mega International, Newport, MN). Sterile forceps are used to place corn seeds prepared as above into every other perforation in the germination pouch. Seeds are fitted snugly into each perforation to ensure they do not shift when moving the pouches. Before and in between treatments forceps are sterilized using ethanol and flame and workspace wiped down with 70% ethanol. For each treatment, three pouches with 15 seeds per pouch are prepared. The germination racks with germination pouches are placed into plastic tubs and covered with perforated plastic wrap to prevent drying. Tubs are incubated at 60% relative humidity, and 22° ° C. day, 18° C. night with 12 hours light and 12 hours dark for 6 days to allow for germination and root length growth. Placement of pouches within racks and racks/tubs within the growth chamber is randomized to minimize positional effect. At the end of 6 days the corn seeds are scored manually for germination, root and shoot length.

Statistical analysis is performed using R or a similar statistical software program.

### Assay of Wheat Seedling Vigor

Seed preparation: The lot of wheat seeds is first evaluated for germination by transfer of 100 seeds and with 8 ml of water to a filter paper lined petri dish. Seeds are incubated for 3 days at 24° C. The process should be repeated with a fresh seed lot if fewer than 95% of the seeds have germinated. Wheat seeds are then surface sterilized by co-incubation with chlorine gas in a 20×30 cm container in a chemical fume hood for 12 hours. Percent germination of 50 seeds, per sterilization batch, is tested as above and confirmed to be greater than 95%.

Optional reagent preparation: 7.5% polyethylene glycol (PEG) is prepared by adding 75 g of PEG to 1000 ml of water, then stirring on a warm hot plate until the PEG is fully dissolved. The solution is then autoclaved.

Preparation of endophyte treatments: Spore solutions are made by rinsing and scraping spores from agar slants which have been growing for about 1 month. Rinsing is done with 0.05% Silwet. Solutions are passed through Miracloth to filter out mycelia. Spores per ml are counted under a microscope using a hemocytometer. The stock suspension is then diluted into 10\hat{ }6 spores/ml utilizing water. 3 μl of spore suspension is used per wheat seed (˜10\hat{ }3 CFUs/seed was obtained). Seeds and spores are combined a 50 ml falcon tube and gently shaken for 5-10 seconds until thoroughly coated. Control treatments are prepared by adding equivalent volumes of sterile water to seeds.

Assay of seedling vigor: Petri dishes are prepared by adding four sheets of sterile heavy weight seed germination paper, then adding either 50 ml of sterile water or, optionally, 50 ml of PEG solution as prepared above, to each plate then allowing the liquid to thoroughly soak into all sheets. The sheets are positioned and then creased so that the back of the plate and one side wall are covered, two sheets are then removed and placed on a sterile surface. Along the edge of the plate across from the covered side wall 15 inoculated wheat seeds are placed evenly at least one inch from the top of the plate and half an inch from the sides. Seeds are placed smooth side up and with the pointed end of the seed pointing toward the side wall of the plate covered by germination paper. The seeds are then covered by the two reserved sheets, and the moist paper layers smoothed together to remove air bubbles and secure the seeds, and then the lid is replaced. For each treatment, at least three plates with 15 seeds per plate are prepared. The plates are then randomly distributed into stacks of 8-12 plates and a plate without seeds is placed on the top. The stacks are incubated at 60% relative humidity, and 22° C. day, 18° C. night with 12 hours light and 12 hours dark for 24 hours, then each plate is turned to a semi-vertical position with the side wall covered by paper at the bottom. The plates are incubated for an additional 5 days, then wheat seeds are scored manually for scored manually for germination, root and shoot length, root and shoot surface area, seedling mass, root and shoot and seedling length.

Statistical analysis is performed using R or a similar statistical software program.

### Example 4. Method of Preparation of Endophytes and Heterologous Disposition of Endophytes on Seeds

Seeds are heterologously disposed to each endophyte according to the following seed treatment protocol.

### Preparation of Seeds

The average weight of seeds is calculated by weighing 3 samples of 100 size selected seeds each and calculating the average weight of a seed. This value is used to calculate the target dose of endophyte per seed. The target dose is generally between 10\hat{ }0-10\hat{ }6 CFU per seed, in some cases at least 10\hat{ }3 CFU per seed, or at least 10\hat{ }4 CFU per seed. Table Z lists the target dose for endophytes of the present invention as applied to larger seeds (for example corn, soy and cotton) and smaller seeds (for example, wheat, rice, barley, oats). Other types of plants may be treated, and dosage determined based on the similarity of those seeds to the seeds listed in Table Z.

TABLE Z
Target doses in for endophytes of
the present invention by crop type.

Target dose (CFU per seed)

| | Large seeds: | Small seeds: |
|---|---|---|
| | Corn/Soy/Cotton | Wheat/Rice/Barley/Oat |
| MIC-67967 | 1.00E+05 | 2.25E+04 |
| MIC-18905 | 1.00E+05 | 2.25E+04 |
| MIC-84302 | 1.00E+05 | 1.00E+04 |
| MIC-54347 | 1.00E+04 | 1.00E+03 |

### Preparation of Bacterial and Fungal Endophytes

MIC-54347 is produced by solid state fermentation. A seed flask containing potato dextrose broth (PDB) is inoculated with MIC-54347 and incubated for 7 days. Soil substrate consisting of 33% millet, 9.4% vermiculite, 9.4% clay, 2.8% wheat bran, 0.6% yeast extract, 45% water is inoculated with the seed culture, and the culture grown for approximately 14 days at 24 C. Total biomass is collected. The total volume of inoculum needed to treat the seeds with the desired dose was calculated based on the target dose. The target dose is generally between 10\hat{ }0-10\hat{ }6 CFU per seed, in some cases at least 10\hat{ }3 CFU per seed, or at least 10\hat{ }4 CFU per seed. The inoculum is diluted with sterile 1×PBS so that the total volume of inoculum per seed is about 2.5 μl/seed for corn, about 1.5 microliters/seed for wheat and soy, and about 1.5 microliters/seed for cotton. Control treatments were prepared using equivalent volumes of sterile 1×PBS. The inoculum solution is combined with a treatment formulation containing talc and mineral oil and is applied to the prepared seeds and mixed well.

MIC-67967, MIC-84302, and MIC-18905 are produced by liquid state fermentation. A seed flask containing trypticase soy broth (TSB) is inoculated with the endophyte and incubated for 24 hours. Liquid fermentation is completed in a bioreactor. Total biomass is collected. The target dose is generally between 10\hat{ }0-10\hat{ }6 CFU per seed, in some cases at least 10\hat{ }3 CFU per seed, or at least 10\hat{ }4 CFU per seed. The biomass suspension is diluted with sterile 1×PBS so that the total volume of inoculum per seed is about 2.5 μl/seed for corn, about 1.5 μl/seed for wheat and soy, and about 1.5 μl/seed for cotton. Control treatments were prepared using equivalent volumes of sterile 1×PBS. For MIC-67967 and MIC-84302 the diluted biomass suspension is applied to the prepared seeds directly and mixed well. For MIC-67967 and MIC-84302 the biomass suspension is dried and combined with a treatment formulation containing talc and mineral oil and is applied to the prepared seeds and mixed well.

### Example 5. Greenhouse Assessment of Improved Plant Characteristics Under Water Deficit

This example describes an exemplary method by which improved plant health of endophyte treated plants may be shown in a growth environment comprising a water deficit.

Greenhouse assay setup: This greenhouse assay is conducted in individual plastic pots, filled with moistened potting soil. This greenhouse assay is conducted using seeds (optionally, chemically treated) coated with one or more endophytes described herein and formulation control (lacking the one or more heterologously disposed endophytes) and untreated controls (lacking formulation and the one or more heterologously disposed endophyte) as described in Example 4. Seeds are placed onto each pot and lightly covered with potting mix. Replicated pots of each treatment are set up and placed on a greenhouse bench using a random block design. For example, 18 replicates are planted for each treatment and control. Plants are monitored daily for emergence and watered as necessary to maintain a moist but not saturated soil surface (for example, plants are watered with 125 ml Hoagland's solution (8 mM N) (Hoagland, D. R. and D. I. Arnon. 1950. The water culture method for growing plant without soil. California Agri. Exp. Sta. Cir. No. 347. University of California Berkley Press, CA., pp: 347) per pot on every Monday, Wednesday and Friday).

The following growth and vigor metrics are measured for each treatment: percentage emergence at Day 4, 5, 7 (for soybean, winter wheat and cotton) or Day 3, 4, 5 (for corn), leaf count (the number of fully expanded leaves on the main stem) at Days 10, 17 and 24.

At Day 14 after planting, the potting mix in each pot is fully saturated (for example, 150 ml Hoagland's solution is added to each pot and the soil given time to fully absorb the solution, before an additional 150 ml Hoagland's solution). On subsequent days plants are observed and assigned a wilt score. Wilt scores range from 1-9 and are more fully described in the following table.

TABLE A
Description of phenotypes for each wilt scores

| Wilt | |
|---|---|
| score | Description of wilt phenotype |
| 9 | Normal no wilting - turgid green healthy |
| 8 | Leaves start losing turgor but are not soft yet |
| | no folding or rolling or change of leaf surface some |
| | small area of leaves becomes pale between the minor veins |
| 7 | Leaves further lose turgor become soft and pale at least |
| | one leaf starts slightly rolling |
| 6 | Leaves are further soft and pale all leaves are rolling |
| | except the center growing leaf |
| 5 | All leaves are very soft and pale with rolling - one |
| | leaf may be completely closed |
| 4 | Whole plant looks very bad - center leaves are very |
| | pale and rolling badly - all leaves may be completely |
| | closed - leaf sheath starts losing turgor |
| 3 | Leaf base is still fresh - leaf sheath loses turgor |
| | 2 lower leaves start drying |
| 2 | Center leaf starts drying - leaf base is not fresh |
| | anymore - all leaves are dried |
| 1 | For any plant that is worse than score 2 - the wilting |
| | score will be 1 |

Watering is withheld until 80% of plants have a wilt score of at least 4. Pots are then fully saturated and a normal watering schedule resumed. Additional vigor and growth metrics may be measured during recovery including shoot height, area of chlorosis, turgor pressure of leaves, number of live leaves, etc. After a recovery period, for example 1 week, plants are gently removed from pots, washed with tap water to remove dirt, and photographed. Optionally, plant tissue is collected for nutrient composition analysis. Plants are put into a paper bag and dried in an oven. Optionally, the plant is separated into shoot and root tissue prior to drying. The dry weight of each individual plant, or shoot or root thereof, is recorded.

### Example 6. Greenhouse Assessment of Improved Plant Characteristics Under Nitrogen Deficit

This example describes an exemplary method by which improved plant health of endophyte treated plants may be shown in a growth environment comprising a nitrogen deficit.

Greenhouse assay setup: This greenhouse assay is conducted in individual plastic pots, filled with moistened potting soil. This greenhouse assay is conducted using seeds (optionally, chemically treated) coated with one or more endophytes described herein and formulation control (lacking the one or more heterologously disposed endophytes) and untreated controls (lacking formulation and the one or more heterologously disposed endophyte) as described in Example 4. Seeds are placed onto each pot and lightly covered with potting mix. Replicated pots of each treatment are set up and placed on a greenhouse bench using a random block design. For example, 18 replicates are planted for each treatment and control. Nitrogen deficit is introduced by reducing the Nitrogen in the Hoagland's solution (3 mM N), which is used to water the plants. Plants are monitored daily for emergence and watered as necessary to maintain a moist but not saturated soil surface (for example, plants are watered with 125 ml Hoagland's solution (3 mM N) per pot on every Monday, Wednesday and Friday).

The following growth and vigor metrics are collected for each treatment: percentage emergence at Day 4, 5, 7 (for soybean, winter wheat and cotton) or Day 3, 4, 5 (for corn), leaf count (the number of fully expanded leaves on the main stem) at Days 10, 17 and 24.

Additional vigor and growth metrics may be collected including shoot height, leaf area, number of chlorotic leaves, chlorophyll content, number of live leaves, etc. At harvest plants are gently removed from pots, washed with tap water to remove dirt, and photographed. Plant tissue is collected for nutrient composition analysis. Plants are put into a paper bag and dried in an oven. Optionally, the plant is separated into shoot and root tissue prior to drying. The dry weight of each individual plant, or shoot or root thereof, is recorded.

### Example 7. Greenhouse Assessment of Improved Plant Characteristics Under Phosphorus Deficit

This example describes an exemplary method by which improved plant health of endophyte treated plants may be shown in a growth environment comprising a phosphorus deficit.

This greenhouse assay is conducted in individual plastic pots, filled with moistened potting soil. This greenhouse assay is conducted using seeds (optionally, chemically treated) coated with one or more endophytes described herein and formulation control (lacking the one or more heterologously disposed endophytes) and untreated controls (lacking formulation and the one or more heterologously disposed endophyte) as described in Example 4. Seeds are placed onto each pot and lightly covered with potting mix. Replicated pots of each treatment are set up and placed on a greenhouse bench using a random block design. For example, 16 replicates are planted for each treatment and control. Phosphorus deficit is introduced by removing Phosphorus from the Hoagland's solution (0 mM P), which is used to water the plants. Plants are monitored daily for emergence and watered as necessary to maintain a moist but not saturated soil surface (for example, plants are watered with 125 ml Hoagland's solution (0 mM P) per pot on every Monday, Wednesday and Friday).

The following growth and vigor metrics are collected for each treatment: percentage emergence at Day 4, 5, 7 (for soybean, winter wheat and cotton) or Day 3, 4, 5 (for corn), leaf count (the number of fully expanded leaves on the main stem) at Days 10, 17 and 24.

Additional vigor and growth metrics may be collected including shoot height, leaf area, coloration of leaves, number of live leaves, etc. At harvest plants are gently removed from pots, washed with tap water to remove dirt, and photographed. Plant tissue is collected for nutrient composition analysis. Plants are put into a paper bag and dried in an oven. Optionally, the plant is separated into shoot and root tissue prior to drying. The dry weight of each individual plant, or shoot or root thereof, is recorded.

### Example 8. Greenhouse Assessment of Improved Plant Health Under Biotic Stress

This example describes an exemplary method by which improved plant health of endophyte treated plants was shown in a growth environment comprising the crop pathogen Rhizoctonia solani or Pythium ultimum, causal agents of seedling damping off disease. This assay may utilize dicots or monocots, though results for soybean, cotton and wheat are described here.

Preparation of pathogen inoculum A stock of Rhizoctonia solani anastomosis group 4 or Pythium ultimum var. ultimum was grown on a standard potato dextrose agar plate. Plugs of fresh mycelium were then transferred into standard potato dextrose broth. After sufficient growth was achieved, the culture was poured though cheesecloth to capture the fungal biomass, which was subsequently rinsed with water. After removing excess rinse water, a roughly equivalent volume of water was added to the fungal biomass before blending to create a slurry. The resulting slurry was further diluted to the required concentration necessary to observe desired level of symptoms.

Greenhouse assay setup The greenhouse assay was conducted in a commercial potting mix. A divot was placed in the center of a pot containing wetted soil using a standardized dibble. An appropriate volume of slurry was added to the center of each divot.

This greenhouse assay was conducted using seeds coated with one or more endophytes described herein and formulation control (lacking the one or more heterologously disposed endophytes) and untreated controls (lacking formulation and the one or more heterologously disposed endophyte) as described in Example 4. Seeds were placed onto each divot after addition of the inoculum. The seeds were then covered with uninoculated soil and again watered. High soil moisture levels were maintained throughout the course of the experiment. Replicates were included in a randomized design to obtain sufficient statistical power for analysis. Plants were grown in a controlled environment until approximately 4 days post emergence of control plants. At this point fresh shoot weight was measured on a per plant basis. The assay was repeated multiple times for some endophytes, results are shown in Tables 6 and 7.

TABLE 6
Greenhouse screening of endophytes with activity against
Pythium, each line in the table represents an experiment

| | | | | % Delta |
|---|---|---|---|---|
| | | | | relative to |
| MIC ID # | Crop | Stressor | Metric | Control |
| MIC-84302 | Winter wheat | Pythium | Shoot Fresh Weight | −1.02 |
| MIC-84302 | Winter wheat | Pythium | Shoot Fresh Weight | −4.07 |
| MIC-67967 | Winter wheat | Pythium | Shoot Fresh Weight | −42.78 |
| MIC-18905 | Winter wheat | Pythium | Shoot Fresh Weight | 136.15 |
| MIC-18905 | Winter wheat | Pythium | Shoot Fresh Weight | 68.75 |
| MIC-18905 | Winter wheat | Pythium | Shoot Fresh Weight | 92.09 |
| MIC-54347 | Winter wheat | Pythium | Shoot Fresh Weight | −9.53 |
| MIC-84302 | Soybean | Pythium | Shoot Fresh Weight | 88.86 |
| MIC-67967 | Soybean | Pythium | Shoot Fresh Weight | 22.32 |
| MIC-67967 | Soybean | Pythium | Shoot Fresh Weight | −1.76 |
| MIC-18905 | Soybean | Pythium | Shoot Fresh Weight | 4.53 |
| MIC-18905 | Soybean | Pythium | Shoot Fresh Weight | 7.21 |
| MIC-18905 | Soybean | Pythium | Shoot Fresh Weight | 43.16 |
| MIC-18905 | Soybean | Pythium | Shoot Fresh Weight | 81.68 |
| MIC-54347 | Soybean | Pythium | Shoot Fresh Weight | 0.4 |
| MIC-54347 | Soybean | Pythium | Shoot Fresh Weight | 14.19 |
| MIC-54347 | Soybean | Pythium | Shoot Fresh Weight | 68.62 |
| MIC-54347 | Soybean | Pythium | Shoot Fresh Weight | 113.51 |
| MIC-54347 | Soybean | Pythium | Shoot Fresh Weight | −4.15 |
| MIC-54347 | Soybean | Pythium | Shoot Fresh Weight | −41.18 |
| MIC-18905 | Cotton | Pythium | Shoot Fresh Weight | 14.23 |
| MIC-18905 | Cotton | Pythium | Shoot Fresh Weight | −41.34 |

TABLE 7
Greenhouse screening of endophytes with activity against
Rhizoctonia, each line in the table represents an experiment

| | | | | | % Delta |
|---|---|---|---|---|---|
| | | | | | relative to |
| MIC # | Crop | Stressor | Metric | Experiment ID | Control |
| MIC-84302 | Winter | Rhizoctonia | Shoot Fresh Weight | FSV0014_13 | 12.45 |
| | wheat | | | | |
| MIC-84302 | Winter | Rhizoctonia | Shoot Fresh Weight | FSV0145_22 | −12.46 |
| | wheat | | | | |
| MIC-67967 | Winter | Rhizoctonia | Shoot Fresh Weight | FSV0032_12 | −14.04 |
| | wheat | | | | |
| MIC-67967 | Winter | Rhizoctonia | Shoot Fresh Weight | FSV0034_14 | −10.37 |
| | wheat | | | | |
| MIC-67967 | Winter | Rhizoctonia | Shoot Fresh Weight | FSV0041_16 | −21.83 |
| | wheat | | | | |
| MIC-18905 | Winter | Rhizoctonia | Shoot Fresh Weight | FSV0040_12 | 72.48 |
| | wheat | | | | |
| MIC-18905 | Winter | Rhizoctonia | Shoot Fresh Weight | FSV0145_22 | −18.22 |
| | wheat | | | | |
| MIC-18905 | Winter | Rhizoctonia | Shoot Fresh Weight | FSV0146_21 | −2.9 |
| | wheat | | | | |
| MIC-54347 | Winter | Rhizoctonia | Shoot Fresh Weight | FSV0008_18 | 1.07 |
| | wheat | | | | |
| MIC-54347 | Winter | Rhizoctonia | Shoot Fresh Weight | FSV0023_16 | 26.72 |
| | wheat | | | | |
| MIC-54347 | Winter | Rhizoctonia | Shoot Fresh Weight | FSV0133_64 | −8.13 |
| | wheat | | | | |
| MIC-54347 | Winter | Rhizoctonia | Shoot Fresh Weight | FSV0142_61 | 143.96 |
| | wheat | | | | |
| MIC-84302 | Soybean | Rhizoctonia | Shoot Fresh Weight | FSV0145_02 | −7.28 |
| MIC-67967 | Soybean | Rhizoctonia | Shoot Fresh Weight | FSV0032_11 | 35.61 |
| MIC-67967 | Soybean | Rhizoctonia | Shoot Fresh Weight | FSV0041_11 | 1.95 |
| MIC-18905 | Soybean | Rhizoctonia | Shoot Fresh Weight | FSV0040_11 | 10.42 |
| MIC-18905 | Soybean | Rhizoctonia | Shoot Fresh Weight | FSV0133_42 | 29.2 |
| MIC-18905 | Soybean | Rhizoctonia | Shoot Fresh Weight | FSV0145_02 | −37.67 |
| MIC-54347 | Soybean | Rhizoctonia | Shoot Fresh Weight | FSV0008_17 | 32.73 |
| MIC-54347 | Soybean | Rhizoctonia | Shoot Fresh Weight | FSV0030_11 | 56.17 |
| MIC-54347 | Soybean | Rhizoctonia | Shoot Fresh Weight | FSV0034_15 | 33.48 |
| MIC-54347 | Soybean | Rhizoctonia | Shoot Fresh Weight | FSV0142_21 | 202.2 |
| MIC-54347 | Soybean | Rhizoctonia | Shoot Fresh Weight | FSV0153_43 | 2.73 |
| MIC-84302 | Cotton | Rhizoctonia | Shoot Fresh Weight | FSV0132_22 | −14.36 |
| MIC-18905 | Cotton | Rhizoctonia | Shoot Fresh Weight | FSV0069_02 | −51.71 |
| MIC-18905 | Cotton | Rhizoctonia | Shoot Fresh Weight | FSV0112_16 | −30.19 |
| MIC-54347 | Cotton | Rhizoctonia | Shoot Fresh Weight | FSV0112_13 | 16.66 |

### Example 9. Soybean Cyst Nematode Preparation

The eggs of Heterodera glycines are extracted from soybean stock culture and are used as inoculum for in vitro, growth chamber, greenhouse, and microplot experiments.

In one embodiment, the following method is used. Eggs are extracted from a 60-day-old soybean stock culture maintained in, e.g., 500 ml polystyrene pots. The soil is gently washed from the soybean roots and cysts and females are dislodged from the roots. Water with the cyst and female suspension is poured through nested 850-μm-pore and 250-μm-pore sieves to separate trash from cysts and females. Cysts and females are ground with a mortar and pestle to release the eggs. Eggs are washed with water, collected on a 25-μm-pore sieve, transferred to two 50 ml centrifuge tubes, and spun for 5 minutes at 1,750 r.p.m. The supernatant liquid is then poured off and a sugar solution added (1 lb. cane sugar, 1 liter water), thoroughly mixing sugar solution and sediment. The suspension is centrifuged at 240 g for 1 minute. The supernatant containing the nematodes is poured on to the 25-μm-pore sieve. After rinsing the sugar away with water, the nematodes are ready for use. For in vitro tests, H. glycines eggs are placed in a modified Baermann funnel (Castillo JD., Lawrence K S., Kloepper J W. Biocontrol of the reniform nematode by Bacillus firmus GB126 and Paecilomyces lilacinus 251 on cotton. Plant Disease. 2013; 97: 967-976) on a Slide Warmer (Model 77) (Marshall Scientific, Brentwood, NH) and incubated at 31° C. for 5 to 7 days to obtain the J2. The J2 are collected on a 25-μm-pore sieve, transferred to 1.5 ml microcentrifuge tubes, centrifuged at 5,000 g for 1 minute, rinsed with sterile distilled water, and centrifuged at 5,000 g for 1 minute. The J2 suspensions are adjusted to 30 to 40 J2 per 10 μl of water. Eggs are enumerated at 40× magnification with an inverted TS100 Nikon microscope and standardized to 2,000 eggs per 500 ml polystyrene pot.

### Example 10. Greenhouse Assessment of Improved Plant Health Under Biotic Stress (Soybean Cyst Nematode)

This example describes an exemplary method by which improved plant health of endophyte treated plants may be shown in a growth environment comprising the crop pest soybean cyst nematode (Heterodera glycines).

Greenhouse assays are conducted using soybean seeds (optionally, chemically treated soybean seeds) coated with one or more of the endophytes described herein and formulation control (lacking the one or more heterologously disposed endophytes) and untreated controls (lacking formulation and the one or more heterologously disposed endophyte) as described in Example 4. Microbe treated soybean seeds are planted, infected with nematodes, maintained, and phenotyped in grow rooms.

In one embodiment, the following method is used. 98 cones are placed in each cone-tainer to obtain the needed number of cone-tainers. Masks are placed over cones and cones are filled with soil. The cone-tainer is place in a deep pan and water is added until the soil in the cones is saturated. Two soybean seeds are planted 2.5 cm deep in each cone-tainer. Each cone-tainer is placed in a growth tub and watered.

One ml containing 2,000 H. glycines eggs is pipetted into each cone-tainer at planting or the desired number of days after planting. Seedlings are thinned to one per cone-tainer after emergence and watered as appropriate.

Phenotyping is performed as follows. The height of each plant is measured, e.g., by placing the ruler on the lip of a cell and measuring the plant's height to the nearest millimeter. The mass of each plant is measured, e.g., by cutting the plant at the soil surface, placing the shoot in the weighing container, allowing the weight to stabilize, and autorecording the mass via the scale's software. The number of H. glycines cysts may be counted after extraction from soybean roots as described herein. The water suspension containing 150 cm\hat{ }3 of soil is poured through nested 75-μm and 25-μm-pore sieves to extract vermiform stages (juveniles and males). Vermiform stages are collected on the 75-μm-pore sieve and centrifuged using, e.g., the sucrose centrifugation-flotation method.

### Example 11. Greenhouse Assessment of Improved Plant Health Under Biotic Stress (Soybean Aphid)

This example describes an exemplary method by which improved plant health of endophyte treated plants may be shown in a growth environment comprising the crop pest soybean aphid (Aphis glycines).

Greenhouse assays are conducted using soybean seeds (optionally, chemically treated soybean seeds) coated with one or more of the endophytes described herein and formulation control (lacking the one or more heterologously disposed endophytes) and untreated controls (lacking formulation and the one or more heterologously disposed endophyte) as described in Example 4. Microbe treated soybean seeds are planted, infected with soybean aphids (Aphis glycines), maintained in grow rooms, and phenotyped.

In one embodiment, the following method is used. 98 cones are placed in each cone-tainer to obtain the needed number of cone-tainers. Masks are placed over cones and cones are filled with potting medium or soil. The cone-tainer is place in a deep pan and water is added until the soil in the cones is saturated. One soybean seed is planted in each cone-tainer. Each cone-tainer is placed in a growth tub and watered.

A community of soybean aphids is maintained on a stock of soybean plants. To prepare for infestation of the experimental plants, leaves are removed from infested soybean plants from the stock community. One or more leaves are examined under a stereoscope to make sure the aphids are alive and vigorous. Infested leaf cutlets are placed in square plates to maintain leaves alive until the treatment plants are infested with aphids. In some embodiments, 20 infested leaf cutlets are used per each 98 cone tray used in the experiment. The infested leaf cutlets are introduced to the growth environment of the experimental plants at planting or the desired number of days after planting, in some embodiments, 9 days after planting. The experimental cone-tainers are infested following an infestation pattern to allow for aphid choice feeding in planta. The infested experimental plants are maintained in their growth environment until phenotyping.

The plants may be phenotyped at one or more times after infestation, for example 1 day, 4 days, 7 days or more after infestation. Measurement of one or more traits of agronomic importance is performed as follows. The height of each plant is measured, e.g., by placing the ruler on the lip of a cell and measuring the plant's height to the nearest millimeter or using an automated tool such as a Phenospex PlantEye 3D laser scanner (Phenospex B.V., Heerlen, The Netherlands). Other traits of agronomic importance may be measured either manually or using a tool such as the Phenospex PlantEye 3D laser scanner, for example the greenness of the plants and the leaf and/or above ground plant area. The mass of each plant may be measured for example via destructive sampling, e.g., by cutting the plant at the soil surface, placing the shoot in the weighing container, allowing the weight to stabilize, and autorecording the mass via the scale's software. The experimental plants may be maintained through their reproductive stages, and traits of agronomic importance such as number of flowers, number of pods and number of seeds per pod may be measured.

### Example 12. Greenhouse Assessment of Improved Plant Health Under Biotic Stress

This example describes an exemplary method by which improved plant health of endophyte treated plants was shown in a growth environment comprising the crop pathogen Fusarium sp., one of the causal agents of seedling damping off disease. This assay may utilize dicots or monocots, including, for example, soybean and wheat as shown here.

Preparation of Fusarium sp. inoculum A stock of Fusarium sp. was grown on a standard potato dextrose agar plate. Plugs of fresh mycelium were then transferred into breathable bag containing a sterile mixture of water and grain such as sorghum or millet. After sufficient growth is achieved, the culture was removed from the bags and dried. After drying the biomass was coarsely ground.

Greenhouse assay setup The greenhouse assay was conducted in a media mixture consisting of a commercial potting mix and a minimum of 50% inert inorganic material. An appropriate volume of ground pathogen was added to the soil mixture to obtain moderate to severe symptoms.

This greenhouse assay was conducted using seeds coated with one or more endophytes described herein and formulation control (lacking the one or more heterologously disposed endophytes) and untreated controls (lacking formulation and the one or more heterologously disposed endophyte) as described in Example 4. A seed was added to the surface of the infested media. The seed was then covered with media lacking pathogen and again watered. High soil moisture levels were maintained throughout the course of the experiment. Replicates were included in a randomized design to obtain sufficient statistical power for analysis. Plants were grown in a controlled environment until approximately 4 days post emergence of control plants. At this point shoot fresh weight was measured on a per plant basis. The assay was repeated multiple times for some endophytes, results are shown in Table 8.

TABLE 8
Greenhouse screening of endophytes with activity against
Fusarium, each line in the table represents an experiment

| | | | | % Delta |
|---|---|---|---|---|
| | | | | relative |
| | | | | to |
| | | | | Stress |
| MIC ID# | Crop | Stressor | Metric | Control |
| MIC-84302 | Winter wheat | Fusarium | Shoot Fresh Weight | 64.34 |
| MIC-84302 | Winter wheat | Fusarium | Shoot Fresh Weight | 46.2 |
| MIC-67967 | Winter wheat | Fusarium | Shoot Fresh Weight | 34.97 |
| MIC-67967 | Winter wheat | Fusarium | Shoot Fresh Weight | −9.15 |
| MIC-67967 | Winter wheat | Fusarium | Shoot Fresh Weight | 11.37 |
| MIC-67967 | Winter wheat | Fusarium | Shoot Fresh Weight | 4.91 |
| MIC-18905 | Winter wheat | Fusarium | Shoot Fresh Weight | 3.79 |
| MIC-18905 | Winter wheat | Fusarium | Shoot Fresh Weight | −15.23 |
| MIC-18905 | Winter wheat | Fusarium | Shoot Fresh Weight | −42.18 |
| MIC-18905 | Winter wheat | Fusarium | Shoot Fresh Weight | 29.41 |
| MIC-54347 | Winter wheat | Fusarium | Shoot Fresh Weight | 41.74 |
| MIC-54347 | Winter wheat | Fusarium | Shoot Fresh Weight | 66.32 |
| MIC-54347 | Winter wheat | Fusarium | Shoot Fresh Weight | −8.71 |
| MIC-54347 | Winter wheat | Fusarium | Shoot Fresh Weight | 29.77 |
| MIC-54347 | Winter wheat | Fusarium | Shoot Fresh Weight | 52.66 |
| MIC-84302 | Soybean | Fusarium | Shoot Fresh Weight | 28.41 |
| MIC-84302 | Soybean | Fusarium | Shoot Fresh Weight | −29.38 |
| MIC-84302 | Soybean | Fusarium | Shoot Fresh Weight | −12.55 |
| MIC-67967 | Soybean | Fusarium | Shoot Fresh Weight | 16.92 |
| MIC-67967 | Soybean | Fusarium | Shoot Fresh Weight | 59.68 |
| MIC-67967 | Soybean | Fusarium | Shoot Fresh Weight | 2.8 |
| MIC-67967 | Soybean | Fusarium | Shoot Fresh Weight | 4.76 |
| MIC-18905 | Soybean | Fusarium | Shoot Fresh Weight | 69.36 |
| MIC-18905 | Soybean | Fusarium | Shoot Fresh Weight | 46.11 |
| MIC-18905 | Soybean | Fusarium | Shoot Fresh Weight | 74.47 |
| MIC-18905 | Soybean | Fusarium | Shoot Fresh Weight | 39.84 |
| MIC-54347 | Soybean | Fusarium | Shoot Fresh Weight | 14.7 |
| MIC-54347 | Soybean | Fusarium | Shoot Fresh Weight | 9.11 |
| MIC-54347 | Soybean | Fusarium | Shoot Fresh Weight | 31.04 |
| MIC-54347 | Soybean | Fusarium | Shoot Fresh Weight | 13.63 |

### Example 13. Field Assessment of Improved Plant Health of Soy Under Biotic Stress

This example describes an exemplary method by which improved plant health of endophyte treated plants may be shown in a growth environment comprising the crop pests root knot nematode (Meloidogyne incognita), Reniform nematode (Rotylenchulus reniformis), and, opportunistically, the fungal pathogen Fusarium virguliforme.

Field trials are conducted using chemically treated soy seeds coated with one or more of the endophytes described herein and formulation control (lacking the one or more heterologously disposed endophytes) and untreated controls (lacking formulation and the one or more heterologously disposed endophyte) as a flowable powder described in Example 4. Plots for in-field assessment harbor populations of root knot nematode and Reniform nematode, respectively, at an approximately 1.0+E04 eggs per gram of fresh root weight. Opportunistically, these plots are infected with natural inoculum of Fusarium virguliforme, the causal agent of Fusarium Sudden Death Syndrome (SDS). Replicate plots, preferably at least 4 replicate plots, are planted per endophyte or control treatment in a randomized complete block design. Each plot consists of a 7.62 m (25 ft.) by 0.76 m (2.5 ft.) row. The following early growth metrics are measured: percent emergence at 14 days post planting, standing count at 28 and 45 days post planting, plant vigor at 14, 28, and 45 days post planting, plant height at 45 days post planting, fresh shoot weight, fresh root weight, disease rating at a 0-3 scale (3 denotes strong disease symptoms) using the split-root scoring system at 45 days post planting, nematode count at 45 days post planting, and yield parameters.

At the end of the field trial employing endophyte treatment and control treatment plants, plants (preferably at least 4 plants) are randomly dug out from each row, kept in a plastic bag, and brought back to lab. For each seedling, shoot and root are separated by cutting the seedling 3 cm from the first branch of the root. The heights of the separated shoot of each plant are measured, followed by fresh shoot weight, and fresh root weight. The main root is vertically split into two halves and discoloration of xylem is scored as described above. To extract and count nematode eggs on root, roots are place in a container prefilled with 100 ml 10% sucrose and incubated on a shaker at room temperature overnight. The supernatant is then collected and nematode eggs are counted under a stereomicroscope.

The percentage of survival plants, fresh root weight, and nematode egg count are plotted as bar graph of mean±95% confidence interval from the mean using the ggplot2 package of R (R Core Team, 2016. R: A language and environment for statistical computing. R Foundation for Statistical Computing, Vienna, Austria. R-project.org/). Plant heights, fresh shoot weight, and disease scores are plotted as jittered dot of mean±nonparametric bootstrap (1000) of 95% confidence interval from the mean using the ggplot2 package of R.

### Example 14. Field Assessment of Improved Plant Health of Cotton Under Biotic Stress

This example describes an exemplary method by which improved plant health of endophyte treated plants may be shown in a growth environment comprising the crop pests root knot nematode (Meloidogyne incognita), Reniform nematode (Rotylenchulus reniformis), and, opportunistically, the fungal pathogen Fusarium virguliforme.

Field trials are conducted using chemically treated cotton seeds coated with one or more of the endophytes described herein and formulation control (lacking the one or more heterologously disposed endophytes) and untreated controls (lacking formulation and the one or more heterologously disposed endophyte) as described in Example 4. Plots for in-field assessment harbor populations of root knot nematode and Reniform nematode, respectively, at an approximately 1.0+E04 eggs per gram of fresh root weight. Opportunistically, these plots are infected with natural inoculum of Fusarium virguliforme, the causal agent of Fusarium SDS. Replicate plots, preferably at least 4 replicate plots, are planted per endophyte or control treatment in a randomized complete block design. Each plot consists of a 7.62 m (25 ft.) by 0.76 m (2.5 ft.) row. The following early growth metrics are measured: percent emergence at 14 days post planting, standing count at 28 and 45 days post planting, plant vigor at 14, 28, and 45 days post planting, plant height at 45 days post planting, fresh shoot weight, fresh root weight, disease rating at a 0-3 scale (3 denotes strong disease symptoms) using the split-root scoring system at 45 days post planting, nematode count at 45 days post planting, and yield parameters.

At the end of the field trial employing endophyte treatment and control treatment plants, plants (preferably at least 4 plants) are randomly dug out from each row, kept in a plastic bag, and brought back to lab for metric measurements. For each seedling, shoot and root are separated by cutting the seedling 3 cm from the first branch of the root. The heights of the separated shoot of each plant are measured, followed by fresh shoot weight, and fresh root weight. The main root is vertically split into two halves and discoloration of xylem is scored as described above. To extract and count nematode eggs on root, roots are placed in a container prefilled with 100 ml 10% sucrose and incubated on a shaker at room temperature overnight. The supernatant is then collected and nematode eggs are counted under a stereomicroscope.

The percentage of survival plants, fresh root weight, and nematode egg count are plotted as bar graph of mean±95% confidence interval from the mean using the ggplot2 package of R (R Core Team, 2016. R: A language and environment for statistical computing. R Foundation for Statistical Computing, Vienna, Austria. R-project.org/). Plant heights, fresh shoot weight, and disease scores are plotted as jittered dot of mean±nonparametric bootstrap (1000) of 95% confidence interval from the mean using the ggplot2 package of R.

### Example 15. Field Assessment of Improved Plant Health of Winter Wheat Under Biotic Stress

This example describes a method for detection of improved plant health of endophyte treated winter wheat in a growth environment comprising the crop pathogens Rhizoctonia spp., Pythium spp., and Fusarium spp (causal agents of damping-off disease).

Field trials were conducted using winter wheat seeds coated with MIC-84302 and untreated controls (lacking formulation and the heterologously disposed endophyte). Rhizoctonia, Fusarium, and Pythium inoculant were applied per standard practice to each seed packet before planting. Five replicate plots were planted per endophyte treatment and control treatment in a randomized complete block design. Each plot consisted of a 6 ft. by 20 ft. block. Irrigation was applied pre-planting and in early season to maximize disease pressure.

Plots were harvested by machine, and yield was calculated by the on-board computer.

TABLE 9
Yield of endophyte treated winter wheat under biotic stress.

| | | | | % Delta |
|---|---|---|---|---|
| | SEQ ID | | | relative to |
| MIC# | NO: | Crop | Metric | Control |
| MIC-84302 | 132-136 | Winter wheat | Yield | 7.3 |

### Example 16. Field Assessment of Improved Plant Health of Corn Under Biotic Stress

This example describes a method for detection of improved plant health of endophyte treated corn in a growth environment comprising the crop pathogen Fusarium spp.

Field trials were conducted using corn seeds coated with MIC-67967, a control treated with chemical fungicide (lacking formulation and the heterologously disposed endophyte), and untreated controls (lacking formulation and the heterologously disposed endophyte). Fusarium inoculant was applied per standard practice to each seed packet before planting, targeting moderate level of disease infestation; enough to affect plant stand, but not to a level resulting in total loss. Five replicate plots were planted per endophyte treatment and control treatment in a randomized complete block design. Each plot consisted of a 25 ft. long, 2-4 row block.

Plots were harvested by machine, and yield was calculated by the on-board computer.

TABLE 10
Yield of endophyte treated corn under biotic stress.

| | | | | % Delta |
|---|---|---|---|---|
| | SEQ ID | | | relative to |
| MIC# | NO: | Crop | Metric | Control |
| MIC-67967 | 127-131 | Corn | Yield | 8.3 |
| MIC-67967 | 127-131 | Corn | Emergence | −8.6 |
| MIC-67967 | 127-131 | Corn | Plant Height | 11.2 |
| MIC-67967 | 127-131 | Corn | Shoot Weight | 33.7 |
| MIC-67967 | 127-131 | Corn | Root Weight | 51.5 |

### Example 17. Field Assessment of Improved Plant Health of Corn Under Biotic Stress

This example describes a method for detection of improved plant health of endophyte treated cotton in a growth environment comprising the crop pathogen Fusarium spp.

Field trials were conducted using cotton seeds coated with MIC-84302, a control treated with chemical fungicide (lacking formulation and the heterologously disposed endophyte), and untreated controls (lacking formulation and the heterologously disposed endophyte). Fusarium inoculant is applied per standard practice to each seed packet before planting, targeting moderate level of disease infestation; enough to affect plant stand, but not to a level resulting in total loss. Five replicate plots are planted per endophyte treatment and control treatment in a randomized complete block design. Each plot consists of a 25 ft. long, 2-4 row block. moderate level of disease infestation; enough to affect plant stand, but not to a level resulting in total loss

Plots were harvested by machine, and yield was calculated by the on-board computer.

TABLE 11
Yield of endophyte treated cotton under biotic stress.

| | | | | % Delta |
|---|---|---|---|---|
| | SEQ ID | | | relative to |
| MIC# | NO: | Crop | Metric | Control |
| MIC-84302 | 132-136 | Cotton | Yield | 46.6 |
| MIC-84302 | 132-136 | Cotton | Shoot weight | 21.1 |
| MIC-84302 | 132-136 | Cotton | Root weight | 33.1 |
| MIC-84302 | 132-136 | Cotton | Plant height | 14.1 |
| MIC-84302 | 132-136 | Cotton | Stand count | 16.1 |

### Example 18. Field Assessment of Improved Plant Health of Soybean Under Biotic Stress

This example describes a method for detection of improved plant health of endophyte treated soybean in a growth environment comprising the crop pathogen Pythium sp., Rhizoctonia sp., and Fusarium sp.

Field trials were conducted using soybean seeds coated with a heterologously disposed endophyte treatment (MIC-54347), a control treated with chemical fungicide (lacking formulation and the heterologously disposed endophyte), and untreated controls (lacking formulation and the heterologously disposed endophyte). Pythium ultimum inoculant was applied in furrow, targeting moderate level of disease infestation; enough to affect plant stand, but not to a level resulting in total loss. Twelve data points were obtained from trials inoculated with the pathogen where stand reduction of at least 5% (or significant loss) occurred in the non-treated control relative to the chemically treated control. At least four replicate plots were planted per endophyte treatment and control treatment in a randomized complete block design. Each plot consisted of approximately a 25 ft. long, 2-4 row block.

Plots are harvested by machine, and yield is calculated by the on-board computer.

Early emergence is the number of emerged plants per acre, measured 0-2 days after the beginning of emergence. Full emergence is the number of emerged plants per acre, measured 10 days after the beginning of emergence. Plant height is the height of five plants per plot, measured 14-21 days after full emergence. Root weight is the weight of roots (cut at the soil line) of five plants per plot, measured 14-21 days after full emergence. Shoot weight is the weight of shoots (cut at soil line) of five plants per plot, measured 17 days after full emergence.

Soybean plants treated with MIC-54347 showed a 13.9% increase in shoot weight over untreated controls, with a 83.5% win-rate.

TABLE 12
Yield of endophyte treated soybean under biotic stress (Pythium)

| | | | | % Delta |
|---|---|---|---|---|
| | SEQ | | | relative to |
| | ID | | | untreated |
| MIC# | NO: | Crop | Metric | control |
| Chemical control | | Soybean | Early emergence | −9.4 |
| Chemical control | | Soybean | Full emergence | 14.1 |
| Chemical control | | Soybean | Plant height | 1.6 |
| Chemical control | | Soybean | Root weight | 17.0 |
| Chemical control | | Soybean | Shoot weight | 7.4 |
| Chemical control | | Soybean | Yield | 2.1 |
| MIC-54347 | 32-126 | Soybean | Early emergence | 1.5 |
| MIC-54347 | 32-126 | Soybean | Full emergence | 7.6 |
| MIC-54347 | 32-126 | Soybean | Plant height | 2.6 |
| MIC-54347 | 32-126 | Soybean | Root weight | 11.8 |
| MIC-54347 | 32-126 | Soybean | Shoot weight | 13.9 |
| MIC-54347 | 32-126 | Soybean | Yield | 3.4 |

Field trials were conducted using soybean seeds coated with a heterologously disposed endophyte treatment (MIC-54347), a control treated with chemical fungicide (lacking formulation and the heterologously disposed endophyte), and untreated controls (lacking formulation and the heterologously disposed endophyte). Rhizoctonia solani inoculant was applied per standard practice to each seed packet before planting, targeting moderate level of disease infestation; enough to affect plant stand, but not to a level resulting in total loss. Twelve data points were obtained from trials inoculated with the pathogen where stand reduction of at least 5% (or significant loss) occurred in the non-treated control relative to the chemically treated control (only six data points were obtained for yield data). At least four replicate plots were planted per endophyte treatment and control treatment in a randomized complete block design. Each plot consisted of approximately a 25 ft. long, 2-4 row block.

Plots are harvested by machine, and yield is calculated by the on-board computer.

Early emergence is the number of emerged plants per acre, measured 0-2 days after the beginning of emergence. Full emergence is the number of emerged plants per acre, measured 10 days after the beginning of emergence. Plant height is the height of five plants per plot, measured 14-21 days after full emergence. Root weight is the weight of roots (cut at the soil line) of five plants per plot, measured 14-21 days after full emergence. Shoot weight is the weight of shoots (cut at soil line) of five plants per plot, measured 14-21 days after full emergence.

TABLE 12
Yield of endophyte treated soybean under
biotic stress (Rhizoctonia).

| | | | | % Delta |
|---|---|---|---|---|
| | SEQ | | | relative to |
| | ID | | | untreated |
| MIC# | NO: | Crop | Metric | control |
| Chemical control | | Soybean | Early emergence | 12.6 |
| Chemical control | | Soybean | Full emergence | 58.3 |
| Chemical control | | Soybean | Plant height | 2.6 |
| Chemical control | | Soybean | Root weight | −6.4 |
| Chemical control | | Soybean | Shoot weight | 13.4 |
| Chemical control | | Soybean | Yield | 34.5 |
| MIC-54347 | 32-126 | Soybean | Early emergence | 2.0 |
| MIC-54347 | 32-126 | Soybean | Full emergence | 10.7 |
| MIC-54347 | 32-126 | Soybean | Plant height | −0.2 |
| MIC-54347 | 32-126 | Soybean | Root weight | 5.8 |
| MIC-54347 | 32-126 | Soybean | Shoot weight | 8.3 |
| MIC-54347 | 32-126 | Soybean | Yield | 17.6 |

Field trials were conducted using soybean seeds coated with a heterologously disposed endophyte treatment (MIC-54347), a control treated with chemical fungicide (lacking formulation and the heterologously disposed endophyte), and untreated controls (lacking formulation and the heterologously disposed endophyte). Fusarium graminearum inoculant was applied in furrow, targeting moderate level of disease infestation; enough to affect plant stand, but not to a level resulting in total loss. Twelve data points were obtained from trials inoculated with the pathogen where stand reduction of at least 5% (or significant loss) occurred in the non-treated control relative to the chemically treated control. At least four replicate plots were planted per endophyte treatment and control treatment in a randomized complete block design. Each plot consisted of approximately a 25 ft. long, 2-4 row block.

Plots are harvested by machine, and yield is calculated by the on-board computer.

Early emergence is the number of emerged plants per acre, measured 0-2 days after the beginning of emergence. Full emergence is the number of emerged plants per acre, measured 10 days after the beginning of emergence. Plant height is the height of five plants per plot, measured 14-21 days after full emergence. Root weight is the weight of roots (cut at the soil line) of five plants per plot, measured 14-21 days after full emergence. Shoot weight is the weight of shoots (cut at soil line) of five plants per plot, measured 14-21 days after full emergence.

Soybean plants treated with MIC-54347 showed a 2.9% (1.77 bu/acre) increase in yield over untreated controls, with a 75% win-rate.

TABLE 12
Yield of endophyte treated soybean under biotic stress (Fusarium).

| | | | | % Delta |
|---|---|---|---|---|
| | SEQ | | | relative to |
| | ID | | | untreated |
| MIC# | NO: | Crop | Metric | control |
| Chemical control | | Soybean | Early emergence | −25.7 |
| Chemical control | | Soybean | Full emergence | −3.1 |
| Chemical control | | Soybean | Plant height | −4.4 |
| Chemical control | | Soybean | Root weight | 5.3 |
| Chemical control | | Soybean | Shoot weight | 9.3 |
| Chemical control | | Soybean | Yield | 3.0 |
| MIC-54347 | 32-126 | Soybean | Early emergence | −18.6 |
| MIC-54347 | 32-126 | Soybean | Full emergence | −3.5 |
| MIC-54347 | 32-126 | Soybean | Plant height | −0.2 |
| MIC-54347 | 32-126 | Soybean | Root weight | 2.2 |
| MIC-54347 | 32-126 | Soybean | Shoot weight | 1.1 |
| MIC-54347 | 32-126 | Soybean | Yield | 2.9 |

### Example 19. Method of Preparation of Endophytes and Heterologous Disposition of Endophytes on Seeds for Field Trials

### Preparation of Endophytes

Bacteria: An agar plug of each bacterial strain is transferred using a transfer tube to 4 ml of potato dextrose broth (PDB) in a 24 well plate and incubated at room temperature at 675 rpm on a shaker for 3 days. After growth of bacteria in broth, 200 μl is transferred into a spectrophotometer reading plate and bacteria OD is read at 600 nm absorbance. All bacteria strains are then normalized to 0.05 OD utilizing PBS 1× buffer.

Fungi: Preparation of molasses broth and potato dextrose agar: Molasses broth is prepared by dissolving 30 g molasses and 5 g yeast extract per liter deionized water in an autoclavable container and autoclaving (15 psi, 121° C.) for 45 min. Potato dextrose agar (PDA) plates are prepared by dissolving 39.0 g PDA powder per liter deionized water in an autoclavable container and autoclaving (15 psi, 121° C.) for 45 min. The agar is allowed to cool to 50-60° C., before pouring into sterile petri plates (30 ml per 90 mm plate). Fungal endophyte treatments may be applied as either a dry or liquid formulation.

Liquid biomass: All equipment and consumables are thoroughly sterilized and procedures performed in a biosafety cabinet. The inoculant is prepared by placing 1 plug from a cryopreserved stock on a fresh PDA plate, sealing the plate with Parafilm® and incubating at room temperature in the dark for 5-10 days. Then ˜5×5 mm plugs are cut from the PDA plates and 10-12 plugs are transferred into flasks containing the sterile molasses broth, covered, secured in a shaker and incubated for at least 10 days with shaking at ˜130 rpm. Then the culture is placed in a blender for 5 seconds and 1 ml of the blended culture is centrifuged and the supernatant is discarded. The pellet is resuspended in 0.5 ml 1× Phosphate Buffered Saline (PBS) to generate inoculum.

Dry biomass: All equipment and consumables are thoroughly sterilized and procedures performed in a biosafety cabinet. The inoculant is prepared by placing 1 plug from a cryopreserved stock on a fresh PDA plate, sealing the plate with Parafilm® and incubating at room temperature in the dark for 5-10 days. Then ˜5×5 mm plugs are cut from the PDA plates and 10-12 plugs are transferred into flasks containing the sterile molasses broth, covered, secured in a shaker and incubated for at least 10 days with shaking at ˜130 rpm. In sterile conditions, the liquid culture is carefully decanted using 150 mm sterile filter paper on a sterilized Buchner funnel over a sterile flask. Once all liquid passes through the funnel, the pellet is rinsed with sterile water until the filtrate runs clear. When dry, the pellet is transferred to a drying cabinet and dried until brittle. The pellet is then ground into a fine powder, and sample is used to generate CFU counts.

### Preparation of Formulation for Seed Treatments

A 2% weight/volume solution of sodium alginate for the seed coatings is prepared by the following method. An Erlenmeyer flask is filled with the appropriate volume of deionized water and warmed to 50 degrees Celsius on a heat plate with agitation using a stir bar. The appropriate mass of sodium alginate powder for the desired final concentration solution is slowly added until dissolved. The solution is autoclaved at 121 degrees Celsius at 15 PSI for 30 minutes to sterilize.

Talc for the powdered seed coatings is prepared by the following method. Talc is aliquoted into bags or 50 ml Falcon tubes and autoclaved in dry cycle (121 degrees Celsius at 15 PSI for 30 minutes) to sterilize.

### Heterologous Disposition of Endophytes on Seeds

Seeds treated were heterologously disposed to each endophyte according to the following seed treatment protocol.

Liquid formulation: Liquid culture is added to the seeds at a rate of 23 (for fungal endophyte treatments) or 8.4 (for bacterial endophyte treatments) ml per kg of seeds, with equivalent volumes of the prepared sodium alginate. Control treatments are prepared using equivalent volumes of sterile broth. The seeds are then agitated to disperse the solution evenly on the seeds. For fungal endophytes, 15 g per kg of seed of talc powder as prepared above is added and the seeds are agitated to disperse the powder evenly on the seeds. Then 16.6 ml (for fungal endophyte treatments) or 2.4 ml (for bacterial endophyte treatments) per kg of seed of Flo-Rite® 1706 (BASF, Ludwigshafen, Germany) is added and the seeds are agitated to disperse the powder evenly on the seeds. Slightly less Flo-Rite® is used for small grains and canola seeds, slightly more Flo-Rite® is used for seeds such as corn, soy, cotton and peanut seeds. The target dose is generally between 10\hat{ }0-10\hat{ }6 CFU per seed, in some cases at least 10\hat{ }3 CFU per seed, or at least 10\hat{ }4 CFU per seed. Treated seeds are allowed to dry overnight in a well-ventilated space before planting.

Dry formulation: The 2% sodium alginate solution prepared above is added to the seeds at a rate of 23 ml per kg of seeds. Equal parts of dry biomass and talc prepared as above are mixed. The solution is applied so that an equivalent of 10 g of powdered dry biomass is applied per kg of seeds. Control treatments are prepared using equivalent volumes of talc. The seeds are then agitated to disperse the solution evenly on the seeds. Then 16.6 ml per kg of seed of Flo-Rite® 1706 (BASF, Ludwigshafen, Germany) is added and the seeds are agitated to disperse the powder evenly on the seeds. Slightly less Flo-Rite® is used for small grains and canola seeds, slightly more Flo-Rite® is used for seeds such as corn soy, cotton and peanut seeds. The target dose is generally between 10\hat{ }0-10\hat{ }6 CFU per seed, in some cases at least 10\hat{ }3 CFU per seed, or at least 10\hat{ }4 CFU per seed. Treated seeds are allowed to dry overnight in a well-ventilated space before planting.

### Example 20. Field Assessment of Improved Plant Characteristics

### Rice

Field trials are conducted, preferably, at multiple locations. In some embodiments, rice seeds are treated with commercial fungicidal and insecticidal treatment. Seeds are heterologously disposed with the endophyte treatments and formulation control (lacking the one or more heterologously disposed endophytes) as described in Example 18, untreated seeds (lacking formulation and the one or more heterologously disposed endophyte) are also planted. Seeds are sown in regularly spaced rows in soil at 1.2 million seeds/acre seeding density. At each location at least 3 replicate plots are planted for each endophyte or control treatments in a randomized complete block design. For example, each plot may consist of seven, 15.24 m (40 ft.) rows.

At the end of the field trial employing endophyte treatment and control treatment plants, plots are harvested, for example, by machine with a 5-ft research combine and yield is calculated by the on-board computer.

### Wheat

Field trials are conducted at multiple locations with multiple plots per location. Wheat seeds (optionally treated with commercial fungicidal and insecticidal treatments) are heterologously disposed with the endophyte treatments as described in Example 18; untreated seeds (lacking formulation and the one or more heterologously disposed endophyte) are also planted. Seeds are sown in regularly spaced rows in soil at 1.2 million seeds/acre seeding density. At each location at least 3 replicate plots are planted for each endophyte or control treatments in a randomized complete block design. Each plot consists of seven, 15.24 m (40 ft.) rows.

Plots are harvested by machine, for example with a 5-ft research combine and yield was calculated by the on-board computer.

### Corn

Field trials are conducted at multiple locations, preferably with multiple plots per location. Plots may be irrigated, non-irrigated (dryland), or maintained with suboptimal irrigation at a rate to target approximately 25% reduction in yield. In some embodiments, corn seeds are treated with commercial fungicidal and insecticidal treatment. Seeds are heterologously disposed with the endophyte treatments as described in Example 18; untreated seeds (lacking formulation and the one or more heterologously disposed endophyte) are also planted. Seeds are sown in regularly spaced rows in soil at planting densities typical for each region. At each location at least 3 replicate plots are planted per endophyte or control treatment in a randomized complete block design. For examples, each plot may consist of four 15.24 m (40 ft.) rows, each separated by 76.2 cm (30 in).

At the end of the field trial employing endophyte treatment and control treatment plants, plots are harvested, for example, by machine with a 5-ft research combine and yield is calculated by the on-board computer. Only the middle two rows of the 4 row plots are harvested to prevent border effects.

### Soy

Field trials were conducted according to the following methodology. Seeds were heterologously disposed with the endophyte treatment (MIC-54347) as described in Example 18, untreated seeds (lacking formulation and the one or more heterologously disposed endophyte) are also planted. At each location at least 3 replicate plots were planted per endophyte or control treatment in a randomized complete block design), a total of 18 data points were collected. Each plot consisted of four 15.24 m (40 ft.) rows, each separated by 76.2 cm (30 in).

At the end of the field trial employing endophyte treatment and control treatment plants, plots were harvested, by machine with a 5-ft research combine and yield is calculated by the on-board computer. Only the middle two rows of the 4 row plots are harvested to prevent border effects.

Treatment with MIC-54347 was associated with a 10.8% decrease in yield where the natural disease pressure in the fields was low, with a win rate of 28%.

### Canola

Field trials are conducted at multiple locations, preferably in diverse geographic regions. Plots may be irrigated, non-irrigated (dryland) or maintained with suboptimal irrigation at a rate to target approximately 25% reduction in yield. In some embodiments, canola seeds are treated with commercial fungicidal and insecticidal treatment. Seeds are heterologously disposed with the endophyte treatments as described in Example 18, untreated seeds (lacking formulation and the one or more heterologously disposed endophyte) are also planted. At each location, at least 3 replicate plots are planted for each endophyte or control treatment in a randomized complete block design.

At the end of the field trial employing endophyte treatment and control treatment plants, plots are harvested, for example, by machine with a 5-ft research combine and yield is calculated by the on-board computer.

### Peanut

Field trials are conducted at multiple locations, preferably in diverse geographic regions. Optionally, plots are non-irrigated (dryland) or maintained with suboptimal irrigation at a rate to target approximately 25% reduction in yield. In some embodiments, peanut seeds are treated with commercial fungicidal and insecticidal treatment. Seeds are heterologously disposed with the endophyte treatments as described in Example 18, untreated seeds (lacking formulation and the one or more heterologously disposed endophyte) are also planted.

At the end of the field trial employing endophyte treatment and control treatment plants, plots are harvested, for example, by machine with a 5-ft research combine and yield is calculated by the on-board computer.

### Example 21. Method of Determining Seed Nutritional Quality Trait Component: Fat

Seed samples from harvested plants are obtained as described in Example 20. Analysis of fat is conducted on replicate samples according to the Association of Official Agricultural Chemists Reference Method AOAC 920.39, of the Official Methods of Analysis of AOAC International, 20th Edition (2016), herein incorporated by reference in its entirety. Samples are weighed onto filter paper, dried, and extracted in hot hexane for 4 hrs. using a Soxlhet system. Oil is recovered in pre-weighed glassware, and % fat is measured gravimetrically. Mean percent changes between the treatment (endophyte-treated seed) and control (seed treated with the formulation but no endophyte) are calculated.

### Example 22. Method of Determining Seed Nutritional Quality Trait Component: Ash

Seed samples from harvested plants are obtained as described in Example 20. Analysis of ash is conducted on replicate samples according to the Association of Official Agricultural Chemists Reference Method AOAC 920.39, of the Official Methods of Analysis of AOAC International, 20th Edition (2016). Samples are weighed into pre-weighed crucibles, and ashed in a furnace at 600° C. for 3 hr. Weight loss on ashing is calculated as % ash. Mean percent changes between the treatment (one or more heterologously disposed endophytes) and control (lacking the one or more heterologously disposed endophytes) with the formulation but no endophyte are calculated.

### Example 23. Method of Determining Seed Nutritional Quality Trait Component: Fiber

Seed samples from harvested plants are obtained as described in Example 20. Analysis of fiber is conducted on replicate samples according to the Association of Official Agricultural Chemists Reference Method AOAC 920.39, of the Official Methods of Analysis of AOAC International, 20th Edition (2016). Samples are weighed into filter paper, defatted and dried, and hydrolyzed first in acid, then in alkali solution. The recovered portion is dried, weighed, ashed at 600° C., and weighed again. The loss on ashing is calculated as % Fiber. Mean percent changes between the treatment (one or more heterologously disposed endophytes) and control (lacking the one or more heterologously disposed endophytes) with the formulation but no endophyte are calculated.

### Example 24. Method of Determining Seed Nutritional Quality Trait Component: Moisture

Seed samples from harvested plants are obtained as described in Example 20. Analysis of moisture is conducted on replicate samples according to the Association of Official Agricultural Chemists Reference Method AOAC 920.39, of the Official Methods of Analysis of AOAC International, 20th Edition (2016). Samples are weighed into pre-weighed aluminum dishes, and dried at 135° C. for 2 hrs. Weight loss on drying is calculated as % Moisture. Mean percent changes between the treatment (one or more heterologously disposed endophytes) and control (lacking the one or more heterologously disposed endophytes) with the formulation but no endophyte are calculated.

### Example 25. Method of Determining Seed Nutritional Quality Trait Component: Protein

Seed samples from harvested plants are obtained as described in Example 20. Analysis of protein is conducted on replicate samples according to the Association of Official Agricultural Chemists Reference Method AOAC 920.39, of the Official Methods of Analysis of AOAC International, 20th Edition (2016). Samples are combusted and nitrogen gas is measured using a combustion nitrogen analyzer (Dumas). Nitrogen is multiplied by 6.25 to calculate % protein. Mean percent changes between the treatment (one or more heterologously disposed endophytes) and control (lacking the one or more heterologously disposed endophytes) with the formulation but no endophyte) are calculated.

### Example 26. Method of Determining Seed Nutritional Quality Trait Component: Carbohydrate

Seed samples from harvested plants are obtained as described in Example 20. Analysis of carbohydrate is determined for replicate samples as a calculation according to the following formula: Total Carbohydrate=100%−% (Protein+Ash+Fat+Moisture+Fiber), where % Protein is determined according to the method of Example 25, % Ash is determined according to the method of Example 22, % Fat is determined according to the method of Example 21, % Moisture is determined according to the method of Example 24, and % Fiber is determined according to the method of Example 23. Mean percent changes between the treatment (one or more heterologously disposed endophytes) and control (lacking the one or more heterologously disposed endophytes) are calculated.

### Example 27. Method of Determining Seed Nutritional Quality Trait Component: Calories

Seed samples from harvested plants are obtained as described in Example 20. Analysis of Calories is determined for replicate samples as a calculation according to the following formula: Total Calories=(Calories from protein)+(Calories from carbohydrate)+Calories from fat), where Calories from protein are calculated as 4 Calories per gram of protein (as determined according to the method of Example 25), Calories from carbohydrate are calculated as 4 Calories per gram of carbohydrate (as determined according to the method of Example 26), and Calories from fat are calculated as 9 Calories per gram of fat (as determined according to the method of Example 21). Mean percent changes between the treatment (one or more heterologously disposed endophytes) and control (lacking the one or more heterologously disposed endophytes) are calculated.

### Example 28. Additional Methods for Creating Synthetic Compositions

### Osmopriming and Hydropriming

One or more endophytes are inoculated onto seeds during the osmopriming (soaking in polyethylene glycol solution to create a range of osmotic potentials) and/or hydropriming (soaking in de-chlorinated water) process. Osmoprimed seeds are soaked in a polyethylene glycol solution containing one or more endophytes for one to eight days and then air dried for one to two days. Hydroprimed seeds are soaked in water for one to eight days containing one or more endophytes and maintained under constant aeration to maintain a suitable dissolved oxygen content of the suspension until removal and air drying for one to two days. Talc and or flowability polymer are added during the drying process.

### Foliar Application

One or more endophytes are inoculated onto aboveground plant tissue (leaves and stems) as a liquid suspension in dechlorinated water containing adjuvants, sticker-spreaders and UV protectants. The suspension is sprayed onto crops with a boom or other appropriate sprayer.

### Soil Inoculation

One or more endophytes are inoculated onto soils in the form of a liquid suspension, either; pre-planting as a soil drench, during planting as an in-furrow application, or during crop growth as a side-dress. One or more endophytes are mixed directly into a fertigation system via drip tape, center pivot or other appropriate irrigation system.

### Hydroponic and Aeroponic Inoculation

One or more endophytes are inoculated into a hydroponic or aeroponic system either as a powder or liquid suspension applied directly to the rockwool substrate or applied to the circulating or sprayed nutrient solution.

### Vector-Mediated Inoculation

One or more endophytes are introduced in power form in a mixture containing talc or other bulking agent to the entrance of a beehive (in the case of bee-mediation) or near the nest of another pollinator (in the case of other insects or birds. The pollinators pick up the powder when exiting the hive and deposit the inoculum directly to the crop's flowers during the pollination process.

### Root Wash

The method includes contacting the exterior surface of a plant's roots with a liquid inoculant formulation containing one or more endophytes. The plant's roots are briefly passed through standing liquid microbial formulation or liquid formulation is liberally sprayed over the roots, resulting in both physical removal of soil and microbial debris from the plant roots, as well as inoculation with microbes in the formulation.

### Seedling Soak

The method includes contacting the exterior surfaces of a seedling with a liquid inoculant formulation containing one or more endophytes. The entire seedling is immersed in standing liquid microbial formulation for at least 30 seconds, resulting in both physical removal of soil and microbial debris from the plant roots, as well as inoculation of all plant surfaces with microbes in the formulation. Alternatively, the seedling can be germinated from seed in or transplanted into media soaked with the microbe(s) of interest and then allowed to grow in the media, resulting in soaking of the plantlet in microbial formulation for much greater time, for example: hours, days or weeks. Endophytic microbes likely need time to colonize and enter the plant, as they explore the plant surface for cracks or wounds to enter, so the longer the soak, the more likely the microbes will successfully be installed in the plant.

### Wound Inoculation

The method includes contacting the wounded surface of a plant with a liquid or solid inoculant formulation containing one or more endophytes. Plant surfaces are designed to block entry of microbes into the endosphere, since pathogens attempt to infect plants in this way. One way to introduce beneficial endophytic microbes into plant endospheres is to provide a passage to the plant interior by wounding. This wound can take a number of forms, including pruned roots, pruned branches, puncture wounds in the stem breaching the bark and cortex, puncture wounds in the tap root, puncture wounds in leaves, puncture wounds seed allowing entry past the seed coat. Wounds can be made using tools for physical penetration of plant tissue such as needles. Microwounds may also be introduced by sonication. Into the wound can then be contacted the microbial inoculant as liquid, as powder, inside gelatin capsules, in a pressurized capsule injection system, or in a pressurized reservoir and tubing injection system, allowing entry and colonization by microbes into the endosphere. Alternatively, the entire wounded plant can be soaked or washed in the microbial inoculant for at least 30 seconds, giving more microbes a chance to enter the wound, as well as inoculating other plant surfaces with microbes in the formulation—for example pruning seedling roots and soaking them in inoculant before transplanting is a very effective way to introduce endophytes into the plant.

### Injection

The method includes injecting microbes into a plant in order to successfully install them in the endosphere. Plant surfaces are designed to block entry of microbes into the endosphere, since pathogens attempt to infect plants in this way. In order to introduce beneficial endophytic microbes to endospheres, we need a way to access the interior of the plant which we can do by puncturing the plant surface with a needle and injecting microbes into the inside of the plant. Different parts of the plant can be inoculated this way including the main stem or trunk, branches, tap roots, seminal roots, buttress roots, and even leaves. The injection can be made with a hypodermic needle, a drilled hole injector, or a specialized injection system, and through the puncture wound can then be contacted the microbial inoculant as liquid, as powder, inside gelatin capsules, in a pressurized capsule injection system, or in a pressurized reservoir and tubing injection system, allowing entry and colonization by microbes into the endosphere.

### Example 29. Identification of Sequence Variants Across Core Genes

Phylogenomic analysis of whole genome sequences of endophytes can be used to identify distinguishing sequence variants. Sets of genes suitable for phylogenomic analysis as well as methods for identifying the same are well known in the art, for example Floutas et al. (2012) The Paleozoic origin of enzymatic lignin decomposition reconstructed from 31 fungal genomes. Science, 336(6089): 1715-9. doi: 10.1126/science. 1221748 and James T Y, Pelin A, Bonen L, Ahrendt S, Sain D, Corradi N, Stajich J E. Shared signatures of parasitism and phylogenomics unite Cryptomycota and microsporidia. Curr Biol. 2013; 23(16):1548-53. doi: 10.1016/j.cub.2013.06.057. Orthologous genes to the reference set are identified in protein data bases derived from the genome of each species. Orthologous genes can be identified in the genomes using methods well known including reciprocal best hits (Ward N, Moreno-Hagelsieb G. Quickly Finding Orthologs as Reciprocal Best Hits with BLAT, LAST, and UBLAST: How Much Do We Miss? de Crécy-Lagard V, ed. PLOS ONE. 2014; 9(7): e101850. doi: 10.1371/journal.pone.0101850) and Hidden Markov Models (HMMs). The best hits are extracted and a multiple sequence alignment generated for each set of orthologous genes. The alignments are used to build phylogenetic trees using methods well known in the art including Bayesian inference and maximum likelihood methods, for example using software tools MrBayes (Huelsenbeck, J. P. & Ronquist (2001) MRBAYES: Bayesian inference of phylogenetic trees. Bioinformatics, 17(8): 754-755) and RAxML (Stamatakis, A. (2014) RAXML version 8: a tool for phylogenetic analysis and post-analysis of large phylogenies. Bioinformatics, 30 (9): 1312-1313. doi: 10.1093/bioinformatics/btu033). Sequence variants which distinguish between closely related species are identified.

### Example 30. Identification of Unique Genes in an Endophyte of Interest

Whole genome analysis of endophytes can be used to identify genes whose presence, absence or over or under representation (“differential abundance”) are associated with desirable phenotypes. To identify genes with differential abundance in the genome of an endophyte of interest, protein sequences predicted from the genomes of the endophyte and closely related species are compared in an all-vs-all pairwise comparison (for example, using BLAST) followed by clustering of the protein sequences based on alignment scores (for example, using MCL: Enright A. J., Van Dongen S., Ouzounis C. A. An efficient algorithm for large-scale detection of protein families. Nucleic Acids Research 30(7): 1575-1584 (2002)). Additional software tools useful for this analysis are well known in the art and include OMA, OrthoMCL and TribeMCL (Roth A C, Gonnet G H, Dessimoz C. Algorithm of OMA for large-scale orthology inference. BMC Bioinformatics. 2008; 9:518. doi: 10.1186/1471-2105-9-518, Enright A J, Kunin V, Ouzounis C A. Protein families and TRIBES in genome sequence space. Nucleic Acids Res. 2003; 31(15):4632-8; Chen F, Mackey A J, Vermunt J K, Roos D S. Assessing performance of orthology detection strategies applied to eukaryotic genomes. PLOS One. 2007; 2(4):e383). The protein clusters are queried to identify clusters with differential abundance of proteins derived from endophytes having desirable phenotypes. Proteins of these clusters define the unique properties of these endophytes, and the abundance of genes encoding these proteins may be used to identify endophytes of the present invention.

### Example 31. In Vitro Assessment of Production of Antibiotic Metabolites

This example describes an exemplary method by which microbes may be shown to produce metabolites that inhibit the growth of hyphal phytopathogens in vitro. Such phytopathogens can be members of the “true” fungi, phylum Eumycota, or from other taxonomic groups with a similar growth habit such as members of the phylum Oomycota. Hyphal growth can be described as organism growth along thread-like structures composed of connected cells. Such growth is found commonly among fungi and oomycetes, and even some genera of bacteria. In this assay, the hyphal growth should be in a roughly uniform, radial manner. This assay is comprised of a Petri plate containing an agar-based media and a hyphal phytopathogen grown concomitantly with either a live test microbe or in the presence of the spent media from a previously grown test microbe.

Testing with Live Endophyte Cultures

Preparation of Hyphal Phytopathogen Petri plates containing a media suitable for the growth of the target hyphal pathogens (Fusarium graminearum, Rhizoctonia solani, Pythium sp.) were inoculated with the target hyphal pathogen. After inoculation on the media-containing Petri plate, the culture was allowed to grow until reaching the edge of the Petri plate.

Preparation of the test sample Microbial samples for testing endophytes MIC-84302, MIC-18905, and MIC-67967 were produced by liquid culture.

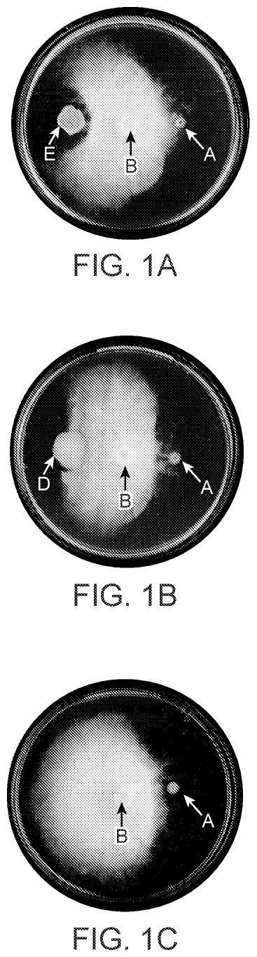

Assay Set-Up Petri dishes, also referred to as test plates, containing solid agar test media (see Use of Multiple Growth Media for a description of media used) were prepared. A sterile instrument was used to remove a test pathogen plug from the hyphal pathogen plate culture and placed centrally on the test plate. Next a test sample was applied to the test plate at a distance such that the test sample and test plate came into physical contact after more than one day of growth. A drop of overnight liquid culture of the endophyte to be tested was applied to each test plate. A drop of Metconazole, a chemical fungicide capable of impeding the growth of Fusarium and Rhizoctonia was applied to each test plate containing those pathogens as a control. A drop of Mefenoxam, a chemical fungicide capable of impeding the growth of Pythium was applied to each test plate containing Pythium. For an example of the relative position of the test sample, pathogen sample, and chemical control refer to FIG. 1C.

Use of Multiple Growth Media Various environmental conditions can result in differential production of metabolites and pathogens grown under various environmental conditions show differential sensitivity to those metabolites, therefore the assay was performed on multiple media types: half strength Potato Dextrose Agar (0.5×PDA), yeast extract peptone dextrose agar (YEPD), tryptic soy agar (TSA), and Reasoner's 2A agar (R2A). Medias were chosen to vary important growth inputs such as carbon source, presence and concentration of various salts, and presence of extracts from different plant species or organs.

Assessment After setting up, hyphal pathogens were allowed to grow for sufficient time such that the hyphal front meets or just passes the test sample. In cases where anti-pathogen metabolites are produced and secreted, a restriction of growth of the hyphal front around the test sample is commonly observed. Often this will also result in an area of clearing around the test sample. In these cases, the morphology of the hyphal pathogen near the test sample will often also be dissimilar from areas away from the test sample. Alternatively, when anti-pathogen metabolites are not produced and secreted, the hyphal pathogen will grow over the test sample with little to no visible effect on growth.

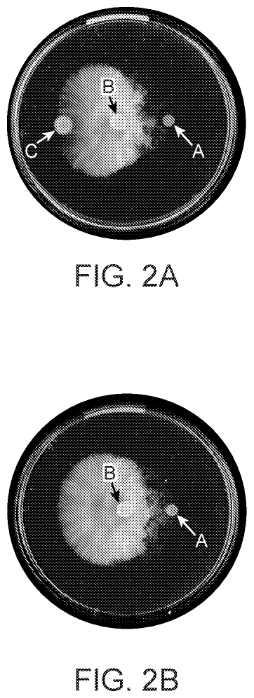

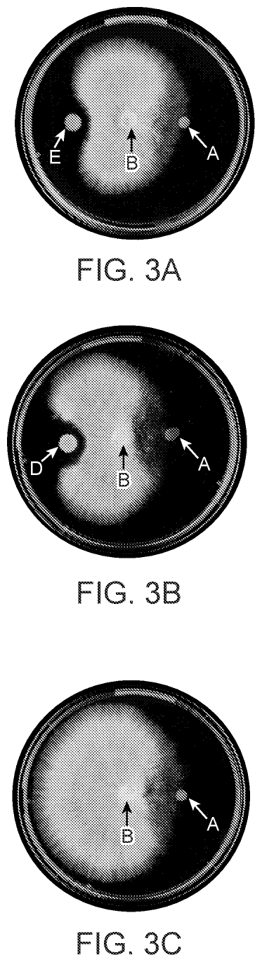

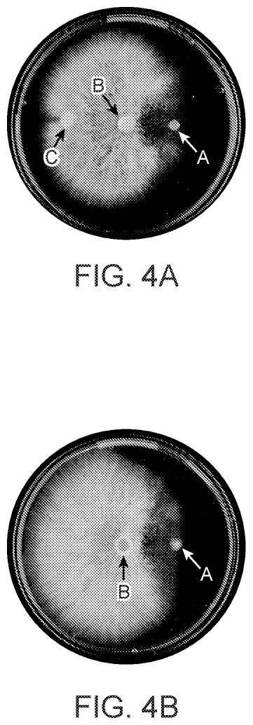

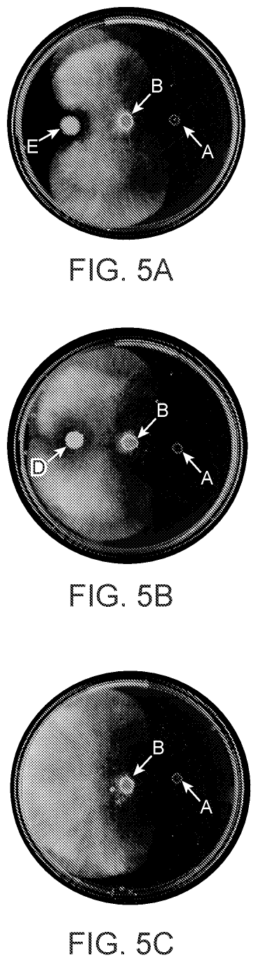

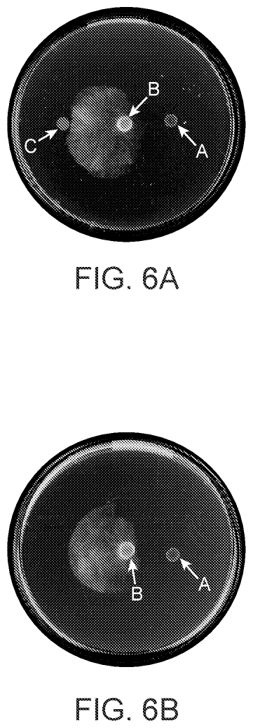

Exemplary images of test plates treated with endophytes, chemical fungicides and pathogens are shown in FIG. 1A-C, FIG. 2A-B, FIG. 3A-C, FIG. 4A-B, FIG. 5A-C, and FIG. 6A-B.

MIC-18905 and MIC-84302 showed significant pathogen-free zones around the test samples of these endophytes on TSA test plates inoculated with Fusarium graminearum (see for example FIG. 1A, FIG. 1B, and FIG. 1C). MIC-67967 showed pathogen-free zones around the test samples of this endophyte on R2A test plates inoculated with Fusarium graminearum (see for example FIG. 2A and FIG. 2B).

MIC-18905 and MIC-84302 showed significant pathogen-free zones around the test samples of these endophytes on 0.5×PDA test plates inoculated with Rhizoctonia solani (see for example FIG. 3A, FIG. 3B, and FIG. 3C). MIC-67967 showed pathogen-free zones around the test samples of this endophyte on 0.5×PDA test plates inoculated with Rhizoctonia solani (see for example FIG. 4A and FIG. 4B).

MIC-18905 and MIC-84302 showed significant pathogen-free zones around the test samples of these endophytes on 0.5×PDA test plates inoculated with Pythium (see for example FIG. 5A, FIG. 5B, and FIG. 5C). MIC-67967 showed significant pathogen-free zones around the test samples of this endophyte on YEPD test plates inoculated with Pythium (see for example FIG. 6A and FIG. 6B).

Testing with Filtered or Dead Endophyte Cultures

Pathogen samples are prepared as described above. A microbial sample for testing, also referred to as a test sample, can be produced in multiple ways. A liquid culture of hyphal or colony forming microbe is grown in liquid culture, and viable material is removed by various methods including, but not limited to, filtration. Alternately, or in addition to filtration a test sample may be autoclaved and a non-viable test sample may be used. This later method of testing a non-viable test sample is used when the test microbe displays a much faster rate of radial growth than the hyphal pathogen being tested, to identify production of antimicrobial metabolites, for example not as a part an active biological process such a mycophagy.

Assay Set-Up A Petri dish containing a solid agar test media is obtained. This will be referred to as the test plate. A sterile instrument is used to remove a test pathogen plug from the hyphal pathogen plate culture and placed on the test plate. For assaying a non-viable test sample, an agar plug is removed from the test plate using a sterile instrument to create a well to hold the test sample. The well is then filled with the non-viable test sample, and the sample is absorbed into the agar media. A chemical compound capable of impeding the growth of the pathogen is included as a control. For an example of the relative position of the test sample, pathogen sample, and chemical control refer to FIG. 1.

Use of Multiple Growth Media Test microbe growth under various environmental conditions are expected to result in differential production of metabolites. Similarly, pathogens grown under various environmental conditions are expected to show differential sensitivity to those metabolites. For this reason, this assay is performed on multiple media types. Medias are chosen to vary important growth inputs such as carbon source, presence and concentration of various salts, and presence of extracts from different plant species or organs.

Assessment After setting up, hyphal pathogens are allowed to grow for sufficient time such that the hyphal front meets or just passes the test sample. In cases where anti-pathogen metabolites are produced and secreted, a restriction of growth of the hyphal front around the test sample is commonly observed. Often this will also result in an area of clearing around the test sample. In these cases, the morphology of the hyphal pathogen near the test sample will often also be dissimilar from areas away from the test sample. Alternatively, when anti-pathogen metabolites are not produced and secreted, the hyphal pathogen will grow over the test sample with little to no visible effect on growth.

Having illustrated and described the principles of the present invention, it should be apparent to persons skilled in the art that the invention can be modified in arrangement and detail without departing from such principles. It is to be understood that while the invention has been described in conjunction with the detailed description thereof, the foregoing description is intended to illustrate and not limit the scope of the invention, which is defined by the scope of the appended claims. Other embodiments, advantages, and modifications are within the scope of the following claims.

1. A synthetic composition, comprising one or more endophytes heterologously disposed to a treatment formulation, wherein the one or more endophytes comprise at least one polynucleotide sequence that is at least 97% identical to SEQ ID NO. 127, and the genomes of the one or more endophytes comprise one or more open reading frames encoding proteins whose amino acid sequences are at least 97% identical to one or more of SEQ ID NOs. 128-131.

2. The synthetic composition of claim 1, wherein the synthetic composition additionally comprises one or more plant element elements.

3. The synthetic composition of claim 2, wherein the one or more plant element elements are seeds.

4. The synthetic composition of claim 2, wherein the one or more plant element elements are soybean, wheat, or corn.

5. The synthetic composition of claim 3, wherein the synthetic composition comprises a least 1E+02 endophytes per seed.

6. The synthetic composition of claim 3, wherein the synthetic composition comprises a least 1E+03 endophytes per seed.

7. The synthetic composition of claim 3, wherein the synthetic composition comprises a least 1E+04 endophytes per seed.

8. The synthetic composition of claim 2, wherein the one or more endophytes are capable of improving one or more traits of agronomic importance in a plant derived from the plant element relative to a plant derived from a reference plant element.

9. The synthetic composition of claim 8, wherein the one or more traits of agronomic importance comprise one or more of biotic stress tolerance, shoot fresh weight, yield, plant height, shoot weight, and or root weight.

10. The synthetic composition of claim 9, wherein the biotic stress is a growth environment comprising one or more pests or pathogens.

11. The synthetic composition of claim 10, wherein the one or more pests or pathogens is a Pythium, Rhizoctonia, or Fusarium species.

12. The synthetic composition of claim 8, wherein:
the synthetic composition additionally comprises one or more soybean plant elements,
the one or more traits of agronomic importance are shoot fresh weight and biotic stress tolerance, wherein biotic stress is a growth environment comprising a Pythium, Rhizoctonia, or Fusarium species.

13. The synthetic composition of claim 8, wherein:
the synthetic composition additionally comprises one or more wheat plant elements,
the one or more traits of agronomic importance are shoot fresh weight and biotic stress tolerance, wherein biotic stress is a growth environment comprising a Fusarium species.

14. The synthetic composition of claim 8, wherein:
the synthetic composition additionally comprises one or more corn plant elements,
the one or more traits of agronomic importance are shoot weight, yield, plant height, root weight, and biotic stress tolerance, wherein biotic stress is a growth environment comprising a Fusarium species.

15. The synthetic composition of claim 1, wherein the one or more endophytes comprise at least one polynucleotide sequence that is 100% identical to SEQ ID NO. 127.

16. The synthetic composition of claim 1, wherein the one or more endophytes are capable of producing proteins having amino acid sequences are at least 97% identical to SEQ ID NOs. 128, 129, 130, and 131.

17. The synthetic composition of claim 1, wherein the one or more endophytes are of the genus Chitinophaga.

18. The synthetic composition of claim 1, wherein the one or more endophytes are of the genus and species Chitinophaga oryzae.

19. A synthetic composition, comprising one or more endophytes heterologously disposed to a treatment formulation, wherein the one or more endophytes comprise at least one polynucleotide sequence that is at least 97% identical to SEQ ID NO. 132, and the genomes of the one or more endophytes comprise one or more open reading frames encoding proteins whose amino acid sequences are at least 97% identical to one or more of SEQ ID NOs. 133-136, wherein the treatment formulation comprises a solid carrier and adherent.

20. The synthetic composition of claim 19, wherein the synthetic composition additionally comprises one or more plant element elements.

21. The synthetic composition of claim 20, wherein the one or more plant element elements are seeds.

22. The synthetic composition of claim 20, wherein the one or more plant element elements are soybean, wheat, or cotton.

23. The synthetic composition of claim 21, wherein the synthetic composition comprises a least 1E+02 endophytes per seed.

24. The synthetic composition of claim 21, wherein the synthetic composition comprises a least 1E+03 endophytes per seed.

25. The synthetic composition of claim 21, wherein the synthetic composition comprises a least 1E+04 endophytes per seed.

26. The synthetic composition of claim 20, wherein the one or more endophytes are capable of improving one or more traits of agronomic importance in a plant derived from the plant element relative to a plant derived from a reference plant element.

27. The synthetic composition of claim 26, wherein the one or more traits of agronomic importance comprise one or more of biotic stress tolerance, shoot fresh weight, yield, plant height, stand count, and or root weight.

28. The synthetic composition of claim 27, wherein the biotic stress is a growth environment comprising one or more pests or pathogens.

29. The synthetic composition of claim 28, wherein the one or more pests or pathogens is a Pythium, or Fusarium species.

30. The synthetic composition of claim 26, wherein:
the synthetic composition additionally comprises one or more soybean plant elements,
the one or more traits of agronomic importance are shoot fresh weight and biotic stress tolerance, wherein biotic stress is a growth environment comprising a Pythium species.

31. The synthetic composition of claim 26, wherein:
the synthetic composition additionally comprises one or more wheat plant elements,
the one or more traits of agronomic importance are shoot fresh weight and biotic stress tolerance, wherein biotic stress is a growth environment comprising a Fusarium species.

32. The synthetic composition of claim 26, wherein:
the synthetic composition additionally comprises one or more cotton plant elements,
the one or more traits of agronomic importance are shoot weight, root weight, plant height, stand count, and biotic stress tolerance, wherein biotic stress is a growth environment comprising a Fusarium species.

33. The synthetic composition of claim 26, wherein:
the synthetic composition additionally comprises one or more wheat plant elements,
the one or more traits of agronomic importance are yield, and biotic stress tolerance, wherein biotic stress is a growth environment comprising a Pythium species, Rhizoctonia species, and Fusarium species.

34. The synthetic composition of claim 19, wherein the one or more endophytes comprise at least one polynucleotide sequence that is 100% identical to SEQ ID NO. 132.

35. The synthetic composition of claim 19, wherein the one or more endophytes are capable of producing proteins having amino acid sequences are at least 97% identical to SEQ ID NOs. 133, 134, 135, and 136.

36. The synthetic composition of claim 19, wherein the one or more endophytes are of the genus Bacillus.

37. The synthetic composition of claim 19, wherein the one or more endophytes are of the genus and species Bacillus velenzensis.

38. The synthetic composition of claim 19, wherein the solid carrier is talc and the adherent is mineral oil.

39. A synthetic composition, comprising one or more endophytes heterologously disposed to a treatment formulation, wherein the one or more endophytes comprise at least one polynucleotide sequence that is at least 97% identical to SEQ ID NO. 27, and the genomes of the one or more endophytes comprise one or more open reading frames encoding proteins whose amino acid sequences are at least 97% identical to one or more of SEQ ID NOs. 28-31.

40. The synthetic composition of claim 39, wherein the synthetic composition additionally comprises one or more plant element elements.

41. The synthetic composition of claim 40, wherein the one or more plant element elements are seeds.

42. The synthetic composition of claim 40, wherein the one or more plant element elements are soybean or wheat.

43. The synthetic composition of claim 41, wherein the synthetic composition comprises a least 1E+02 endophytes per seed.

44. The synthetic composition of claim 41, wherein the synthetic composition comprises a least 1E+03 endophytes per seed.

45. The synthetic composition of claim 41, wherein the synthetic composition comprises a least 1E+04 endophytes per seed.

46. The synthetic composition of claim 40, wherein the one or more endophytes are capable of improving one or more traits of agronomic importance in a plant derived from the plant element relative to a plant derived from a reference plant element.

47. The synthetic composition of claim 46, wherein the one or more traits of agronomic importance comprise one or more of biotic stress tolerance and or shoot fresh weight.

48. The synthetic composition of claim 47, wherein the biotic stress is a growth environment comprising one or more pests or pathogens.

49. The synthetic composition of claim 48, wherein the one or more pests or pathogens is a Pythium, Rhizoctonia, or Fusarium species.

50. The synthetic composition of claim 46, wherein:
the synthetic composition additionally comprises one or more soybean plant elements,
the one or more traits of agronomic importance are shoot fresh weight and biotic stress tolerance, wherein biotic stress is a growth environment comprising a Pythium or Fusarium species.

51. The synthetic composition of claim 46, wherein:
the synthetic composition additionally comprises one or more wheat plant elements,
the one or more traits of agronomic importance are shoot fresh weight and biotic stress tolerance, wherein biotic stress is a growth environment comprising a Pythium or Rhizoctoina species.

52. The synthetic composition of claim 39, wherein the one or more endophytes comprise at least one polynucleotide sequence that is 100% identical to SEQ ID NO. 27.

53. The synthetic composition of claim 39, wherein the one or more endophytes are capable of producing proteins having amino acid sequences are at least 97% identical to SEQ ID NOs. 28, 29, 30, and 31.

54. The synthetic composition of claim 39, wherein the one or more endophytes are of the genus Pseudomonas.

55. The synthetic composition of claim 39, wherein the one or more endophytes are of the genus and species Pseudomonas glycinis.

56. The synthetic composition of claim 39, wherein the solid carrier is talc and the adherent is mineral oil.

57. A synthetic composition, comprising one or more endophytes heterologously disposed to a treatment formulation, wherein the one or more endophytes comprise at least one polynucleotide sequence that is at least 97% identical to SEQ ID NO. 32 or SEQ ID NOs. 59-94, and the genomes of the one or more endophytes comprise one or more open reading frames encoding proteins whose amino acid sequences are at least 97% identical to one or more of SEQ ID NOs. 95-127.

58. The synthetic composition of claim 57, wherein the synthetic composition additionally comprises one or more plant element elements.

59. The synthetic composition of claim 58, wherein the one or more plant element elements are seeds.

60. The synthetic composition of claim 58, wherein the one or more plant element elements are soybean, cotton, or wheat.

61. The synthetic composition of claim 59, wherein the synthetic composition comprises a least 1E+02 endophytes per seed.

62. The synthetic composition of claim 59, wherein the synthetic composition comprises a least 1E+03 endophytes per seed.

63. The synthetic composition of claim 59, wherein the synthetic composition comprises a least 1E+04 endophytes per seed.

64. The synthetic composition of claim 58, wherein the one or more endophytes are capable of improving one or more traits of agronomic importance in a plant derived from the plant element relative to a plant derived from a reference plant element.

65. The synthetic composition of claim 64, wherein the one or more traits of agronomic importance comprise one or more of biotic stress tolerance, shoot fresh weight, root weight, shoot weight, yield, early emergence, full emergence, and or plant height.

66. The synthetic composition of claim 65, wherein the biotic stress is a growth environment comprising one or more pests or pathogens.

67. The synthetic composition of claim 66, wherein the one or more pests or pathogens is a Pythium, Rhizoctonia, or Fusarium species.

68. The synthetic composition of claim 64, wherein:
the synthetic composition additionally comprises one or more soybean plant elements,
the one or more traits of agronomic importance are shoot fresh weight, root weight, shoot weight, yield, and biotic stress tolerance, wherein biotic stress is a growth environment comprising a Pythium species, a Rhizoctonia species, or Fusarium species.

69. The synthetic composition of claim 64, wherein:
the synthetic composition additionally comprises one or more wheat plant elements,
the one or more traits of agronomic importance are shoot fresh weight and biotic stress tolerance, wherein biotic stress is a growth environment comprising a Fusarium or Rhizoctoina species.

70. The synthetic composition of claim 57, wherein the one or more endophytes comprise at least one polynucleotide sequence that is 100% identical to SEQ ID NO. 32 or SEQ ID NOs. 59-94.

71. The synthetic composition of claim 57, wherein the one or more endophytes are capable of one or more producing proteins having amino acid sequences are at least 97% identical to one or more of SEQ ID NOs. 95-127.

72. The synthetic composition of claim 57, wherein the genome of the one or more endophytes comprises at least one polynucleotide region having at least 97% sequence identity to one or more polynucleotide sequences selected from the group consisting of SEQ ID NOs. 61, 71, 72, 75, 76, 80, 81, 82, 85, 87, 88, and 89, and at least one polynucleotide region having at least 97% sequence identity to one or more polynucleotide sequences selected from the group consisting of SEQ ID NOs. 59, 60, 63, 64, 65, and 84, and at least one polynucleotide region having at least 97% sequence identity to one or more polynucleotide sequences selected from the group consisting of SEQ ID NOs. 67, 70, 79, and 83, and at least a polynucleotide region having at least 97% sequence identity to SEQ ID NO. 77, and at least one polynucleotide regions having at least 97% sequence identity to one or more polynucleotide sequences selected from the group consisting of SEQ ID NOs. 62, 66, 68, 69, 73, 74, 78, and 86.

73. The synthetic composition of claim 57, wherein the genome of the one or more endophytes comprises at least one polynucleotide region having 100% sequence identity to one or more polynucleotide sequences selected from the group consisting of SEQ ID NOs. 61, 71, 72, 75, 76, 80, 81, 82, 85, 87, 88, and 89, and at least one polynucleotide region having 100% sequence identity to one or more polynucleotide sequences selected from the group consisting of SEQ ID NOs. 59, 60, 63, 64, 65, and 84, and at least one polynucleotide region having 100% sequence identity to one or more polynucleotide sequences selected from the group consisting of SEQ ID NOs. 67, 70, 79, and 83, and at least a polynucleotide region having 100% sequence identity to SEQ ID NO. 77, and at least one polynucleotide regions having 100% sequence identity to one or more polynucleotide sequences selected from the group consisting of SEQ ID NOs. 62, 66, 68, 69, 73, 74, 78, and 86.

74. The synthetic composition of claim 57, wherein the one or more endophytes are of the genus Trichoderma.

75. The synthetic composition of claim 57, wherein the one or more endophytes are of the genus and species Trichoderma hamatum.

76. The synthetic composition of claim 57, wherein the treatment formulation comprises a solid carrier and adherent.

77. The synthetic composition of claim 57, wherein the solid carrier is talc and the adherent is mineral oil.

78. A method of improving plant health, comprising applying a one or more heterologously disposed endophytes to a plant element, wherein the one or more endophytes comprise at least one polynucleotide sequence that is at least 97% identical to SEQ ID NO. 127, and the genomes of the one or more endophytes comprise one or more open reading frames encoding proteins whose amino acid sequences are at least 97% identical to one or more of SEQ ID NOs. 128-131.

79. The method of claim 78, wherein the one or more plant element elements are soybean, wheat, or corn.

80. The method of claim 78, wherein the one or more plant element elements are seeds.

81. The method of claim 80, wherein the one or more heterologously disposed endophytes are present in an average abundance of a least 1E+02 endophytes per seed.

82. The method of claim 80, wherein the one or more heterologously disposed endophytes are present in an average abundance of a least 1E+03 endophytes per seed.

83. The method of claim 80, wherein the one or more heterologously disposed endophytes are present in an average abundance of 1E+04 endophytes per seed.

84. The method of claim 78, wherein the one or more endophytes are capable of improving one or more traits of agronomic importance in a plant derived from the plant element relative to a plant derived from a reference plant element.

85. The method of claim 84, wherein the one or more traits of agronomic importance comprise one or more of biotic stress tolerance, shoot fresh weight, yield, plant height, shoot weight, and or root weight.

86. The method of claim 85, wherein the biotic stress is a growth environment comprising one or more pests or pathogens.

87. The method of claim 86, wherein the one or more pests or pathogens is a Pythium, Rhizoctonia, or Fusarium species.

88. The synthetic composition of claim 84, wherein:
the one or more plant elements are soybean plant elements,
the one or more traits of agronomic importance are shoot fresh weight and biotic stress tolerance, wherein biotic stress is a growth environment comprising a Pythium, Rhizoctonia, or Fusarium species.

89. The method of claim 84, wherein:
the one or more plant elements are wheat plant elements,
the one or more traits of agronomic importance are shoot fresh weight and biotic stress tolerance, wherein biotic stress is a growth environment comprising a Fusarium species.

90. The method of claim 84, wherein:
the one or more plant elements are corn plant elements,
the one or more traits of agronomic importance are shoot weight, yield, plant height, root weight, and biotic stress tolerance, wherein biotic stress is a growth environment comprising a Fusarium species.

91. The method of claim 78, wherein the one or more endophytes comprise at least one polynucleotide sequence that is 100% identical to SEQ ID NO. 127.

92. The method of claim 78, wherein the one or more endophytes are capable of producing proteins having amino acid sequences are at least 97% identical to SEQ ID NOs. 128, 129, 130, and 131.

93. The method of claim 78, wherein the one or more endophytes are of the genus Chitinophaga.

94. The method of claim 78, wherein the one or more endophytes are of the genus and species Chitinophaga oryzae.

95. A method of improving plant health, comprising applying a one or more heterologously disposed endophytes to a plant element, wherein the one or more endophytes comprise at least one polynucleotide sequence that is at least 97% identical to SEQ ID NO. 132, and the genomes of the one or more endophytes comprise one or more open reading frames encoding proteins whose amino acid sequences are at least 97% identical to one or more of SEQ ID NOs. 133-136.

96. The method of claim 95, wherein the one or more plant element elements are seeds.

97. The method of claim 95, wherein the one or more plant element elements are soybean, wheat, or cotton.

98. The method of claim 95, wherein the one or more heterologously disposed endophytes are present in an average abundance of a least 1E+02 endophytes per seed.

99. The method of claim 95, wherein the one or more heterologously disposed endophytes are present in an average abundance of a least 1E+03 endophytes per seed.

100. The method of claim 95, wherein the one or more heterologously disposed endophytes are present in an average abundance of a least 1E+04 endophytes per seed.

101. The method of claim 95, wherein the one or more endophytes are capable of improving one or more traits of agronomic importance in a plant derived from the plant element relative to a plant derived from a reference plant element.

102. The method of claim 101, wherein the one or more traits of agronomic importance comprise one or more of biotic stress tolerance, shoot fresh weight, yield, plant height, stand count, and or root weight.

103. The method of claim 102, wherein the biotic stress is a growth environment comprising one or more pests or pathogens.

104. The method of claim 103, wherein the one or more pests or pathogens is a Pythium, or Fusarium species.

105. The method of claim 101, wherein:
the one or more plant elements are soybean plant elements,
the one or more traits of agronomic importance are shoot fresh weight and biotic stress tolerance, wherein biotic stress is a growth environment comprising a Pythium species.

106. The method of claim 101, wherein:
the one or more plant elements are wheat plant elements,
the one or more traits of agronomic importance are shoot fresh weight and biotic stress tolerance, wherein biotic stress is a growth environment comprising a Fusarium species.

107. The method of claim 101, wherein:
the one or more plant elements are cotton plant elements,
the one or more traits of agronomic importance are shoot weight, root weight, plant height, stand count, and biotic stress tolerance, wherein biotic stress is a growth environment comprising a Fusarium species.

108. The method of claim 101, wherein:
the one or more plant elements are wheat plant elements,
the one or more traits of agronomic importance are yield, and biotic stress tolerance, wherein biotic stress is a growth environment comprising a Pythium species, Rhizoctonia species, and Fusarium species.

109. The method of claim 95, wherein the one or more endophytes comprise at least one polynucleotide sequence that is 100% identical to SEQ ID NO. 132.

110. The method of claim 95, wherein the one or more endophytes are capable of producing proteins having amino acid sequences are at least 97% identical to SEQ ID NOs. 133, 134, 135, and 136.

111. The method of claim 95, wherein the one or more endophytes are of the genus Bacillus.

112. The method of claim 95, wherein the one or more endophytes are of the genus and species Bacillus velenzensis.

113. The method of claim 95, wherein the one or more endophytes are applied in a treatment formulation comprising a solid carrier and an adherent.

114. The method of claim 113, wherein the solid carrier is talc and the adherent is mineral oil.

115. A method of improving plant health, comprising applying a one or more heterologously disposed endophytes to a plant element, wherein the one or more endophytes comprise at least one polynucleotide sequence that is at least 97% identical to SEQ ID NO. 27, and the genomes of the one or more endophytes comprise one or more open reading frames encoding proteins whose amino acid sequences are at least 97% identical to one or more of SEQ ID NOs. 28-31.

116. The method of claim 115, wherein the one or more plant element elements are seeds.

117. The method of claim 115, wherein the one or more plant element elements are soybean or wheat.

118. The method of claim 115, wherein the one or more heterologously disposed endophytes are present in an average abundance of a least 1E+02 endophytes per seed.

119. The method of claim 115, wherein the one or more heterologously disposed endophytes are present in an average abundance of a least 1E+03 endophytes per seed.

120. The method of claim 115, wherein the one or more heterologously disposed endophytes are present in an average abundance of a least 1E+04 endophytes per seed.

121. The method of claim 115, wherein the one or more endophytes are capable of improving one or more traits of agronomic importance in a plant derived from the plant element relative to a plant derived from a reference plant element.

122. The method of claim 121, wherein the one or more traits of agronomic importance comprise one or more of biotic stress tolerance and or shoot fresh weight.

123. The method of claim 122, wherein the biotic stress is a growth environment comprising one or more pests or pathogens.

124. The method of claim 123, wherein the one or more pests or pathogens is a Pythium, Rhizoctonia, or Fusarium species.

125. The method of claim 121, wherein:
the one or more plant elements are soybean plant elements,
the one or more traits of agronomic importance are shoot fresh weight and biotic stress tolerance, wherein biotic stress is a growth environment comprising a Pythium or Fusarium species.

126. The method of claim 121, wherein:
the one or more plant elements are wheat plant elements,
the one or more traits of agronomic importance are shoot fresh weight and biotic stress tolerance, wherein biotic stress is a growth environment comprising a Pythium or Rhizoctoina species.

127. The method of claim 115, wherein the one or more endophytes comprise at least one polynucleotide sequence that is 100% identical to SEQ ID NO. 27.

128. The method of claim 115, wherein the one or more endophytes are capable of producing proteins having amino acid sequences are at least 97% identical to SEQ ID NOs. 28, 29, 30, and 31.

129. The method of claim 115, wherein the one or more endophytes are of the genus Pseudomonas.

130. The method of claim 115, wherein the one or more endophytes are of the genus and species Pseudomonas glycinis.

131. The method of claim 115, wherein the one or more endophytes are applied in a treatment formulation comprising a solid carrier and an adherent.

132. The method of claim 115, wherein the solid carrier is talc and the adherent is mineral oil.

133. A method of improving plant health, comprising applying a one or more heterologously disposed endophytes to a plant element, wherein the one or more endophytes comprise at least one polynucleotide sequence that is at least 97% identical to SEQ ID NO. 32 or SEQ ID NOs. 59-94, and the genomes of the one or more endophytes comprise one or more open reading frames encoding proteins whose amino acid sequences are at least 97% identical to one or more of SEQ ID NOs. 95-127.

134. The method of claim 133, wherein the one or more plant element elements are seeds.

135. The method of claim 133, wherein the one or more plant element elements are soybean or wheat.

136. The method of claim 134, wherein the one or more heterologously disposed endophytes are present in an average abundance of a least 1E+02 endophytes per seed.

137. The method of claim 134, wherein the one or more heterologously disposed endophytes are present in an average abundance of a least 1E+03 endophytes per seed.

138. The method of claim 134, wherein the one or more heterologously disposed endophytes are present in an average abundance of a least 1E+04 endophytes per seed.

139. The method of claim 133, wherein the one or more endophytes are capable of improving one or more traits of agronomic importance in a plant derived from the plant element relative to a plant derived from a reference plant element.

140. The method of claim 139, wherein the one or more traits of agronomic importance comprise one or more of biotic stress tolerance, shoot fresh weight, root weight, yield, early emergence, full emergence, and or plant height.

141. The method of claim 140, wherein the biotic stress is a growth environment comprising one or more pests or pathogens.

142. The method of claim 141, wherein the one or more pests or pathogens is a Pythium, Rhizoctonia, or Fusarium species.

143. The method of claim 139, wherein:
the one or more plant elements are soybean plant elements,
the one or more traits of agronomic importance are shoot fresh weight, root weight, shoot weight, yield, and biotic stress tolerance, wherein biotic stress is a growth environment comprising a Pythium species, a Rhizoctonia species, or Fusarium species.

144. The method of claim 139, wherein:
the one or more plant elements are wheat plant elements,
the one or more traits of agronomic importance are shoot fresh weight and biotic stress tolerance, wherein biotic stress is a growth environment comprising a Fusarium or Rhizoctoina species.

145. The method of claim 133, wherein the one or more endophytes comprise at least one polynucleotide sequence that is 100% identical to SEQ ID NO. 32 or SEQ ID NOs. 59-94.

146. The method of claim 133, wherein the one or more endophytes are capable of one or more producing proteins having amino acid sequences are at least 97% identical to one or more of SEQ ID NOs. 95-127.

147. The method of claim 133, wherein the genomes of the one or more endophytes comprises at least one polynucleotide region having at least 97% sequence identity to one or more polynucleotide sequences selected from the group consisting of SEQ ID NOs. 61, 71, 72, 75, 76, 80, 81, 82, 85, 87, 88, and 89, and at least one polynucleotide region having at least 97% sequence identity to one or more polynucleotide sequences selected from the group consisting of SEQ ID NOs. 59, 60, 63, 64, 65, and 84, and at least one polynucleotide region having at least 97% sequence identity to one or more polynucleotide sequences selected from the group consisting of SEQ ID NOs. 67, 70, 79, and 83, and at least a polynucleotide region having at least 97% sequence identity to SEQ ID NO. 77, and at least one polynucleotide regions having at least 97% sequence identity to one or more polynucleotide sequences selected from the group consisting of SEQ ID NOs. 62, 66, 68, 69, 73, 74, 78, and 86.

148. The method of claim 133, wherein the genome of the one or more endophytes comprises at least one polynucleotide region having 100% sequence identity to one or more polynucleotide sequences selected from the group consisting of SEQ ID NOs. 61, 71, 72, 75, 76, 80, 81, 82, 85, 87, 88, and 89, and at least one polynucleotide region having 100% sequence identity to one or more polynucleotide sequences selected from the group consisting of SEQ ID NOs. 59, 60, 63, 64, 65, and 84, and at least one polynucleotide region having 100% sequence identity to one or more polynucleotide sequences selected from the group consisting of SEQ ID NOs. 67, 70, 79, and 83, and at least a polynucleotide region having 100% sequence identity to SEQ ID NO. 77, and at least one polynucleotide regions having 100% sequence identity to one or more polynucleotide sequences selected from the group consisting of SEQ ID NOs. 62, 66, 68, 69, 73, 74, 78, and 86.

149. The method of claim 133, wherein the one or more endophytes are of the genus Trichoderma.

150. The method of claim 133, wherein the one or more endophytes are of the genus and species Trichoderma hamatum.

151. The method of claim 133, wherein the one or more endophytes are applied in a treatment formulation comprising a solid carrier and an adherent.

152. The method of claim 151, wherein the solid carrier is talc and the adherent is mineral oil.
